(19) United States
(12) Patent Application Publication
(10) Pub. No.: US 2011/0300147 A9
(43) Pub. Date: Dec. 8, 2011

(54) Inhibiting tumor cell invasion, metastasis and angiogenesis

(75) Inventor: Cheng Liu, Carlsbad, CA (US)

(73) Assignee: The Scripps Research institute, La Jolla, CA (US)

(21) Appl. No.: 12/156,123

(22) Filed: May 29, 2008

Related U.S. Application Data
(63) Continuation of application No. PCT/US2006/045788, filed on Nov. 26, 2006.
(60) Provisional application No. 60/740,575, filed on Nov. 29, 2005.

(51) Int. Cl. A61K 39/395; A61P 35/00; A61K 38/08; C07K 7/06; C07K 16/00

(52) U.S. Cl. 424/139.1; 530/329; 530/387.9; 514/20.1

(57) ABSTRACT

The present invention relates to new compositions and methods useful for preventing, treating and diagnosing metastatic and/or invasive cancer and undesirable angiogenesis. For example, the invention relates to inhibitors of proteases that are specifically expressed in tumors, prodrugs activated in the tumor microenvironment and methods for using those inhibitors and prodrugs to inhibit angiogenesis and tumor cell invasion.

### RELATED APPLICATIONS

This application is a U.S. national stage filing under 35 U.S.C. 111(a) of International Application No. PCT/US2006/045788 filed Nov. 29, 2006 and published in English as WO 2007/064759 on Jun. 7, 2007, which claims the benefit of the filing date of U.S. Provisional Application Ser. No. 60/740,575, filed Nov. 29, 2005; which applications and publication are incorporated herein by reference and made a part hereof.

This application is also related to PCT Application Ser. No. PCT/US2004/017157 filed May 28, 2004, which claims benefit of U.S. Application Ser. No. 60/474,840 filed May 29, 2003.

### STATEMENT OF GOVERNMENT RIGHTS

The invention was made with the support of a grant from the Government of the United States of America (CDMRP Grant Numbers W81XWH-05-1-0091 and W81XWH-05-1-0318 from the Department of Defense). The Government may have certain rights to the invention.

### FIELD OF THE INVENTION

The present invention relates to methods for treating and/or inhibiting tumor cell invasion, metastasis and/or angiogenesis as well as increasing apoptosis in cancer cells by administering asparaginyl endopeptidase inhibitors. In some embodiments, the invention relates to inhibitors of proteases that are expressed under the hypoxic conditions of the tumor microenvironment. In other embodiments, the invention relates to prodrug agents that become activated by the proteases that are expressed in the tumor microenviroment. The prodrugs become active within the tumor microenvironments of primary and metastatic tumor sites, for example, at the surface of, cancerous cells and tumor stomal cells that express proteases.

### BACKGROUND OF THE INVENTION

The following description includes information that may be useful in understanding the present invention. It is not an admission that any of the information provided herein is prior art, or relevant, to the presently described inventions, or that any publication or document that is specifically or implicitly referenced is prior art.

According to the National Cancer Institute, since 1990 over 17 million people have been diagnosed with cancer, and an additional 1,334,100 new cancer cases are expected to be diagnosed in 2003. About 556,500 Americans are expected to die of cancer in 2003, more than 1500 people every day. Cancer is therefore the second leading cause of death in the United States, exceeded only by heart disease. The National Institutes of Health estimate the overall costs of cancer in the year 2002 at $171.6 billion (Cancer Facts & Figures, 2003). Clearly, cancer is an enormous problem, and more effective cancer treatments are needed.

Two characteristic features of malignant cells are the ability to invade normal tissues and the ability to spread to distant sites. Tumor metastasis and invasion are the main cause of cancer mortality. Malignant cells can spread by several routes including direct local invasion, by the lymphatics or by capillaries. Local invasion is accomplished by an increase of tumor cell mobility and by production of proteases that destroy the normal extracellular matrix and basement membranes. Once the tumor cells escape from their normal boundaries, they are free to enter the circulation through the capillaries and the lymphatic system. The need for methods to prevent tumor invasion and metastasis is critical and constitutes a major goal in the effort to develop effective therapeutic interventions against cancer.

In addition, many cancer cells are capable of inducing angiogenesis. To form blood vessels, angiogenic endothelial cells share some of the same biochemical mechanisms that are used by cancer cells to invade local tissues.

Current cancer treatments generally involve the use of surgery, radiation therapy, and/or chemotherapy. However, these treatments all involve serious side effects. For example, surgery can be complicated by bleeding, damage to internal organs, adverse reactions to anesthesia or other medicines, pain, infection, and slow recovery. Radiation therapy can damage normal cells and can cause fatigue. For many people, chemotherapy is the best option for controlling their cancer. However, chemotherapy can also damage normal cells such as bone marrow and blood cells, cells of the hair follicles, and cells of the reproductive and digestive tracts. Chemotherapy can also cause nausea, vomiting, constipation, diarrhea, fatigue, changes to the nervous system, cognitive changes, lung damage, reproductive and sexual problems, liver, kidney, and urinary system damage, and, especially with the use of the chemotherapeutic agent doxorubicin, heart damage. Long-term side effects of chemotherapy can include permanent organ damage, delayed development in children, nerve damage, and blood in the urine. Thus, the use of the chemotherapy for cancer treatment is not without serious side effects.

Most agents currently administered to a patient are not targeted to the site where they are needed, resulting in systemic delivery of the agent to cells and tissues of the body where the agent is unnecessary, and often undesirable. Such systemic delivery may result in adverse side effects, and often limits the dose of an agent (e.g., cytotoxic agents and other anti-cancer agents) that can be administered. Accordingly, a need exists for agents and methods that specifically target cancerous cells and tissues.

Thus, it would be desirable to be able to direct various agents to cancer cells and to the tumor microenvironment so as to be able to decrease the dosage of the agents given and to decrease the systemic toxicity and side effects associated with these agents.

### SUMMARY OF THE INVENTION

According to the invention, the tumor microenvironment creates conditions that induce expression of certain genes, including proteases that are active almost exclusively in the tumor microenvironment. For example, an unexpectedly high level expression of asparaginyl endopeptidases, including legumain, is present in a wide variety of cancer cells, particularly those involved in metastasis. Other proteases that are active in the tumor microenvironment include prostate specific membrane antigen (PSMA)(a carboxypeptidase), fibroblast activation protein (FAP) (a serine peptidase), cathepsin B (a cysteine protease), cathepsin X (a cysteine protease), urokinase-type plasminogen activator (uPA)(a serine protease), tissue factor VIIa (TF VIIa)(a serine protease), matriptase (a membrane-bound serine protease) and Factor XIII. As described herein, tumor-specific protease expression occurs early in the development of cancer cell invasion, just as metastasis begins, and under the hypoxic conditions associated with invasive tumor growth. Moreover, legumain is directly involved in and actually inhibits the cascade of activities that leads to cellular apoptosis, particularly in cancer cells where legumain is highly expressed.

The tumor specific proteases described herein are cell surface associated proteases. These proteases function in protease networks that play critical roles in modulating extracellular matrix proteins. For instance, certain cancers may employ more cysteine proteases than matalloproteinas or serine protease and verse versa. However the matrix modifying function of each of these proteases is indispensable for tumor metastasis and invasive growth.

According to the invention, the combined use of protease activity imaging agents, protease inhibitors, and/or prodrugs described herein represents an integrated precision-guided cancer therapeutic system. The combined use of protease inhibitor and prodrugs are also envisioned, although in some embodiments the prodrug and protease inhibitor targeting same protease are used at different times during the therapeutic regimen.

Also as shown herein, legumain activity is substantially increased on the surface of tumor cells by cell-surface association with integrins, indicating that integrins are co-factors for legumain. Moreover, legumain can activate metalloproteinases (e.g. MMP-2) and cathepsins (e.g., cathepsins B, H and L), which are all proteases involved in promoting tumor cell invasion and metastasis. In addition, asparaginyl endopeptidase expression is also associated with reduced cancer cell apoptosis and increased angiogenesis. Therefore, expression of, and activity by, certain proteases, including legumain, PSMA, FAP, cathepsin B, cathepsin X, uPA, tissue factor VIIa, matriptase (a membrane-bound serine protease) and Factor XIII are cancer and angiogenesis markers and constitute indicia of tumor cell metastasis. The invention therefore provides agents to treat undesirable angiogenesis, tumor cell invasion, tumor cell metastasis and other such cancerous conditions, particularly those conditions involving cells and tissues that express these proteases.

Many tumor cells are largely resistant to chemotherapy, for example, because the chemotherapeutic agents employed are only active against a subset of the tumor cells that comprise a cancerous condition. According to the invention, stromal cells in the tumor microenvironment, such as endothelial cells or tumor associated macrophages (TAMs), can be targeted by the agents of the invention to effectively treat these drug-resistant tumor cell types. This strategy is also effective for reducing the expression and/or activity of molecules in the tumor microenvironment that attract TAMs and other tumor-associated cells that facilitate tumor growth and invasion. TAMs consist of a polarized M2 (CD206+, F4/80+) macrophage population. TAMs also possess poor antigen presenting capacity and effectively suppress T cell activation. In fact, TAMs actually promote tumor cell proliferation and metastases by producing a wide range of growth factors, pro-angiogenesis factors, metalloproteinases and the like. TAMs also partake in circuits that regulate the function of fibroblasts in the tumor stroma and are particularly abundantly expressed in the tumor stroma.

According to the invention, TAMs express high levels of certain proteases, including legumain, in the tumor microenvironment. In contrast, classical macrophages of the M1 phenotype, that perform key immune-surveillance functions, do not express legumain. Consequently, targeted elimination of TAMs does not interfere with the biological functions of normal (MI) macrophages, including cytotoxicity and antigen presentation. Thus, one aspect of the invention involves targeting legumain-expressing TAMs with prodrugs and/or proteases inhibitors to destroy TAMs and/or inhibit their function.

TAM and endothelial cells are non-transformed cells therefore will not develop drug resistance that is common among malignant cancers. Thus, low dosages of the prodrugs and/or protease inhibitors can be employed when targeting these TAM and endothelial cells. This will down regulate a wide variety of tumor growth factors, pro-angiogenesis factors and enzymes released by these macrophages and lead to inhibition of tumor angiogenesis as well as invasive growth and metastasis.

One aspect of the invention is a method of treating cancer in a mammal by administering to the mammal an effective amount of a prodrug or an inhibitor of a protease that is expressed in the tumor microenvironment. According to the invention, treatment of cancer can involve killing tumor cells, reducing the growth of tumor cells and reducing the growth or function of tumor stromal cells in a mammal. Examples of stromal cells that can be treated by the methods of the invention include tumor-associated macrophages and endothelial cells. Treatment of cancer can also involve promoting apoptosis of cells that express legumain. As shown herein, expression of legumain inhibits apoptosis and cancer cells that exhibit high levels of legumain expression resist apoptosis. Treatment of cancer can also involve inhibiting angiogenesis of a tumor in a mammal.

Another aspect of the invention is a protease-activated prodrug that is tumoricidal in vivo, wherein the protease is a protease that is expressed in the tumor microenvironment (e.g., under the hypoxic conditions of tumor microenvironments). These protease-activated prodrugs have reduced side effects and toxicity relative to currently available chemotherapeutics. While the present prodrugs are useful for treating cancer, they are also useful to treat other conditions and cellular environments that express proteases. For example, certain non-transformed cells support tumor growth and invasion and, as described herein also express proteases when present in the tumor microenvironment. Hence, the present prodrugs can be used to target and kill not only cancer cells but also the cells that support tumor growth and tumor cell metastasis.

A prodrug compound of the invention includes a drug molecule linked to a peptide, wherein the peptide has an amino acid sequence that is specifically recognized by a protease expressed in a tumor microenvironment. Thus, the peptide includes at least two linked amino acids, wherein at least one of the two linked amino acids is an amino acid that is specifically recognized by a tumor-specific protease and forms part of a cleavage site for the protease. For example, legumain is a protease that is specifically expressed in tumor cells and cells that support tumor growth and metastasis (e.g. tumor associated macrophages). Legumain is an asparaginyl protease that specifically recognizes asparagine-containing peptides and cleaves peptides that contain asparagine (Asn). Some of the prodrugs of the invention are therefore designed to be activated by legumain. Legumain cleaves the peptide of the present prodrugs at the site of the Asn to generate an active drug from the prodrug. Prior to cleavage, the prodrug is substantially non-toxic to normal animal cells, whereas after cleavage, the drug is an active drug that can have a beneficial effect upon an animal to which it is administered.

Prodrugs of the invention have the general structure:

R₁-peptide-drug

wherein R₁ is hydrogen, a hydrophilic group, a hydrophobic group, a photosensitizing agent, a label or an amino protecting group; and drug is any therapeutic agent. In some embodiments, the R₁ groups of the present prodrugs are not hydrophobic groups or labels. The R₁ and drug moieties can be linked directly to the peptide or they can be linked to the peptide through a linker or spacer molecule. Such a linker of spacer can be an alkylene, a sugar or an oligosaccharide.

Specific examples of prodrug compounds of the invention include, for example, taxol, paclitaxel, doxorubicin containing prodrugs, including those shown below:

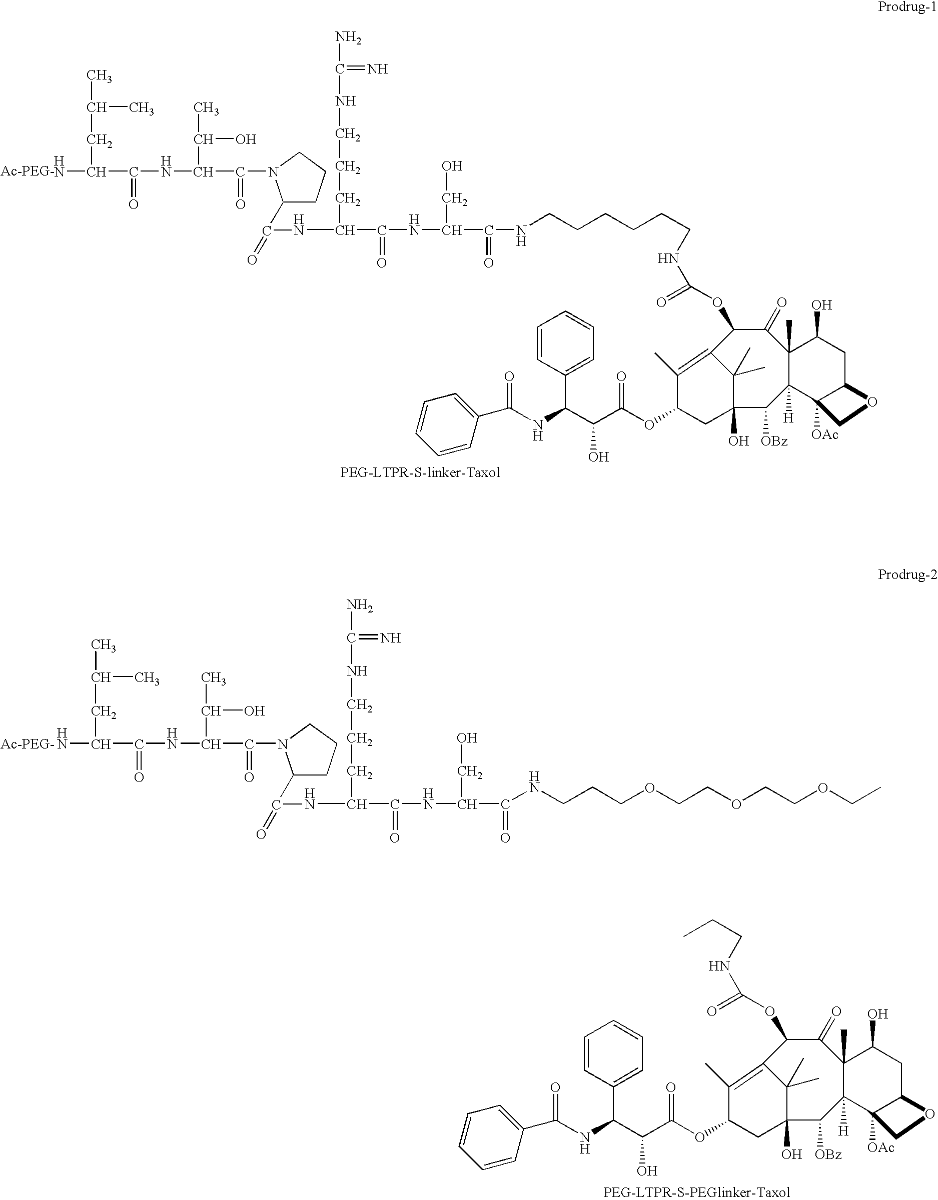

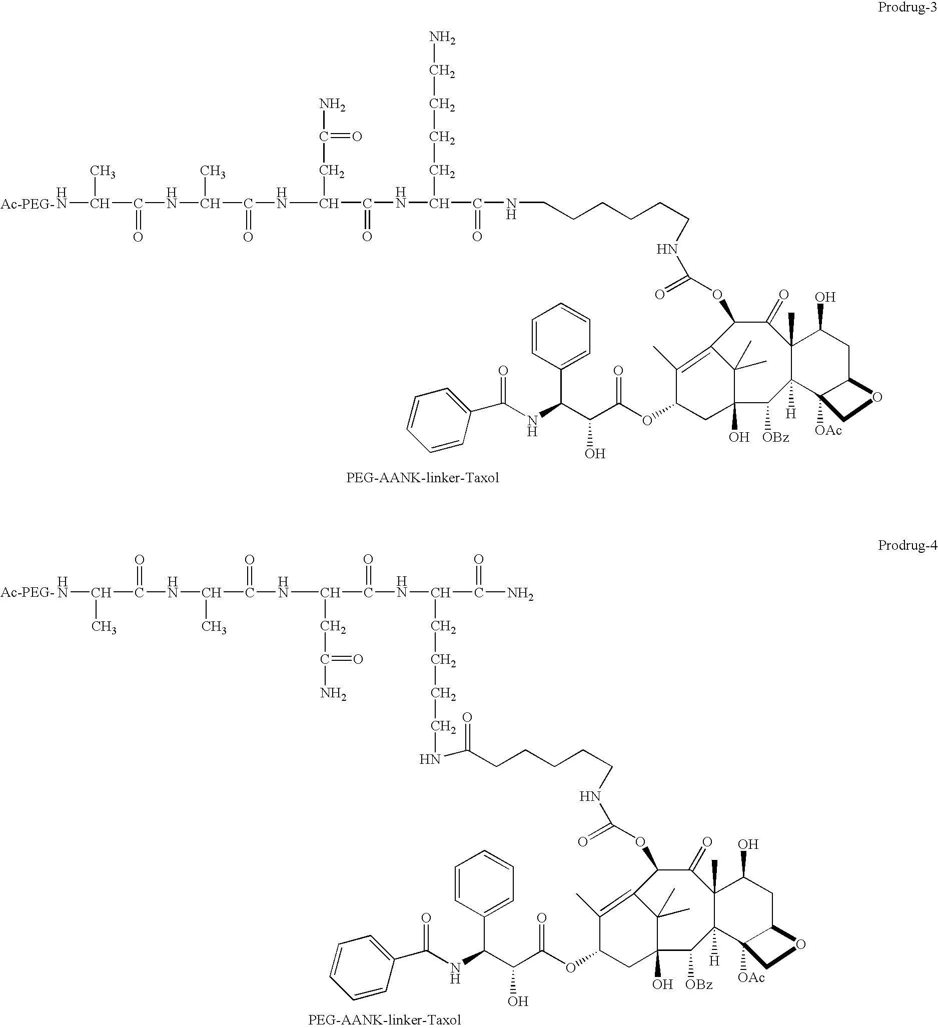

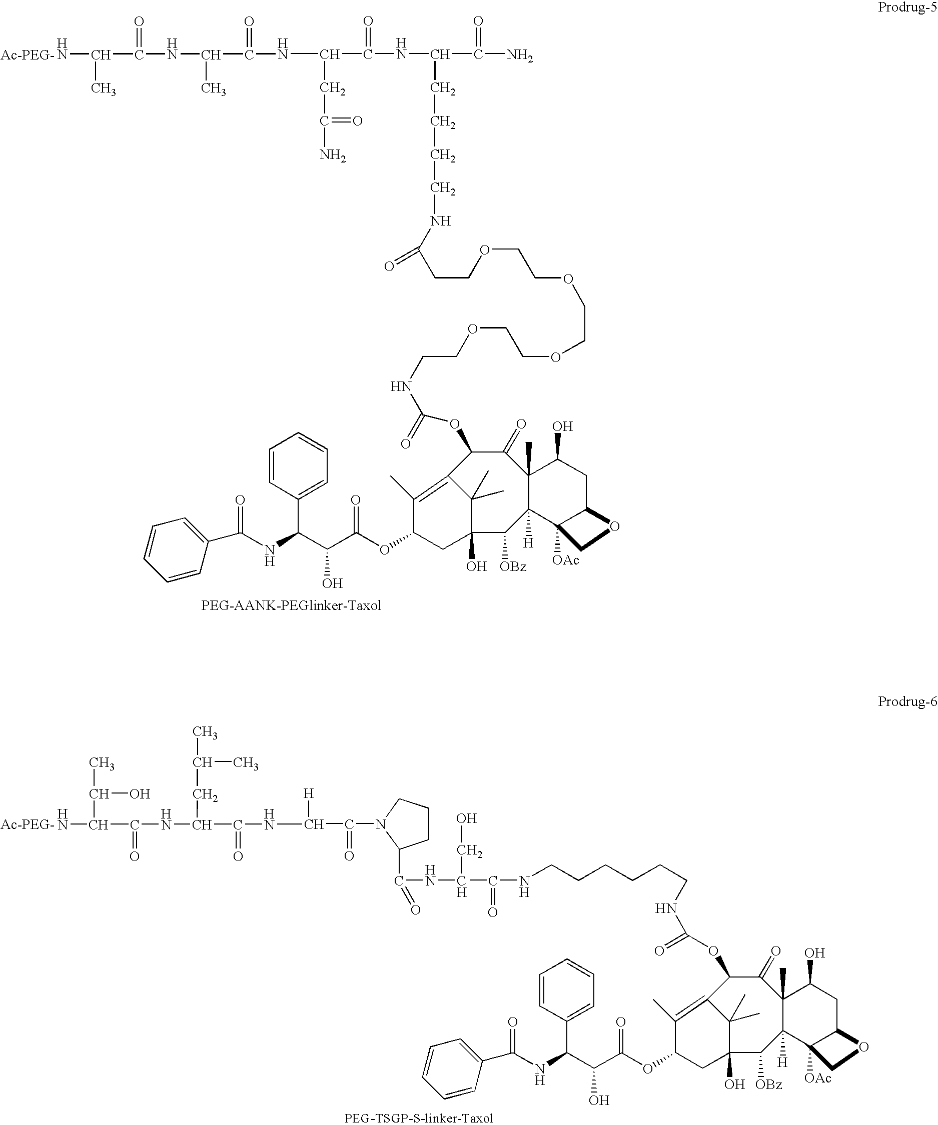

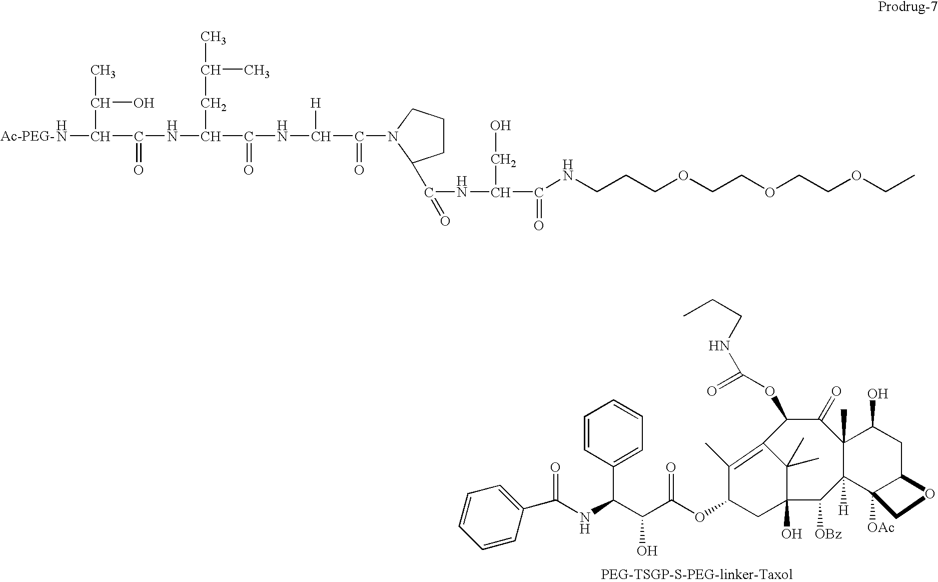

Prodrug-1 and prodrug-2 are activated by serine protease TF VIIa. Prodrug-3, prodrug-4 and prodrug-5 are activated by legumain. Additional legumain prodrugs include LEG-2 (N-Succinyl-β-alanyl-L-threoinyl-L-Asparaginyl-L-Leucyl-Doxorubicin) (SEQ ID NO:24) and LEG-3 (N-Succinyl-β-alanyl-L-alanyl-L-Asparaginyl-L-Leucyl-Doxorubicin) (SEQ ID NO:25), whose structures are shown in FIG. 9A. In some instances, the prodrugs of the invention do not include LEG-2 or LEG-3, because the inventor has filed a separate application on these compounds. Prodrug-6 and prodrug-7 are activated by fibroblast activation protein (FAP).

The drug employed is any drug whose action is diminished or blocked by attachment of a peptide to the drug. The ability of the drug to enter cells is diminished, inhibited or blocked by attachment of the peptide and hydrophilic groups. Such hydrophilic groups are generally included to facilitate water-solubility and cell impermeability. Hydrophilic groups are generally attached to the peptide so that the function of the drug is not inhibited or blocked by the hydrophilic group once the peptide is cleaved from the prodrug to yield the drug.

In some embodiments, the drug can be a cytotoxin or a photosensitizing agent. Such a cytotoxin can be aldesleukin, 5-aminolevulinic acid, bleomycin sulfate, camptothecin, carboplatin, carmustine, cisplatin, cladribine, lyophilized cyclophosphamide, non-lyophilized cyclophosphamide, cytarabine, dacarbazine, dactinomycin, daunorubicin, diethylstilbestrol, epoetin alfa, esperamycin, etidronate, etoposide, filgrastim, floxuridine, fludarabine phosphate, fluorouracil, goserelin, granisetron hydrochloride, idarubicin, ifosfamide, immune globulin, interferon alpha-2a, interferon alpha-2b, leucovorin calcium, leuprolide, levamisole, mechiorethamine, medroxyprogesterone, melphalan, methotrexate, mitomycin, mitoxantrone, octreotide, ondansetron hydrochloride, paclitaxel, pamidronate, pegaspargase, plicamycin, protoporphyrin IX, sargramostim, streptozocin, taxol, thiotepa, teniposide, vinblastine, or vincristine. In some embodiments, the drug is doxorubicin, 5-aminolevulinic acid, protoporphyrin IX, taxol or paclitaxel.

In one embodiment, the prodrug is activated by asparaginyl proteases (e.g., legumain) and has a peptide amino acid sequence comprising SEQ ID NO:3:

R₁-(Xaa1)ₙ-Xaa2-Asn-(Xaa3)-drug

wherein:
R₁ is hydrogen, a hydrophilic group, a hydrophobic group, a photosensitizing agent, a label or an amino protecting group;
n is an integer of about 0 to about 50;
Xaa1 and Xaa2 are separately any amino acid;
Xaa3 is either nothing or an amino acid that has no substantial effect on the activity of the drug; and
the drug employed is a drug whose action is diminished or blocked by attachment of a peptide to the drug.
   In some embodiments, the R₁ groups of the present prodrugs are not hydrophobic groups or labels. For example, when cytotoxic drugs are part of the prodrug, a hydrophilic group is preferably used for R₁ to limit cell uptake by non-target cells.

Examples of peptide sequences that may be used in the prodrugs of the invention include amino acid sequence Asn-Leu, Ala-Asn-Leu, Thr-Asn-Leu, Ala-Ala-Asn-Leu (SEQ ID NO:5), Ala-Thr-Asn-Leu (SEQ ID NO:6), and Boc-Ala-Ala-Asn-Leu (SEQ ID NO:4). Examples of prodrugs provided by the invention include Boc-Ala-Ala-Asn-Leu-doxorubicin (SEQ ID NO:7), succinyl-Ala-Ala-Asn-Leu-doxorubicin (SEQ ID NO:8), N-(-t-Butoxycarbonyl-Ala-Thr-Asn-Leu)doxorubicin (SEQ ID NO:9), N-(Succinyl-Ala-Thr-Asn-Leu)doxorubicin (SEQ ID NO: 10), N-(-t-Butoxycarbonyl-Ala-Asn-Leu)doxorubicin (SEQ ID NO: 11), N-(Succinyl-Ala-Asn-Leu)doxorubicin (SEQ ID NO: 12), N-(-t-Butoxycarbonyl-Thr-Leu)doxorubicin (SEQ ID NO: 13), N-(Succinyl-Thr-Leu)doxorubicin (SEQ ID NO:14),

As described herein a hydrophilic R₁ group (sometimes abbreviated herein as “Hyd”) facilitates prodrug and inhibitor water solubility and inhibits cell uptake and tissue retention of the prodrug before activation and of the inhibitor before binding to a protease (e.g. the legumain:integrin complex). A variety of hydrophilic protecting groups can be utilized. Hydrophilic R₁ groups of the invention can be sugars (monosaccharides and disaccharides), dicarboxylic acids (e.g., succinate, malate, fumarate, oxaloacetate, citrate, isocitrate), glycans, polyalkylene oxides, lower alkyl carboxylates, carboxyalkyls, carboxyalkylene carboxylates, charged amino acids (e.g., any of the hydrophilic, acidic, basic and polar amino acids described herein) and the like. In some embodiments the protecting group is a hydrophilic amino protecting group. Specific examples of R₁ groups that can be used include glucuronide, succinyl, polyethylene glycol (PEG) or glutathione. Hydrophobic groups can used if cellular uptake of the drug is desired. Hydrophobic groups that can be used include those listed herein.

The invention also provides a tumor-specific protease inhibitor. In general, the tumor-specific protease inhibitor has the following structure.

R₁-peptide-Y

More specific examples of the present tumor-specific inhibitors include inhibitors of formulae III, IV, V or VI:

| | | |
|---|---|---|
| R₁-(Xaa4)ₙ-Asn-Y | (SEQ ID NO:26) | III |
| R₁-(Xaa4)ₙ-Xaa5-Y | (SEQ ID NO:27) | IV |
| R₁-Xaa4-azaAsn-Y | | V |
| R₁-Xaa4-azaXaa5-Y | | VI |

wherein:
wherein R₁ is hydrogen, a hydrophilic group, a hydrophobic group, a photosensitizing agent, a label or an amino protecting group;
n is an integer of about 2 to about 5;
each Xaa4 and Xaa5 is an amino acid or an amino acid mimetic;
Y is alkyl, alkenyl epoxide, fluoromethylketone or a Michael acceptor, optionally substituted with 1-3 halo or hydroxy, alkylamino, dialkylamino, alkyldialkylamino, or cycloalkyl, alkylcycloalkyl, alkenylcycloalkyl, aryl; (C₅-C₁₂)arylalkyl or (C₅-C₁₂)arylalkenyl,
wherein the aryl groups of the arylalky or arylalkenyl can be 0-4 heteroatoms selected from N, O and S, and are optionally substituted with halo, cyano, nitro, haloalkyl, amino, aminoalkyl, dialkylamino, alkyl, alkenyl, alkynyl, alkoxy, haloalkoxy, carboxyl, carboalkoxy, alkylcarboxamide, (C₅-C₆)aryl, —O—(C₅-C₆)aryl, arylcarboxamide, alkylthio or haloalkylthio; and
wherein each of the inhibitors of formulae III, IV, V and VI bind to a protease expressed in a tumor microenvironment.

Examples of asparaginyl endopeptidase inhibitors (AEPIs) that may be used in the methods of the invention include the following:

AEPI-1 is R₁-Ala-Ala-AzaAsn-(S,S)-EPCOOEt, a compound of the structure:

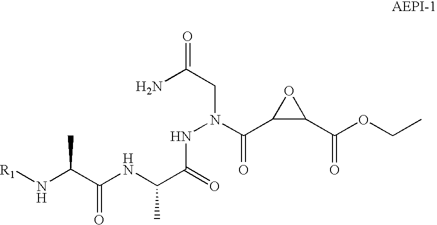

AEPI-2 is R₁-Ala-Ala-AzaAsn-CH═CH—COOEt, for example, a compound of the structure:

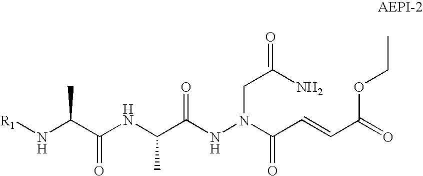

AEPI-3 is R₁-Ala-Ala-AzaAsn-CH═CH—COOBzl, for example, a compound of the structure:

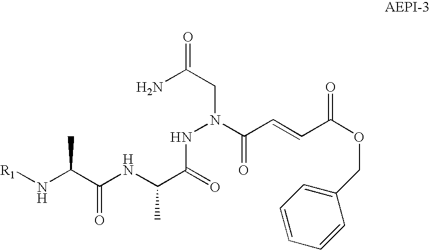

AEPI-4 is R₁-Ala-Ala-AzaAsn-CH═CH—CON(CH₃)Bzl, for example, a compound of the structure:

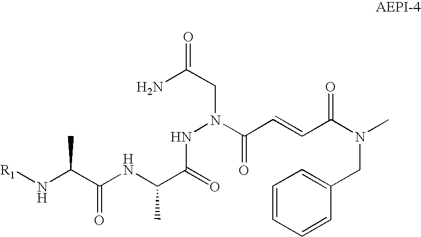

AEPI-5 is N-acetyl-Ala-Ala-AzaAsn-(S,S)-EPCOOEt, a compound of the structure:

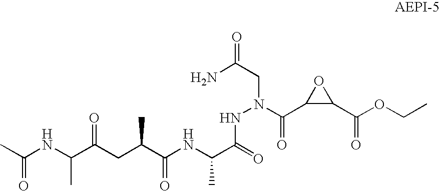

AEPI-6 is N-succinyl-Ala-Ala-AzaAsn-(S,S)-EPCOOEt, a compound of the structure:

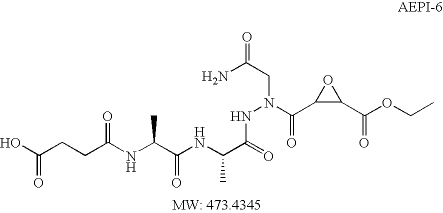

AEPI-7 is N-hepatanoyl-Ala-Ala-AzaAsn-(S,S)-EPCOOEt, a compound of the structure:

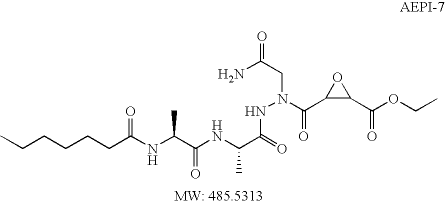

AEPI-8 is 3-(N-carbamoylmethyl-N′-{fluorescein]-methylsulfanyl}-N-propionyl)-Ala-Ala-AzaAsn-(S,S)-EPCOOEt, a compound of the structure:

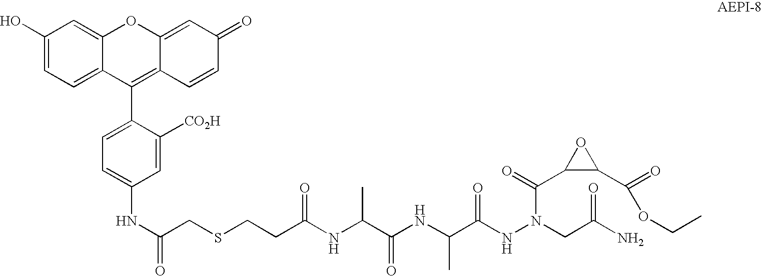

AEPI-9 is N-triethylamino-Ala-(N-isopropanoyl-Ala-AzaAsn-(S,S)-EPCOOEt), a compound of the structure:

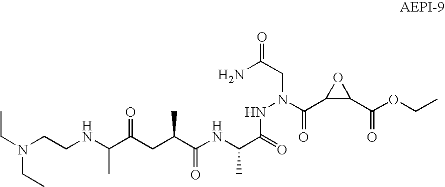

wherein R₁ is hydrogen, a hydrophilic group, a hydrophobic group, a photosensitizing agent, a label or an amino protecting group; Ep is epoxy; Et is ethyl; and Bzl is benzyl.

Inhibitors of PSMA that can be used in the invention include the following:

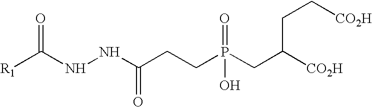

wherein R₁ is hydrogen, a hydrophilic group, a hydrophobic group, a photosensitizing agent, a label or an amino protecting group.

Inhibitors of fibroblast activation protein (FAP) that can be used in the invention include the following:

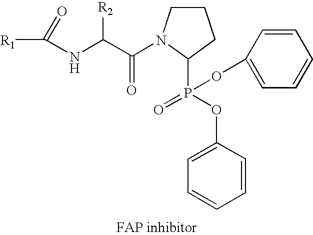

wherein R₁ is hydrogen, a hydrophilic group, a hydrophobic group, a photosensitizing agent, a label or an amino protecting group; and R₂ is hydrogen, hydroxymethylene (CH₂OH), lower alkyl (e.g., methyl, ethyl, propyl, isopropyl (CH(CH₃)₂), butyl, isobutyl), or benzyl.

Cathepsin B inhibitors useful in the invention include the following:

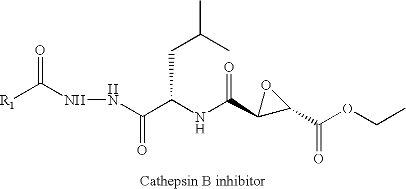

wherein R₁ is hydrogen, a hydrophilic group, a hydrophobic group, a photosensitizing agent, a label or an amino protecting group.

Cathepsin X inhibitors useful in the invention include the following:

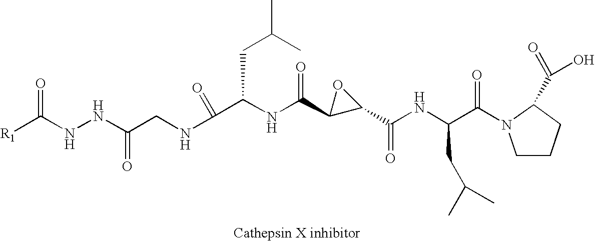

wherein R₁ is hydrogen, a hydrophilic group, a hydrophobic group, a photosensitizing agent, a label or an amino protecting group.

Urokinase-type plasminogen activator (uPA) inhibitors useful in the invention include the following:

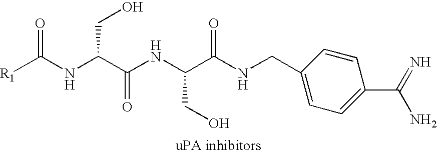

wherein R₁ is hydrogen, a hydrophilic group, a hydrophobic group, a photosensitizing agent, a label or an amino protecting group.

Tissue factor VIIa (TF VIIa) inhibitors useful in the invention include the following:

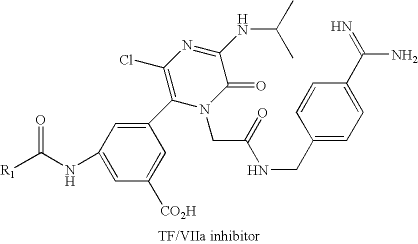

wherein R₁ is hydrogen, a hydrophilic group, a hydrophobic group, a photosensitizing agent, a label or an amino protecting group.

Matriptase inhibitors useful in the invention include the following.

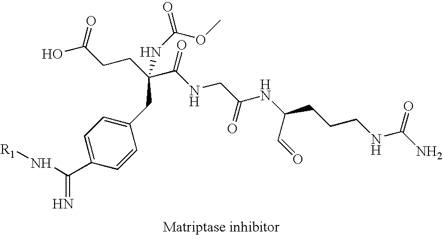

wherein R₁ is hydrogen, a hydrophilic group, a hydrophobic group, a photosensitizing agent, a label or an amino protecting group.

Factor XIII inhibitors useful in the invention include the following.

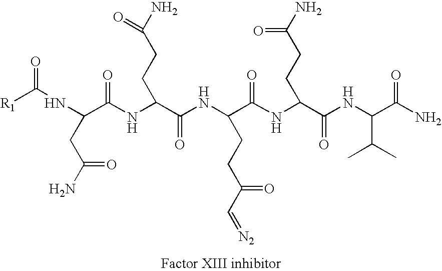

wherein R₁ is hydrogen, a hydrophilic group, a hydrophobic group, a photosensitizing agent, a label or an amino protecting group.

In some embodiments, the inhibitor is linked to a photosensitizing agent. While any available photosensitizing agent can be used (e.g., any of the photosensitizing agents contemplated for use with the present prodrugs), specific examples of photosensitizing agents include chlorin e6 and aluminum phthalocyanine tetrasulfonate (AlPcS4). Examples of inhibitors with chlorin e6 and AlPcS4 are shown below.

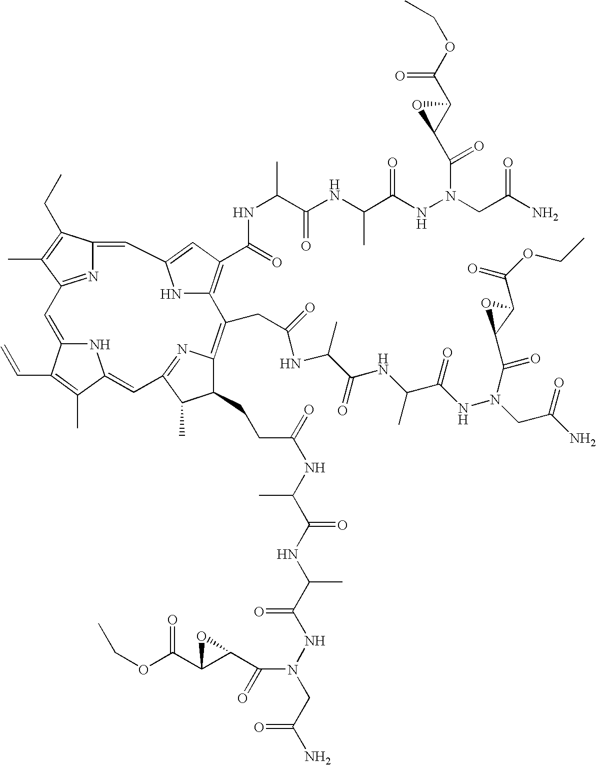

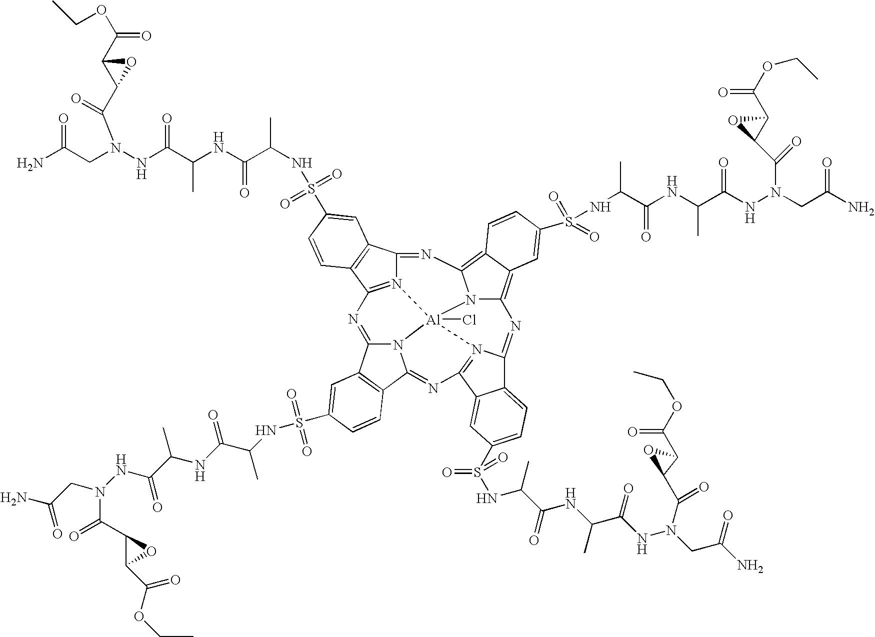

Hydrophilic R₁ groups of the invention can be sugars (monosaccharides and disaccharides), dicarboxylic acids (e.g., succinate, malate, fumarate, oxaloacetate, citrate, isocitrate), glycans, polyalkylene oxides, lower alkyl carboxylates, carboxyalkyls, carboxyalkylene carboxylates, and the like.

Hydrophobic R₁ groups of the invention can be alkyl, aryl, alkylene aryl, arylalkyl, hydrophobic amino protecting agents, beta-alanyl and related hydrophobic groups.

Any convenient amino protecting group available in the art can be used in the invention including, for example, carbobenzyloxy (Cbz), tert-butyloxycarbonyl (BOC), 9-fluorenylmethyloxycarbonyl (FMOC) and benzyl groups.

A variety of labels can be used with the inhibitors of the invention to generate imaging agents or reagents for detection of cancer. Such labels can be fluorophores, radioisotopes, metals, enzymes, enzyme substrates, luminescent moieties, and the like. One example of a label that may used is gadolinium or a gadolinium complex. For example, the following gadolinium complex can be used as a label:

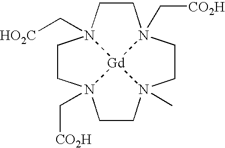

In other embodiments, the legumain inhibitor can be cystatin, stefin, a peptide including the sequence Ala-Leu-β-Asn-Ala-Ala (SEQ ID NO: 15) or an antibody that inhibits legumain activity.

Another aspect of the invention is a pharmaceutical composition that includes at least one of the prodrug compounds of the invention or at least one of the protease inhibitors of the invention and a pharmaceutically acceptable carrier. In some embodiments, the carrier is a liposome. Combinations of the present prodrug compounds and/or protease inhibitors can also be included in the compositions of the invention.

Another aspect of the invention is a method of detecting and treating cancer in a mammal, comprising administering to the mammal an inhibitor of the invention, wherein R₁ is a label, to detect whether the mammal has cancer and to detect which type of tumor specific protease is associated with the cancer, and administering a prodrug of the invention to treat the cancer, wherein the prodrug has a cleavage site for the tumor specific protease associated with the cancer.

Another aspect of the invention is a method for treating a mammal having, or suspected of having cancer. The method includes administering to the mammal a prodrug compound and/or a protease inhibitor of the invention in amounts and at intervals effective to prevent, reduce, or eliminate one or more of the symptoms associated with cancer. The cancer can be an invasive or metastatic cancer. The cancer can also be a tumor that is prone to angiogenesis. Cancers that can be treated by the invention include solid tumors and cancers as well as cancers associated with particular tissues, including breast cancer, colon cancer, lung cancer, prostate cancer, ovarian cancer, cancer of the central nervous system, carcinomas, leukemias, lymphomas, melanomas, fibrosarcomas, neuroblastoma, and the like. The cancer can, for example, be autoimmune deficiency syndrome-associated Kaposi's sarcoma, cancer of the adrenal cortex, cancer of the cervix, cancer of the endometrium, cancer of the esophagus, cancer of the head and neck, cancer of the liver, cancer of the pancreas, cancer of the prostate, cancer of the thymus, carcinoid tumors, chronic lymphocytic leukemia, Ewing's sarcoma, gestational trophoblastic tumors, hepatoblastoma, multiple myeloma, non-small cell lung cancer, retinoblastoma, or tumors in the ovaries.

The invention also provides a method for inhibiting cancer metastasis and/or tumor cell invasion in an animal, including administering a protease inhibitor compound and/or prodrug of the invention to the animal in amounts and at intervals effective to prevent, reduce, or eliminate cancer metastasis and/or tumor cell invasion.

The invention also provides a method for inhibiting cell migration in an animal that includes administering a protease inhibitor compound and/or prodrug of the invention to the animal in amounts and at intervals effective to prevent, reduce, or eliminate cancer cell migration.

The invention also provides a method of killing a cell in a tissue, including contacting the cell with a prodrug of the invention in amounts and at intervals effective to kill the cell, wherein the tissue includes cells that express legumain.

The invention also provides a method for treating cancer in animal that includes administering to the animal a protease inhibitor compound or a prodrug of the invention in amounts and at intervals effective to prevent, reduce, or eliminate one or more symptoms of cancer in the animal.

The invention also provides a method for inhibiting cancer metastasis in a tissue that includes contacting the tissue with a protease inhibitor compound or prodrug of the invention in amounts and at intervals effective to prevent, reduce, or eliminate cancer metastasis.

The invention also provides a method for inhibiting cancer cell migration in a tissue that includes contacting the tissue with a protease inhibitor compound or prodrug of the invention in amounts and at intervals effective to prevent, reduce, or eliminate cancer cell migration.

The invention also provides a method for treating inflammation in an animal, which includes administering to the mammal a protease inhibitor compound or prodrug of the invention in amounts and at intervals effective to prevent, reduce, or eliminate one or more symptoms associated with inflammation.

The invention also provides a method for delivering a drug to a cell in a tumor microenvironment of a mammal, which includes administering to the mammal an effective amount of a prodrug of the invention.

The invention also provides a method for diagnosing cancer in a tissue that includes contacting the tissue with of an agent that specifically binds to a protease that is expressed in a tumor microenvironment, and detecting whether the agent binds to the tissue. The invention also provides a method for diagnosing cancer in an animal that includes administering to the animal an agent that specifically binds to a protease that is expressed in a tumor microenvironment, and detecting whether the agent accumulates in a tissue. For example, the protease can be selected from the group legumain, PSMA, FAP, Cathepsin B, Cathepsin X, uPA, TF VIIa, matriptase and Factor XIII. These methods can further include diagnosing the patient as having or not having cancer and monitoring the progression of a cancer.

The invention also provides a method for imaging a tissue that expresses a protease. For example, the protease can be selected from the group legumain, PSMA, FAP, Cathepsin B, Cathepsin X, uPA, TF VIIa, matriptase and Factor XII. The method includes contacting a test tissue suspected of expressing the protease with of an agent that specifically binds to the protease, and detecting whether the agent binds to the test tissue. The method can further include quantifying and comparing amounts of the agent bound to the test tissue with amounts of the agent bound to a control tissue that does not express the protease. The agent can be protease inhibitor-imaging agent or a labeled antibody that specifically binds to the protease.

In another embodiment, the prodrug and inhibitor compounds of the invention can be used for the manufacture of a medicament useful for treating diseases such as cancer.

### BRIEF DESCRIPTION OF THE FIGURES

FIG. 1A-C shows that legumain expression is induced by hypoxia and occurs early in metastatic sites. FIG. 1A illustrates a Western blot analysis of legumain expression in cells under normoxic and hypoxic conditions. FIG. 1B shows a hematoxylin and eosin stained section of 4T1 breast carcinoma lung metastasis. FIG. 1C provides results of an immunohistochemical analysis of frozen section of 4T1 lung metastasis. Legumain is stained green (lighter areas), CD31 is stained red, and nuclei are stained blue in the original.

FIG. 2A-E shows that legumain forms a legumain:αvβ3 integrin protease complex. FIG. 2A shows that legumain is immunoprecipitated with a panel of anti-integrin antibodies. The anti-integrin antibody type is indicated above each lane of the western blot. FIG. 2B shows that legumain is co-precipitated from cell lysates using anti-αvβ3 integrin antibodies and αvβ3 integrin is co-precipitated from cell lysates with anti-legumain antibodies. FIG. 2C illustrates the location of legumain:αvβ3 integrin complexes in MBA-MA231 cells. The αvβ3 integrin is red, legumain is green, nuclei are blue and the legumain:αvβ3 complexes are yellow. These legumain:αvβ3 complexes are formed intracellularly in vesicles and transported to cell surfaces. FIG. 2D illustrates extensive formation of legumain:αvβ3 integrin complexes in Panc-1 human pancreatic carcinoma grown in nude mice. The αvβ3 is red, legumain is green, cell nuclei are blue and the legumain:αvβ3 co-localization is yellow. FIG. 2E provides a schematic representation of legumain:αvβ3 complexes and the types of intracellular trafficking and cell surface proteolysis that occur.

FIG. 3A-E shows that integrin αvβ3 is co-factor of legumain activity. FIG. 3A shows that binding of αvβ3 integrin increases the amidolytic activity of legumain in a dose dependent manner. FIG. 3B provides a comparison of asparaginyl endopeptidases activity of legumain (dark bars) and legumain: αvβ₃ complexes (open bars). FIG. 3C shows that αvβ3 integrin shifts the pH dependency of legumain to a higher pH. FIG. 3D shows that αvβ3 integrin increases legumain activity towards its physiologic substrate MMP2 as assessed using zymogram. FIG. 3E provides a schematic illustration of MMP2 activation by legumain. Legumain not only activates MMP2 by removing its propeptide, it also cleaves MMP2 between the MMP2 catalytic domain and the hemopexin-like repeats. Hemopexin can inhibit angiogenesis, indicating that legumain has a regulatory role in vessel development.

FIG. 4A-G shows that legumain is an important regulator of hypoxia induced endothelial invasion and tube formation. FIG. 4A shows the structures of four asparaginyl endopeptidases inhibitors (AEPIs) that legumain inhibitory activity. The four AEPI's have the following chemical names: AEPI-1 is Cbz-Ala-Ala-Aza-Asn-(S,S)-EPCOOEt; AEPI-2 is Cbz-Ala-Ala-AzaAsn-CH═CH—COOEt; AEPI-3 is Cbz-Ala-Ala-AzaAsn-CH═CH—COOBzl; and AEPI-4 is Cbz-Ala-Ala-AzaAsn-CH═CH—CON(CH3)Bzl. FIG. 4B illustrates the cytotoxicity of AEPIs in wild type 293 cells and in 293 cells that express legumain. FIG. 4C shows that the four AEPIs have strong inhibitory activity as assessed by a legumain amidolytic assay. FIG. 4D shows that increasing concentrations of AEPI-1 increasingly inhibit HUVEC tube formation in vitro. FIG. 4E shows that addition of recombinant legumain protein promotes HUVEC tube formation. FIG. 4F illustrates HUVEC matrigel tube formation under normal and hypoxic conditions, with and without legumain inhibitors cystatin and aza-peptide epoxide. FIG. 4G illustrates the number of migrating cells per field as detected in invasion assays of HUVEC under normal and hypoxic conditions, with and without legumain inhibitors, cystatin and aza-peptide epoxide.

FIG. 5A-D shows that AEPI-1 inhibits angiogenesis in vivo. FIG. 5A shows that AEPI-1 inhibits mouse aorta vessel sprouting in a dose-dependent manner. FIG. 5B further illustrates that AEPI-1 inhibits the extension of mouse aorta vessel sprouts after the sprouts had already formed. FIG. 5C shows that AEPI-1 inhibits angiogenesis within FGF-2 and cancer cell containing Matrigel plugs that were injected subcutaneously into mice. Vessel density was quantified by hemoglobin content in the plugs.

FIG. 6A-D shows that AEPI-1 suppresses tumor invasive growth. FIG. 6A shows that legumain promotes tumor cell invasion in vitro. FIG. 6B illustrates the human breast cancer model established in nude mouse. The tumor is similar to a human infiltrating ductal carcinoma with apparent glandular differentiation. The tumors grow aggressively and are invading surrounding local muscles. FIG. 6C shows that AEPI-1 treatment of tumors suppresses cancer cell differentiation and invasive growth. FIG. 6D shows that AEPI-1 treatment of tumors dramatically reduces tumor volume as assessed at 14 days post cancer cell injection.

FIG. 7A-C illustrates legumain expression in cancer cells and cell surface association of legumain. FIG. 7A provides results of a Western blot analysis of cultured tumor cells and corresponding in vivo tumor derived cells. FIG. 7B shows a flow cytometry analysis of cell surface legumain in single cell suspensions obtained from CT26 tumors with (bottom panel) and without (top panel) collagenase treatment. FIG. 7C shows flow cytometry analysis of single cell suspensions prepared from tumor, bone marrow, spleen, and kidney tissues as well as cultured CT26 tumor cells for cell surface legumain. The percentage of cells positive for surface legumain is plotted in the bar graph insert.

FIG. 8A-E shows that legumain is expressed by stromal cells in tumors. FIG. 8A illustrates that double staining with anti-legumain antibody (green) and anti-CD31 antibody (red) identifies endothelial cells. Tumor vascular endothelial cells expressing legumain in tumor are indicated by arrows (400×). FIG. 8B illustrates that double staining of anti-legumain antibody (green) and anti-CD68 antibody (red) identifies legumain expressing TAMs (arrows) (600×). TAM cells expressing legumain are indicated by arrows. FIG. 8C illustrates that double staining of anti-legumain (green) with anti-collagen I antibody (red) (400×). Co-localization of legumain with collagen I in the extracellular matrix is indicated by yellow. FIG. 8D provides a two dimensional analysis of single cell suspensions prepared from 4T1 in vivo mouse mammary tumors following mechanical dissociation and of 4T1 tumor cells from tissue culture. Legumain was observed on the surface of viable endothelial cells and tumor-associated macrophages as identified by anti-legumain antibodies and anti-CD31 antibodies or anti-CD14 antibodies, respectively. FIG. 8E illustrates two dimensional flow cytometry of single cell suspensions prepared from 4T1 tumors using collagenase digestion. Legumain-associated endothelial cells constituted the group of cells that are legumain⁺ and CD31⁺. Legumain-associated TAMs constituted legumain⁺ and CD 14⁺ cells.

FIG. 9A-E illustrates the structures and activities of LEG family compounds. FIG. 9A shows the structures of three legumain prodrugs (LEG-2, LEG-3 and LEG-4). LEG-2 (N-Succinyl-β-alanyl-L-threoinyl-L-Asparaginyl-L-Leucyl-Doxorubicin) (SEQ ID NO:24) and LEG-3 (N-Succinyl-β-alanyl-L-alanyl-L-Asparaginyl-L-Leucyl-Doxorubicin) (SEQ ID NO:25) are oligopeptidic derivatives of doxorubicin that are cell impermeable and can be hydrolyzed to leucine-doxorubicin by extracellular legumain. LEG-4 (N-Succinyl-β-alanyl-L-Asparaginyl-L-Leucyl-Doxorubicin) is similar but is not subject to legumain hydrolysis. FIG. 9B illustrates the cytotoxicity of LEG compounds using legumain⁺293 cells and control wild-type 293 cells. FIG. 9C illustrates legumain-mediated LEG activation and inhibition by cystatin. Bar (1) Untreated control; bar (2) Dox treated; bar (3) Dox plus cystatin; bar (4) LEG-3 treated; bar (5) LEG-3 plus cystatin; bar (6) LEG-2 treated; and bar (7) LEG-2 plus cystatin. FIG. 9D illustrates cellular uptake of LEG-3 compared to Dox. FIG. 9E shows localization of Dox in cell nuclei is visualized by auto-fluorescence (red). In contrast, LEG-3 is not internalized by legumain negative cells exposed to LEG-3 and they lack nuclear positivity for end product Dox despite presence of extracellular fluorescent signal of the Dox present in the LEG-3.

FIG. 10A-C illustrates the in vivo distribution and pharmacokinetics of LEG-3. FIG. 10A shows the plasma pharmacokinetics of LEG-3 compared to Dox. When Dox was administered, the plasma concentration very rapidly declined and then a low Dox concentration was detected that slowly cleared. In contrast, the plasma concentration of LEG-3 remained higher than that of Dox for a longer period of time. FIG. 10B shows accumulation of LEG-3 (dark bars) and Dox (light bars) in organs and tumors of mice bearing CT26 colon carcinomas. FIG. 10C illustrates the presence of Dox and LEG-3 in cells as visualized with auto-fluorescence of Dox (red in original, lighter areas in general).

FIG. 11A-B shows that LEG-3 lacks the in vivo toxicity of Dox. FIG. 11A illustrates the myelosuppression of LEG-3 and Dox in mice as assessed by determination of the numbers of peripheral blood leukocytes. FIG. 11B illustrates the toxicity of LEG-3 in cardiac tissues as demonstrated by the presence of vacuolar degeneration of myocytes in hematoxylin and eosin stained cardiac sections. Chronic Dox treatment resulted in large numbers of vacuoles. In contrast, vacuoles were notably absent in the cells of LEG-3 treated mice. TUNEL analysis of cardiac tissue from mice treated with Dox also demonstrated marked apoptosis of myocytes (apoptotic nuclei are indicated by arrows). This was very infrequent in LEG-3 treated mice.

FIG. 12A-F illustrates the in vivo specificity and efficacy of LEG-3 in syngeneic mouse tumor models. FIG. 12A illustrates the tumor volume after in vivo treatment with LEG-4 in mice bearing CT26 tumors (n=8). FIG. 12B illustrates the tumor volume after in vivo treatment with LEG-2 in mice bearing CT26 tumors (n=8). FIG. 12C illustrates the tumor volume after in vivo treatment with LEG-3 in mice bearing CT26 tumors (n=8). FIG. 12D illustrates the tumor volume after in vivo treatment with LEG-3 in A/J mice bearing C1300 neuroblastomas (n=8). FIG. 12E graphically illustrates the survival of CT26 tumor bearing mice treated with LEG-3 compared to untreated CT26 tumor bearing mice. FIG. 12F graphically illustrates the survival of C1300 bearing mice treated with LEG-3 compared to untreated C 1300 tumor bearing mice.

FIG. 13A illustrates the efficacy of LEG-3 in human xenograft tumor models compared to Dox. FIG. 13A graphically illustrates the tumor volume after in vivo treatment with LEG-3 (49.4 μmol/kg) (n=8) or Dox (1.72 μmol/kg) (n=8) in mice bearing HT1080 human fibrosarcoma tumors compared with control mice bearing HT1080 tumors that were untreated. FIG. 13B graphically illustrates the tumor volume after in vivo treatment with LEG-3 (49.4 μmol/kg) (n=8) or Dox (1.72 μmol/kg) (n=8) in mice bearing MDA-PCa-2b human prostate carcinomas compared to control mice bearing MDA-PCa-2b human prostate carcinomas that were untreated. FIG. 13C graphically illustrates survival of mice bearing HT1080 human fibrosarcoma tumors that were treated or not treated with Dox or LEG-3. FIG. 13D graphically illustrates survival of mice bearing MDA-PCa-2b tumors that were treated or not treated with Dox or LEG-3. FIG. 13E graphically illustrates gross toxicity of Dox and LEG-3 as assessed by weight loss and resistance to death in mice.

FIG. 14A-C illustrates that LEG-3 treatment kills tumor-associated macrophages (TAMs) and reduces the level of angiogenic factors in tumors. FIG. 14A shows immunohistochemically stained sections of CT26 tumors from mice that were treated with LEG-3. Legumain stained green in the original and TAMs are identified by CD68 staining, which was red. FIG. 14B provides an example of angiogenic factor arrays. FIG. 14C illustrates the levels of various angiogenic factors during LEG-3 treatment as a function of time.

FIG. 15 illustrates the efficacy of the liposome-encapsulated LEG-3 prodrug. Liposome LEG-3 had substantial tumor suppressive effect in 4T1 syngeneic murine breast carcinoma models. LEG-3 was administered in a dosage of 40 umol/kg and liposome-encapsulated legubicin was administered at a dosage level of 5 umol/kg.

FIG. 16A-E illustrates that AEPI-1 suppresses tumor metastasis and invasive growth. FIG. 16A shows that while legumain promoted tumor cell invasion in vitro (second bar from left), AEPI-1 inhibited hypoxia induced breast cancer cell migration (fourth bar from left). FIG. 16B shows that AEPI-1 inhibits the growth of 4T1 urine mammary carcinoma. FIG. 16C shows that systemic administration of AEPI-1 inhibited legumain activity in 4T1 tumors, but does not affect legumain activity in kidney, indicating that AEPI-1 is substantially impermeable to cells and therefore not affecting intracellular legumain. FIG. 16D shows that AEPI-1 inhibited experimental lung metastasis of 4T1 mammary carcinoma. FIG. 16E shows that AEPI-1 inhibited spontaneous lung metastasis of 4T1 mammary carcinoma.

FIG. 17A-D shows that LEG-3 suppresses experimental and spontaneous metastasis. FIG. 17A shows that when experimental lung metastasis was induced in mice using 4T1 metastatic breast cancer cells the treatment group, which received LEG-3, exhibited substantially no metastasis but the control group exhibited high levels of metastasis (p<0.005, n=8). FIG. 17B shows that mouse 4T1 mammary carcinoma growth was suppressed by LEG-3 (n=8). FIG. 17C shows that LEG-3 treatment significantly reduced spontaneous lung metastasis in 4T1 tumors (p<0.005). FIG. 17D shows that animal survival was substantially improved by LEG-3 treatment.

FIG. 18 is a synthetic scheme for making paclitaxel prodrugs.

FIG. 19A-G shows that legumain protects cells against apoptosis. FIG. 19A shows inhibition of cathepsin B auto-activation by legumain under different pH conditions. FIG. 19B shows legumain inhibition of cathepsin B auto-activation is dose dependent. FIG. 19C shows that legumain inhibits cathepsin B activation of Bid. FIG. 19D shows that legumain does not inhibit cathepsin B activity towards small peptidyl substrates. FIG. 19E illustrates cathepsin B co-immunoprecipitation with anti-legumain antibodies and legumain coprecipitation with anti-cathepsin B antibodies from cell lysates. FIG. 19F illustrates that legumain binds to immobilized cathepsin B in a dose dependent manner and such binding can be competed off by excess soluble cathepsin B.

FIG. 20 illustrates that legumain plays a key role in modulating the activities of caspase 9, which in turn modulates caspase 3 activity. FIG. 20A shows that legumain cleaves both pro and active caspase 9. FIG. 20B shows that legumain cleavage of caspase 9 resulted in loss of caspase 9 activity. However, the loss of caspase 9 activity was inhibited by AEPI-1. FIG. 20C shows that legumain inactivates pro and active caspase 9 by cleavage in apoptotic cell lysate. Legumain also inactivated caspase 9 in a reconstituted apoptosome formation assay, which led to reduction of caspase 3 activation (FIG. 20D) and reduction of caspase 3 activity (FIG. 20E).

FIG. 21A-E illustrate the effects of intracellular inhibition of legumain by using a variety of AEPIs. FIG. 21A shows the percentages of legumain activities in MDA MB231 breast cancer cells after treatment with AEPI-1, AEPI-2, AEPI-3, AEPI-4 and AEPI-5. Note that AEPI-5 exhibits the greatest inhibition, which indicates that small size of AEPI-5 may facilitate cell entry. FIG. 21B illustrates the percentage of cancer cells that survive combined treatment with tumor necrosis factor-α (TNF-α) and one of the following legumain inhibitors: AEPI-1, AEPI-2, AEPI-3, AEPI-4 and AEPI-5. These data indicate that the combination of TNF-α and AEPI-5 leads to higher levels of cancer cell death, indicating that inhibition of legumain can promote TNF-α induced apoptosis. FIG. 21C shows that legumain-expressing 293 (293L) cells are more resistant to UV compared to 293 control cells that do not express high levels of legumain. FIG. 21D illustrates the percentage of dead cells after UV treatment as a function of AEPI-5 concentration. These data indicate that AEPI-5 treatment sensitizes legumain-expressing 293 cells to UV-induced apoptosis. FIG. 21E illustrates the percentage of dead legumain-expressing 293 (293L) cells with or without AEPI-5 treatment as a function of TNF-α concentration. These data indicate that AEPI-5 sensitizes MDA-MB-231 human breast cancer cells to TNF-α induced cell death.

FIG. 22 schematically illustrates that legumain contributes to tumor progression by promoting tumor cell invasion and metastasis through binding to cell surface integrins and activation of both MMP-2 and cathepsin L. Legumain also protects cells from programmed cell death by catalytic inactivation of caspase 9 and by preventing Bid activation by cathepsin B through binding and modulating cathepsin B activity.

### DETAILED DESCRIPTION OF THE INVENTION

The invention provides compositions and methods for targeting agents to cancerous cells and to the tumor microenvironment, including primary as well as metastatic tumor sites.

In one embodiment, the agent is an inhibitor of a protease that is expressed in the tumor microenvironment. Proteases are involved in many phases of tumor cell biology. A protease that is over-expressed in or on tumor cells can contribute to tumor progression through processing of signaling molecules and their receptors, providing growth signals, digesting extracellular matrices to facilitate cell migration and other processes that contribute to the growth and spreading of tumor cells. Proteases that are active in the tumor microenvironment include legumain (an asparaginyl protease), prostate specific membrane antigen (PSMA)(a carboxypeptidase), fibroblast activation protein (FAP) (a serine peptidase), cathepsin B (a cysteine protease), cathepsin X (a cysteine protease), urokinase-type plasminogen activator (uPA)(a serine protease), tissue factor VIIa (TF VIIa)(a serine protease), matriptase (a membrane-bound serine protease) and Factor XIII. The invention provides specific inhibitors for reducing the activities of these proteases and thereby curtailing the growth and metastasis of tumor cells.

In another embodiment, the agent is a prodrug that is substantially non-toxic to normal, noncancerous cells but that is activated by proteases expressed by cell in the tumor microenvironment (e.g., tumor cells and tumor-associated macrophages). The prodrugs of the invention are highly cytotoxic to tumor cells and to tumor-associated cells. However, in many embodiments, the prodrugs are processed at the cell surface of tumor cells, and are substantially non-toxic to normal cells because the prodrugs do not enter the cells. When the drug or chemotherapeutic agent is cleaved from the prodrug by proteases expressed in the tumor microenvironment (e.g., on the tumor cell surface), the drug or chemotherapeutic agent is released into the tumor microenvironment and can be taken up not only by cancer/tumor cells but also by other tumor-associated cells that support the growth of the tumor.

Cancer markers frequently are not expressed by all cancer cells all the time. Thus, release of the drug or chemotherapeutic agent just outside of a tumor cell gives rise to a “bystander” effect upon nearby tumor cells, stromal cells, tumor-associated macrophages, endothelial cells, vascular cells and other cells that support the growth and development of the tumor. Without such a bystander effect, targeted therapies will often cause tumor cell resistance to the drug or chemotherapeutic agent by “selection” of cancerous tumor cells that do not express the target of the drug or chemotherapeutic agent. Thus, the prodrugs of the invention are specifically designed to be activated by tumor cell-associated proteases present in the tumor microenvironment and attack all cells in the tumor microenvironment.

According to the invention, the present prodrugs and methods often cause tumor-associated macrophages and endothelial cells to die first because, unlike cancer cells, they are not drug resistant. Tumor-associated macrophages provide angiogenic factors and growth factors. Therefore, the outcome of the present methods is an anti-angiogenic effect that results in tumor cell death by starvation. Data provided herein illustrates that the present prodrugs, agents and methods do inhibit angiogenesis.

The agents used in the prodrugs of the invention may be therapeutic agents, cytotoxic agents, photosensitizing agents, chemotherapeutic agents or agents useful for imaging and diagnosis.

### Proteases

According to the invention, certain proteases are expressed by tumor cells and tumor-associated macrophages in response to the unique conditions present in the tumor microenvironment. These protease are specifically expressed and/or secreted by cells in the tumor microenvironment. The proteases therefore exhibit very low or substantially no extracellular activity in normal tissues. Such proteases include legumain (an asparaginyl protease), prostate specific membrane antigen (PSMA)(a carboxypeptidase), fibroblast activation protein (FAP) (a serine peptidase), cathepsin B (a cysteine protease), cathepsin X (a cysteine protease), urokinase-type plasminogen activator (uPA)(a serine protease tissue factor) tissue factor VIIa (TF VIIa)(a serine protease), matriptase (a membrane-bound serine protease) and Factor XIII.

Legumain. According to the invention, legumain, a novel asparaginyl endopeptidase, is preferentially expressed in tumors. As shown herein, legumain was detected in membrane-associated vesicles concentrated at the invadopodia of tumor cells, and, unexpectedly, on cell surfaces where it co-localizes with integrins. In fact, integrin is a cell surface receptor for legumain. Cells that over-expressed legumain possessed increased migratory and invasive activity in vitro, and adopted an invasive and metastatic phenotype in vivo. Accordingly, legumain has a role in tumor invasion and metastasis.

Legumain is a lysosomal protease, and a member of the C13 family of cysteine proteases (Chen et al., 1997). Legumain is evolutionarily conserved and is present in plants, invertebrate parasites, as well as in mammals. An example of an amino acid sequence for a preproprotein of a human legumain can be found in the National Center for Biotechnology Information (NCBI) database (http://www.ncbi.nlm.nih.gov/) at accession number NP 005597 (gi: 21914881), and is reproduced below (SEQ ID NO: 1).

| | |
|---|---|
| 1 | MVWKVAVFLS VALGIGAIPI DDPEDGGKHW VVIVAGSNGW |
| 41 | YNYRHQADAC HAYQIIHRNG IPDEQIVVMM YDDIAYSEDN |
| 81 | PTPGIVINRP NGTDVYQGVP KDYTGEDVTP QNFLAVLRGD |
| 121 | AEAVKGIGSG KVLKSGPQDH VFIYFTDHGS TGILVFPNED |
| 161 | LHVKDLNETI HYMYKHKMYR KMVFYIEACE SGSMMNHLPD |
| 201 | NINVYATTAA NPRESSYACY YDEKRSTYLG DWYSVNWMED |
| 241 | SDVEDLTKET LHKQYHLVKS HTNTSHVMQY GNKTISTMKV |
| 281 | MQFQGMKRKA SSPVPLPPVT HLDLTPSPDV PLTIMKRKLM |
| 321 | NTNDLEESRQ LTEEIQRHLD ARHLIEKSVR KIVSLLAASE |
| 361 | AEVEQLLSER APLTGHSCYP EALLHFRTHC FNWHSPTYEY |
| 401 | ALRHLYVLVN LCEKPYPLHR IKLSMDHVCL GHY |

An example of a nucleotide sequence for a human legumain that encodes SEQ ID NO: 1 can found in the NCBI database at accession number NM_{—}005606 (gi: 21914880). This nucleotide sequence is reproduced below (SEQ ID NO:2).

| | |
|---|---|
| 1 | GGCACGAGGG AGGCTGCGAG CCGCCGCGAG TTCTCACGGT |
| 41 | CCCGCCGGCG CCACCACCGC GGTCACTCAC CGCCGCCGCC |
| 81 | GCCACCACTG CCACCACGGT CGCCTGCCAC AGGTGTCTGC |
| 121 | AATTGAACTC CAAGGTGCAG AATGGTTTGG AAAGTAGCTG |
| 161 | TATTCCTCAG TGTGGCCCTG GGCATTGGTG CCATTCCTAT |
| 201 | AGATGATCCT GAAGATGGAG GCAAGCACTG GGTGGTGATC |
| 241 | GTGGCAGGTT CAAATGGCTG GTATAATTAT AGGCACCAGG |
| 281 | CAGACGCGTG CCATGCCTAC CAGATCATTC ACCGCAATGG |
| 321 | GATTCCTGAC GAACAGATCG TTGTGATGAT GTACGATGAC |
| 361 | ATTGCTTACT CTGAAGACAA TCCCACTCCA GGAATTGTGA |
| 401 | TCAACAGGCC CAATGGCACA GATGTCTATC AGGGAGTCCC |
| 441 | GAAGGACTAC ACTGGAGAGG ATGTTACCCC ACAAAATTTC |
| 481 | CTTGCTGTGT TGAGAGGCGA TGCAGAAGCA GTGAAGGGCA |
| 521 | TAGGATCCGG CAAAGTCCTG AAGAGTGGCC CCCAGGATCA |
| 561 | CGTGTTCATT TACTTCACTG ACCATGGATC TACTGGAATA |
| 601 | CTGGTTTTTC CCAATGAAGA TCTTCATGTA AAGGACCTGA |
| 641 | ATGAGACCAT CCATTACATG TACAAACACA AAATGTACCG |
| 681 | AAAGATGGTG TTCTACATTG AAGCCTGTGA GTCTGGGTCC |
| 721 | ATGATGAACC ACCTGCCGGA TAACATCAAT GTTTATGCAA |
| 761 | CTACTGCTGC CAACCCCAGA GAGTCGTCCT ACGCCTGTTA |
| 801 | CTATGATGAG AAGAGGTCCA CGTACCTGGG GGACTGGTAC |
| 841 | AGCGTCAACT GGATGGAAGA CTCGGACGTG GAAGATCTGA |
| 881 | CTAAAGAGAC CCTGCACAAG CAGTACCACC TGGTAAAATC |
| 921 | GCACACCAAC ACCAGCCACG TCATGCAGTA TGGAAACAAA |
| 961 | ACAATCTCCA CCATGAAAGT GATGCAGTTT CAGGGTATGA |
| 1001 | AACGCAAAGC CAGTTCTCCC GTCCCCCTAC CTCCAGTCAC |
| 1041 | ACACCTTGAC CTCACCCCCA GCCCTGATGT GCCTCTCACC |
| 1081 | ATCATGAAAA GGAAACTGAT GAACACCAAT GATCTGGAGG |
| 1121 | AGTCCAGGCA GCTCACGGAG GAGATCCAGC GGCATCTGGA |
| 1161 | TGCCAGGCAC CTCATTGAGA AGTCAGTGCG TAAGATCGTC |
| 1201 | TCCTTGCTGG CAGCGTCCGA GGCTGAGGTG GAGCAGCTCC |
| 1241 | TGTCCGAGAG AGCCCCGCTC ACGGGGCACA GCTGCTACCC |
| 1281 | AGAGGCCCTG CTGCACTTCC GGACCCACTG CTTCAACTGG |
| 1321 | CACTCCCCCA CGTACGAGTA TGCGTTGAGA CATTTGTACG |
| 1361 | TGCTGGTCAA CCTTTGTGAG AAGCCGTATC CACTTCACAG |
| 1401 | GATAAAATTG TCCATGGACC ACGTGTGCCT TGGTCACTAC |
| 1441 | TGAAGAGCTG CCTCCTGGAA GCTTTTCCAA GTGTGAGCGC |
| 1481 | CCCACCGACT GTGTGCTGAT CAGAGACTGG AGAGGTGGAG |
| 1521 | TGAGAAGTCT CCGCTGCTCG GGCCCTCCTG GGGAGCCCCC |
| 1561 | GCTCCAGGGC TCGCTCCAGG ACCTTCTTCA CAAGATGACT |
| 1601 | TGCTCGCTGT TACCTGCTTC CCCAGTCTTT TCTGAAAAAC |
| 1641 | TACAAATTAG GGTGGGAAAA GCTCTGTATT GAGAAGGGTC |
| 1681 | ATATTTGCTT TCTAGGAGGT TTGTTGTTTT GCCTGTTAGT |
| 1721 | TTTGAGGAGC AGGAAGCTCA TGGGGGCTTC TGTAGCCCCT |
| 1761 | CTCAAAAGGA GTCTTTATTC TGAGAATTTG AAGCTGAAAC |
| 1801 | CTCTTTAAAT CTTCAGAATG ATTTTATTGA AGAGGGCCGC |
| 1841 | AAGCCCCAAA TGGAAAACTG TTTTTAGAAA ATATGATGAT |
| 1881 | TTTTGATTGC TTTTGTATTT AATTCTGCAG GTGTTCAAGT |
| 1921 | CTTAAAAAAT AAAGATTTAT AACAGAACCC AAAAAAAAAA |
| 1961 | AAAAAAAAAA AAAAAAAAAA A |

A genomic nucleotide sequence for human legumain can be found in the NCBI database at accession number NT 026437 (gi: 29736559). See website at ncbi.nlm.nih.gov. Naturally occurring allelic variants of legumain nucleic acids and proteins are also contemplated. An allelic variant is an alternate form of sequence which may have a substitution, deletion or addition at one or more positions, which does not substantially alter the function of the legumain.

Murine legumain shares about 83% sequence identity with the human protein (Barrett et al., 2001). An amino acid sequence for murine legumain can be found in the NCBI database at accession number 089017 (gi: 21617821).

Importantly, the protease specificity of legumain is highly restricted. In particular, legumain cleaves polypeptide sequences on the N-terminal side of asparagine. Hence, legumain requires an asparagine at the P1 site of a substrate in order to cleave a protein or peptide.

As described herein, immunohistochemical analysis of human tumors by high density tissue arrays shows that legumain is highly expressed by a majority of tumors. A high percent of breast carcinomas, colon carcinomas, and central nerve system neoplasms expressed legumain at high levels in vivo. Moreover, data provided herein show that legumain expression is highest in prostate cancers. Importantly, legumain expression is very low or substantially absent in the normal tissues. Legumain is over-expressed in vivo by neoplastic cells as well as endothelial cells and tumor associated macrophages (TAM) in the tumor microenvironment. Interestingly, legumain was not detectable in cancerous cells maintained in culture even though those cells can generate tumors in vivo. However, once these tumor-generating cells are injected into an animal and form a tumor, these tumor cells express high levels of legumain. These observations indicate that legumain gene expression is induced by the in vivo tumor environment.

Data provided herein shows that legumain inhibits cathepsin B auto-activation. Moreover, legumain activates cathepsin L. These data are consistent with legumain knockout study in which the activation of cathepsin H, L was abolished, but cathepsin B activity is actually enhanced (Shirahama-Noda et al., Biosynthetic processing of cathepsins and lysosomal degradation are abolished in asparaginyl endopeptidase-deficient mice. J. BIOL. CHEM. 278(35):33194-99 (2003)). The expression of legumain is highly regulated, in contrast to cathepsin B and D, which are ubiquitously expressed.

Importantly, cathepsins are effectors of invasive growth and angiogenic switch during multistage tumorigenesis (Joyce et al., Cathepsin cysteine proteases are effectors of invasive growth and angiogenesis during multistage tumorigenesis, CANCER CELL. 5(5):443-53 (2004)). Legumain exists in intracellular membranous vesicles indicating it is a lysosomal protease. However, it was also observed in tumors on cell surfaces. Legumain positive vesicles were often concentrated at the invadopodia of tumor cells (Liu, Sun et al. 2003). Membrane trafficking pathways are necessary for delivery of the molecules necessary for pseudopodia protrusion, such as adhesion molecules, actin-binding proteins and proteases.

Data provided herein also indicate that legumain has an anti-apoptotic function. In particular, legumain suppresses mitochondria mediated apoptosis by depleting pro-caspase 9 and inactivating caspase 9 in apoptosomes, therefore reducing caspase 3 activation. This is of concern because legumain is expressed so specifically in tumor cells. Hence, the high levels of legumain that are found in the tumor microenvironment serve to inhibit tumor cell death.

The invention provides therapeutic and diagnostic compositions of prodrugs and other agents that can be targeted to tissues having cancerous cells. In particular, the invention provides tumor environment specific protease inhibitors and prodrugs. Data obtained using prodrug agents designated legubicin and LEG-3, indicate that the present prodrugs exhibit reduced toxicity and enhanced tumoricidal activity in vivo.

Prostate Specific Membrane Antigen. Prostate-specific membrane antigen (PSMA), a type II transmembrane glycoprotein, is overexpressed in prostate cancer. PSMA is a unique cell surface marker, negatively regulated by androgen and extensively used for imaging of hormone refractory carcinomas and metastatic foci. PSMA is a carboxypeptidase with two important enzymatic functions, namely, folate hydrolase and NAALADase. PSMA also exhibits an endocytic function, in which it spontaneously recycles through endocytic vesicles. PSMA is overexpressed at various stages of prostate cancer, including androgen-sensitive and -independent disease, increased in expression with early relapse after therapy.

Fibroblast activation protein (FAP). Fibroblast activation protein (FAP) is a type II cell surface serine protease expressed by fibroblastic cells in areas of active tissue remodeling, including in tumor stroma. Normal adult tissues are generally devoid of detectable FAP, although some foetal mesenchymal tissues transiently express the molecule. FAP has been implicated in tumorigenesis and is selectively expressed by the reactive stroma of epithelial tumors. Most of the common types of epithelial cancers, including >90% of breast, non-small-cell lung, and colorectal carcinomas, contain FAP-reactive stromal fibroblasts. These FAP⁺ fibroblasts accompany newly formed tumor blood vessels, forming a distinct cellular compartment interposed between the tumor capillary endothelium and the basal aspect of malignant epithelial cell clusters. While FAP⁺ stromal fibroblasts are found in both primary and metastatic carcinomas, most benign and premalignant epithelial lesions, such as fibroadenomas of the breast and colorectal adenomas, only rarely contain FAP⁺ stromal cells.

Cathepsin B. Cathepsin B is a lysosomal cysteine proteinase that participates in processes of tumor growth, invasion and metastasis (Kos, J. and Lah, T. T., Oncology Reports 5: 1349-1361, 1996). Cathepsin B is translocated to the plasma membrane or secreted either as a pro-form or as an active enzyme from tumor cells where it takes part in the degradation of the components of extracellular matrix and basement membrane, which is deemed a crucial step in the metastatic process (Sloane et al., Biochemical and Molecular Aspects of Selected Cancers, T. G. Pretlow and T. P. Pretlow eds., Academic Press, New York, pp. 411-465, 1994). Cathepsin B activity is controlled in vivo by endogenous inhibitors of cysteine proteinases—such as the intracellular stefins A and B and extracellular cystatins, kininogens and α2-macroglobulin. Increased levels of cathepsin B in tumors are often not offset by a corresponding increase in cysteine proteinase inhibitors, which can lead to an uncontrolled proteolysis of the extracellular matrix. In clinical studies of breast, head and neck, colorectal and lung cancers, increased cathepsin B activity in the tumor tissue correlated with more aggressive tumor behavior, early relapse and shorter overall survival (Kos, J. and Lah, T. T., Oncology Reports 5: 1349-1361, 1996). Significantly increased levels of cathepsin B have also been found in sera of patients with breast, colorectal, liver, pancreatic and melanoma cancers (Kos et al., Int. J. Biol. Markers, 15:84-89, 2000).

Cathepsin X Cathepsin X is a cysteine protease that exhibits carboxypeptidase activity. Patients with H. pylori gastritis showed significantly higher cathepsin X mRNA (2.5-fold) and protein (1.6-fold) expression than H. pylori-negative patients. Krueger et al., Up-regulation of cathepsin X in Helicobacter pylori gastritis and gastric cancer. J. Pathol. 207: 32-42 (2005). Cathepsin X is also up-regulated in gastric cancer (3-12-fold) compared to non-neoplastic mucosa (id.). Cathepsin X is predominantly expressed by macrophages in the mucosal stroma and in glands of the antral mucosa. In addition, tumor cells stained for cathepsin X in 26 (68%) patients with gastric carcinoma. In general, staining was significantly more common (20 vs. 6 patients) and more intense (3.55 vs. 0.83) in intestinal type gastric cancer than in the diffuse type. In vitro cell culture experiments revealed that intercellular signaling between pathogenicity island (PAI)-positive H. pylori-infected epithelial cells and macrophages via soluble factors in the culture medium seems to be responsible for increased expression of cathepsin X in monocytes. Cathepsin X up-regulation was directly associated with higher invasiveness during in vitro testing (id.).

Urokinase-type plasminogen activator. Urokinase-type plasminogen activator (uPA) is a serine protease which has been implicated in various biological processes, including fibrinolysis, (Carmeliet et al., 1994, Nature 369: 419-424; Carmeliet et al., 1996, Haemostasis 26: 132-153; Pinsky et al., 1998, J. Clin. Invest. 102: 919-928; Bugge et al., 1996, Proc. Natl. Acad. Sci. USA 93: 5899-5904) angiogenesis, (Odekon et al., 1992, J. Cell, Physiol. 150: 258-263; Bacharach et al., 1992, Proc. Natl. Acad. Sci. USA 89: 10686; Pepper et al., 1990, J. Cell Biol. 111: 743-755; Goldberg et al., eds., 1997, In: Regulation of Angiogenesis Birkhauser Verlag, Basel, pp. 391-411) neointima and aneurysm formation, (Clowes et al., 1990, Circ. Res. 67: 61-67; Carmeliet et al., 1997, Circ. Res. 81: 829-839; Shireman et al., 1997, J. Vasc. Surg. 25: 157-164; Noda-Heiny et al., 1995, Arterioscler. Thromb. Vasc. Biol. 15: 37-43; Lijnen et al., 1998, Arterioscler. Thromb. Vasc. Biol. 18: 1035-1045) chemotaxis, (Pedersen et al., 1996, Br. J. Haematol. 95: 45-51) and wound healing (Carmeliet et al., 1998, J. Cell Biol. 140: 233-245). Certain of these activities may require the proteolytic activity of uPA, (Kirschheimer et al., 1987, Fed. Am. Soc. of Exper. Biol. and Med. Journal 1: 125-128; DePetro et al., 1994, Exp. Cell Res. 213: 186-194) whereas others involve intracellular signaling through the urokinase receptor (uPAR) (Pedersen et al., 1996, Br. J. Haematol. 95: 45-51; Degryse et al., 1999, Blood 94: 649-662).

Proteases of the plasminogen activator system, including the urokinase-type plasminogen activator (uPA), play a determining role in the invasive growth of tumors as well as the development of tumor metastasis. The uPA-receptor (uPAR; CD87) also has a role due to its ability to focus and activate the uPA enzyme system on the cell surface. Moreover, urokinase-type plasminogen activator receptors (uPAR) are selectively overexpressed in glioblastoma multiforme, an aggressive form of brain cancer (Mori et al. (2000) J. Neuroonc. 46:115-123). In addition, uPAR expression is correlated with the invasive activity of glioma cells. uPAR also is overexpressed in a number of other tumors, including cancers of the breast, skin, colon, ovaries, thyroid, stomach, liver, and prostate (see Fabbrini et al. (1997) FASEB J. 11:1169-1176; and Rajagopal and Kreitman (2000) J. Biol. Chem. 275:7566-7573). Furthermore, uPAR is expressed on the endothelial cells that make up tumor microvasculature. Thus, uPAR is a useful target for delivery of chemotherapeutic agents by its ligand uPA (e.g. a uPA-related prodrug). Moreover, inhibition of uPA can inhibit the intracellular signaling events that contribute to tumor cell growth and metastasis.

Tissue factor VIIa (TF VIIa). Tissue factor VIIa is a serine protease complex that participates in blood coagulation by activating factor X and/or factor IX. Factor VIIa is produced from its precursor, factor VII, which is synthesized in the liver and secreted into the blood where it circulates as a single chain glycopeptide. Inhibitors of TF VIIa inhibit both primary and metastatic tumor growth in mice. These results indicate that proteolytic activity of TF VIIa promotes tumor growth and angiogenesis through proangiogenic mechanism that acts independently of hemostasis. Hembrough et al., Cancer Research 63:2997-3000 (2003).

Matriptase. Matriptase is a membrane-bond serine protease that is over-expressed in a variety of epithelial-derived tumors, including breast, prostate, ovarian, colon, and oral carcinomas. Moreover, women with breast and ovarian cancer have poor prognoses if their tumors contain high levels of matriptase. In addition, researchers have found that increased expression of matriptase is associated with more serious forms of cervical cancer.

Factor XIII Factor XIII is an enzyme (EC 2.3.2.13) of the transglutaminase family that is similar to papain family of systeine proteases. Like other transglutaminases, it can crosslinks proteins between an ε-N of a lysine residue and a glutamine residue in two protein chains, creating a bond that is highly resistant to proteolysis (protein degradation). Examples of inhibitor of Factor XIII are derivatives of 1,2,4-thiadiazoles, which can be included in the prodrugs and inhibitors of the invention.

### Inhibitors of Tumor-Associated Proteases

The present invention also provides inhibitors of tumor-associated protease and methods of using these inhibitors, for example, in the treatment of cancer. Thus, the inhibitors can inhibit cancer cell growth or tumor progression or tumor metastasis or invasion, by inhibiting the expression or enzymatic activity of a tumor-associated protease. Moreover, the protease inhibitors can promote apoptosis in tumor cells, inhibit angiogenesis in tumors and affect tumor extracellular matrix processing. These effects not only reduce tumor growth but also enhance the penetration and/or retention of other therapeutic agents.

In another embodiment, the protease inhibitor may be used to deliver a drug to a cell that expresses the inhibitor. When a protease inhibitor is used to deliver a drug to a cell, the inhibitor preferably does not substantially block or inhibit the activity of the drug.

Legumain and other cysteine protease inhibitors are available in the art. See, e.g. Asgian, J. L., et al., Aza-peptide epoxides: a new class of inhibitors selective for clan CD cysteine proteases. J Med Chem, 2002. 45(23): p. 4958-60; Niestroj, A. J., et al., Inhibition of mammalian legumain by michael acceptors and AzaAsn-halomethylketones. Biol Chem, 2002. 383(7-8): p. 1205-14; and U.S. Pat. No. 6,004,933, which are incorporated herein by reference. The invention contemplates using any such inhibitors as blocking or delivery agent in legumain-expressing cells.

The invention is therefore directed to a protease inhibitor having the structure:

R₁-peptide-Y

More specific examples of the protease inhibitors of the invention include compounds of formulae III, IV, V or VI:

| | | |
|---|---|---|
| R₁-(Xaa4)ₙ-Asn-Y | (SEQ ID NO:26) | III |
| R₁-(Xaa4)ₙ-Xaa5-Y | (SEQ ID NO:27) | IV |
| R₁-Xaa4-azaAsn-Y | | V |
| R₁-Xaa4-azaXaa5-Y | | VI |

wherein:
wherein R₁ is hydrogen, a hydrophilic group, a hydrophobic group, a photosensitizing agent, a label or an amino protecting group;
n is an integer of about 2 to about 5;
each Xaa4 and Xaa5 is an amino acid or an amino acid mimetic;
Y is alkyl, alkenyl epoxide, fluoromethylketone or a Michael acceptor, wherein the alkyl is optionally substituted with 1-3 halo or hydroxy, alkylamino, dialkylamino, alkyldialkylamino, or cycloalkyl, alkylcycloalkyl, alkenylcycloalkyl, aryl; (C₅-C₁₂)arylalkyl or (C₅-C₁₂)arylalkenyl,
wherein the aryl groups of the arylalky or arylalkenyl can be 0-4 heteroatoms selected from N, O and S, and are optionally substituted with halo, cyano, nitro, haloalkyl, amino, aminoalkyl, dialkylamino, alkyl, alkenyl, alkynyl, alkoxy, haloalkoxy, carboxyl, carboalkoxy, alkylcarboxamide, (C₅-C₆)aryl, —O—(C₅-C₆)aryl, arylcarboxamide, alkylthio or haloalkylthio; and
wherein each of the inhibitors of formulae III, IV, V and VI bind to a protease expressed in a tumor microenvironment.

Michael acceptors include functional groups that bond covalently to active-site nucleophiles. Such Michael acceptors can form the basis for potent and selective protease inhibitors. Examples of Michael acceptors useful in the inhibitors of the invention include N-Ac-L-Phe-NHCH₂CH═CH-E and R—NHCH₂CH═CHCOOCH₃ where E is an electron withdrawing group and R is an amino acid (e.g. Asn or azaAsn). Examples of suitable E electron withdrawing groups include CO₂CH₃, SO₂CH₃, CO₂H, CN, CONH₂ and C₆H₄-p-NO₂.

Examples of asparaginyl endopeptidase inhibitors (AEPIs) that may be used to inhibit legumain and other asparaginyl endopeptidases in the methods of the invention include peptides with the following sequences:

R₁-Ala-Ala-AzaAsn-EP-COOEt

R₁-Ala-Ala-Asn-EP-COOR₃

R₁-Ala-AzaAsn-EP-COOEt.

R₁-Ala-AzaAsn-EP-COOR₃

R₁-Xaa4-Xaa4-AzaAsn-EP-COOEt

R₁-Xaa4-Xaa4-AzaAsn-EP-COOR₃

R₁-Xaa4-AzaAsn-EP-COOEt

R₁-Xaa4-AzaAsn-EP-COOR₃

R₁-AzaAsn-EP-COOEt

R₁-AzaAsn-EP-COOR₃

wherein R₁ is as defined herein; each Xaa4 is separately an amino acid; AzaAsn is azido-asparagine; EP is epoxy; and R₃ is an aryl alcohol, an aliphatic alcohol or a heterocyclic alcohol.

Further examples of asparaginyl endopeptidase inhibitors (AEPIs) that may be used to inhibit legumain and other asparaginyl endopeptidases in the methods of the invention include the following compounds.

AEPI-1 is R₁-Ala-Ala-AzaAsn-(S,S)-EPCOOEt, a compound of the structure:

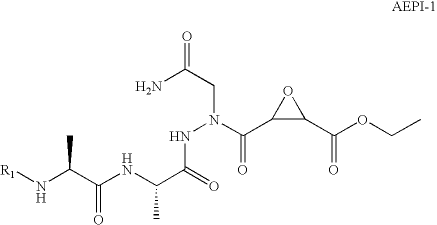

AEPI-2 is R₁-Ala-Ala-AzaAsn-CH═CH—COOEt, for example, a compound of the structure:

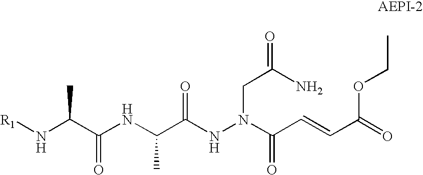

AEPI-3 is R₁-Ala-Ala-AzaAsn-CH═CH—COOBzl, for example, a compound of the structure:

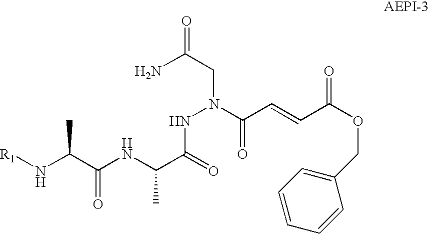

AEPI-4 is R₁-Ala-Ala-AzaAsn-CH═CH—CON(CH₃)Bzl, for example, a compound of the structure:

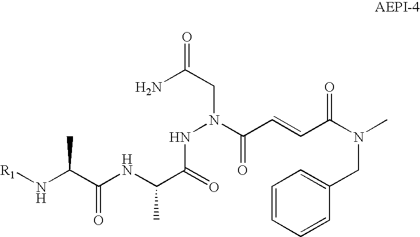

AEPI-5 is N-acetyl-Ala-Ala-AzaAsn-(S,S)-EPCOOEt, a compound of the structure:

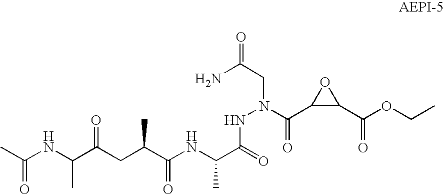

AEPI-6 is N-succinyl-Ala-Ala-AzaAsn-(S,S)-EPCOOEt, a compound of the structure:

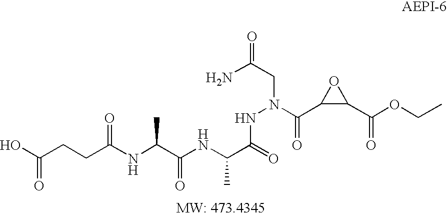

AEPI-7 is N-hepatanoyl-Ala-Ala-AzaAsn-(S,S)-EPCOOEt, a compound of the structure:

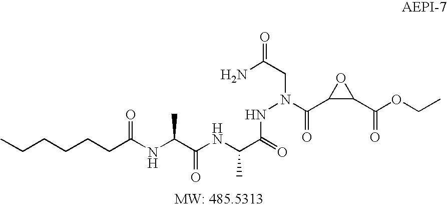

AEPI-8 is 3-(N-carbamoylmethyl-N′-{fluorescein]-methylsulfanyl}-N-propionyl)-Ala-Ala-AzaAsn-(S,S)-EPCOOEt, a compound of the structure:

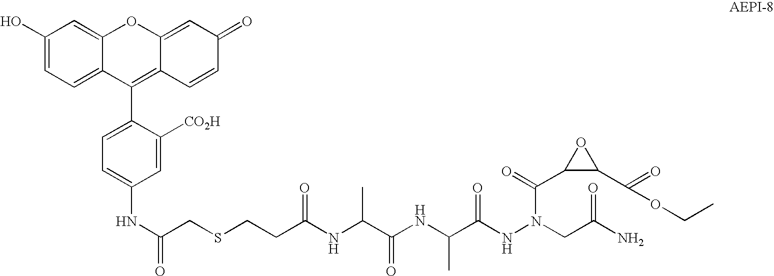

AEPI-9 is N-triethylamino-Ala-(N-isopropanoyl-Ala-AzaAsn-(S,S)-EPCOOEt), a compound of the structure:

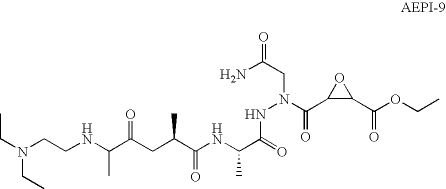

wherein R₁ is hydrogen, a hydrophilic group, a hydrophobic group, a photosensitizing agent, a label or an amino protecting group; Ep is epoxy; Et is ethyl; and Bzl is benzyl.

Inhibitors of PSMA that can be used in the invention include the following:

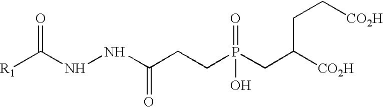

R₁ is hydrogen, a hydrophilic group, a hydrophobic group, a photosensitizing agent, a label or an amino protecting group.

Inhibitors of fibroblast activation protein (FAP) that can be used in the invention include the following:

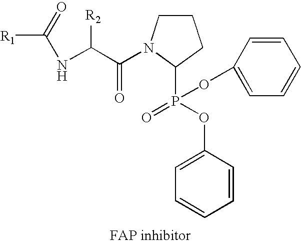

R₁ is hydrogen, a hydrophilic group, a hydrophobic group, a photosensitizing agent, a label or an amino protecting group; and R₂ is hydrogen, hydroxymethylene (CH₂OH), lower alkyl (e.g., methyl, ethyl, propyl, isopropyl (CH(CH₃)₂), butyl, isobutyl), or benzyl.

Cathepsin B inhibitors useful in the invention include the following:

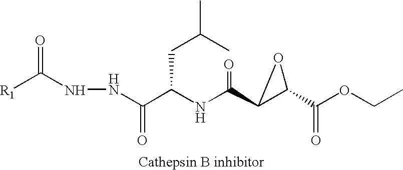

wherein R₁ is hydrogen, a hydrophilic group, a hydrophobic group, a photosensitizing agent, a label or an amino protecting group.

Cathepsin X inhibitors useful in the invention include the following:

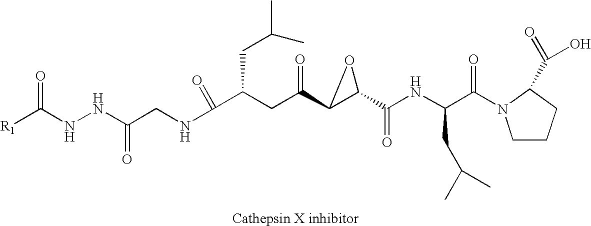

wherein R₁ is hydrogen, a hydrophilic group, a hydrophobic group, a photosensitizing agent, a label or an amino protecting group.

Urokinase-type plasminogen activator (uPA) inhibitors useful in the invention include the following:

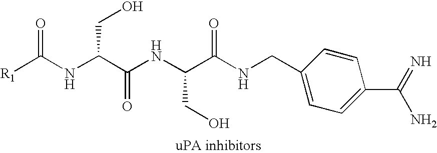

wherein R₁ is hydrogen, a hydrophilic group, a hydrophobic group, a photosensitizing agent, a label or an amino protecting group.

Tissue factor VIIa (TF VIIa) inhibitors useful in the invention include the following:

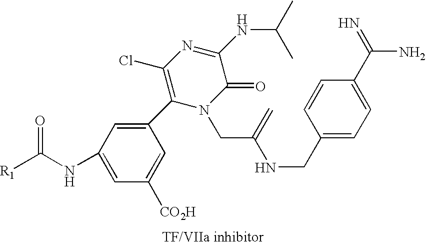

wherein R₁ is hydrogen, a hydrophilic group, a hydrophobic group, a photosensitizing agent, a label or an amino protecting group.

Matriptase inhibitors useful in the invention include the following.

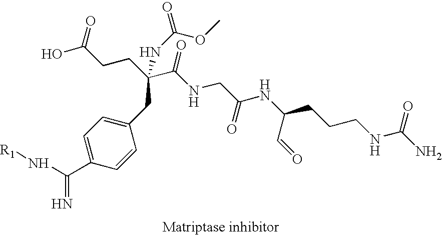

wherein R₁ is hydrogen, a hydrophilic group, a hydrophobic group, a photosensitizing agent, a label or an amino protecting group.

Factor XIII inhibitors useful in the invention include the following:

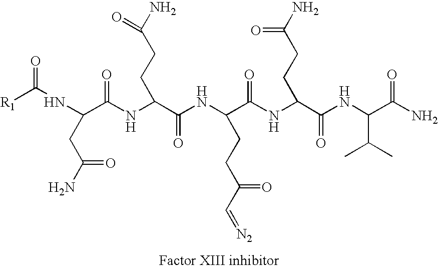

wherein R₁ is hydrogen, a hydrophilic group, a hydrophobic group, a photosensitizing agent, a label or an amino protecting group.

As described herein, insome embodiments R₁ is a photosensitizing agent. Examples of photosensitizing agents that can be used with the inhibitors are described herein, for example, under the section entitled “Drugs.” Essentially any photosensitizing agent can be used, including those contemplated for use with the prodrugs of the invention.

Specific examples of inhibitors with photosentizing agents (e.g., chlorin e6 and include the following.

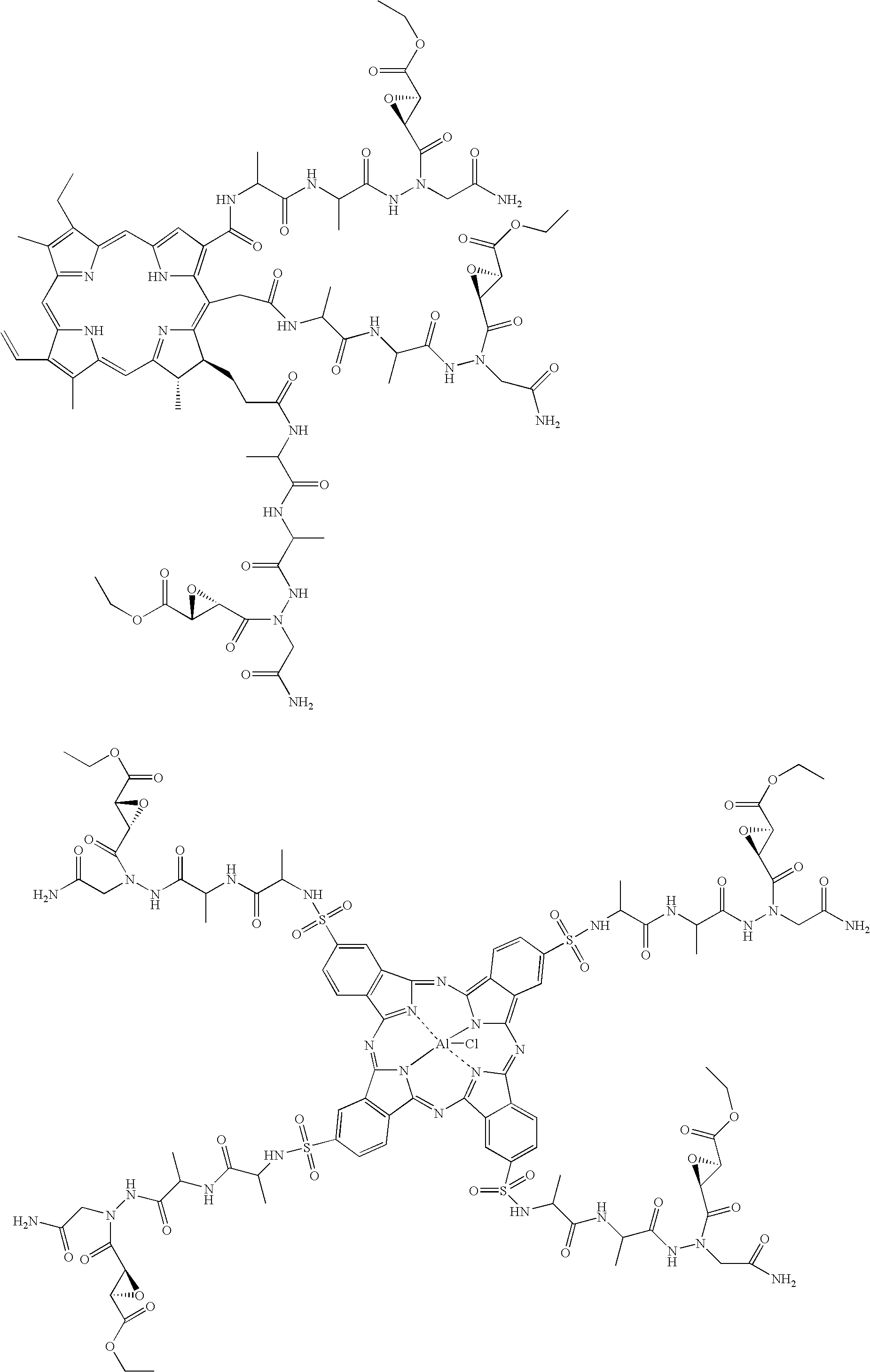

The photosensitizing agents can be linked to the peptides by methods available in the art. For example, chlorine e6 can be linked to a peptide inhibitor using the following procedures.

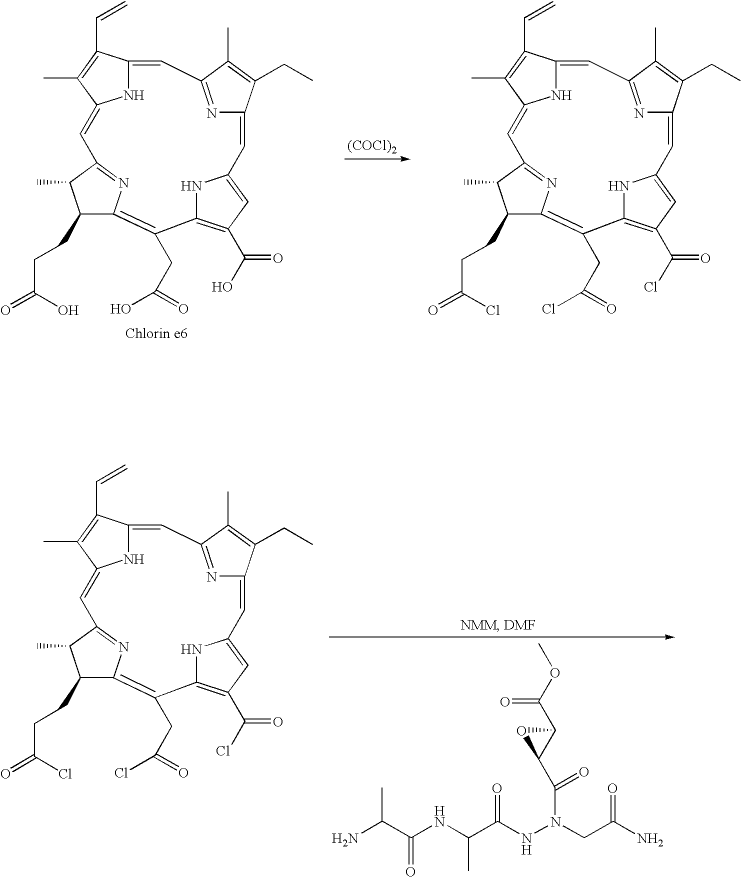

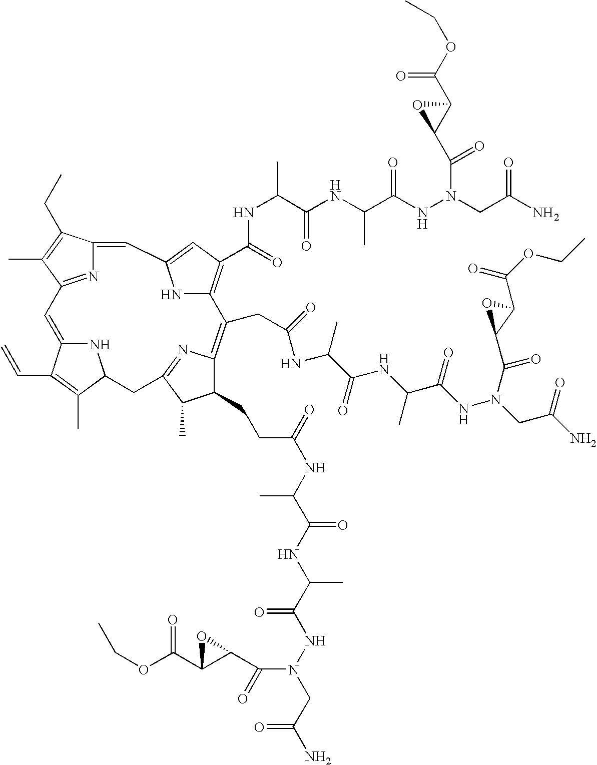

In addition, the aluminum phthalocyanine tetrasulfonate (AlPcS4) group can be prepared for linkage to the peptide inhibitors using procedures available in the art, for example, generally as follows.

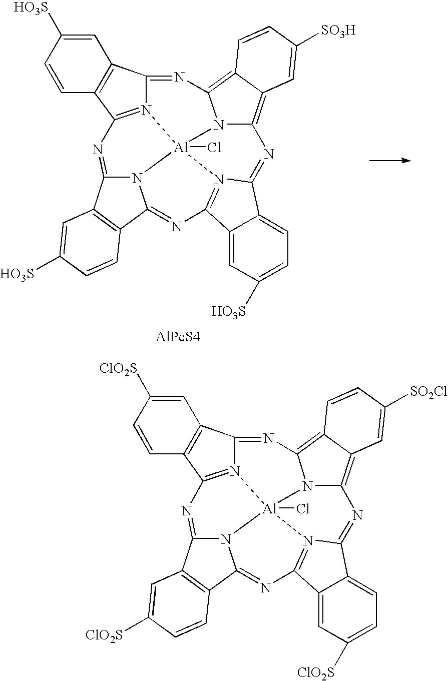

One or more peptides are linked to the AlPcS4 moeity by replacing the chloride of the SO₂Cl group(s).

Hydrophilic R₁ groups of the invention can be sugars (monosaccharides and disaccharides), dicarboxylic acids (e.g., succinate, malate, fumarate, oxaloacetate, citrate, isocitrate), glycans, polyalkylene oxides, lower alkyl carboxylates, carboxyalkyls, carboxyalkylene carboxylates, charged amino acids and the like.

Hydrophobic R₁ groups of the invention can be alkyl, aryl, alkylene aryl, arylalkyl, hydrophobic amino protecting agents, beta-alanyl and related hydrophobic groups.

Any convenient amino protecting group available in the art can be used in the invention including, for example, carbobenzyloxy (Cbz), tert-butyloxycarbonyl (BOC), 9-fluorenylmethyloxycarbonyl (FMOC) and benzyl groups.

A variety of labels can be used with the inhibitors of the invention to generate imaging agents or reagents for detection of cancer and such labels are discussed hereinbelow.

In other embodiments, the legumain inhibitor can be a legumain catalytic inhibitor, such as cystatin, stefin, Tissue Inhibitor of Metalloproteinase 2 (TIMP-2) and a peptide having the sequence Ala-Leu-β-Asn-Ala-Ala (SEQ ID NO: 15).

The Ala-Leu-β-Asn-Ala-Ala (SEQ ID NO: 15) peptide is a synthetic suicide inhibitor that is useful as a legumain inhibitor, where the beta linkage is present to prevent hydrolysis. The SEQ ID NO: 15 peptide will then remain bound to the legubicin catalytic site and block its activity.

The cystatins constitute a superfamily of evolutionarily related proteins that are all composed of at least one 100-120-residue domain with conserved sequence motifs. The single-domain human members of this superfamily are of two major types. The type 1 cystatins (or stefins) A and B contain approximately 100 amino acid residues, lack disulfide bridges, and are synthesized without signal peptides. Cystatins of type 2 are secreted proteins of approximately 120 amino acid residues (Mᵣ 13,000-14,000) and contain at least two characteristic intrachain disulfide bonds. The type 2 cystatins include the human cystatins C, D, S, SN, and SA, which are all products of genes located in the cystatin multigene locus on-chromosome 20. Two recently identified type 2 cystatins, cystatin E/M and cystatin F (also called leukocystatin), are also secreted low molecular weight proteins but are more atypical in that they are glycoproteins and show only 30-35% sequence identity in alignments with the classical type 2 cystatins. They are, however, still functional inhibitors of family C1 cysteine peptidases. It has been shown that the cystatin inhibition of cysteine peptidases of the papain family is due to a tripartite wedge-shaped structure with very good complementarity to the active site clefts of such enzymes.

The three parts of the cystatin polypeptide chain included in the enzyme-binding domain are the N-terminal segment, a central loop-forming segment with the motif Gln-Xaa-Val-Xaa-Gly (SEQ ID NO:28), and a second C-terminal hairpin loop typically containing a Pro-Trp pair.

Both types of cystatins inhibit legumain (cystatin C, E/M, F). Sequence alignments and molecular modeling have indicated that a loop located on the opposite side to the papain-binding surface, between the α-helix and the first strand of the main β-pleated sheet of the cystatin structure, may be involved in legumain binding.

Human cystatin C can inhibit legumain, as shown for example by Alvarez-Fernandez et al., J. Biol. Chem. 274: 19195-203 (1999). Several sequences for cystatin C are available, for example, in the NCBI database database. See website at ncbi.nlm.nih.gov. One such human cystatin C sequences has accession number CAA29096 (gi: 755738), and is provided below for easy reference (SEQ ID NO: 17).

| | |
|---|---|
| 1 | MAGPLRAPLL LLAILAVALA VSPAAGSSPG KPPRLVGGPM |
| 41 | DASVEEEGVR RALDFAVGEY NKASNDMYHS RALQVVRARK |
| 81 | QIVAGVNYFL DVELGRTTCT KTQPNLDNCP FHDQPHLKRK |
| 121 | AFCSFQIYAV PWQGTMTLSK STCQDA |

The first 26 amino acids of this cystatin C polypeptide is a signal peptide (MAGPLRAPLL LLAILAVALA VSPAAG, SEQ ID NO:18) which when cleaved yields a mature cystatin C polypeptide of the following sequence (SEQ ID NO:19).

| | |
|---|---|
| 27 | SSPG KPPRLVGGPM |
| 41 | DASVEEEGVR RALDFAVGEY NKASNDMYHS RALQVVRARK |
| 81 | QIVAGVNYFL DVELGRTTCT KTQPNLDNCP FHDQPHLKRK |
| 121 | AFCSFQIYAV PWQGTMTLSK STCQDA |

As used herein, a mutated cystatin is a cystatin C polypeptide that lacks the N-terminal 10 ten amino acids or contains alanine at the Arg-8 position (R8A mutant), or contains alanine at the Leu-9 position (L9A mutant), or contains alanine at the Val-10 position (V10A) of the mature cystatin C polypeptide. In some embodiments, the mutated cystatin has all three mutations (R8A and L9A and V10A). Other mutations can also be present within the legumain binding region, but not in the region involved in cathepsin binding. These cystatin mutants will inhibit legumain specifically without inhibitory activity against cathepsin B or other cathepsins.

Stefin B, also called cystatin B, can also inhibit cysteine proteinases such as legumain. Several sequences for cystatin B (stefin B) are available, for example, in the ncbi database database. See website at ncbi.nlm.nih.gov. One such human cystatin B sequences has accession number NP000091 (gi: 4503117), and is provided below for easy reference (SEQ ID NO:20).

| | |
|---|---|
| 1 | MMCGAPSATQ PATAETQHIA DQVRSQLEEK ENKKFPVFKA |
| 41 | VSFKSQVVAG TNYFIKVHVG DEDFVHLRVF QSLPHENKPL |
| 81 | TLSNYQTNKA KHDELTYF |

In other embodiments, the invention provides anti-sense RNA or DNA molecules to modulate legumain expression, legumain translation and/or the degradation of legumain transcripts. For example, an anti-sense RNA or DNA that can hybridize to a legumain nucleic acid can be used as an anti-sense RNA or DNA for diminishing the expression of legumain. The legumain nucleic acid can have SEQ ID NO:2 or can have a sequence related to a genomic nucleotide sequence for human legumain that can be found in the NCBI database at accession number NT 026437 (gi: 29736559). See website at ncbi.nlm.nih.gov.

The degradation of legumain mRNA may also be increased upon exposure to small duplexes of synthetic double-stranded RNA through the use of RNA interference (siRNA or RNAi) technology. Scherr, M et al. Curr Med Chem 2003 10:245; Martinez, L A et al. 2002 PNAS 99: 14849. A process is therefore provided for inhibiting expression of legumain in a cell. The process includes introduction of RNA with partial or fully double-stranded character into the cell or into the extracellular environment. Inhibition is specific to legumain RNA because a nucleotide sequence from a portion of the legumain gene is chosen to produce inhibitory RNA. This process is effective in producing inhibition of gene expression.

The pSuppressor Neo vector for expressing hairpin siRNA, commercially available from IMGENEX (San Diego, Calif.), can be used to generate siRNA for inhibiting legumain expression. The most critical part of the construction of the siRNA expression plasmid is the selection of the target region of the mRNA, which is currently a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26(2): p. 199-213. Accordingly for synthesis of synthetic siRNA, a target region may be selected preferably 50 to 100 nucleotides downstream of the start codon. The 5′ and 3′ untranslated regions and regions close to the start codon should be avoided as these may be richer in regulatory protein binding sites. The ideal sequence for a synthetic siRNA is 5′-AA(N19)UU, where N is any nucleotide in the mRNA sequence and should be approximately 50% G-C content. The selected sequence(s) can be compared to others in the human genome database to minimize homology to other known coding sequences (Blast search, for example, through the NCBI website).

However, for designing oligonucleotides for the expression vector, AA and UU dimers in the sequence are not needed. For the expression vector, siRNA can be designed to produce hairpin RNAs, in which both strands of an siRNA duplex would be included within a single RNA molecule. The individual motif can be 19-21 nucleotides long and correspond to the coding region of the legumain gene. However, Paddison and Hannon, 2002 have suggested use of 18-28 nucleotides. Paddison, P. J. and G. J. Hannon, RNA interference: the new somatic cell genetics? Cancer Cell, 2002. 2(1): p. 17-23; Paddison, P. J., et al., Short hairpin RNAs (shRNAs) induce sequence-specific silencing in mammalian cells. Genes Dev, 2002. 16(8): p. 948-58. The two motifs that form the inverted repeat are separated by a spacer of 4-9 nucleotides to permit formation of a hairpin loop. The transcriptional termination signal for 5 T's is added at the 3′ end of the inverted repeat.

The siRNA insert can be prepared by synthesizing and annealing of two complementary oligonucleotides, and directly ligated this insert into the vector DNA. The resultant legumain suppressing vector DNA can be used to generate cell line that stably incorporates this vector and selection for retention of the construct can be achieved by selection of a linked marker. Such cell line is useful for preparing siRNA molecules for use in inhibiting legumain.

Mixtures and combinations of such siRNA molecules are also contemplated by the invention. These compositions can be used in the methods of the invention, for example, for treating or preventing cancer or metastasis. These compositions are also useful for modulating (e.g. decreasing) legumain expression.

The siRNA provided herein can selectively hybridize to RNA in vivo or in vitro. A nucleic acid sequence is considered to be “selectively hybridizable” to a reference nucleic acid sequence if the two sequences specifically hybridize to one another under physiological conditions or under moderate stringency hybridization and wash conditions. In some embodiments the siRNA is selectively hybridizable to an RNA (e.g. a legumain RNA) under physiological conditions. Hybridization under physiological conditions can be measured as a practical matter by observing interference with the function of the RNA. Alternatively, hybridization under physiological conditions can be detected in vitro by testing for siRNA hybridization using the temperature (e.g. 37° C.) and salt conditions that exist in vivo.

Moreover, as an initial matter, other in vitro hybridization conditions can be utilized to characterize siRNA interactions. Exemplary in vitro conditions include hybridization conducted as described in the Bio-Rad Labs ZetaProbe manual (Bio-Rad Labs, Hercules, Calif.); Sambrook et al., Molecular Cloning: A Laboratory Manual, 2nd ed., Cold Spring Harbor Laboratory Press, (1989), or Sambrook et al., Molecular Cloning: A Laboratory Manual, 3rd ed., Cold Spring Harbor Laboratory Press, (2001)), expressly incorporated by reference herein. For example, hybridization can be conducted in 1 mM EDTA, 0.25 M Na₂ HPO₄ and 7% SDS at 42° C., followed by washing at 42° C. in 1 mM EDTA, 40 mM NaPO₄, 5% SDS, and 1 mM EDTA, 40 mM NaPO₄, 1% SDS. Hybridization can also be conducted in 1 mM EDTA, 0.25 M Na₂ HPO₄ and 7% SDS at 60° C., followed by washing in 1 mM EDTA, 40 mM NaPO₄, 5% SDS, and 1 mM EDTA, 40 mM NaPO₄, 1% SDS. Washing can also be conducted at other temperatures, including temperatures ranging from 37° C. to at 65° C., from 42° C. to at 65° C., from 37° C. to at 60° C., from 50° C. to at 65° C., from 37° C. to at 55° C., and other such temperatures.

The siRNA employed in the compositions and methods of the invention may be synthesized either in vivo or in vitro. In some embodiments, the siRNA molecules are synthesized in vitro using methods, reagents and synthesizer equipment available to one of skill in the art. Endogenous RNA polymerases within a cell may mediate transcription in vivo, or cloned RNA polymerase can be used for transcription in vivo or in vitro. For transcription from a transgene or an expression construct in vivo, a regulatory region may be used to transcribe the siRNA strands.

Depending on the particular sequence utilized and the dose of double stranded siRNA material delivered, the compositions and methods may provide partial or complete loss of function for the target gene (legumain). A reduction or loss of gene expression in at least 99% of targeted cells has been shown for other genes. See, e.g., U.S. Pat. No. 6,506,559. Lower doses of injected material and longer times after administration of the selected siRNA may result in inhibition in a smaller fraction of cells.

The siRNA may comprise one or more strands of polymerized ribonucleotide; it may include modifications to either the phosphate-sugar backbone or the nucleoside. The double-stranded siRNA structure may be formed by a single self-complementary RNA strand or two complementary RNA strands. siRNA duplex formation may be initiated either inside or outside the cell. The siRNA may be introduced in an amount that allows delivery of at least one copy per cell. Higher doses of double-stranded material may yield more effective inhibition.

Inhibition is sequence-specific in that nucleotide sequences corresponding to the duplex region of the RNA are targeted for genetic inhibition. siRNA containing nucleotide sequences identical to a portion of the target gene is preferred for inhibition. However, siRNA sequences with insertions, deletions, and single point mutations relative to the target sequence have also been found to be effective for inhibition. Thus, sequence identity may optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

The siRNA may be directly introduced into the cell (i.e., intracellularly); or introduced extracellularly into a cavity, interstitial space, into the circulation of an organism, introduced orally, or may be introduced by bathing an organism in a solution containing siRNA. Methods for oral introduction include direct mixing of siRNA with food of the organism, as well as engineered approaches in which a species that is used as food is engineered to express an siRNA, then fed to the organism to be affected. Physical methods of introducing nucleic acids include injection directly into the cell or extracellular injection into the organism of an siRNA solution.

The siRNA may also be delivered in vitro to cultured cells using transfection agents available in the art such as lipofectamine or by employing viral delivery vectors such as those from lentiviruses. Such in vitro delivery can be performed for testing purposes or for therapeutic purposes. For example, cells from a patient can be treated in vitro and then re-administered to the patient.

The advantages of using siRNA include: the ease of introducing double-stranded siRNA into cells, the low concentration of siRNA that can be used, the stability of double-stranded siRNA, and the effectiveness of the inhibition. The ability to use a low concentration of a naturally-occurring nucleic acid avoids several disadvantages of anti-sense interference.

### Prodrugs

In some embodiments of the invention, agents are designed to contain a drug compound that is activated following cleavage by a protease that is expressed in the tumor microenvironment. Because the proteases exhibit very low or substantially no extracellular activity in normal tissues, the prodrugs are only cleaved and activated in the tumor microenvironment. Hence, highly toxic compounds can be used in the present prodrugs and these toxins exhibit substantially no toxicity in normal tissues. Only upon cleavage by the tumor-specific proteases are the toxins or drugs activated.

The invention is therefore directed to a prodrug compound, including a drug molecule linked to a peptide, wherein the peptide has an amino acid sequence that can be cleaved by a tumor-specific protease. As described above, such proteases include legumain (an asparaginyl protease), prostate specific membrane antigen (PSMA)(a carboxypeptidase), fibroblast activation protein (FAP) (a serine peptidase), cathepsin B (a cysteine protease), cathepsin X (a cysteine protease), urokinase-type plasminogen activator (uPA)(a serine protease) and tissue factor VIIa (TF VIIa)(a serine protease).

Thus, the peptide includes at least two linked amino acids, wherein at least one of the two linked amino acids is an amino acid that is specifically recognized by a tumor-specific protease and forms part of a cleavage site for the protease. For example, legumain is a protease that is specifically expressed in tumor cells and cells that support tumor growth and metastasis (e.g. tumor associated macrophages). Legumain is an asparaginyl protease that specifically recognizes asparagine-containing peptides and cleaves peptides that contain asparagine (Asn). Some of the prodrugs of the invention are therefore designed to be activated by legumain. Legumain cleaves the peptide of the present prodrugs at the site of the Asn to generate an active drug from the prodrug. Prior to cleavage, the prodrug is substantially non-toxic to normal animal cells, whereas after cleavage, the drug is an active drug that can have a beneficial effect upon an animal to which it is administered.

Prodrugs of the invention have the general structure:
R₁-peptide-drug

wherein R₁ is hydrogen, a hydrophilic group, a hydrophobic group, a photosensitizing agent, a label or an amino protecting group. In some embodiments, where it is desirable to prevent cellular uptake of the prodrug prior to cleavage, the R₁ groups of the present prodrugs are not hydrophobic groups. Moreover, where drug delivery rather than detection is desired, R₁ is not a label.

The R₁ and drug moieties can be linked directly to the peptide or they can be linked to the peptide through a linker or spacer molecule. Such a linker of spacer can be an alkyl, a sugar or a oligosaccharide.

The term “drug” as used herein, refers to any medicinal substance used in humans or other animals. Encompassed within this definition are chemotherapeutic agents, cytotoxic agents, compound analogs, hormones, photosensitizing agents, antimicrobials, neurotransmitters, etc. In general, the prodrugs of the invention include drug molecules whose activity is diminished when attached to R₁-peptide.

For example, in some embodiments the prodrug can have a structure similar to that provided for SEQ ID NO:3 or SEQ ID NO:23:

| | |
|---|---|
| R₁-(Xaa1)ₙ-Xaa2-Asn-(Xaa3)-drug | (SEQ ID NO:3) |
| R₁-(Xaa1)ₙ-Xaa2-Xaa3-drug | (SEQ ID NO:23) |

wherein:
R₁ is as defined herein;
n is an integer of about 0 to about 50;
Xaa1 and Xaa2 are separately any amino acid;
Xaa3 is either nothing or an amino acid that has no substantial effect on the activity of the drug; and
the drug employed is a drug whose action is diminished or blocked by attachment of a peptide to the drug.

Specific examples of prodrug compounds of the invention include, for example:

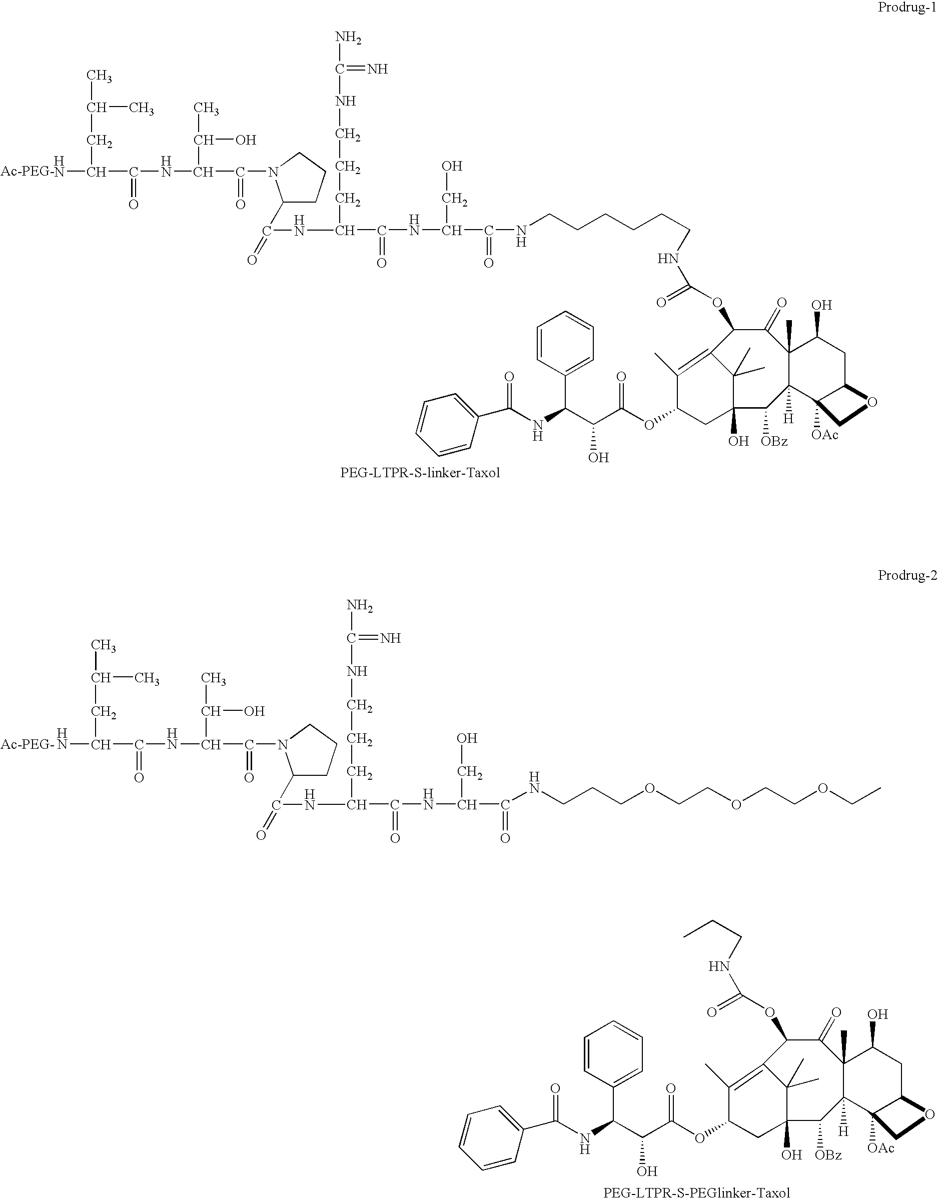

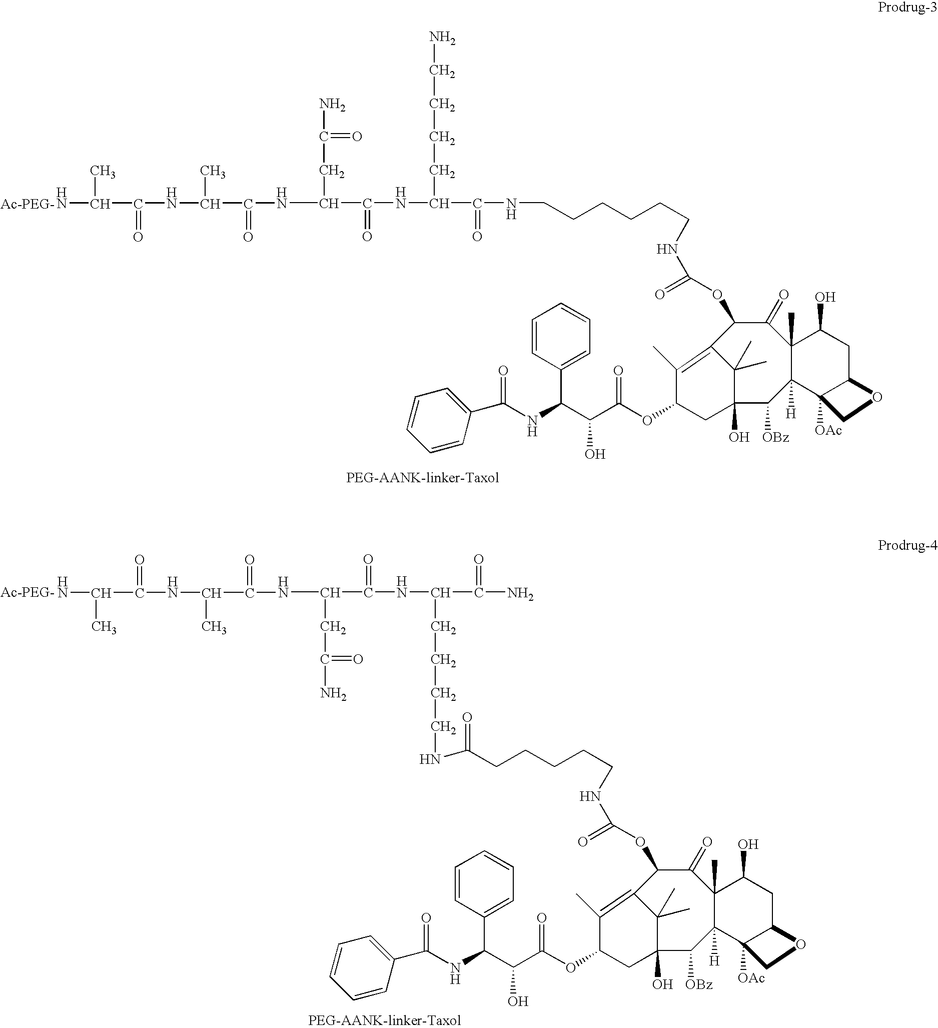

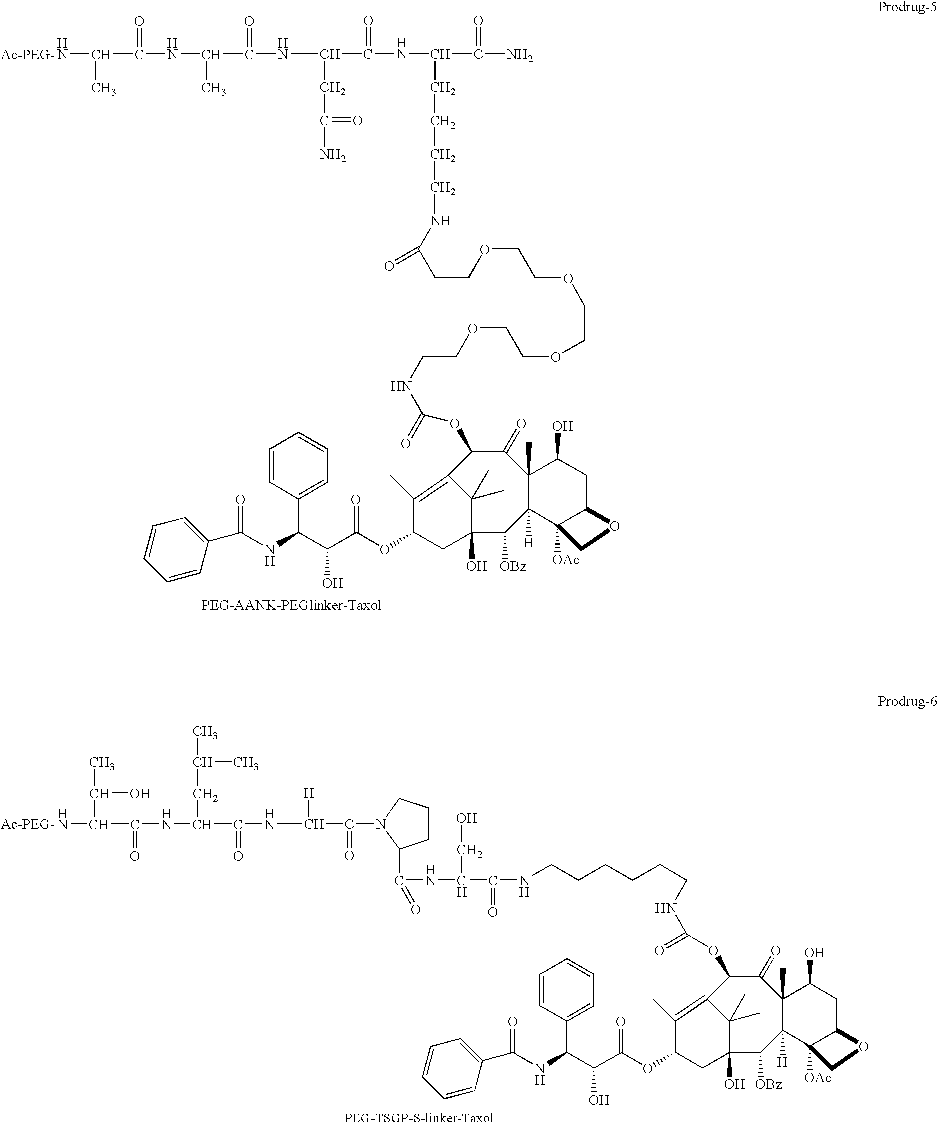

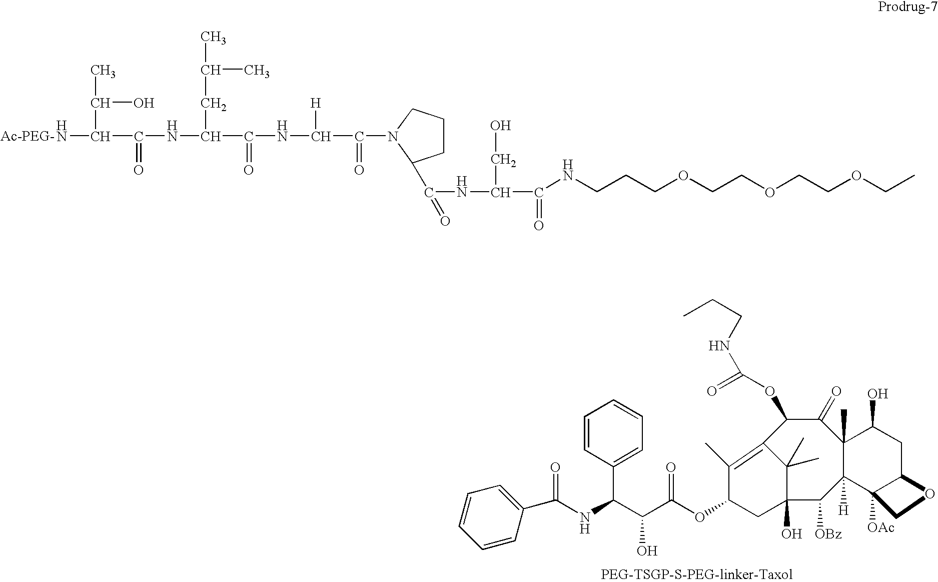

Prodrug-1 and prodrug-2 are activated by serine protease TF VIIa. Prodrug-3, prodrug-4 and prodrug-5 are activated by legumain. Prodrug-6 and prodrug-7 are activated by fibroblast activation protein (FAP).

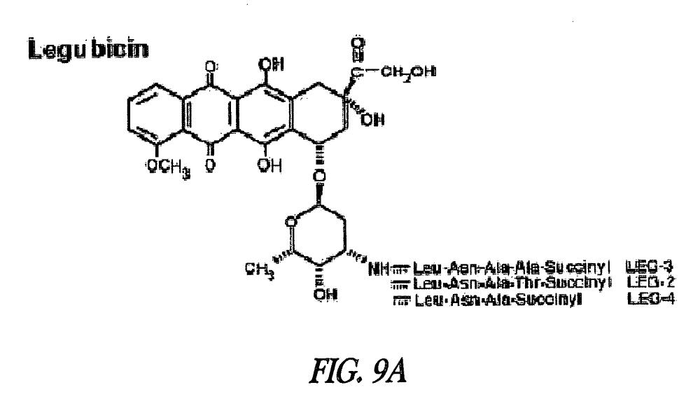

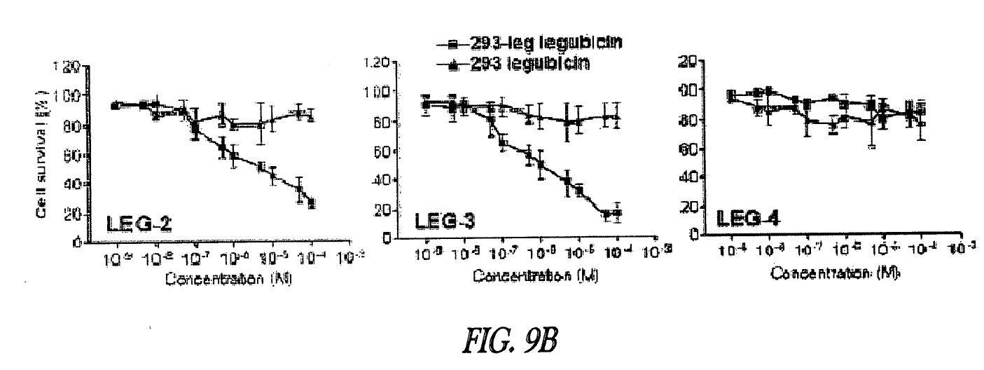

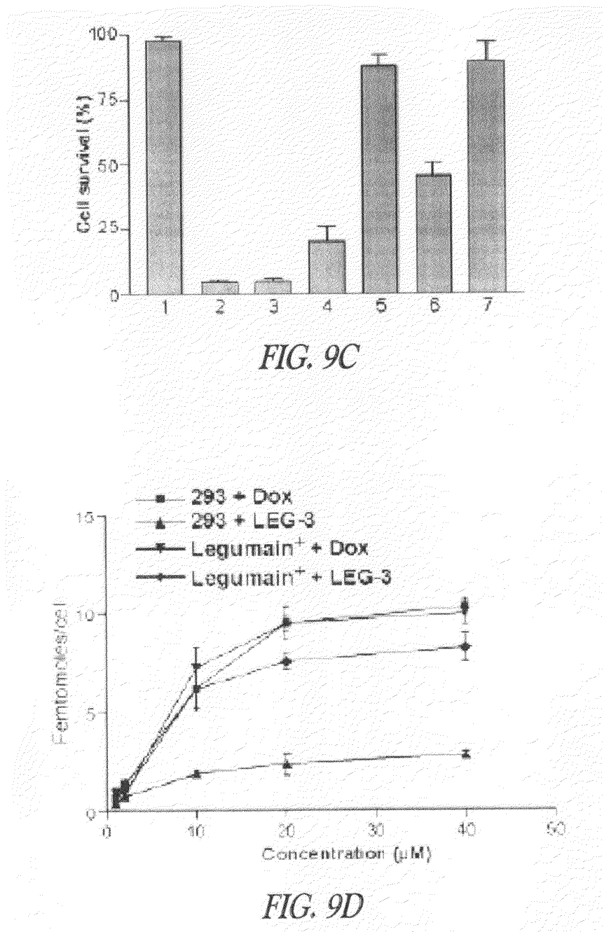

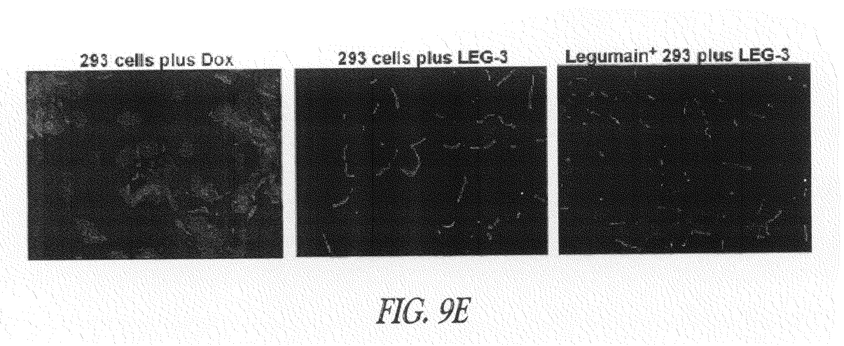

Additional legumain prodrugs include LEG-2 (N-Succinyl-β-alanyl-L-threoinyl-L-Asparaginyl-L-Leucyl-Doxorubicin) (SEQ ID NO:24) and LEG-3 (N-Succinyl-β-alanyl-L-alanyl-L-Asparaginyl-L-Leucyl-Doxorubicin) (SEQ ID NO:25), whose structures are shown in FIG. 9A. Legubicin is another prodrug, which contains a cytotoxic agent, doxorubicin, linked to a peptidyl sequence (Boc-Ala-Ala-Asn-Leu, SEQ ID NO:4). The structure of legubicin is provided below as formula IA.

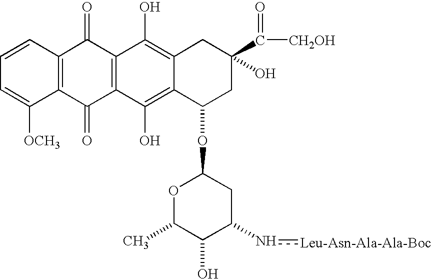

Note that in legubicin, an amino group in doxorubicin is attached to the C-terminus of the peptide Boc-Ala-Ala-Asn-Leu (SEQ ID NO:4).

In some instances, the prodrugs of the invention do not include LEG-2 or LEG-3 or legubicin, because the inventor has filed a separate application on these compounds.

In general, the intact prodrug is non-toxic. For example, data provided herein and in related application show that intact legubicin is not significantly cytotoxic. However, legubicin does become toxic after the amino acid sequence of the linked peptide (e.g., Boc-Ala-Ala-Asn-Leu (SEQ ID NO:4)) is cleaved by legumain. Legumain can cleave legubicin between the leucine and the asparagine of SEQ ID NO:4, thereby releasing doxorubicin-Leu to act as a cytotoxin on the cells that express legumain.

In another example, LEG-3 is another prodrug that contains the cytotoxic agent, doxorubicin, linked to a peptidyl sequence succinyl-Ala-Ala-Asn-Leu-doxorubicin (SEQ ID NO:8). The structure of LEG-3 is shown below as formula IB (note that the C-terminus of the peptidyl sequence is to the left and the N-terminus is to the right).

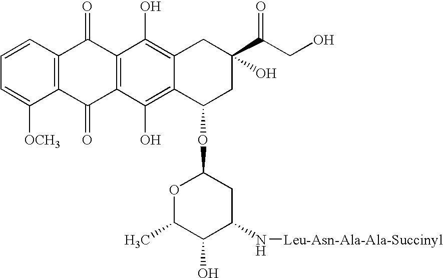

LEG-3 is essentially non-toxic to normal, non-cancerous cells that do not express asparaginyl endopeptidases. One beneficial characteristic of LEG-3 is that it is water-soluble and does not penetrate cell membranes or accumulate in normal, non-cancerous tissues. The succinyl group of LEG-3 tends to make LEG-3 more hydrophilic than legubicin. Accordingly, LEG-3 has even less toxicity than legubicin.

In general, while a peptide could be linked to the —CO—CH₂—OH moiety of doxorubicin to generate a prodrug having formula II, such a prodrug construct is less desirable than a doxorubicin prodrug having a linkage at the amino position shown in formula IA or IB. For example, a less desirable doxorubicin prodrug having formula II is shown below.

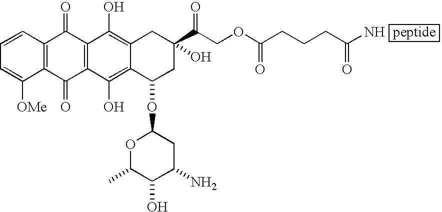

While doxorubicin prodrugs having formula II can be properly targeted to legumain-expressing cells, such prodrugs appear to more toxic than prodrugs having linkages like those shown in formulae IA and IB. Hence, linkage of peptides to the heterocyclic ring of doxorubicin is preferred.

Further examples of prodrugs with a doxorubicin and hydrophilic succinyl, polyethylene glycol, glutathione and glucuronide R₁ groups are shown below.

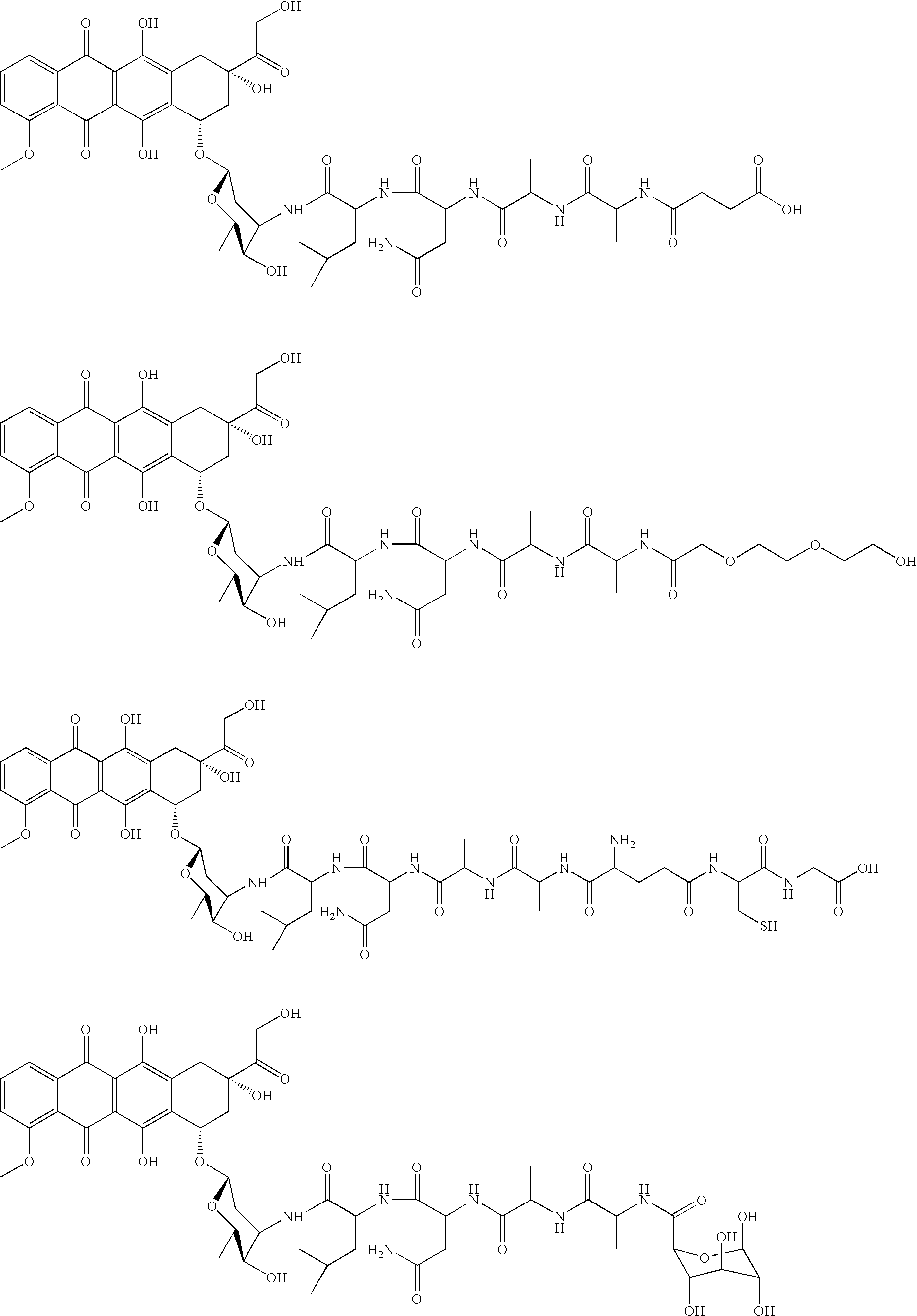

Prodrugs that include paclitaxel are also highly desirable. Several examples of paclitaxel-containing prodrugs are shown above. Such prodrugs can, for example, have any of the following structures and/or structural features.

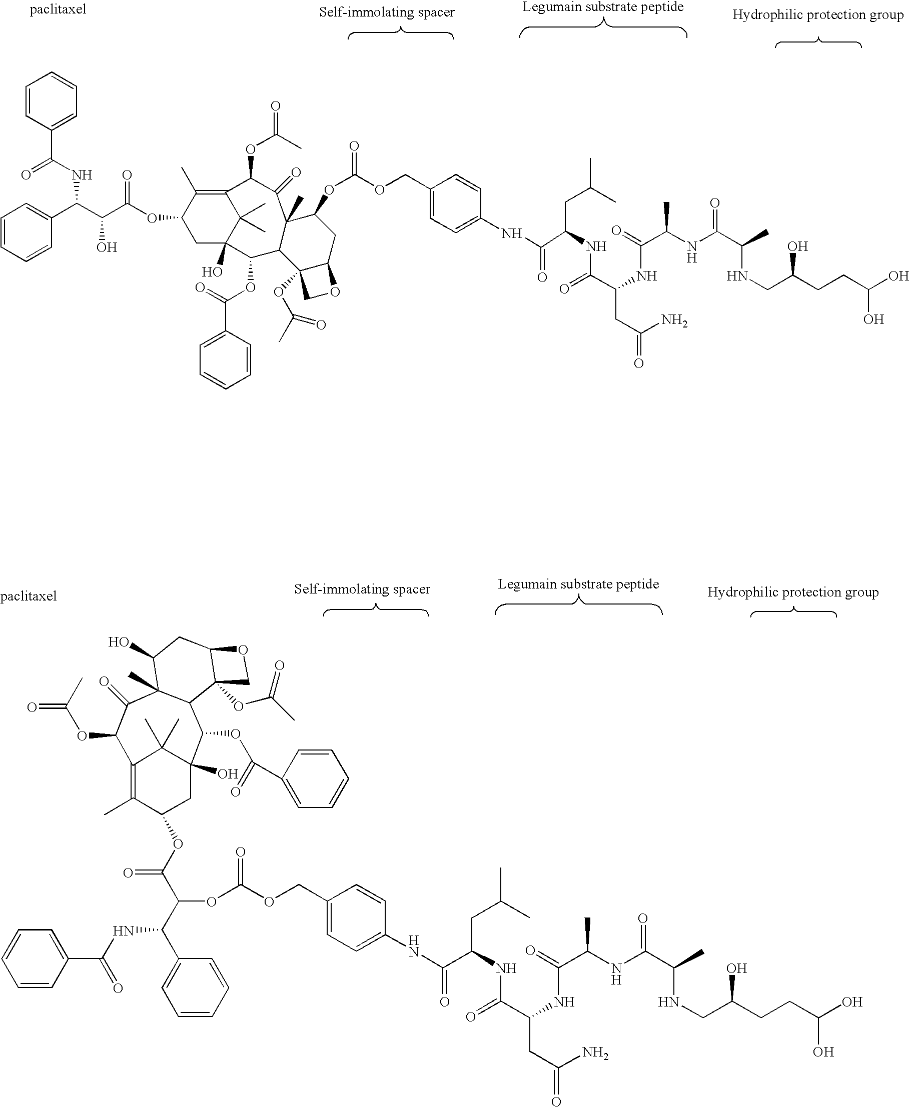

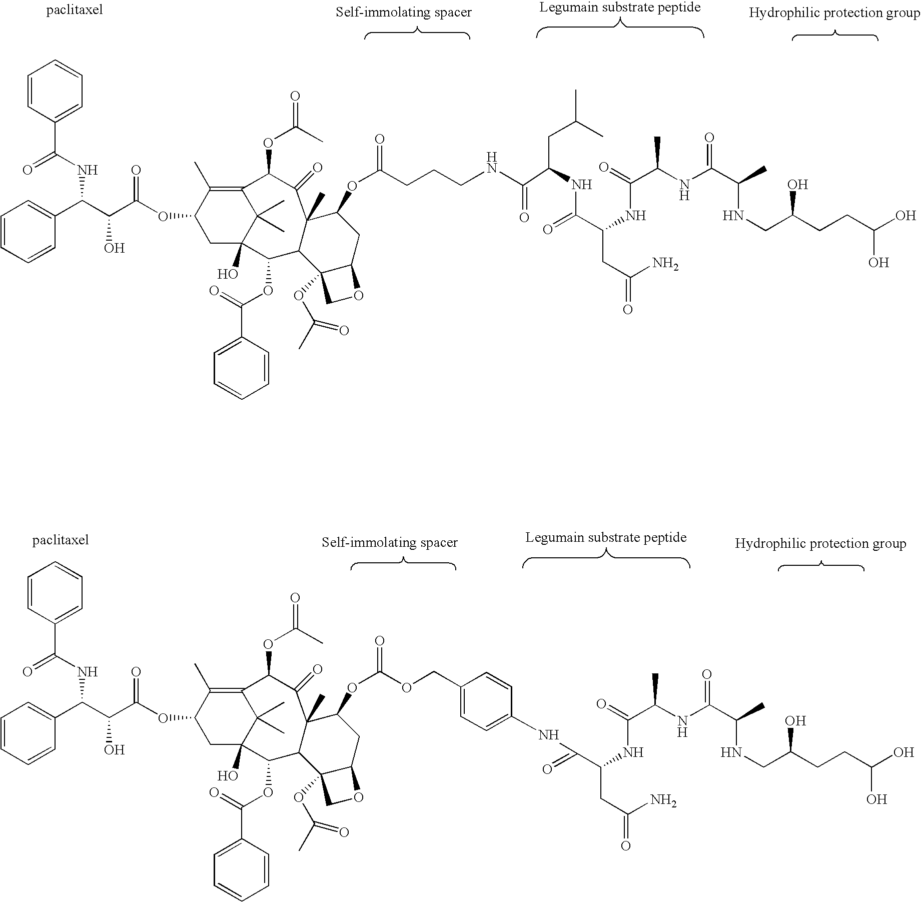

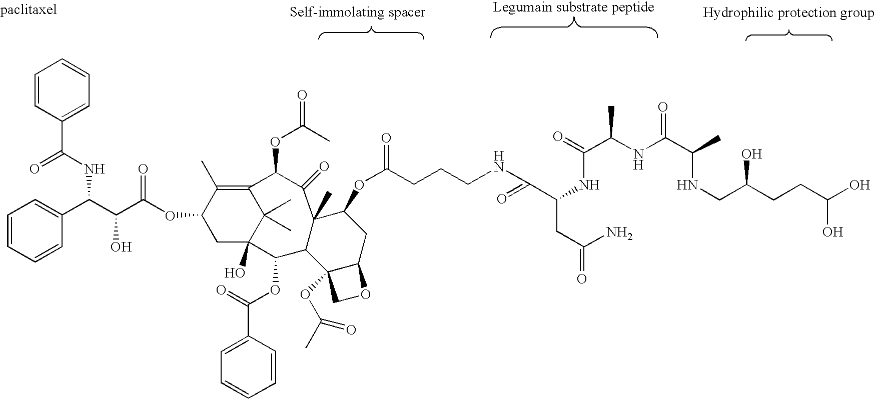

As provided herein, peptides linked to drugs can have a variety of sequences and a variety of lengths to form the prodrugs of the invention. Hence, any peptide can be used so long as the peptide sequence contains a cleavage site for a tumor-specific protease (e.g. Asn for legumain) at a position sufficiently near the drug such that the drug is active after cleavage of the prodrug. Generally, peptides are used that block or inhibit some or all of the drug's activity.

In some embodiments where legumain is the tumor-associated protease, the linked peptide can have an amino acid sequence that includes the sequence Asn-Leu. In other embodiments, the legumain target peptide can have an amino acid sequence that includes the sequence Ala-Asn-Leu. Further embodiments can have peptides with an amino acid sequence that include the sequence Thr-Asn-Leu, or the sequence Ala-Ala-Asn-Leu (SEQ ID NO:5), or the sequence Ala-Thr-Asn-Leu (SEQ ID NO:6). In some embodiments, the peptide further can have an N-β-alanyl terminus, an N-terminal Boc or an N-terminal succinyl residue.

Specific examples of doxorubicin prodrugs contemplated by the invention include the following:

(SEQ ID NO:7)

Boc-Ala-Ala-Asn-Leu-doxorubicin.

(SEQ ID NO:8)

Succinyl-Ala-Ala-Asn-Leu-doxorubicin.

(SEQ ID NO:9)

Boc-Ala-Thr-Asn-Leu-doxorubicin.

(SEQ ID NO:10)

Succinyl-Ala-Thr-Asn-Leu-doxorubicin.

(SEQ ID NO:11)

Boc-Ala-Asn-Leu-doxorubicin.

(SEQ ID NO:12)

Succinyl-Ala-Asn-Leu-doxorubicin.

(SEQ ID NO:13)

Boc-Thr-Leu-doxorubicin.

(SEQ ID NO:14)

Succinyl-Thr-Leu-doxorubicin.

(SEQ ID NO:21)

Succinyl-Ala-Ala-Asn-Leu-N₂H₃

In many embodiments, the agents and prodrugs of the invention are substantially non-immunogenic to the animal to which they are administered. The term “substantially non-immunogenic” means that the agent or prodrug can be administered to the animal on more than one occasion without causing a significant immune response. Such a significant immune response can be seen, for example, if a foreign protein or antibody was administered to the animal and a significant humoral or cellular immune response was initiated.

### Peptides

As described above, the prodrugs of the invention can include a peptide and the peptide shields or modulates the drug, toxin or therapeutic agent so that it is substantially inactive. For example, a peptide can be linked to a cytotoxic agent to modulate the cytotoxicity of the cytotoxic agent. In other embodiments, a peptide can be linked to a drug to modulate the activity of the drug. In other embodiments, a diagnostic agent can include a peptide that is linked to a label or a reporter molecule. Similarly, the inhibitors of the invention include peptidyl sequences that are recognized and bound by proteases.

The peptides employed can have amino acid sequences comprised of any available amino acid, although in some embodiment the peptide has an asparagine residue at a desired cleavage site. Amino acids included in the peptides can be genetically encoded L-amino acids, naturally occurring non-genetically encoded L-amino acids, synthetic L-amino acids or D-enantiomers of any of the above. The amino acid notations used herein for the twenty genetically encoded L-amino acids and common non-encoded amino acids are conventional and are as shown in Table 1. These amino acids can be linked together, for example, by peptidyl linkages, intersubunit linkages, or other intersubunit linkages that are consistent with enzyme-substrate or receptor-ligand binding interactions.

TABLE 1

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-Alanine | | bAla |
| 2,3-Diaminopropionic | | Dpr |
| acid | | |
| α-Aminoisobutyric acid | | Aib |
| N-Methylglycine | | MeGly |
| (sarcosine) | | |
| Ornithine | | Orn |
| Citrulline | | Cit |
| t-Butylalanine | | t-BuA |
| t-Butylglycine | | t-BuG |
| N-methylisoleucine | | MeIle |
| Phenylglycine | | Phg |
| Cyclohexylalanine | | Cha |
| Norleucine | | Nle |
| Naphthylalanine | | Nal |
| Pyridylalanine | | |
| 3-Benzothienyl alanine | | |
| 4-Chlorophenylalanine | | Phe(4-Cl) |
| 2-Fluorophenylalanine | | Phe(2-F) |
| 3-Fluorophenylalanine | | Phe(3-F) |
| 4-Fluorophenylalanine | | Phe(4-F) |
| Penicillamine | | Pen |
| 1,2,3,4-Tetrahydro- | | Tic |
| isoquinoline-3- | | |
| carboxylic acid | | |
| β-2-thienylalanine | | Thi |
| Methionine sulfoxide | | MSO |
| Homoarginine | | hArg |
| N-acetyl lysine | | AcLys |
| 2,4-Diamino butyric | | Dbu |
| acid | | |
| ρ-Aminophenylalanine | | Phe(pNH₂) |
| N-methylvaline | | MeVal |
| Homocysteine | | hCys |
| Homoserine | | hSer |
| ε-Amino hexanoic acid | | Aha |
| δ-Amino valeric acid | | Ava |
| 2,3-Diaminobutyric acid | | Dab |

Certain amino acids which are not genetically encoded and which can be present in agents of the invention include, but are not limited to, β-alanine (b-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); .beta.-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer). These amino acids also fall into the categories defined above.

The classifications of the above-described genetically encoded and non-encoded amino acids are summarized in Table 2, below. It is to be understood that Table 2 is for illustrative purposes only and does not purport to be an exhaustive list of amino acid residues which may comprise the peptides and peptide analogues described herein. Other amino acid residues which are useful for making the peptides and peptide analogues described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE 2

| Classification | Genetically Encoded | Genetically Non-Encoded |
|---|---|---|
| Hydrophobic | | |
| Aromatic | F, Y, W | Phg, Nal, Thi, Tic, Phe(4- |
| | | Cl), Phe(2-F), Phe(3-F), |
| | | Phe(4-F), Pyridyl Ala, |
| | | Benzothienyl Ala |
| Apolar | M, G, P | |
| Aliphatic | A, V, L, I | t-BuA, t-BuG, MeIle, Nle, |
| | | MeVal, Cha, bAla, MeGly, |
| | | Aib |
| Hydrophilic | | |
| Acidic | D, E | |
| Basic | H, K, R | Dpr, Orn, hArg, Phe(p- |
| | | NH₂), DBU, A₂ BU |
| Polar | Q, N, S, T, Y | Cit, AcLys, MSO, hSer |
| Cysteine-Like | C | Pen, hCys, β-methyl Cys |

Peptides described herein may be synthesized by methods available in the art, including recombinant DNA methods and chemical synthesis. Chemical synthesis may generally be performed using standard solution phase or solid phase peptide synthesis techniques, in which a peptide linkage occurs through the direct condensation of the α-amino group of one amino acid with the carboxy group of the other amino acid with the elimination of a water molecule. Peptide bond synthesis by direct condensation, as formulated above, requires suppression of the reactive character of the amino group of the first and of the carboxyl group of the second amino acid. The masking substituents must permit their ready removal, without inducing breakdown of the labile peptide molecule.

In solution phase synthesis, a wide variety of coupling methods and protecting groups may be used (see Gross and Meienhofer, eds., “The Peptides: Analysis, Synthesis, Biology,” Vol. 1-4 (Academic Press, 1979); Bodansky and Bodansky, “The Practice of Peptide Synthesis,” 2d ed. (Springer Verlag, 1994)). In addition, intermediate purification and linear scale up are possible. Those of ordinary skill in the art will appreciate that solution synthesis requires consideration of main chain and side chain protecting groups and activation method. In addition, careful segment selection may be necessary to minimize racemization during segment condensation. Solubility considerations are also a factor.

Solid phase peptide synthesis uses an insoluble polymer for support during organic synthesis. The polymer-supported peptide chain permits the use of simple washing and filtration steps instead of laborious purifications at intermediate steps. Solid-phase peptide synthesis may generally be performed according to the method of Merrifield et al., J. Am. Chem. Soc. 85:2149, 1963, which involves assembling a linear peptide chain on a resin support using protected amino acids. Solid phase peptide synthesis typically utilizes either the Boc or Fmoc strategy, which are now well known in the art.

Those of ordinary skill in the art will recognize that, in solid phase synthesis, deprotection and coupling reactions must go to completion and the side-chain blocking groups must be stable throughout the entire synthesis. In addition, solid phase synthesis is generally most suitable when peptides are to be made on a small scale.

The peptides can be directly linked to drugs or R₁ groups. Alternatively, the peptides can be indirectly linked to drugs or R₁ groups through a linker or spacer moiety. Such linkers and spacers can be alkylene group, sugars, oligosaccharides or polyalkylene glycols.

### Drugs

According to the invention, any drug useful for modulating, treating, killing or otherwise affecting the physiological state of a tumor cell can be attached to a tumor-specific peptide substrate or an agent that can bind to a tumor-specific protease. Attachment of drugs to the peptide substrate of a tumor-specific protease generates a prodrug of the invention. Similarly, attachment of a drug to an agent that can bind to a tumor-specific protease permits delivery and accumulation of the drug in tumor cells.

While the invention is directed to attaching any drug to a peptide substrate or an agent that can bind a tumor-specific protease, in some embodiments, the drug is a cytotoxic agent or an effector molecule. The cytotoxic agents and effector molecules useful in the practice of the invention include cytotoxins, photosensitizing agents and chemotherapeutic agents. These agents include, but are not limited to, folate antagonists, pyrimidine antimetabolites, purine antimetabolites, 5-aminolevulinic acid, alkylating agents, platinum anti-tumor agents, anthracyclines, DNA intercalators, epipodophyllotoxins, DNA topoisomerases, microtubule-targeting agents, vinca alkaloids, taxanes, epothilones and asparaginases. Further information can be found in Bast et al., CANCER MEDICINE, edition 5, which is available free as a digital book. See website at ncbi.nlm.nih.gov/books/by.fcgi?call=bv View.ShowTOC&rid=cmed. TOC&depth=2.

Folic acid antagonists are cytotoxic drugs used as antineoplastic, antimicrobial, anti-inflammatory, and immune-suppressive agents. While several folate antagonists have been developed, and several are now in clinical trial, methotrexate (MTX) is the antifolate with the most extensive history and widest spectrum of use. MTX is an essential drug in the chemotherapy regimens used to treat patients with acute lymphoblastic leukemia, lymphoma, osteosarcoma, breast cancer, choriocarcinoma, and head and neck cancer, as well as being an important agent in the therapy of patients with nonmalignant diseases, such as rheumatoid arthritis, psoriasis, and graft-versus-host disease.

Pyrimidine antimetabolites include fluorouracil, cytosine arabinoside, 5-azacytidine, and 2′,2′-difluoro-2′-deoxycytidine. Purine antimetabolites include 6-mercatopurine, thioguanine, allopurinol (4-hydroxypyrazolo-3,4-d-pyrimidine), deoxycoformycin (pentostatin), 2-fluoroadenosine arabinoside (fludarabine; 9-β-d-arabinofuranosyl-2-fluoradenine), and 2-chlorodeoxyadenosine (Cl-dAdo, cladribine). In addition to purine and pyrimidine analogues, other agents have been developed that inhibit biosynthetic reactions leading to the ultimate nucleic acid precursors. These include phosphonacetyl-L-aspartic acid (PALA), brequinar, acivicin, and hydroxyurea.

Alkylating agents and the platinum anti-tumor compounds form strong chemical bonds with electron-rich atoms (nucleophiles), such as sulfur in proteins and nitrogen in DNA. Although these compounds react with many biologic molecules, the primary cytotoxic actions of both classes of agents appear to be the inhibition of DNA replication and cell division produced by their reactions with DNA. However, the chemical differences between these two classes of agents produce significant differences in their anti-tumor and toxic effects. The most frequently used alkylating agents are the nitrogen mustards. Although thousands of nitrogen mustards have been synthesized and tested, only five are commonly used in cancer therapy today. These are mechlorethamine (the original “nitrogen mustard”), cyclophosphamide, ifosfamide, melphalan, and chlorambucil. Closely related to the nitrogen mustards are the aziridines, which are represented in current therapy by thiotepa, mitomycin C, and diaziquone (AZQ). Thiotepa (triethylene thiophosphoramide) has been used in the treatment of carcinomas of the ovary and breast and for the intrathecal therapy of meningeal carcinomatosis. The alkyl alkane sulfonate, busulfan, was one of the earliest alkylating agents. This compound is one of the few currently used agents that clearly alkylate through an SN2 reaction. Hepsulfam, an alkyl sulfamate analogue of busulfan with a wider range of anti-tumor activity in preclinical studies, has been evaluated in clinical trials but thus far has demonstrated no superiority to busulfan. Busulfan has a most interesting, but poorly understood, selective toxicity for early myeloid precursors. This selective effect is probably responsible for its activity against chronic myelocytic leukemia (CML).

Photosensitizing agents induce cytotoxic effects on cells and tissues. Upon exposure to light the photosensitizing compound may become toxic or may release toxic substances such as singlet oxygen or other oxidizing radicals that are damaging to cellular material or biomolecules, including the membranes of cells and cell structures, and such cellular or membrane damage can eventually kill the cells. A range of photosensitizing agents can be used, including psoralens, porphyrins, chlorines, aluminum phthalocyanine with 2 to 4 sulfonate groups on phenyl rings (e.g., AlPcS₂ₐ or AlPcS₄) and phthalocyanins. Such drugs become toxic when exposed to light. In one embodiment, the photosensitizing agent is an amino acid called 5-aminolevulinic acid, which is converted to protoporphyrin IX, a fluorescent photosensitizer. The structure of 5-aminolevulinic acid is shown below.

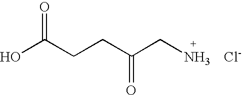

5-Aminolevulinic acid has been approved for treating skin and esophagus cancers and is in clinical trial for brain tumor detection and therapy. A major problem with currently available 5-aminolevulinic acid formuations is that normal cells also absorb 5-aminolevulinic acid. However, the present invention solves this problem by using 5-aminolevulinic acid in a prodrug conjugated to a protease substrate peptide. This prodrug is selectively cleaved in tumor microenvironment by one of the tumor-specific proteases described herein. Thus, uptake by normal, non-cancer cells is significantly decreased when the 5-aminolevulinic acid is placed in a prodrug of the invention. According to the invention, the 5-aminolevulinic acid prodrug is designed so that the 5-aminolevulinic acid is released by protease cleavage with its structure intact so that the 5-aminolevulinic acid is metabolically converted to protoporphyrin IX. Light therapy is used to activate the photosensitizing agent. For example, laser treatment can be used. Alternatively, light rods can be inserted into the flesh.

Topoisomerase poisons are believed to bind to DNA, the topoisomerase, or either molecule at or near the region of the enzyme involved in the formation of the DNA protein covalent linkage. Many topoisomerase poisons, such as the anthracyclines and actinomycin D, are relatively planar hydrophobic compounds that bind to DNA with high affinity by intercalation, which involves stacking of the compound between adjacent base pairs. Anthracyclines intercalate into double-stranded DNA and produce structural changes that interfere with DNA and RNA syntheses. Several of the clinically relevant anthracyclines are shown below.

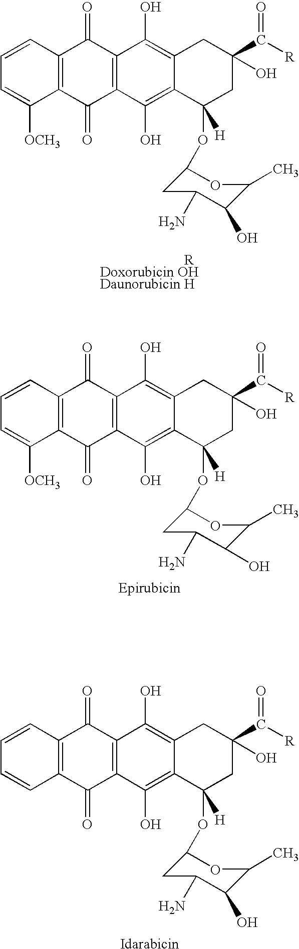

Non-intercalating topoisomerase-targeting drugs include epipodophyllotoxins such as etoposide and teniposide. Etoposide is approved in the United States for the treatment of testicular and small cell lung carcinomas. Etoposide phosphate is more water soluble than etoposide and is rapidly converted to etoposide in vivo. Other non-intercalating topoisomerase-targeting drugs include topotecan and irinotecan.

Unique classes of natural product anticancer drugs have been derived from plants. As distinct from those agents derived from bacterial and fungal sources, the plant products, represented by the Vinca and Colchicum alkaloids, as well as other plant-derived products such as paclitaxel (Taxol) and podophyllotoxin, do not target DNA. Rather, they either interact with intact microtubules, integral components of the cytoskeleton of the cell, or with their subunit molecules, the tubulins. Clinically useful plant products that target microtubules include the Vinca alkaloids, primarily vinblastine (VLB), vincristine (VCR), vinorelbine (Navelbine, VRLB), and a newer Vinca alkaloid, vinflunine (VFL; 20′,20′-difluoro-3′,4′-dihydrovinorelbine), as well as the two taxanes, paclitaxel and docetaxel (Taxotere). The structure of paclitaxel is provided below.

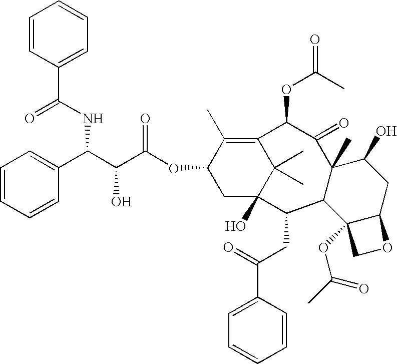

Preferably a paclitaxel moiety is linked to the peptide by C10 and/or C2 hydroxyl moiety.

Hence, examples of drugs that can be used to form prodrugs of the invention include, but are not limited to, aldesleukin, 5-aminolevulinic acid, asparaginase, bleomycin sulfate, camptothecin, carboplatin, carmustine, cisplatin, cladribine, cyclophosphamide (lyophilized), cyclophosphamide (non-lyophilized), cytarabine (lyophilized powder), dacarbazine, dactinomycin, daunorubicin, diethylstilbestrol, doxorubicin (doxorubicin, 4′-epidoxorubicin, 4- or 4′-deoxydoxorubicin), epoetin alfa, esperamycin, etidronate, etoposide, N,N-bis(2-chloroethyl)-hydroxyaniline, 4-hydroxycyclophosphamide, fenoterol, filgrastim, floxuridine, fludarabine phosphate, fluorocytidine, fluorouracil, fluorouridine, goserelin, granisetron hydrochloride, idarubicin, ifosfamide, interferon alpha-2a, interferon alpha-2b, leucovorin calcium, leuprolide, levamisole, mechiorethamine, medroxyprogesterone, melphalan, methotrexate, mitomycin, mitoxantrone, muscarine, octreotide, ondansetron hydrochloride, oxyphenbutazone, paclitaxel, pamidronate, pegaspargase, plicamycin, salicylic acid, salbutamol, sargramostim, streptozocin, taxol, terbutaline, terfenadine, thiotepa, teniposide, vinblastine, vindesine and vincristine. Other drugs and toxic effector molecules for use in the present invention are disclosed, for example, in WO 98/13059; Payne, 2003; US 2002/0147138 and other references available to one of skill in the art.

Peptides can be conjugated to chemotherapeutic agents, drugs, reporter molecules, labels, cytotoxic agents and other entities by using peptidyl amino groups, carboxylate groups or side chain moieties of the peptidyl amino acids to form covalent linkages with such chemotherapeutic agents, drugs, reporter molecules, labels, cytotoxic agents and other agents. Amino acids can be conjugated to such entities by any method available to one of skill in the art. For example, functional groups present on the side chains of amino acids in the peptides can be combined with functional groups in the entity to which the peptide is conjugated. Functional groups that can form covalent bonds include, for example, —COOH and —OH; —COOH and —NH₂; and —COOH and —SH. Pairs of amino acids that can be used to conjugate proteins to the present peptides include, Asp and Lys; Glu and Lys; Asp and Arg; Glu and Arg; Asp and Ser; Glu and Ser; Asp and Thr; Glu and Thr; Asp and Cys; and Glu and Cys. Other examples of amino acid residues that are capable of forming covalent linkages with one another include cysteine-like amino acids such Cys, hCys, β-methyl-Cys and Pen, which can form disulfide bridges with one another. Other pairs of amino acids that can be used for conjugation and cyclization of the peptide will be apparent to those skilled in the art.

The groups used to conjugate a peptide to another agent need not be a side group on an amino acid. Examples of functional groups capable of forming a covalent linkage with the amino terminus of a peptide include, for example, carboxylic acids and esters. Examples of functional groups capable of forming a covalent linkage with the carboxyl terminus of a peptide include —OH, —SH, —NH₂ and —NHR where R is (C₁-C₆) alkyl, (C₁-C₆) alkenyl and (C₁-C₆) alkynyl.

The variety of reactions between two side chains with functional groups suitable for forming such linkages, as well as reaction conditions suitable for forming such linkages, will be apparent to those of skill in the art. Preferably, the reaction conditions used to conjugate the peptides to other entities are sufficiently mild so as not to degrade or otherwise damage the peptide. In particular, some embodiments require a functional protease recognition site (e.g. an intact asparagine for legumain), so conditions should be adjusted to minimize damage to such sites. Suitable groups for protecting the various functionalities as necessary are well known in the art (see, e.g., Greene & Wuts, 1991, 2nd ed., John Wiley & Sons, NY), as are various reaction schemes for preparing such protected molecules.

Methods for linking peptides to other entities are available in the art. See Spatola, 1983, “Peptide Backbone Modifications” In: Chemistry and Biochemistry of Amino Acids Peptides and Proteins (Weinstein, ed.), Marcel Dekker, New York, p. 267 (general review); Morley, 1980, Trends Pharm. Sci. 1:463-468; Hudson et al., 1979, Int. J. Prot. Res. 14:177-185 (—CH₂ NH—, —CH₂ CH₂—); Spatola et al., 1986, Life Sci. 38:1243-1249 (—CH₂—S); Hann, 1982, J. Chem. Soc. Perkin Trans. I. 1:307-314 (—CH═CH—, cis and trans); Almquist et al., 1980, J. Med. Chem. 23:1392-1398 (—COCH₂—); Jennings-White et al., Tetrahedron. Lett. 23:2533 (—COCH₂—); European Patent Application EP 45665 (1982) CA:97:39405 (—CH(OH)CH₂—); Holladay et al., 1983, Tetrahedron Lett. 24:4401-4404 (—C(OH)CH₂—); and Hruby, 1982, Life Sci. 31:189-199 (—CH₂—S—).

Preferably a paclitaxel is linked to the peptide by C10 and/or C2 hydroxyl moiety. Preferably doxorubicin, 4′-epidoxorubicin, 4- or 4′-deoxydoxorubicin or a compound such an etoposide, N,N-bis(2-chloroethyl)-hydroxyaniline, 4-hydroxycyclophosphamide, vindesine, vinblastine, vincristine, terfenadine, terbutaline, fenoterol, salbutamol, muscarine, oxyphenbutazone, salicylic acid, p-aminosalicylic acid, 5-fluorouracil, 5-fluorocytidine, 5-fluorouridine, methotrexate, diclofenac, flufenamic acid, 4-methylaminophenazone, theophylline, nifedipine, mitomycin C, mitoxantrone, camptothecin, m-AMSA, taxol, nocodazole, colchicine, cyclophosphamide, rachelmycin, cisplatin, melphalan, bleomycin, nitrogen mustard, phosphoramide mustard, quercetin, genistein, erbstatin, tyrphostin, rohitukin derivative ((−)-cis-5,7-dihydroxy-2-(2-chlorophenyl)-8-[4-(3-hydroxy-1-methyl)-piperidinyl]-4H-1-benzopyran-4-one; EP 89119710.5), retinoic acid, butyric acid, phorbol ester, DMSO, aclacinomycin, progesterone, buserelin, tamoxifen, mifepristone, onapristone, N-(4-aminobutyl)-5-chloro-2-naphthalenesulfonamide, pyridinyloxazol-2-one, quinolyl-oxazol-2-one, isoquinolyl-oxazol-2-one, staurosporine, ethanolamine, verapamil, forskolin, 1,9-dideoxyforskolin, quinine, quinidine, reserpine, methyl 18-O-(3,5-dimethoxy-4-hydroxybenzoyl)reserpate, lonidamine, buthionine-sulfoximine, diethyl dithiocarbamate, cyclosporin A, azathioprine, chlorambucil, N-(4-trifluoromethylphenyl)-2-cyano-3-hydroxycrotonamide (WO 91/17748). 15-deoxyspergualine, FK 506, ibuprofen, indomethacin, aspirin, sulfasalazine, penicillamine, chloroquine, dexamethasone, prednisolone, lidocaine, propafenone, procaine, mefenamic acid, paracetamol, 4-aminophenazone, muskosine, orciprenaline, isoprenaline, amiloride, p-nitrophenyl guanidinobenzoate or their derivatives additionally substituted by one or more hydroxyl, amino or imino groups.

### Cancer Treatment

According to the invention, the prodrug compounds and inhibitors described herein are useful for preventing, treating or diagnosing cancer. One aspect of the invention is a protease inhibitor-based imaging agent to detect active protease activity in vivo and to identify the actual proteases involved in particular cancers. This is fundamentally distinct from other imaging agents (for example, that employ antibodies). The protease activity imaging agent will detect active protease in the tumor microenvironment. That information will determine what protease inhibitor or combination of protease inhibitors will be affective in a given cancer. It will also determine the appropriate prodrug or combination of prodrugs will be effective in a given cancer.

As used herein, the term “cancer” includes solid mammalian tumors as well as hematological malignancies. The terms “tumor cell(s)” and “cancer cell(s)” are used interchangeably herein.

“Solid mammalian tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin.

The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS.

In addition, a cancer at any stage of progression can be treated, such as primary, metastatic, and recurrent cancers. The invention can also be used to treat autoimmune deficiency syndrome-associated Kaposi's sarcoma, cancer of the adrenal cortex, cancer of the cervix, cancer of the endometrium, cancer of the esophagus, cancer of the head and neck, cancer of the liver, cancer of the pancreas, cancer of the prostate, cancer of the thymus, carcinoid tumors, chronic lymphocytic leukemia, Ewing's sarcoma, gestational trophoblastic tumors, hepatoblastoma, multiple myeloma, non-small cell lung cancer, retinoblastoma, or tumors in the ovaries. A cancer at any stage of progression can be treated or detected, such as primary, metastatic, and recurrent cancers. Information regarding numerous types of cancer can be found, e.g., from the American Cancer Society (www.cancer.org), or from, e.g., Wilson et al. (1991) Harrison's Principles of Internal Medicine, 12th Edition, McGraw-Hill, Inc. Both human and veterinary uses are contemplated.

As used herein the terms “normal mammalian cell” and “normal animal cell” are defined as a cell that is growing under normal growth control mechanisms (e.g., genetic control) and that displays normal cellular differentiation and normal migration patterns. Cancer cells differ from normal cells in their growth patterns, migration and in the nature of their cell surfaces. For example cancer cells tend to grow continuously and chaotically, without regard for their neighbors, and can sometimes migrate to distal sites to generate tumors in other areas of the body.

The present invention is directed, in some embodiments, to methods of treating cancer in an animal, for example, for human and veterinary uses, which include administering to a subject animal (e.g., a human), a therapeutically effective amount of an agent (e.g. a prodrug or a legumain inhibitor) of the present invention.

Treatment of, or treating, cancer is intended to include the alleviation of or diminishment of at least one symptom typically associated with the disease. The treatment also includes alleviation or diminishment of more than one symptom. The treatment may cure the cancer, e.g., it may substantially kill the cancer cells and/or it may arrest or inhibit the growth of the cancerous tumor. The treatment can also promote apoptosis of cancer cells. Thus, legumain is directly involved in and actually inhibits the cascade of activities that leads to cellular apoptosis, particularly in cancer cells where legumain is highly expressed. Accordingly, use of the present inhibitors counteracts this effect and promotes apoptosis in cells that express legumain (e.g. tumor cells).

The tumor specific proteases described herein are cell surface associated proteases. These proteases function in protease networks that play critical roles in modulating extracellular matrix proteins. For instance, certain cancers may employ more cysteine proteases than matalloproteinas or serine protease and verse versa. However the matrix modifying function of each of these proteases is indispensable for tumor metastasis and invasive growth.

Anti-cancer activity can be evaluated against varieties of cancers using methods available to one of skill in the art. Anti-cancer activity, for example, is determined by identifying the lethal dose (LD 100) or the 50% effective dose (ED50) or the dose that inhibits growth at 50% (GI50) of an agent of the present invention that prevents the growth of a cancer. In one aspect, anti-cancer activity is the amount of the agents that kills 50% or 100% of the cancer cells, for example, when measured using standard dose response methods.

The present invention also provides a method of evaluating a therapeutically effective dosage for treating a cancer with an agent of the invention that includes determining the LD100 or ED50 of the agent in vitro. Such a method permits calculation of the approximate amount of agent needed per volume to inhibit cancer cell growth or to kill 50% to 100% of the cancer cells. Such amounts can be determined, for example, by standard microdilution methods.

In some embodiments, the agents of the invention can be administered in multiple doses over a period of one to seven days.

The term “animal,” as used herein, refers to an animal, such as a warm-blooded animal, which is susceptible to or has a disease associated with protease expression, for example, cancer. Mammals include cattle, buffalo, sheep, goats, pigs, horses, dogs, cats, rats, rabbits, mice, and humans. Also included are other livestock, domesticated animals and captive animals. The term “farm animals” includes chickens, turkeys, fish, and other farmed animals. Mammals and other animals including birds may be treated by the methods and compositions described and claimed herein.

### Apoptosis

Both tumor and tumor stromal cells have to survive under substantially lower oxygen tension within the tumor micro-environment in contrast to adjacent normal tissues. As described herein legumain expression is induced by hypoxia and occurs early during tumor development. Over-expression of legumain protects cells from multiple programmed cell death pathways. Also as demonstrated herein, during TNF induced cell death, lysosomal proteases are diffused into cytoplasm and legumain exerts an anti-apoptotic activity by depleting procaspase 9 and inactivating caspase 8 in death receptor complex and caspase 9 in apoptosomes. Pro-legumain can be activated by capspase 3. In addition, legumain bind directly to cathepsin B and suppress its auto-activation and activity toward Bid activation. Therefore, the presence of legumain serves as a brake for the caspase cascade and serves to depress apoptosis in cancer cells. These findings indicate legumain plays a critical role in mitochondria and death receptor mediated apoptosis.

Inhibition of legumain activity and expression sensitize tumor cells to natural death cues and chemotherapeutic agents, indicative of a novel modality of cancer intervention. Thus, the high affinity cell permeable inhibitors of legumain (asparaginyl endopeptidases inhibitors, AEPI) that are described herein are useful for promoting apoptosis in cancer cells. Data provided herein demonstrates these inhibitors sensitize tumor cells to apoptosis and make tumor cells more susceptible to a variety of cancer therapeutic interventions including chemotherapeutic agents and radiation.

### Other Therapeutic Methods

In addition to expression in cancer cells, legumain is expressed in monocytes. Such monocyte expression is differentially regulated by GM-CSF and M-CSF. Legumain is also expressed in tumor associated macrophages. Recently, legumain, has been identified as an inhibitor of osteoclast formation and is associated with bone resorption. Choi, S. J., et al., Osteoclast inhibitory peptide 2 inhibits osteoclast formation via its C-terminal fragment. J Bone Miner Res, 2001. 16(10): p. 1804-11.

Accordingly, the invention also contemplates inhibiting or killing tumor associated macrophages and osteoclast activity, for example, to prevent and treat osteoporosis. Methods for inhibiting osteoclast activity or for preventing or treating osteoporosis in an animal involve administering to the animal an agent that inhibits legumain. Any inhibitor of legumain can be utilized, for example, any of the inhibitors described herein.

As described herein, legumain is expressed in monocytes. Monocytes and macrophage originate from multipotential progenitor cells in bone marrow and play a pivotal role in host defense to pathogens, wound healing, angiogenesis, and various types of chronic inflammatory responses. Under chemokine and other cytokine induction monocytes migrate to tissues and differentiate into macrophages. Macrophages in various tissue and disease states vary in their morphology and function and have been given different names, e.g. Kupffer cells in liver, microglial cells in the central nervous system, and foam cells in atherosclerotic lesions. GM-CSF and M-CSF independently induce proliferation and differentiation of monocytes into distinct subsets of macrophages. Legumain is not detectable in freshly isolated unstimulated monocytes, but is up-regulated by both GM-CSF and M-CSF. Hashimoto, S., et al., Serial analysis of gene expression in human monocytes and macrophages. Blood, 1999. 94(3): p. 837-44. M-CSF induced macrophages express a greater amount of legumain than GM-CSF induced macrophages.

According to the invention, legumain can influence monocyte/macrophage migration, infiltration, and antigen processing. M-CSF is a potent chemoattractant for cells of monocytes and macrophage lineages. Wang, J. M., et al., Induction of monocyte migration by recombinant macrophage colony-stimulating factor. J Immunol, 1988. 141(2): p. 575-9. GM-CSF lacks chemotactic and chemokinetic effects, but enhances monocyte transendothelial migration in response to C5a or monocyte chemoattractant protein-1. Shang, X. Z. and A. C. Issekutz, Enhancement of monocyte transendothelial migration by granulocyte-macrophage colony-stimulating factor: requirement for chemoattractant and CD11a/CD18 mechanisms. Eur J Immunol, 1999. 29(11): p. 3571-82. Hence, legumain expression may be involved in increasing or modulating the migratory and infiltration activities of monocytes and/or macrophages.

The invention provides a method for modulating the migration and/or infiltration of cells that includes contacting the cells with legumain or an inhibitor of legumain. In general, increased levels of legumain can stimulate cellular migration and/or infiltration whereas inhibition of legumain can decrease cellular migration and/or infiltration. Such methods may be used in vitro or in vivo. Such methods may be useful not only for treating and preventing cancer but for treating and preventing inflammatory diseases, autoimmune diseases and atherosclerosis.

### Imaging and Detection

In one embodiment, the protease inhibitors, prodrugs and methods provided herein can be used to diagnose the location, extent, and pathologic composition of cancer anywhere within the body of a mammal. For example, detection of an agent (e.g., an inhibitor or antibody) capable of binding to or becoming activated by legumain can provide information regarding the location, shape, extent and pattern of the cancer. A reporter molecule, label or signaling compound can be attached to agents and inhibitors that can bind to, or be activated by, legumain. Such conjugates can then be used in vivo or in vitro to image, locate or otherwise detect the tissue to which the agent binds. The reporter molecule, label or signaling compound that is linked to the agent or inhibitor will, of course, depend on the ultimate application of the invention. Labels employed with the inhibitors and agents of the invention can be fluorophores, radioisotopes, metals, enzymes, enzyme substrates, luminescent moieties, and the like.

Where the aim is to provide an image of the tumor, one of skill in the art may desire to use a diagnostic agent that is detectable upon imaging, such as a paramagnetic, radioactive or fluorogenic agent. Such agents are available in the art, for example, as described and disclosed in U.S. Pat. No. 6,051,230 which is incorporated by reference herein in its entirety. Many diagnostic agents are known in the art to be useful for imaging purposes, as are methods for their attachment to peptides and antibodies (see, e.g., U.S. Pat. Nos. 5,021,236 and 4,472,509, both incorporated herein by reference).

In the case of paramagnetic ions, one of skill may choose to use, for example, ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being preferred. One example of a label that may used is gadolinium or a gadolinium complex. For example, the following gadolinium complex can be used as a label:

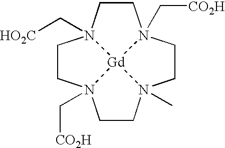

Gadolinium-containing agents can, for example, be synthesized as follows.

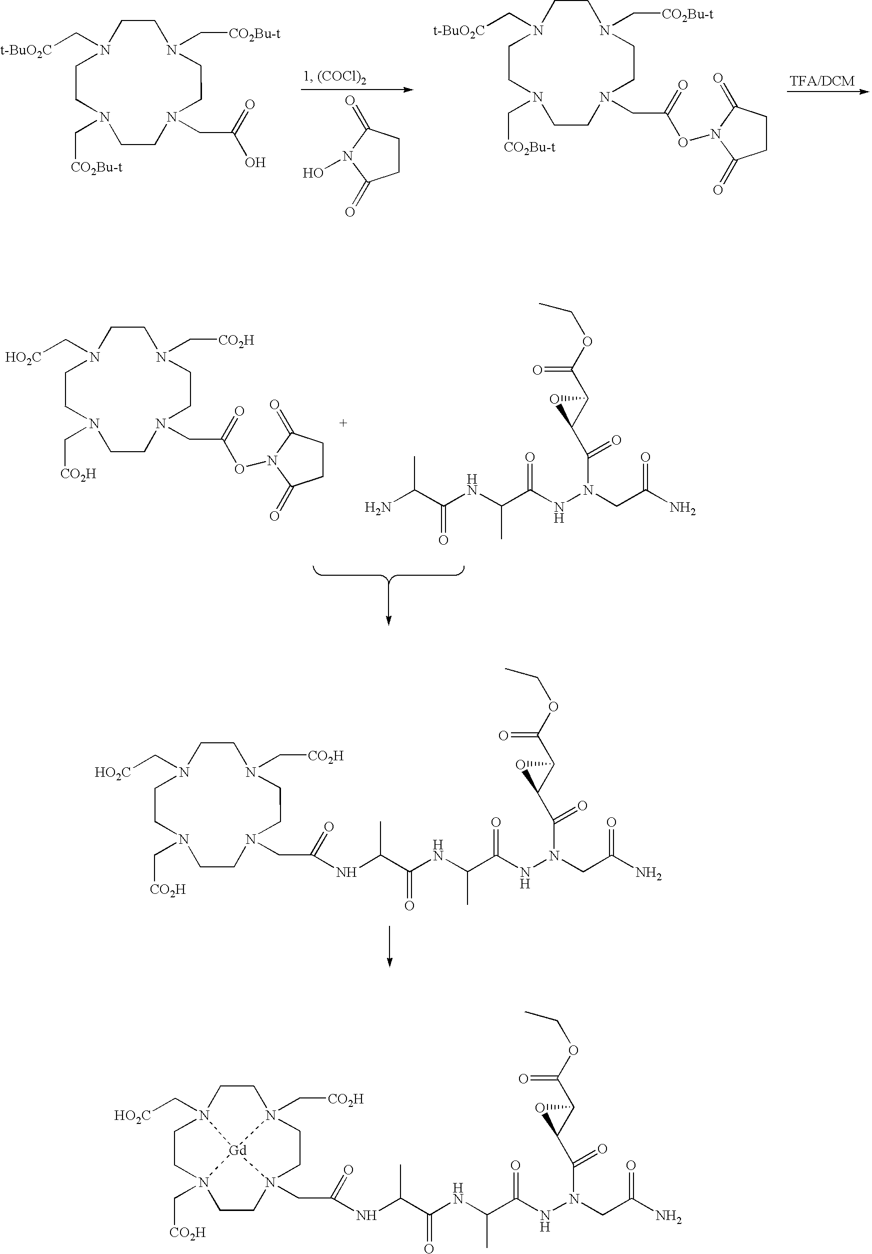

In some embodiments, boron may also be used a label. For example, the following structure is a boron and gadolinium bifunctional targeting entity.

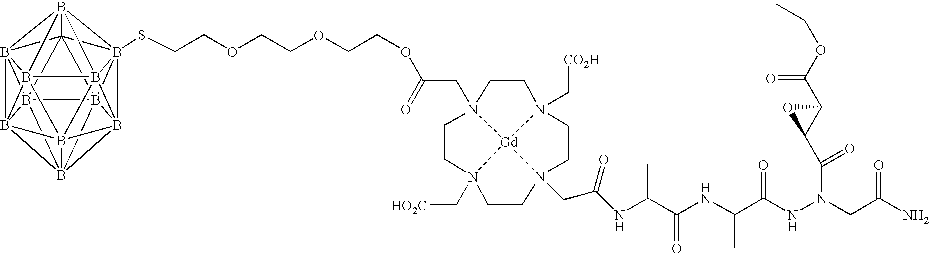

When boron-10 and gadolinium-157 are used in such a boron-gadolinium bifunctional targeting entity, tumors can be detected using magnetic resonance imaging (MRI) and then treated using neutron capture therapy. See, e.g., Takahashi et al., Synthesis and in vivo biodistribution of BPA-Gd-DTPA complex as a potential MRI contrast carrier for neutron capture therapy, BIOORGANIC & MEDICINAL CHEMISTRY 13: 735-743 (2005). Hence, this bifunctional boron-gadolinium agent is useful for treating cancers as well as detecting them.

Ions useful in other contexts, such as X-ray imaging, include but are not limited to lanthanum (III), gold (III), lead (II), and especially bismuth (III). Moreover, in the case of radioactive isotopes for therapeutic and/or diagnostic application, one might mention iodine¹³¹, iodine¹²³, iodine¹²⁵, technicium⁹⁹, indium¹¹¹, phosphorus³², rhenium¹⁸⁸, rhenium¹⁸⁶, gallium⁶⁷, sulfur³⁵, copper⁶⁷, yttrium⁹⁰, tritium³ or astatine²¹¹.

In some embodiments, agents and inhibitors may be conjugated with a dye or fluorescent moiety or intermediate such as biotin. Such conjugates can, for example, be used with infrared spectroscopy to detect and locate the tissues to which the agents bind.

In general, an assay for identifying a tumor via a tumor-specific protease involves incubating a test sample under conditions that permit binding of the protease to a diagnostic agent, and measuring whether such binding has occurred. In some embodiments, the extent of binding between the diagnostic agent and the protease may be detected. Such information may be used to detect and assess the extent, spread or size of a cancerous tumor. A reporter molecule can be attached to any molecule that stably binds to the protease and that can be detected. For example, the reporter molecule can be attached to one of the protease inhibitors described herein or to an antibody that binds to one of the proteases described herein that is labeled as described above with paramagnetic ions, ions, radioactive isotopes, fluorescent dyes (e.g., fluorescein, rhodamine), enzymes and the like. It is understood that the choice of a reporter molecule will depend upon the detection system used.

Specific examples of imaging agents that can be used in the invention include the following compounds:

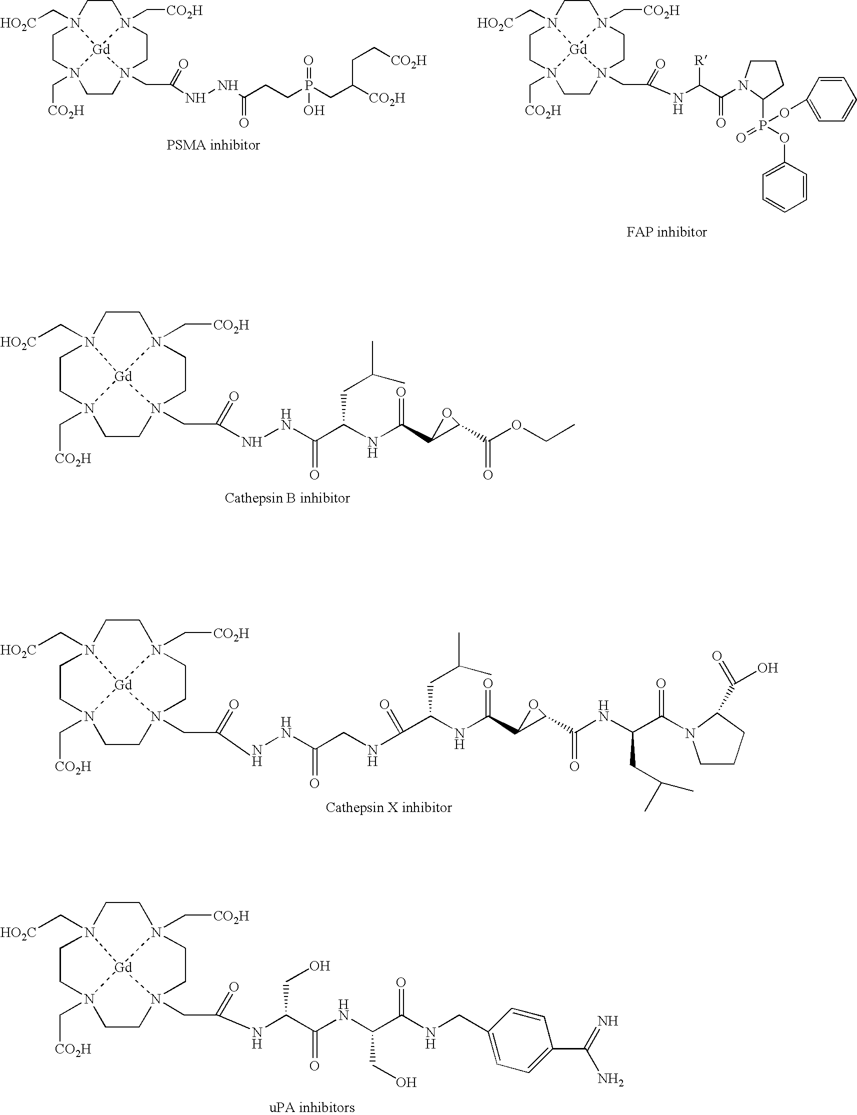

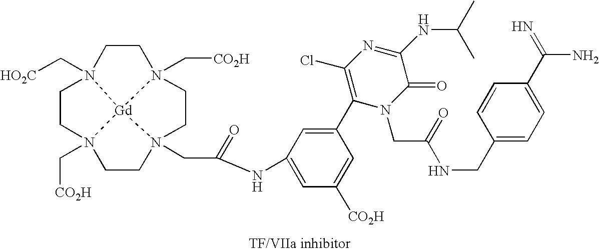

### Anti-Legumain Antibodies

The invention provides antibody preparations directed against legumain, for example, antibodies capable of binding a polypeptide having SEQ ID NO: 1. For example, in some embodiments, the antibody can bind a legumain epitope that includes the following sequence CGMKRASSPVPLPP (SEQ ID NO: 16). This sequence (SEQ ID NO: 16) is near the protease cleavage site that activates legumain. Therefore, this antibody preferentially recognizes activated legumain compared to legumain proenzyme. Such antibodies are desirable to detect -activated legumain proteases, which are associated with pathological cancer and tumor cells. Moreover, antibody preparations of the invention can serve as inhibitors of legumain activity and therefore act as therapeutic agents.

Methods are provided to prepare and screen for antibodies that preferentially recognize activated proteases, for example, asparaginyl endopeptidases. A peptide sequence (e.g. SEQ ID NO: 16) that is near the proteolytic cleavage site is used as antigen to raise polyclonal or monoclonal antibodies. The resultant antibodies are selected for binding to the selected peptide sequence, for binding to the activated protease and then for inhibition of the activated protease. The antibody recognition site at the free end of the peptide is only be available in activated proteases, hence desirable antibodies bind to the activated protease and exhibit substantially less binding to the non-activated protease. Inhibitory antibodies are selected by screening the antibodies for inhibition of proteolysis during activity assays.

Antibody molecules belong to a family of plasma proteins called immunoglobulins, whose basic building block, the immunoglobulin fold or domain, is used in various forms in many molecules of the immune system and other biological recognition systems. A typical immunoglobulin has four polypeptide chains, containing an antigen binding region known as a variable region and a non-varying region known as the constant region.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82, 4592-4596 (1985).

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody which includes the variable domain complementarity determining regions (CDR), and the like forms, all of which fall under the broad term “antibody”, as used herein. The present invention contemplates the use of any specificity of an antibody, polyclonal or monoclonal, and is not limited to antibodies that recognize and immunoreact with a specific antigen. In preferred embodiments, in the context of both the therapeutic and screening methods described below, an antibody or fragment thereof is used that is immunospecific for an antigen or epitope of the invention.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments that are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

Antibody fragments contemplated by the invention are therefore not full-length antibodies but do have similar or improved immunological properties relative to an anti-legumain antibody. Such antibody fragments may be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more. In general, an antibody fragment of the invention can have any upper size limit so long as it binds with specificity to legumain, e.g. a polypeptide having SEQ ID NO: 1.

Antibody fragments retain some ability to selectively bind with its antigen. Some types of antibody fragments are defined as follows:

(1) Fab is the fragment that contains a monovalent antigen-binding fragment of an antibody molecule. A Fab fragment can be produced by digestion of whole antibody with the enzyme papain to yield an intact light chain and a portion of one heavy chain.

(2) Fab′ is the fragment of an antibody molecule can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

(3) (Fab′)₂ is the fragment of an antibody that can be obtained by treating whole antibody with the enzyme pepsin without subsequent reduction. F(ab′)₂ is a dimer of two Fab′ fragments held together by two disulfide bonds.

(4) Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv including only three CDRs specific for an antigen) has the ability to recognize and bind antigen, although at a lower affinity than the entire binding site.

(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further includes a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269-315 (1994).

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad. Sci. USA 90: 6444-6448 (1993).

Methods for preparing polyclonal antibodies are available to those skilled in the art. See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1 (1992), which are hereby incorporated by reference.

Methods for preparing monoclonal antibodies are likewise available to one of skill in the art. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology, Vol. 10, pages 79-104 (Humana Press (1992).

Methods of in vitro and in vivo manipulation of monoclonal antibodies are also available to those skilled in the art. For example, monoclonal antibodies to be used in accordance with the present invention may be made by the hybridoma method first described by Kohler and Milstein, Nature 256, 495 (1975), or may be made by recombinant methods, e.g., as described in U.S. Pat. No. 4,816,567. The monoclonal antibodies for use with the present invention may also be isolated from phage antibody libraries using the techniques described in Clackson et al. Nature 352: 624-628 (1991), as well as in Marks et al., J. Mol. Biol. 222: 581-597 (1991). Another method involves humanizing a monoclonal antibody by recombinant means to generate antibodies containing human specific and recognizable sequences. See, for review, Holmes, et al., J. Immunol., 158:2192-2201 (1997) and Vaswani, et al., Annals Allergy, Asthma & Immunol., 81:105-115 (1998).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates that the antibody preparation is a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al. Proc. Natl. Acad. Sci. 81, 6851-6855 (1984).

Methods of making antibody fragments are also known in the art (see for example, Harlow and Lane, Antibodies: A Laboratory Manual, Cold Spring Harbor Laboratory, New York, (1988), incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, in U.S. Pat. No. 4,036,945 and No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be non-covalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., Methods: a Companion to Methods in Enzymology, Vol. 2, page 97 (1991); Bird, et al., Science 242:423-426 (1988); Ladner, et al, U.S. Pat. No. 4,946,778; and Pack, et al., Bio/Technology 11:1271-77 (1993).

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., Nature 321, 522-525 (1986); Reichmann et al., Nature 332, 323-329 (1988); Presta, Curr. Op. Struct. Biol. 2, 593-596 (1992); Holmes, et al., J. Immunol., 158:2192-2201 (1997) and Vaswani, et al., Annals Allergy, Asthma & Immunol., 81:105-115 (1998).

The invention also provides methods of mutating antibodies to optimize their affinity, selectivity, binding strength or other desirable property. A mutant antibody refers to an amino acid sequence variant of an antibody. In general, one or more of the amino acid residues in the mutant antibody is different from what is present in the reference antibody. Such mutant antibodies necessarily have less than 100% sequence identity or similarity with the reference amino acid sequence. In general, mutant antibodies have at least 75% amino acid sequence identity or similarity with the amino acid sequence of either the heavy or light chain variable domain of the reference antibody. Preferably, mutant antibodies have at least 80%, more preferably at least 85%, even more preferably at least 90%, and most preferably at least 95% amino acid sequence identity or similarity with the amino acid sequence of either the heavy or light chain variable domain of the reference antibody. One method of mutating antibodies involves affinity maturation using phage display.

The invention is therefore directed to a method for selecting antibodies and/or antibody fragments or antibody polypeptides with desirable properties. Such desirable properties can include increased binding affinity or selectivity for the epitopes of the invention

The antibodies and antibody fragments of the invention are isolated antibodies and antibody fragments. An isolated antibody is one that has been identified and separated and/or recovered from a component of the environment in which it was produced. Contaminant components of its production environment are materials that would interfere with diagnostic or therapeutic uses for the antibody, and may include enzymes, hormones, and other proteinaceous or nonproteinaceous solutes. The term “isolated antibody” also includes antibodies within recombinant cells because at least one component of the antibody's natural environment will not be present. Ordinarily, however, isolated antibody will be prepared by at least one purification step

If desired, the antibodies of the invention can be purified by any available procedure. For example, the antibodies can be affinity purified by binding an antibody preparation to a solid support to which the antigen used to raise the antibodies is bound. After washing off contaminants, the antibody can be eluted by known procedures. Those of skill in the art will know of various techniques common in the immunology arts for purification and/or concentration of polyclonal antibodies, as well as monoclonal antibodies (see for example, Coligan, et al., Unit 9, Current Protocols in Immunology, Wiley Interscience, 1991, incorporated by reference).

In some embodiments, the antibody will be purified as measurable by at least three different methods: 1) to greater than 95% by weight of antibody as determined by the Lowry method, and most preferably more than 99% by weight; 2) to a degree sufficient to obtain at least 15 residues of N-terminal or internal amino acid sequence by use of a spinning cup sequentator; or 3) to homogeneity by SDS-PAGE under reducing or non-reducing conditions using Coomasie blue or, preferably, silver stain.

Methods of Detecting and Isolating Agents that can Modulate Tumor-Specific Proteases

The invention further provides screening assays that are useful for generating or identifying therapeutic agents for prevention and treatment of cancer and assays for generating or identifying agents that modulate tumor-specific protease activity. In particular, the tumor-specific protease substrates, tumor-specific protease inhibitors, tumor-specific protease nucleic acids and tumor-specific protease identified herein may be used in a variety of assays for detecting tumor-specific protease and for identifying factors that interact with tumor-specific protease nucleic acids or with the tumor-specific proteases. For example, in some embodiments, assays can be performed to assess whether a potential prodrug can be activated by cleavage by a tumor-specific protease.

Prodrug conversion assay may be carried out in a variety of ways. For example, a cytotoxic agent can be linked to a peptide that has a tumor-specific protease cleavage site as described above. Cultured non-cancerous cells and cancerous cells (e.g., those that express tumor-specific protease) may then be separately exposed to the prodrug. After a suitable exposure time, the cultures are observed to ascertain whether the cancerous cells are preferentially killed or inhibited in their growth. The culture fluids may also be assayed to determine whether the prodrug has been cleaved in a manner that is consistent with tumor-specific protease cleavage. Before such assays are performed, prodrug candidates can be screened to ascertain whether they are efficiently cleaved by a tumor-specific protease. In some embodiments, the cancer cells employed overexpress a tumor-specific protease.

Further assays can be performed to assess the in vivo toxicity and in vivo efficacy for treating cancer. Suitable animal models and tumor cell lines can be used for these purposes. For example, mice, rats or other model animals with a propensity for developing cancer can be employed. Alternatively, small tumors or tumor cells or cancer cells that overexpress a tumor-specific protease can be transplanted into normal animals. Some of the animals that received tumors, tumor cells or cells that express a tumor specific protease are then treated with the prodrug. Other of those animals can be treated with the cytotoxic agent that forms part of the prodrug. Tumor growth and physical signs can be monitored daily including any gross evidence of tumor necrosis, local tumor ulceration as well as evidence of toxicity including mobility, response to stimulus, eating, and weight of each animal. Prodrugs that effectively reduce or eliminate tumors while having minimal negative effects on the health, lifespan and tissue integrity of the model animal are selected for development as a prodrug.

Assays may be used to identify agents that can interact with a cancer cell of interest. A wide variety of assays may be used for this purpose. See, for example, the assays carried out within the National Cancer Institute's “In Vitro Cell Line Screening Project.” In general, such an assay can involve contacting a cancer cell of interest with at least one agent and observing whether the agent kills the cancer cell and/or has other deleterious effects upon that cell.

Methods available in the art can also be used for determining whether the agents of the invention interact with the membrane of a cancer cell of interest. For example, the agent can be labeled with a reporter molecule that permits detection of the agent. After labeling, the agents can be contacted with the cancer cell of interest for a time and under conditions that permit binding or association of the agent to cellular membranes. The cells can be washed with physiological solutions to remove unbound or unassociated agents, and the cells can then be observed to ascertain whether the reporter molecule is bound or associated with the cells or the cellular membranes. In another embodiment, one of skill in the art can test whether the agent(s) can penetrate the membranes of selected cancer cells. This may be done by examining whether the reporter molecule remains associated with the cellular membranes of the cancer cell or whether the reporter molecule becomes associated with the interior of the cell.

Reporter molecules that can be employed include any detectable compound or molecule that is conjugated directly or indirectly to an agent of the invention. The label may itself be detectable (e.g., radioisotope labels or fluorescent labels) or, in the case of an enzymatic label, may catalyze chemical alteration of a substrate compound or composition that is detectable.

Deleterious effects upon the cancer cell of interest can also be detected as an indication of an interaction between an agent of the invention and the cell. Such deleterious effects can involve any evidence that the agent has had an adverse or cytotoxic effect upon the cell. For example, one of skill in the art can test whether the agent(s) kill the cancer cell, cause membrane depolarization, cause permeabilization of the membranes of the cell, or tend to lyse the cancer cells.

Pluralities of assays can be performed in parallel with different agents at different concentrations to obtain a differential response to the various concentrations. Typically, at least one control assay is included in the testing. Such a control can be a negative control involving exposure of the cancer cells of interest to a physiologic solution containing no agents. Another control can involve exposure of the cancer cell of interest to an agent that has already been observed to adversely affect the cancer cell of interest, or a second cell that is related to the cell of interest. Another control can involve exposing a cell of interest to a known therapeutic compound that has a desired affect on the cancer cell of interest, for example, an anti-cancer agent with known efficacy at a particular concentration or dosage. One of skill in the art can readily select control compounds and conditions that facilitate screening and analysis of the effects of the cyclic peptides on a cancer cell of interest.

Any cell type can be assayed by these methods. For example, any mammalian or other animal cancer cell type can be screened to assess whether the agents of the invention can selectively interact therewith. Mammalian or other animal cells can also be screened to ascertain whether the agents of the invention selectively interact therewith and/or to determine whether the agents of the invention do not interact, bind, lyse, kill or otherwise adversely affect the viability of the mammalian or other animal cell.

Conditions for screening include conditions that are used by one of skill in the art to grow, maintain or otherwise culture cell types of interest. Cancer cell types of interest should be assayed under conditions where they would be healthy but for the presence of the agents. Controls can be performed where the cell types are maintained under the selected culture conditions and not exposed to an agent, to assess whether the culture conditions influenced the viability of the cells. One of skill in the art can also perform the assay on cells that have been washed in simple physiological solutions, such as buffered saline, to eliminate, or test for, any interaction between the agents or cells and the components in the culture media. However, culture conditions for the assays generally include providing the cells with the appropriate concentration of nutrients, physiological salts, buffers and other components typically used to culture or maintain cells of the selected type. A variety of other reagents may be included in the screening assay. These include reagents like salts, neutral proteins, albumin, and serum (e.g. fetal calf serum) that are used to mimic the physiologic state of the cell types of interest. Conditions and media for culturing, growing and maintaining cells are available to one of skill in the art.

The selected reagents and components are added to the assay in the order selected by one of skill in the art. In general, the agents are added last to start the assay. Assays are performed at any suitable temperature, typically between 4° C. and 40° C. For example, the temperature may generally range from about room temperature (about 20° C.) to about 37° C. Incubation periods are selected to ascertain the optimal range of activity, or to insure that the agents do not adversely affect the cell type of interest. However, incubation times can be optimized to facilitate rapid high-throughput screening. Typically, incubation times are between about one minute and about five days, such as a range from about 30 minutes to about 3 days.

Agents having the desired activity in vitro may be tested for activity and/or lack of toxicity in vivo, in an appropriate animal model. Such animal models include primates as well as mice, rats, rabbits, cats, dogs, pigs, goats, cattle or horses. For example, the mouse is a convenient animal model for testing whether agents of the invention have toxic effects and/or to determine whether the agents can inhibit the growth of a cancer cell.

One of skill in the art can readily perform in vivo evaluation of the agents of the invention. For toxicity testing, a series of agents at different test dosages can be separately administered to different animals. A single dose or, a series of dosages can be administered to the animal. A test period is selected that permits assessment of the effects of the agent(s) on the animal. Such a test period can run from about one day to about several weeks or months.

The effect of a agent(s) on an animal can be determined by observing whether the agent adversely affects the behavior (e.g., lethargy, hyperactivity) and physiological state of the animal over the course of test period. The physiological state of the animal can be assessed by standard procedures. For example, during the test period one of skill in the art can draw blood and collect other bodily fluids to test, for example, for various enzymes, proteins, metabolites, and the like. One of skill in the art can also observe whether the animal has bloating, loss of appetite, diarrhea, vomiting, blood in the urine, loss of consciousness, and a variety of other physiological problems. After the test period, the animal can be sacrificed and anatomical, pathological, histological and other studies can be performed on the tissues or organs of the animal.

In general, to determine whether one or more agents of the invention can inhibit cancer cell growth, mice are infected with the selected cancer and a selected test dosage of one or more agents is administered shortly thereafter. Mice are observed over the course of several days to several weeks to ascertain whether the agents protect the mice from the cancer. At the end of the test period, mice can be sacrificed and examined to ascertain whether the agent has optimally protected the mice from cancer and/or to determine whether any adverse side effects have occurred.

Controls are used to establish the effects of the cancer when the agent is not administered. Other controls can also be performed, for example, to determine the safety and efficacy of the present agents compared to that of known anti-cancer compounds.

Binding assays between tumor-specific proteases and other agents may be carried out in several formats, including cell-based binding assays, solution-phase assays and immunoassays. In general, test samples or compounds are incubated with a tumor-specific protease for a specified period of time followed by measurement of binding between the tumor-specific protease and the test sample or compound. A label or reporter molecule attached to the tumor-specific protease, test sample or compound may be detected by use of microscopy, fluorimetry, a scintillation counter, or any available immunoassay. Binding can also be detected by labeling a tumor-specific protease in a competitive radioimmunoassay. Alternatively, a tumor-specific protease may be modified with an unlabeled epitope tag (e.g., biotin, peptides, His₆, FLAG, myc etc.) and bound to proteins such as streptavidin, anti-peptide or anti-protein antibodies that have a detectable label as described above. Additional forms of tumor-specific proteases containing epitope tags may be used in solution and immunoassays.

Methods for identifying compounds or molecules that interact with tumor-specific proteases are also encompassed by the invention. In general, an assay for identifying compounds or molecules that interact with a tumor-specific protease involves incubating the tumor-specific protease with a test sample that may contain such a compound or molecule under conditions that permit binding of the compound or molecule to the tumor-specific protease, and measuring whether binding has occurred. Tumor-specific proteases may be purified or present in mixtures, such as in cultured cells, tissue samples, body fluids or culture medium. Assays may be developed that are qualitative or quantitative. Quantitative assays can be used for determining the binding parameters (affinity constants and kinetics) of the compound or molecule for the tumor-specific protease and for quantifying levels of biologically active compounds and molecules in mixtures. Assays may also be used to evaluate the binding of a compound or molecule to fragments, analogs and derivatives of a tumor-specific protease and to identify new tumor-specific protease family members.

The compound or molecule in a test sample may be substantially purified or present in a crude mixture. Binding compounds and molecules may be nucleic acids, proteins, peptides, carbohydrates, lipids or small molecular weight organic compounds. The compounds and molecules may be further characterized by their ability to increase or decrease tumor-specific protease activity in order to determine whether they act as an agonist or an antagonist.

Tumor-specific proteases may be purified or be present in mixtures, such as in cultured cells, tissue samples, body fluids or culture medium. Assays may be developed that are qualitative or quantitative, with the latter being useful for determining the conversion rate or the binding parameters (affinity constants and kinetics) of the agent in its interaction with a tumor-specific protease and for quantifying levels of the tumor-specific protease in mixtures. Assays may also be used to detect fragments, analogs and derivatives of a tumor-specific protease and to identify new tumor-specific protease family members.

Tumor-specific protease nucleic acids are also useful for identification of factors that interact with the tumor-specific protease promoter and that modulate tumor-specific protease expression. Such factors may be intracellular proteins such as DNA binding proteins that interact with regulatory sequences that control tumor-specific protease transcription, for example, the tumor-specific protease promoters. As an example, hybrid constructs may be used that include a nucleic acid encoding the legumain promoter fused to a nucleic acid encoding a marker protein. The legumain promoter can be found within the genomic nucleotide sequence for human legumain that is available in the NCBI database at accession number NT 026437 (gi: 29736559). See website at ncbi.nlm.nih.gov. The marker protein can be any marker protein available to one of skill in the art. For example, the marker protein can be luciferase, green fluorescence protein (GFP) or CAT.

Such hybrid constructs are used for in vitro or in vivo transcription assays to identify factors that modulate tumor-specific protease expression. Factors that depress or diminish tumor-specific protease expression are particularly useful. Expression or transcription levels can be assessed using any method available to one of skill in the art for measuring RNA levels. For example, RNA levels can be assessed by northern analysis, reverse transcriptase analysis, reverse transcriptase coupled with polymerase chain reaction (RT-PCR) analysis and other methods. Chemical libraries can be screened using such methods for small molecule compounds that block tumor-specific protease transcription.

### Compositions

The prodrugs and inhibitor compounds of the invention as well as antibodies and inhibitors of tumor-specific proteases can be formulated as pharmaceutical compositions and administered to a mammalian host, such as a human patient in a variety of forms adapted to the chosen route of administration. Routes for administration include, for example, oral, parenteral, intraperitoneal, intravenous and intraarterial routes.

Solutions of the agents or their salts can be prepared in water or saline, and optionally mixed with a nontoxic surfactant. Formulations for intravenous or intraarterial administration may include sterile aqueous solutions that may also contain buffers, liposomes, diluents and other suitable additives.

Prodrugs and inhibitors with hydrophilic R₁ groups are designed to be water soluble and can be formulated in aqueous carriers or in solid dosage forms for dissolution in an aqueous environment (e.g. an aqueous environment in vivo).

The pharmaceutical dosage forms suitable for injection or infusion can include sterile aqueous solutions or dispersions comprising the active ingredient that are adapted for administration by encapsulation in liposomes. In some embodiments, the liposomes contain a somewhat more hydrophobic prodrug or asparaginyl endopeptidase inhibitor. Thus, reduced numbers of hydrophilic R₁ groups may be present on the prodrug or inhibitor. Instead, a less hydrophilic protecting group (e.g. t-BOC) may be used instead of a hydrophilic R₁ group. Alternatively, no hydrophilic R₁ or protecting groups may be used. For example, in some embodiments, prodrugs or inhibitors may be used in the liposome compositions. Such liposome-prodrug or liposome-inhibitor compositions may have hydrophobic R₁ groups or hydrogen as the R₁ group. The ultimate dosage form should be sterile, fluid and stable under the conditions of manufacture and storage.

Sterile injectable solutions are prepared by incorporating the agents and inhibitors in the required amount in the appropriate solvent with various of the other ingredients, as required, followed by filter sterilization.

Useful dosages of the agents, prodrugs and inhibitors can be determined by comparing their in vitro activity, and in vivo activity in animal models. Methods for the extrapolation of effective dosages in mice, and other animals, to humans are known to the art; for example, see U.S. Pat. No. 4,938,949. The compound can be conveniently administered in unit dosage form.

The desired dose may conveniently be presented in a single dose or as divided doses administered at appropriate intervals, for example, as two, three, four or more sub-doses per day. The sub-dose itself may be further divided, for example, into a number of discrete loosely spaced administrations; such as multiple oral, intraperitoneal or intravenous doses. For example, it is desirable to administer the present compositions intravenously over an extended period, either by continuous infusion or in separate doses.

In some instances, the agents, prodrugs and inhibitors can be administered orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, they may be combined with one or more excipients and used in the form of ingestible tablets, buccal tablets, troches, capsules, elixirs, suspensions, syrups, wafers, and the like. Such compositions and preparations may contain at least 0.1% of active compound. The percentage of the compositions and preparations may, of course, be varied. The amount of compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use.

In some embodiments, the prodrugs or inhibitors are linked to polyethylene glycol (PEG). For example, one of skill in the art may choose to link a drug to PEG to form the following pegylated prodrug:

Drug-(linker)-protease cleavage site-(linker)-PEG wherein the linker can be a covalent bond, an amino acid, a peptidyl sequence, a sugar residue, a glycol chain or an alkylene chain.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

The therapeutically effective amount of prodrug, compound, or inhibitor necessarily varies with the subject and the disease or physiological problem to be treated. As one skilled in the art would recognize, the amount can be varied depending on the method of administration. The amount of the agent or inhibitor for use in treatment will vary not only with the route of administration, but also the nature of the condition being treated and the age and condition of the patient and will be ultimately at the discretion of the attendant physician or clinician.

The pharmaceutical compositions of the invention can include an effective amount of at least one of the agents of the invention, or two or more different agents of the invention. These compositions also include a pharmaceutically effective carrier.

The compositions of the invention can also include other therapeutic agents, for example, other chemotherapeutic agents, anti-inflammatory agents, analgeisics, vitamins, and the like.

The invention will be described with reference to the following non-limiting examples.

### Example 1

### Legumain is Expressed in Tumors

Example 1 demonstrates that legumain is over-expressed in human tumors.

### Materials and Methods

Reagents and cell lines. Rabbit polyclonal antisera against human legumain, as well as 293 cells stably expressing human legumain, were kindly provided by Dr. D. Roodman (Department of Medicine and Hematology, University of Texas Health Science Center, San Antonio, Tex.). A legumain substrate peptide was synthesized by and purchased from Bachem (King of Prussia, Pa.). Doxorubicin was purchased from Sigma (St. Louis, Mo.). Costar migration chambers were obtained from Corning Incorporated (Corning, N.Y.). Vitrogen was obtained from Cohesion Technologies (Palo Alto, Calif.). Mouse monoclonal antibody specific for human integrin β1 was obtained from Dr. R. Klemke (The Scripps Research Institute). The DMEM media was obtained from Invitrogen (Carlsbad, Calif.). The CT26 murine colon carcinoma cell line, the C1300 mouse neuroblastoma cell line anf human HT1080 fibrosarcoma cells were purchased from the American Type Culture Collection (ATCC). The 293 cells used to construct tetracycline-regulated cell lines expressing legumain were obtained from Stratagene (La Jolla, Calif.). Multiple tumor tissue arrays were provided by Cooperative Human Tissue Network, National Cancer Institute.

Rapid isolation of tumor endothelial cells and mRNA extraction. CD31 antibody coated Dynabeads were prepared by mixing 300 μl bead suspension with 500 μl PBSA (PBS, 1% BSA). Biotinylated anti mouse CD31 antibody (20 μg) was added to the suspension and association of antibody to beads was for 20 minutes at 4° C. The beads were washed 3 times with PBS to remove unbound antibody. CT26 tumors grown to ˜1.5 cm greatest diameter were surgically removed and cooled to 4° C. for following steps, and the tumor minced into 1 mm³ bits with sterile scissors. The minced tumor was gently pressed through metal meshes and filtered through a 40 μm Falcon cell strainer (Becton Dickinson, Franklin Lakes, N.J.) to rapidly recover the single cell suspensions. Streptavidin conjugated paramagnetic Dynabeads (Dynal, Lake Success, N.Y.) coated with biotinylated anti-mouse CD31 antibody (Mec 13.3, PharMingen, La Jolla, Calif.) were immediately added to the single cell suspensions. Capture by beads of CD31 positive cells was conducted at 4° C. for 20 minutes with gentle agitation. Beads with bound CD31 positive cells were recovered with a magnetic trap column and washed three times with cold phosphate buffered saline (PBS). Unbound CD31 negative cells were collected separately and were recovered by centrifugation at 1000 rpm for 3 minutes. Both CD31 positive and CD31 negative cells were used for mRNA extraction (Qiagene mRNA direct kit). The concentration of mRNA was quantified with RiboGreen RNA quantification reagents (Molecular Probes, Eugene, Oreg.).

Differential gene expression profiling using restriction fragment differential display. Five hundred ng mRNA was used for differential profiling using the displayPROFILE method (Display Systems Biotech, Vista, Calif.). The mRNA was first used to synthesize double stranded cDNA. The resultant double stranded DNA was digested with Taq I and adaptors were ligated onto the fragment ends. Display primer was used to PCR amplify the gene fragment profiles, which were then displayed on a 6% sequencing gel. Differentially displayed bands were cut from the sequencing gel and extracted with 50 μl water for 15 min in a boiling water bath. The fragments were reamplified with the same set of primers and then electrophoresed on 4% agarose gels. The amplified fragments were recovered from the gels and cloned into a pCRII vector by the Topo cloning method (Invitrogen, Carlsbad, Calif.). The vectors were then sequenced and BLAST searches performed with NCBI database to identify genes.

Histological and immunohistochemical analysis. Immunohistochemical staining was performed on both formalin fixed and unfixed frozen 5 μm thick sections on poly-L-lysine slides. For endothelial identification, biotinylated rat anti-mouse CD31 monoclonal antibody (MEC 13.3) was used with fluorescein conjugated streptavidin as the secondary reporting reagent. Rabbit anti-legumain antisera were prepared by immunization with purified human legumain produced in E. coli (Choi et al., 1999). The antisera recognized both mouse and rat legumain in frozen sections, as well as human legumain in formalin fixed sections. For staining of legumain in both frozen and formalin fixed sections, rabbit polyclonal anti-legumain antisera was used at 1:500 dilution followed by biotinylated anti-rabbit IgG as the second antibody. The reaction was visualized with Texas-red conjugated streptavidin and the slides were analyzed by laser scanning confocal microscope (Bio-Rad, Hercules, Calif.). For chromogenic staining, the rabbit polyclonal anti-legumain antibody was followed by a biotinylated goat anti rabbit antisera (Vector, Burlingame, Calif.). Streptavidin conjugated peroxidase was used and developed with the substrate BAD (Vector, Burlingame, Calif.).

Western BlotAnalysis. Proteins were dissolved in 2×SDS sample buffer for SDS PAGE analysis using gradient (8-16%) Tris-glycine gels. Following electrophoresis, the proteins were transferred to nitrocellulose membranes, and blocked with non-fat milk. The anti-legumain antisera was used as the first antibody and was incubated with membrane for one hour (1:1,000 dilution). The blot was washed three times with PBS, incubated with streptavidin-peroxidase for 15 min and developed by the ECL method (Sigma, St. Louis, Mo.).

Statistical analysis. Statistical significance of data in this and other Examples was determined by the two-tailed Student's t test.

### Results

Data on the expression patterns of Legumain is described below and shown in PCT Application Ser. No. PCT/US2004/017157 filed May 28, 2004, which is incorporated by reference herein in its entirety.

Over-expression of legumain in solid tumors. Use of restriction fragment differential display (Wrang et al., 2001; Gravesen et al., 2000; Zhang et al., 1998; Theissen et al., 1997) demonstrated that legumain is highly expressed in vivo in the CT26 murine colon carcinoma. Immunohistochemical studies of the CT26 tumor indicated that legumain is expressed by both tumor cells and, frequently, by tumor associated endothelial cells, both intracellularly and on the cell surface. Legumain over-expression in mouse tumors was confirmed by Western blot analysis. Legumain was also expressed by some normal mouse tissues. Legumain expression was not detected in the CT26 cell line in culture that was used to generate the syngeneic mouse colon carcinoma model, and legumain expression was not detected in other tumor cell lines in culture that were tested.

The surprising and unexpected up-regulation during tumor development in vivo suggests an in vivo environmental response. Legumain appears to be a stress responsive gene, because, although not detectable in cultured cells under typical tissue culture conditions, its expression was markedly elevated in cells subjected to environmental stress, such as serum starvation or in vivo growth.

To characterize legumain expression in normal human tissues and tumors, human tumor tissue arrays were analyzed immunohistochemically with anti-legumain antisera. While legumain expression was sparse in normal tissues, its expression was highest in the kidney. Legumain was also detected in liver and spleen, agreeing with published results (Chen et al., 1997; Chen et al., 1998).

Notably, legumain was highly expressed in the majority of human tumor tissue panels analyzed, which encompassed a wide variety of solid tumors (Table 3). Expression was highest for prostate carcinomas and positive for most breast and colon carcinoma specimens. All central nervous system malignancies were also positive for legumain expression.

TABLE 3
Legumain detection in human tumors.

| Carcinoma | Number | Number | Percentage | Degree of |
|---|---|---|---|---|
| Type | analyzed | positive | positive | positivity |
| Breast | 43 | 43 | 100% | +++ |
| carcinoma | | | | |
| Colon carcinoma | 34 | 32 | 95% | +++ |
| Lung carcinoma | 24 | 14 | 58% | +++ |
| Prostate | 56 | 42 | 75% | ++++ |
| carcinoma | | | | |
| Ovarian | 23 | 17 | 73% | ++ |
| carcinoma | | | | |
| CNS tumors | 8 | 8 | 100% | ++ |
| Lymphoma | 14 | 8 | 57% | + |
| Melanoma | 12 | 5 | 41% | + |

Cellular distribution of legumain. Legumain was most abundantly visualized associated within intracellular membranous vesicles, consistent with a proposed function as a lysosomal protease. The delivery of membranous vesicles containing proteases, adhesion molecules, and actin binding proteins toward the leading edge of migratory cells has been implicated in cell locomotion (Nabi et al., 1999; Bretscher et al., 1998). The legumain positive membranous vesicles were often concentrated at the invadopodia of tumor cells. Unexpectedly, legumain was also observed in apparent association with cell surfaces (serum starved BEND3 cells), as well as with the actin cortex.

Double staining of legumain+ 293 cells with antibody against integrin β1 and anti-legumain antibody revealed the presence of legumain inside cells in a granular organelle resembling aggregated lysosomes, and also on the cell surface colocalized with β1 integrins. The binding of legumain to β1 integrins is further described below. Legumain has an RGD sequence motif that might facilitate legumain association with cell surface integrins.

Therefore, legumain is highly expressed by most human tumors. A high percentage of breast carcinomas, colon carcinomas, and central nerve system neoplasms strongly expressed legumain, with the highest expression found in prostate tumors. In contrast, legumain was weakly expressed or not observed in the normal tissues of tumor derivation. Furthermore, legumain expression was negative for the cell lines in culture that were used to generate the in vivo tumors, even though legumain was readily detected after those cell lines had been placed in vivo. These results are indicative of induction of gene expression by the in vivo tumor environment.

### Example 2

### Legumain and Cell Migration, Tumor Invasion, and Metastasis

Example 2 discloses that legumain promotes cell migration and over-expression and is associated with enhanced tissue invasion and metastasis.

### Materials and Methods

Cell invasion and mobility assays. Cell migration and invasion assays were performed as described with modifications (Albini et al., 1987). Stock solutions (15 mg/ml) of Matrigel basement membrane matrix (Becton Dickinson, Bedford, Mass.) were stored at −80° C. in 100 μl aliquots. After thawing on ice, the stock was diluted 1:50 with cold serum-free culture media and immediately applied to each membrane insert (8 μm pore) that formed the upper chambers of the multi-well invasion assay plate. The Matrigel was incubated overnight in a sterile laminar tissue culture hood. The membranes were hydrated for 2 hours with 250 μl of serum-free medium and excess medium was removed by aspiration. Medium containing 10% FBS was added to the bottom of each well. A suspension of 10⁵ cells in 150 μl of serum-free medium was added to the upper chamber and incubated for 12 hours at 37° C., 5% CO₂. At the indicated times, the membrane inserts were removed from the plate and the non-invading cells were removed from the upper surface. Membrane associated cells were stained with 0.09% crystal violet for 30 minutes and washed twice with PBS. The invading cells were counted microscopically. Cell mobility assays were performed in a similar manner except the membrane inserts were not coated with Matrigel, and duration was shortened. In some assays, protease inhibitors were added to the invasion chamber at the beginning of the incubation.

Zymogram. Control 293 cells and legumain+ 293 cells were plated into 96 wells plates at a cell density of 4,000 cells/well. The cells were allowed to attach overnight, then were serum starved for four hours. Zymogen forms of metalloproteinase 2 or 9 (Chemicon, Temecula, Calif.) were added at concentration of 0.1 μg/well with 50 μl reaction buffer (39.5 mM citric acid, 121 mM Na₂HPO₄, pH 5.8, 1 mM EDTA, and 0.8% Na₂Cl) and the reactions were continued for 10 minutes. The reactants were collected and mixed with an equal volume of SDS sample buffer and held at room temperature for 10 minutes then applied to a zymogram gel (10% Tris-Glycine gel with 0.1% gelatin substrate). After electrophoresis, the gel was washed briefly and incubated with 2.5% (v/v) Triton X-100 at room temperature for 30 minutes with gentle agitation. Digestion of the incorporated gelatin by activated collagenase was conducted in buffer (50 nM Tris, pH 7.25, 200 mM NaCl, 10 nM CaCl₂, 0.05% Brij-35, 0.02% NaN₃) overnight. The gel was stained with Coomassie Blue R250 (Novex, San Diego, Calif.) and the presence of a protease was readily observed as a clear band.

### Results

Data on the effects of Legumain on cell migration and cell invasion is described below and is also shown in PCT Application Ser. No. PCT/US2004/017157 filed May 28, 2004, which is incorporated by reference herein in its entirety.

Legumain expression promotes cell migration and invasion. The effect of legumain expression on cell migration and invasion was investigated. In an in vitro migration assay, legumain+ 293 cells exhibited increased migration in comparison with wild type 293 cells. The enhanced migration was inhibited by cystatin, a known inhibitor of legumain protease function, and weakly by TIMP-2 protein, but not by E64.

Next, control 293 cells and legumain+ 293 cells were evaluated in a modified Boyden chamber invasion assay. Legumain+ cells exhibited increased invasion of extracellular matrix. Such increased invasion was inhibited by cystatin, but to only a limited extent by TIMP-2. Again E64 was without effect. These experiments were repeated with a 293 cell line in which the transcription of legumain was conditionally regulated by tetracycline. Comparable results were obtained (data not shown).

Legumain expression correlates with tumor invasion and metastasis. To explore the effects of legumain expression in vivo, legumain+ 293 cells and control 293 cells were injected subcutaneously into the backs of WEHI nude mice. Tumors appeared after 2-3 weeks. The initial rates of primary tumor growth were comparable in mice receiving either legumain+ 293 cells or control 293 cells. However, while there was prominent legumain expression in the legumain+ 293 tumors only weak but positive legumain expression was detectable in control 293 tumors, despite an absence of legumain expression by these cells in culture. These results were similar to the observations for the CT26 colon carcinoma cells. Histological analysis of more advanced tumors suggested a lower rate of apoptosis for legumain+ 293 tumors compared to control 293 tumors.

In contrast to control 293 tumors, legumain+ 293 tumors frequently metastasized in vivo. At necropsy, 50% of legumain+ 293 tumor bearing mice had metastatic nodules in distant organs, predominantly in the lung and liver. In contrast, no metastatic nodules were observed in distant organs within animals that received control 293 cells. The increased invasion and metastasis associated with legumain over-expression is consistent with legumain-facilitated tumor metastasis and progression. The more invasive legumain+ 293 tumors frequently invaded muscles and frequently lacked the well defined pseudo-encapsulation observed with control 293 tumors. This more invasive tumor behavior was evident in early as well as later stage tumors.

Activation of progelatinase A by legumain. Tumor cell surface-associated proteases may degrade extracellular matrix proteins, for example, gelatinase A and cathepsins. For example, the activation of progelatinase A requires cleavage of an asparaginyl bond (Nagase et al., 1997). Therefore a gelatin zymogram study was performed to evaluate whether cell surface bound legumain can convert the 72 kDa zymogen to the 62 kDa active enzyme was examined. Cells expressing legumain, as well as control cells, were deposited in 96 well plates. After attachment and serum starvation, the culture media was removed. Zymogen forms of gelatinase A and B were incubated with the cells for 10 minutes, and the reaction products were analyzed by zymography. Generation of the 62 kDa active enzyme was observed for cells expressing legumain, and the activation was inhibited by cystatin. Legumain alone did not degrade gelatin, indicating that legumain expression in cells is needed for such degradation. Moreover, no effect of legumain on zymogen gelatinase B was detected.

### Discussion

Proteases have been implicated in many aspects of tumor cell biology (Chang et al., 2001). Thus, a protease that is highly expressed by tumor cells or tumor vascular endothelial cells might contribute to tumor cell progression through processing of signaling molecules and their receptors, thereby influencing cellular responses. Such effects might also result in diminished apoptosis (Hanahan et al., 2000), thereby enhancing tumor growth.

Evidence presented herein not only shows atypical expression, but also illustrates participation of legumain in effector functions. In particular, legumain is an apparent regulator of cellular behavior in migration and tissue invasion because cells that highly express legumain exhibited enhanced migratory and invasive properties. A correlation between tumor invasion and metastasis with the presence of cysteine endopeptidases (particularly cathepsins B and L) has been observed (Mai et al., 2000).

Hydrolysis of asparaginyl bonds is prominent in the post-translational processing of cathepsin B, D, and H (Chen et al., 1997; Chen et al., 1998; Yonezawa et al., 1988), which are cysteine proteases. Legumain might therefore activate the local cysteine protease zymogens to form the active two chain protease forms. In addition to the established plasminogen/plasmin system and the metalloproteinase system, a cysteine protease cascade may represent an additional tumor invasion/metastasis cascade. As described, a 62 kDa activated gelatinase A enzyme was observed in cells expressing legumain, and such activation was inhibited by cystatin, however legumain had no effect on zymogen gelatinase B. Hence, legumain activates the gelatinase A zymogen, an important mediator of extracellular matrix degradation. The activation mechanism of gelatinase A by legumain differs from that involved with the membrane type matrix metalloproteinases (Itoh et al., 1998). This may be important for tumor cell adaptation to a more invasive and metastatic phenotype.

Legumain-promoted cell migration and invasion can be partially inhibited by cystatin and TIMP-2. The inhibition of mammalian legumain by cystatin is due to a novel second reactive site (Alvarez-Fernandez et al., 1999). Another cysteine inhibitor, E64, has no affect on legumain or cell migration. Hence, the increased cell migration in legumain+ cells is not due to a member of the papain family of cysteine proteases that are characteristically susceptible to E64 inhibition.

Analysis by site-directed mutagenesis of the catalytic residues of mammalian legumain suggests a catalytic dyad exists with the motif His-Gly-spacer-Ala-Cys. The presence of this motif is also found in the catalytic sites of the caspases, the aspartate-specific endopeptidases central to the process of apoptosis in animal cells, as well as in the families of clostripain and gingipain which are arginyl/lysyl endopeptidases of pathogenic bacteria. However, legumain is notably distinct from other lysosomal cysteine proteases. In particular, its catalytic activity is unique in that it is the only asparaginyl endopeptidase identified to date. Moreover, the sequence for legumain is conserved through evolution. Its conservation and unique enzymatic activity indicate legumain may have a significant biologic function.

Animal tumor models generated with cells over-expressing legumain had more vigorous and invasive growth and metastasis in vivo behavior than similar tumor cells that did not overexpress legumain. These results indicate that the proteolytic function of legumain may activate other protease zymogens. The inhibitory effect of cystatins on tumor cells (Sexton et al., 1997; Coulibaly et al., 1999) is consistent with the involvement of legumain and perhaps other cysteine proteases in tumor invasion and metastasis.

Tumor invasion and metastasis are critical determinants of cancer lethality, linked to 90% of human cancer deaths (Sporn et al., 1996). Invasion and metastasis are considered to be associated properties of tumor cells as they utilize similar processes involving physical attachment of cells to their environment through cell adhesion molecules (CAMs) and activation of extracellular proteases (Hanahan et al., 2000). Increased expression of proteases and down regulation of protease inhibitors is commonly observed in tumors (Yano et al., 2001; Chamber et al., 1997). Notably, cell surface proteases are often associated with invasive and metastatic tumor cells (Chang et al., 2001). Some proteases are linked to other properties of tumors such as angiogenesis (Stetler-Stevenson et al., 1999) and growth signaling (Werb et al., 1997) as perhaps with legumain.

Protease zymogens are dependent on limited proteolytic activation for conversion to the functional state. Protease cascades are characteristic of many biologic pathways, such as the coagulation, apoptosis, and complement cascades. Similar cascades appear to be involved in tumor invasion and metastasis. Characterization of the later is complicated by the diversity of neoplasms. However, comprehensive profiling of protease expression and function may advance understanding of tumor invasion and metastasis.

Some metalloproteinase inhibitors have demonstrated tumor stasis activities in animal models. Similarly, legumain represents a target for inhibition of growth and metastasis based on results showing that up-regulation of legumain expression is associated with tumor growth and the unique restricted specificity of legumain.

Legumain functions both extracellularly and intracellularly. Therefore, a cell-permeable inhibitor might extend the efficacy observed with cystatin, as the latter is cell-impermeable and has shown limited inhibition of in vitro cell migration and invasion.

Tumor cells with higher legumain levels appear to be more resistant to apoptosis. Although the precise molecular pathway has yet to be defined for this effect, lysosomal proteases are known to participate as effector enzymes in apoptosis (Foghsgaard et al., 2001; Castino et al., 2002). Thus, the sub-cellular localization of legumain may determine its targets and thereby its effects on the apoptosis cascades.

### Example 3

### Tumoricidal Effects of a Prodrug

Legumain's unique functional properties and high level of expression in a wide range of human tumors makes it a potential candidate target for enzymatic activation of a prodrug that can help eradicate tumors.

The integrity of the amino group of doxorubicin is essential for function. It has been shown that doxorubicin tolerates the addition of a leucine residue at this site. However incorporation of additional amino acids abolishes cytotoxic activity (de Jong et al., 1992; Denmeade et al., 1998).

In this Example, a prototype prodrug was synthesized by addition of an asparaginyl endopeptidase substrate peptide to doxorubicin. Upon exposure to legumain, the agent was converted to an active cytotoxic leucine-doxorubicin molecule. The prodrug had markedly reduced toxicity compared to doxorubicin, but it was effectively tumoricidal in a murine colon carcinoma model where it was presumably cleaved to form the leucine-doxorubicin cytotoxin. Therefore, according to the invention, legumain is a new target for tumoricidal prodrug development and therapy.

### Materials and Methods

Prodrug Synthesis. N-(-t-Butoxycarbonyl-L-alanyl-L-alanyl-L-asparaginyl-L-leucyl)doxorubicin (SEQ ID NO:7) has the following structure.

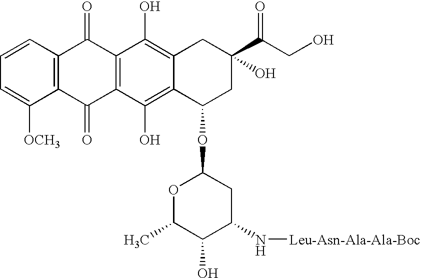

Note that the amino acid sequence shown above is written with the C-terminus to the left and the N-terminus to the right. Thus, the doxorubicin moiety is attached to the C-terminus of the peptide.

N-(-t-Butoxycarbonyl-L-alanyl-L-alanyl-L-asparaginyl-L-leucyl)doxorubicin (SEQ ID NO:7) was synthesized as follows. To cold (0° C.) solution of t-Butoxycarbonyl-L-alanyl-L-alanyl-L-asparaginyl-L-leucine (SEQ ID NO:29) (43 mg, 95 μmol) and 4-Methylmorpholine (20 μL, 200 μmol) in 5 mL DMF was added O-Benzotriazol-1-N,N,N′,N′-tetramethyluronium hexafluorophosphate (HBTU) (54 mg, 142.5 μmol). After 10 min, doxorubicin hydrochloride (50 mg, 86 μmol) was added and the mixture was stirred for 2 h at room temperature in the dark. The solution was diluted with 30 mL of EtOAc and washed with water. The solvent was evaporated, and solids were dried over MgSO₄ and purified over silica gel using CHCl₃/MeOH (90/10) while protected from light to yield 65 mg of compound 1 (75% yield). ¹H NMR (600 MHz, CD₃OD, δ): 0.82 (d, 3H, J=6.1), 0.88 (d, 3H, J=6.6), 1.28-1.35 (m, 9H), 1.43 (s, 9H), 1.59-1.74 (m, 4H), 2.05 (m, 1H), 2.17 (m, 1H), 2.36 (d, 1H, J=14.5), 2.67 (m, 1H), 2.79 (m, 1H), 2.91 (d, 1H, J=18.0), 3.04 (d, 1H, J=18.0), 3.62 (m, 1H), 4.01-4.04 (m, 4H), 4.11 (m, 1H), 4.22-4.32 (m, 3H), 4.59 (dd, 1H, J=5.9, 7.2), 4.74 (d, 2H, J=4.4), 5.08 (s, 1H), 5.39 (d, 1H, J=3.1), 7.51 (d, 1H, J=8.8), 7.78 (dd, 1H, J=7.9, 7.9), 7.86 (d, 1H, J=7.5). Preparation HRMS (MALDI) calculated for C₄₈H₆₄N₆O₁₈[M+Na]⁺ is 1035.4169, and found is 1035.4234. The compounds were purified by semi-preparative HPLC.

Cytotoxic assays. The WST-1 cell proliferation reagent (Roche Molecular Chemicals, Germany) was used to determine cell proliferation by quantification of cellular metabolic activity. Control 293 cells and legumain+ 293 cells were cultivated in microtiter plates (5×10³ cells per well in 100 μl) and were incubated with serial concentrations of legubicin or doxorubicin for 48 h. Subsequently, 10 μl of WST-1 solution (1 mg/ml WST-1, 25 μM -methyldibenzopyrazine methyl sulfate) was added per well, and mixtures were incubated for an additional 4 h. The tetrazolium salt WST-1 (4-[3-(4-lodophenyl)-2-(4-nitrophenyl)-2H-5-tetrazolio-1,3-benzene disfonate) was cleaved by the mitochondrial succinate-tetrazolium-reductase system to formazan in cells which directly correlates to the number of metabolically viable cells in the culture. The amount of formazan salt was quantified in three replicates by absorbance at 450 nm using a micro-plate reader (Molecular Devices, Palo Alto, Calif.). All results were derived from replicate experiments with similar results.

Animal models. The CT26 syngeneic murine colon carcinoma model was generated and maintained in The Scripps Research Institute animal facility. Balb/C mice aged 4 to 6 weeks from the breeding colony were inoculated with 500,000 syngeneic CT26 tumor cells per site subcutaneously in the back. Treatment was initiated when the tumors reached 4 mm in diameter through bolus intraperitoneal injection of the indicated reagents. Treatment was repeated at 2 day intervals. The human 293 tumor models were generated in WEHI nude mice (The Scripps Research Institute breeding colony). Either legumain+ 293 cells or control 293 cells (10⁶ cells/site) were inoculated subcutaneously into the backs of mice. Tumor growth and physical signs were monitored daily including any gross evidence of tumor necrosis, local tumor ulceration as well as evidence of toxicity including mobility, response to stimulus, eating, and weight of each animal. These procedures have been reviewed and approved by the Institutional Animal Care and Use Committee at The Scripps Research Institute. The work was conducted in The Scripps Research Institute facilities which are accredited by the Association for the Assessment and Accreditation of Laboratory Animal Care. The Scripps Research Institute maintains an assurance with the Public Health Service, is registered with the United States Department of Agriculture and is in compliance with all regulations relating to animal care and welfare.

### Results

Model prodrug activation by legumain. The functional capacity of tumor cell-associated legumain was explored based on the novel asparaginyl specific endopeptidase activity of legumain. The amino group of doxorubicin is critical for function. However a leucine residue can be added with retention of cytotoxicity. The prodrug, N-(-t-Butoxycarbonyl-L-alanyl-L-alanyl-L-asparaginyl-L-leucyl) doxorubicin (SEQ ID NO:7), was synthesized by addition of an asparaginyl endopeptidase substrate peptide Boc-Ala-Ala-Asn-Leu (SEQ ID NO:30) to the amino group of doxorubicin through a peptide bond at carboxy terminus of leucine. Upon cleavage by legumain, the prodrug is converted to a leucine-doxorubicin molecule, thereby regaining cytotoxic function. In addition, the presence of the Boc at the amino terminus prevents aminopeptidase hydrolysis of the peptidyl component. This prodrug is designated Legubicin.

First, the cytotoxic activity of doxorubicin and legubicin upon activation by legumain was analyzed in vitro using legumain+ 293 cells and control 293 cells. The effect of doxorubicin on both 293 cell types was similar, with legumain+ cells only slightly more resistant to doxorubicin. In contrast, the cytotoxic effect of legubicin on control 293 cells was less than 1% of that of doxorubicin, indicating peptide conjugation to form the prodrug had abolished the cytotoxic effect of the doxorubicin. The dose responsive curve of legubicin on control 293 cells was parallel to that of doxorubicin on both 293 cells, suggesting that the residual cytotoxicity of legubicin may result from slight (˜1%) doxorubicin contamination. In contrast, a profound cytotoxic effect of legubicin was observed for legumain+ 293 cells. The dose response curve of legumain+ cells challenged with legubicin also differed from that for these cells exposed to doxorubicin.

Tumoricidal effect of legubicin in vivo. The in vivo effects of legubicin on normal and tumor bearing hosts and efficacy in tumor eradication was investigated using the CT26 murine syngeneic colon carcinoma model. Legubicin was very well tolerated in mice with much reduced toxicity compared to doxorubicin. Intraperitoneal injection of legubicin at a dosage of 5 mg/kg three times at 2 day intervals induced complete growth arrest of the tumors with little evidence of toxicity, as most readily evidenced by the absence of weight loss. In contrast, doxorubicin failed to produce similar anti-tumor efficacy at doses approaching its maximum tolerable dose (MTD). When doxorubicin was administered by the same protocol and dosage as for legubicin, toxicity was fatal.

A single injection of 5 mg/kg legubicin induced more profound tumoricidal effects than a comparable dose of doxorubicin, as observed by histology. TUNEL assay analysis of tumor tissues revealed a higher apoptotic index for legubicin than for doxorubicin treatment. Surprisingly, in organs that do express legumain, such as kidney and liver, no injury was evident (not shown). These observations indicate that legubicin has significantly improved safety and therapeutic indices compared to doxorubicin.

### Discussion

The high level of legumain expression by tumor cells, coupled with its unusual and highly specific substrate requirement for catalytic function, makes it an attractive candidate for prodrug conversion in a therapeutic mode. Current cancer chemotherapeutic agents have significant undesirable cytotoxicity. A promising approach to increase therapeutic delivery to tumor cells is to exploit enzymes that are more highly expressed by tumor cells and thereby achieve local prodrug activation to the active compound. Peptide conjugates of doxorubicin designed for activation by plasmin (de Groot et al., 1999; Chakravarty et al., 1983) and cathepsins (Satchi et al., 2001; Dubowchik et al., 1998a and 1998b) have been suggested. However, those conjugates are relatively deficient in target selectivity since plasmin generation is not tumor selective.

The doxorubicin prodrug exemplified herein was synthesized by incorporating a peptide extension to the amino group of doxorubicin. This agent, designated legubicin, was analyzed for cytotoxicity on cells not expressing legumain where it was less than 1% as toxic as doxorubicin. However, on cells expressing legumain, legubicin was profoundly cytotoxic, consistent with its conversion to leucine-doxorubicin.

Intraperitoneal administration of legubicin at 5 mg/kg resulted in complete arrest of tumor growth without identifiable toxicity, such as weight loss, in contrast to doxorubicin treated mice. Legubicin administration produced profound tumor cell apoptosis as indicated by TUNEL assay. Unexpectedly, in organs containing cells that normally express legumain, such as kidney and liver, no injury was evident. Thus, legumain activation of this prodrug may require conditions not present in normal tissue. Prodrug activation may be carried out by secreted or cell surface associated legumain; whereas legumain appear to be localized in lysosomal vesicles in normal tissues. Legumain requires an acidic environment for optimal catalytic activity, which may not be present in normal tissues. Legubicin also appears to have an improved therapeutic index compare to its parent doxorubicin. Whereas clinical use of doxorubicin is limited by its toxicity, a prodrug that preserves activity, but that has reduced toxicity is an attractive alternative.

### Example 4

### Analysis of Substrate Specificity of Legumain Using Phage Displayed Substrate Libraries

Legumain demonstrated an uniquely restricted specificity requiring an asparagine residue at the P1 position. However it does not hydrolyze all asparaginyl carboxyl peptide bonds of all protein substrates. Chen, J. M., et al., Cloning, isolation, and characterization of mammalian legumain, an asparaginyl endopeptidase. J Biol Chem, 1997. 272(12): p. 8090-8; 47; Mathieu, M. A., et al., Substrate specificity of schistosome versus human legumain determined by P1-P3 peptide libraries. Mol Biochem Parasitol, 2002. 121(1): p. 99-105. Many asparagine residues may not be accessible and able to form transition state analogues in most proteins. Amino acid preferences for parasite legumain indicate legumain has some degree of preference for the P2 and P3 positions, however it can accept all residues except proline at the P1′ position. Mathieu, et al. (2002); Schwarz, G., et al., Characterization of legumain. Biol Chem, 2002. 383(11): p. 1813-6.

The substrate specificity of mammalian legumain will be explored using combinatorial phage display peptide libraries that will be constructed by inserting a legumain recognition site between a Tissue Factor extracellular domain (TF₁₋₂₁₈) and fusion with a second gene III in M13 phage. TF₁₋₂₁₈ is particularly resistant to proteolysis. The legumain substrates library will contain two random amino acids flanking each side of an asparagine residue (XXNXX). Such a library has a modest library size of 1.6×10⁵ different sequence combinations. DNA encoding these peptide sequences will be synthesized and cloned into the phage vector display TF₁₋₂₁₈ as a fusion protein with the second phage gene III coat protein.

The phage displayed substrate library will be immobilized in 96 well plates through anti-TF antibody, and recombinant legumain expressed by Pichia yeast will be used for proteolysis to release the susceptible phage from the plate. These phage will be plated and amplified. Individual phage will be selected and sequenced to detect the susceptible residues surrounding the asparagine residue. The motifs of highest frequency represent the more favorable sequences for legumain recognition. The identified peptide sequences will be confirmed with synthetic peptides and its binding kinetics will be characterized.

### Example 5

### Hypoxia Induces Legumain Expression and Localization of Legumain on the Cell Surface is Needed for Tumor Invasion and Angiogenesis

This Example illustrates that inhibition of legumain not only inhibits tumor cell invasion but also inhibits angiogenesis by tumor cells.

### Materials and Methods:

The materials and methods employed are generally described in the other Examples provided in this application.

The four AEPI's employed in some of the experiments described herein have the following structures: AEPI-1 is Cbz-Ala-Ala-AzaAsn-(S,S)-EPCOOEt; AEPI-2 is Cbz-Ala-Ala-AzaAsn-CH═CH—COOEt; AEPI-3 is Cbz-Ala-Ala-AzaAsn-CH═CH—COOBzl; and AEPI-4 is Cbz-Ala-Ala-AzaAsn-CH═CH—CON(CH₃)Bzl. The structures of these inhibitors are shown below:

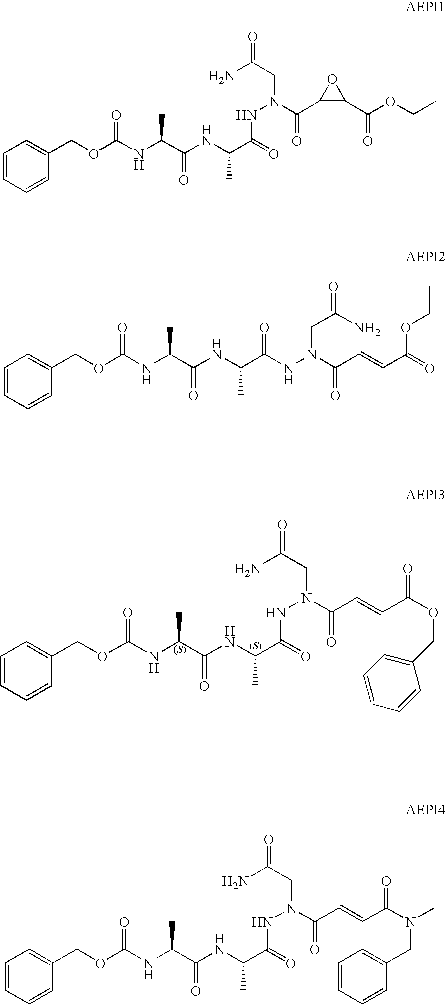

### Results

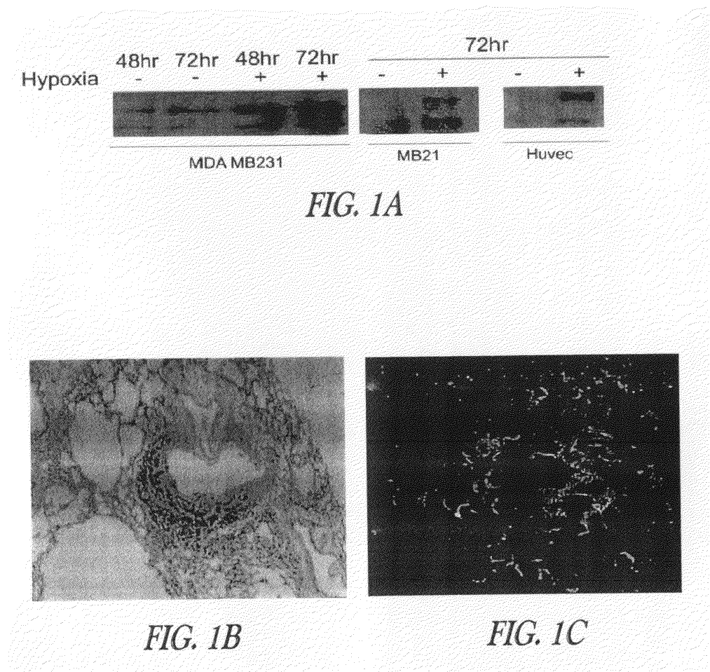

Legumain expression is induced by hypoxia. Legumain expression in human tumor cells and endothelial cells was examined by western blot analysis under both normoxic and hypoxic culture conditions, because hypoxia occurs early in the development of primary and metastatic tumors. In both AMD-MB231 human breast cancer cells and MB21 human melanoma cells, legumain expression was induced in cells cultured under hypoxic conditions (1% O₂), and its level continued to rise as the duration hypoxic exposure increased (FIG. 1A). HUVECs expressed very little legumain under normal culture conditions, but legumain expression was significantly enhanced when these cells were cultured under hypoxic conditions for 72 hours (FIG. 1A).

The 4T1 metastatic mouse mammary carcinoma metastasizes spontaneously to lung (FIG. 1B). When legumain expression was examined in lung metastatic sites, such expression was observed very early in the development of metastatic tumors. Legumain expression was observed even when only a small number of tumor cells were present in metastatic sites (FIG. 1C). These data indicate that up-regulation of legumain expression occurs early during tumor development.

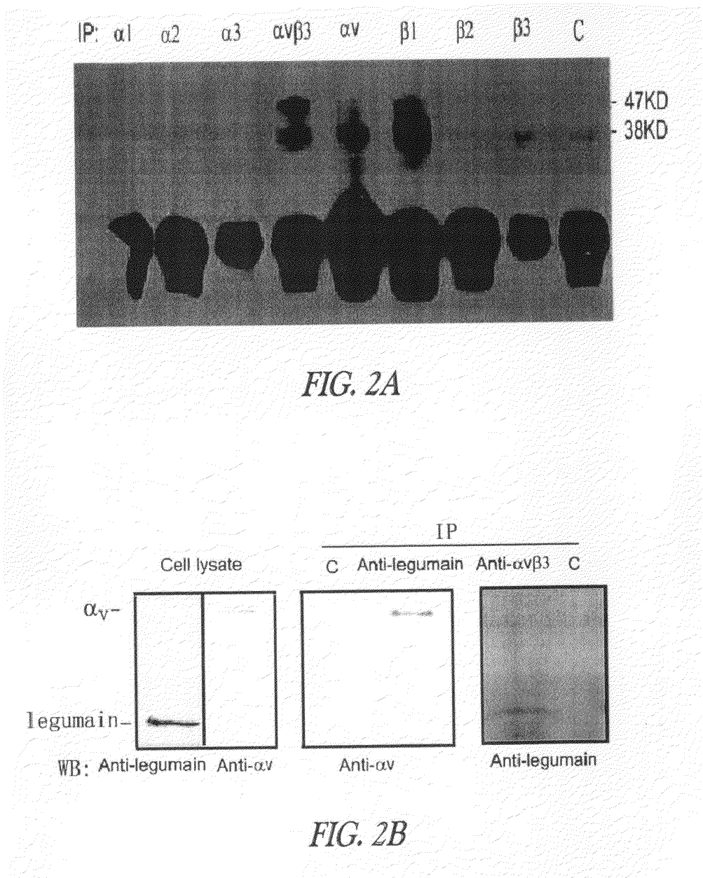

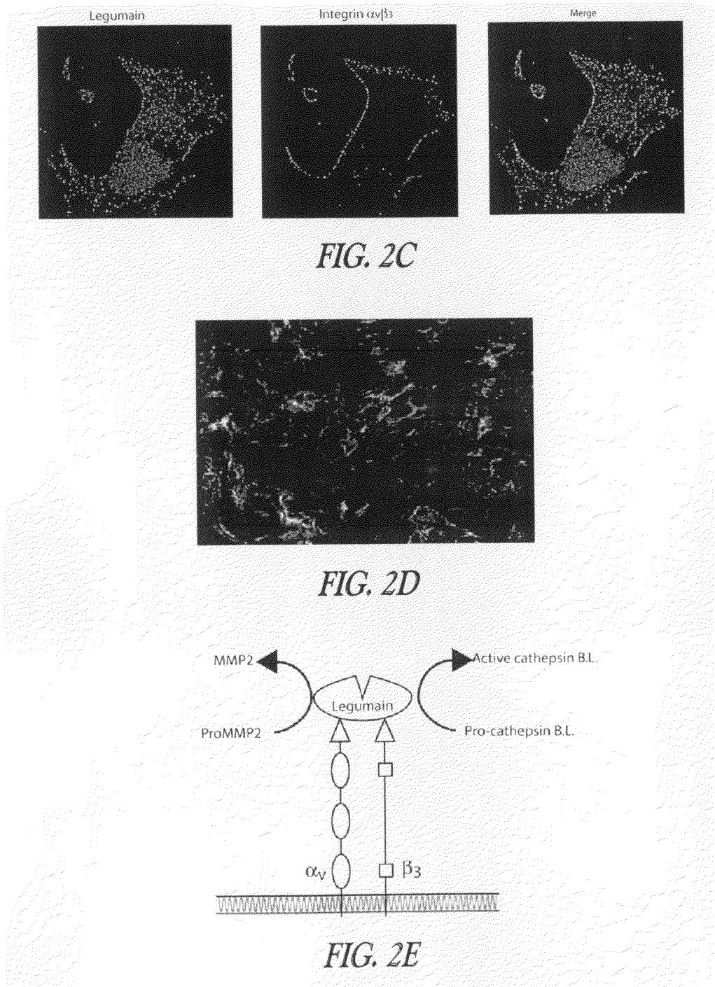

αᵥβ₃ integrins are cell surface receptors for legumain. Legumain is distributed intracellularly and is presented on cell surfaces in the tumor microenvironment (see previous Examples). However, legumain contains a RGD domain that is usually present in proteins that bind to integrins. To determine whether legumain binds to integrins, immunoprecipitation experiments were performed using a panel of anti-integrin antibodies on MDA-MB231 cell lysates, because these cells express low level of legumain in culture. Legumain was co-precipitated with anti-αv, αvβ3, β3, and β1 antibodies (FIG. 2A). These data indicate that αvβ3 integrin binds to legumain.

To further characterize the interaction between legumain and integrin, co-immunoprecipitation was performed with anti-legumain antibodies and detection was performed with anti-av integrin antibodies (FIG. 2B). Again, legumain was detected when anti-αvβ3 antibodies were used as the immunoprecipitating antibody (FIG. 2B).

Next, immunohistochemical staining of MDA-MB-231 human mammary carcinoma cells was performed with both anti-αvβ3 and anti-legumain antibodies (FIG. 2C). Very little legumain and αvβ3 integrin was expressed in cells cultured under normal conditions. However, the levels of legumain and αvβ3 integrins were both dramatically elevated under hypoxic conditions. These experiments also demonstrated that legumain and integrin were transported to cell surfaces where these proteins co-localized extensively (FIG. 2D). In migrating cells, the legumain:αvβ3 complex are predominantly present at the leading edge of the cells (FIG. 2C).

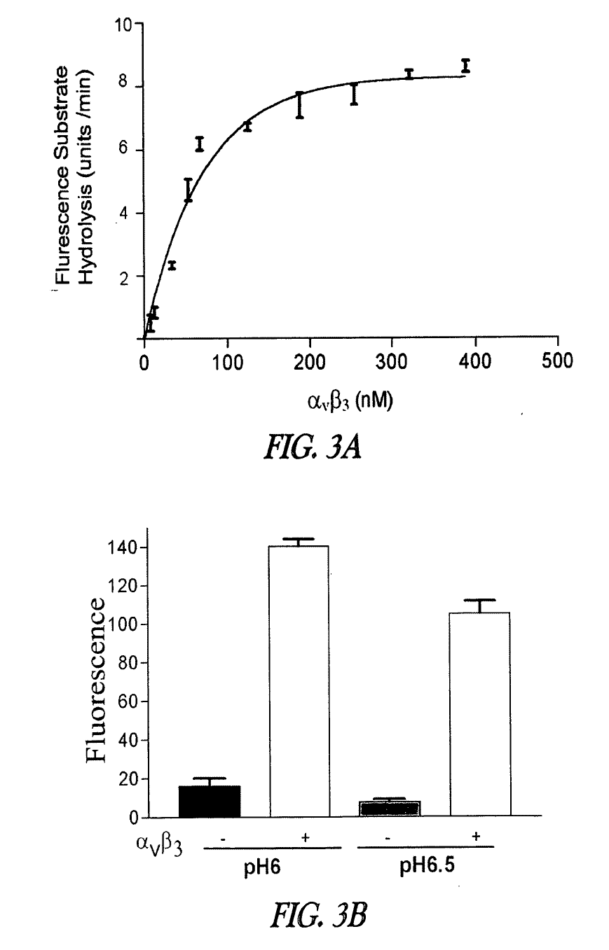

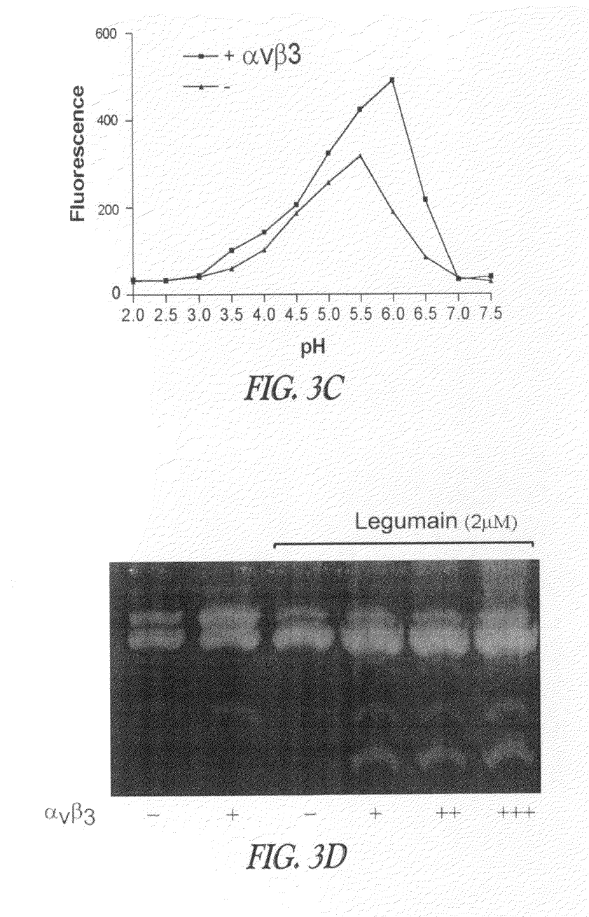

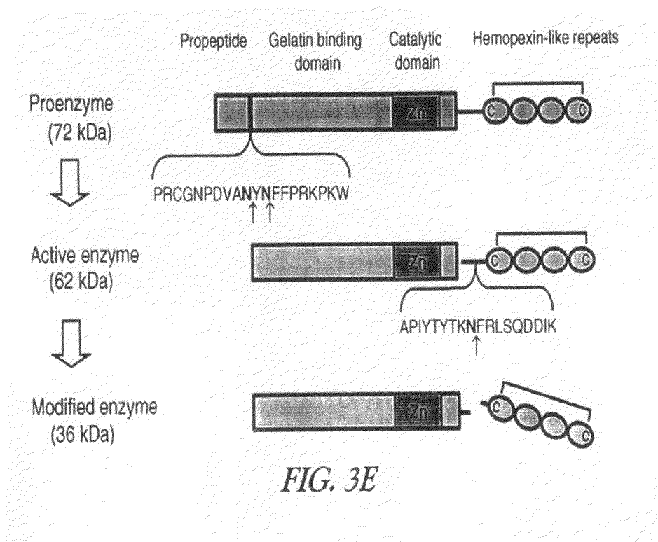

αᵥβ₃ integrin is a co-factor promoting legumain proteolytic activity. The effects of integrin binding upon legumain enzymatic activity were determined by observing hydrolysis of a substrate of legumain to generate a fluorescent product. FIG. 3A shows that as the concentration of αvβ3 integrin increases legumain amidolytic activity dramatically increases. These data suggest that αvβ3 integrin is not only a cell surface receptor of legumain, it is also a co-factor that increases legumain activity. In particular, binding of legumain to αvβ3 integrin can increase legumain activity nearly 100 fold (FIG. 3B).

In addition, binding to αvβ₃ also affects the pH dependency of legumain (FIG. 3C). The activity of legumain: αvβ₃ complexes were measured under different pH conditions and compared to that of uncomplexed legumain. As shown in FIG. 3C, binding of legumain to αvβ₃ integrin shifts its peak activity from pH 5.2 to pH 6.0. Typically free legumain is inactive at pH 7.0 (FIG. 3C). However, unlike free legumain, the legumain: αvβ₃ integrin complex was still active at pH values that were almost as high as pH 7.0 (FIG. 3C). There, integrin binding significantly increases legumain activity at the mildly acidic conditions that exist in the extracellular space of the tumor microenvironment. Moreover, the cell-surface location, the enhanced amidolytic activity of integrin-bound legumain and the shift in pH dependency indicates that the complex between legumain and αvβ3 integrin is likely the primary site in the tumor microenvironment where the prodrugs of the invention are activated.

Asparaginyl endopeptidase activity of legumain is critical for angiogenesis. The activity of legumain:αvβ3 against the physiological substrate of legumain (metalloproteinase-2, MMP2) was then evaluated. As shown in FIG. 3D, legumain not only removes the pro-MMP2 propeptide but also cleaves MMP2 between its proteolytic domain and its integrin binding hemopexin domain (FIG. 3E). Therefore, the legumain: αvβ₃ complex not only activates MMP-2 but it also generates a hemopexin fragment, which is a known inhibitory molecule for angiogenesis (FIG. 3D-E). These results indicate that the legumain:αvβ3 complex is an important modulator of pericellular proteolysis during tumor invasive growth and angiogenesis.

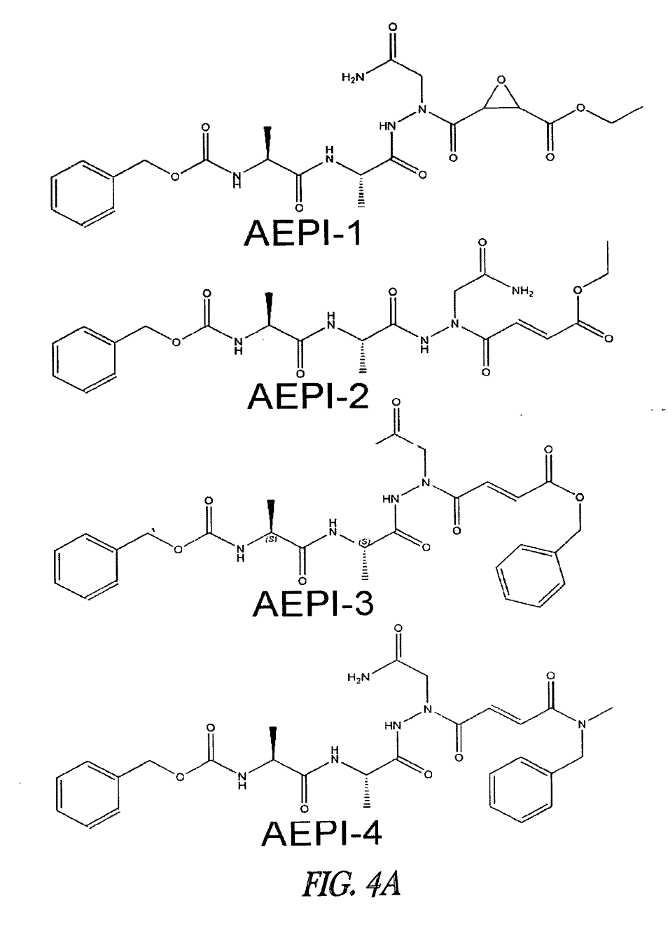

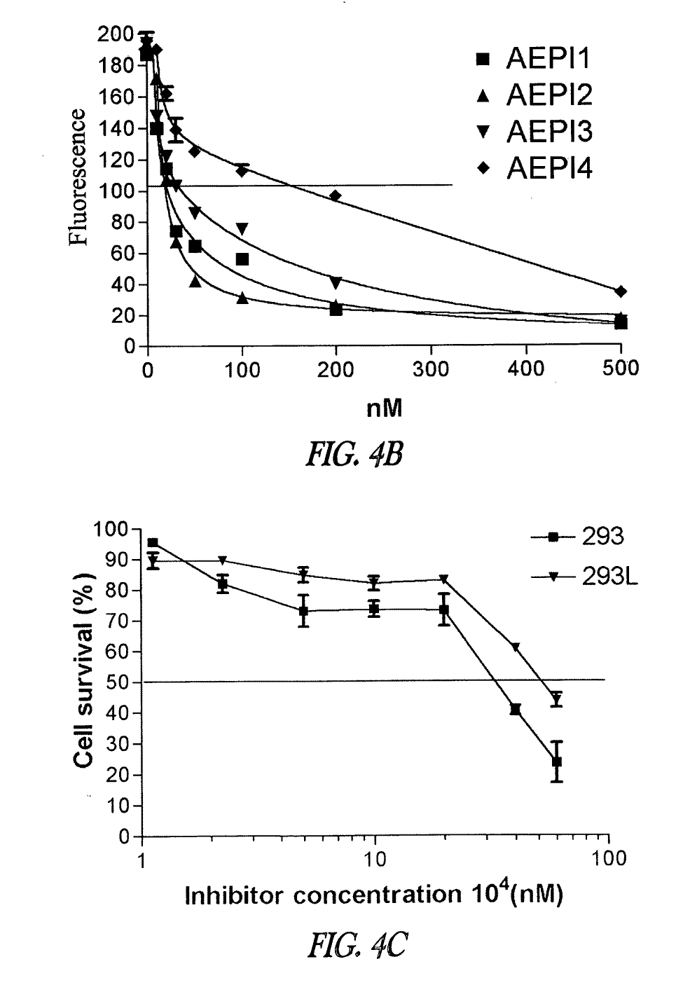

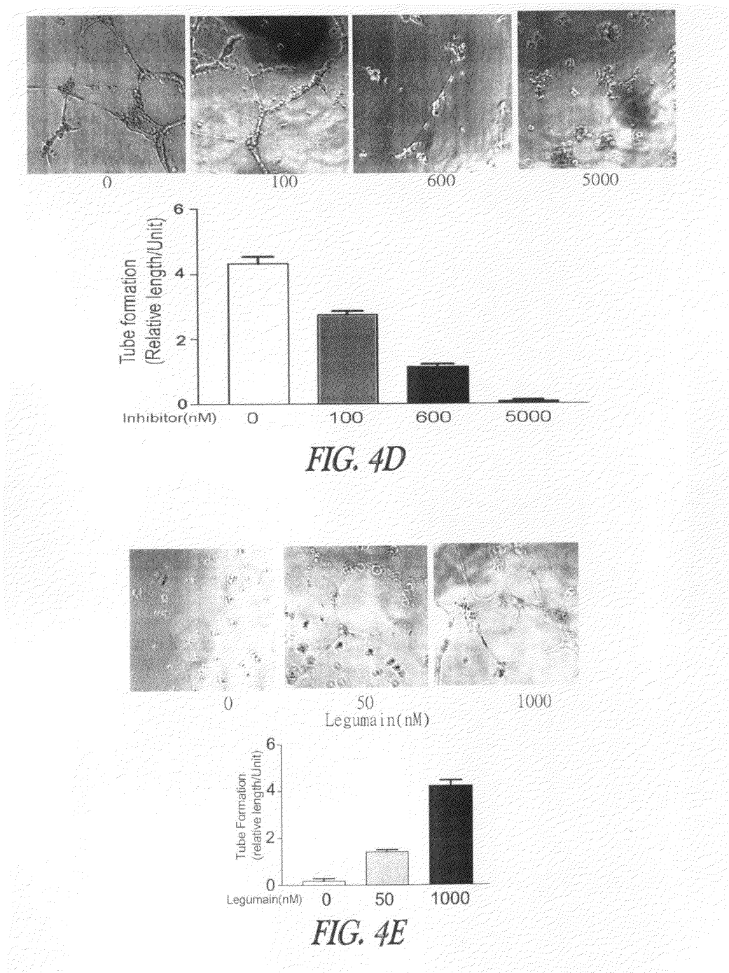

To further evaluate the role of asparaginyl endopeptidases in angiogenesis, four asparaginyl endopeptidases inhibitors (AEPI) were tested using recombinant legumain in an amidolytic activity assay with fluorescent substrate. FIG. 4A graphically illustrates the effects that AEPI-1 to AEPI-4 (also referred to as legumain inhibitors LI-1 to LI-4) have on asparaginyl endopeptidase activity. Both AEPI-1 and AEPI-2 had IC₅₀ values of about 20 nM and 21 nM, respectively. The IC₅₀ value for AEPI-3 was 34 nM, while the IC₅₀ value of AEPI-4 was 158 nM. The cytotoxicity of AEPI-1 was also assessed in wild type 293 cells (CC₅₀, 320 μM) and in 293 cells that recombinantly expressed legumain (CC₅₀, 430 μM). No apparent cytotoxicity was observed until almost 100 μM AEPI-1 was used in these assays (FIG. 4B).

The effect of AEPI-1 on endothelial cell function was assessed in vitro in a matrigel endothelial cell tube formation assay. AEPI-1 suppressed HUVEC tube formation in a dose dependent manner (FIG. 4C). Inhibition of tube formation was apparent at AEPI-1 concentrations of 100 nM and was completely inhibited when 600 nM AEPI-1 was used. In contrast, addition of recombinant legumain dramatically promoted and accelerated tube formation (FIG. 4D). HUVEC tube formation was observed as early as 5 hours after assay initiation when legumain was added. Under normal conditions 24 hours were required for the vascular tubes to form. AEPI-1 also inhibited hypoxia induced tube formation and endothelial cell invasion in an invasion assay (FIGS. 4E and 4F).

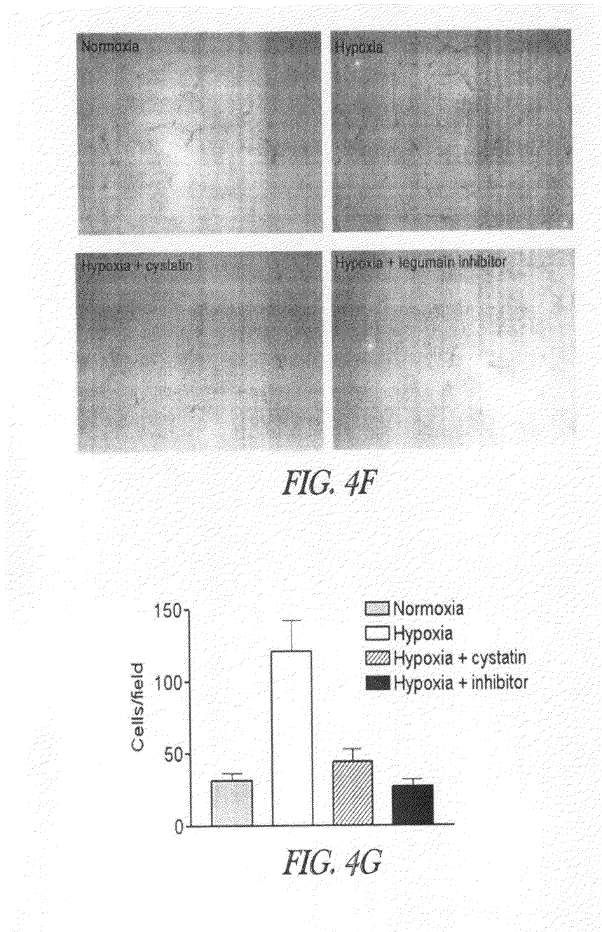

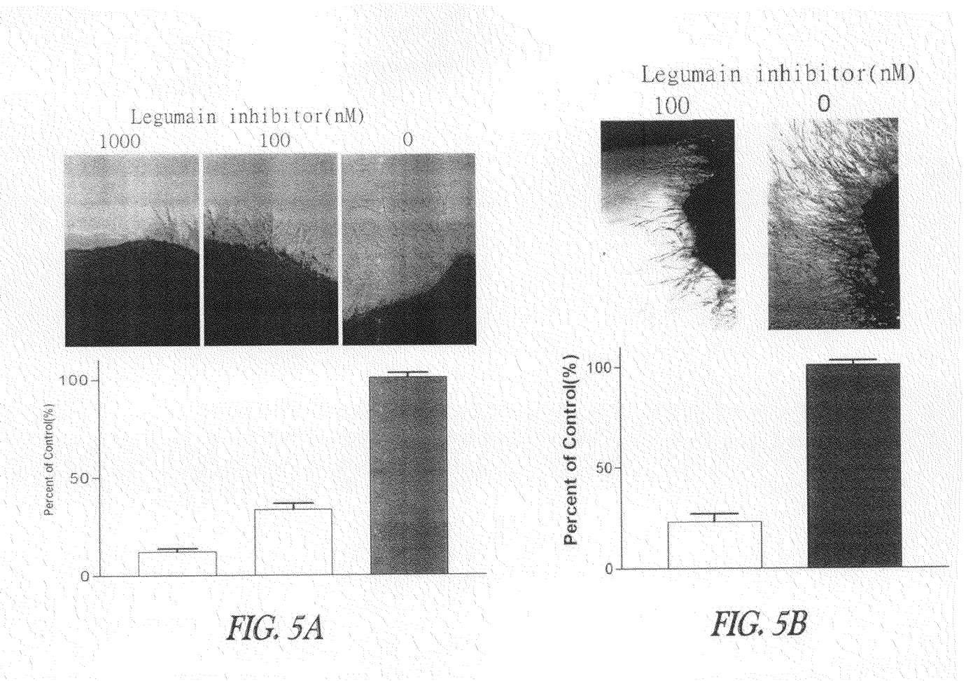

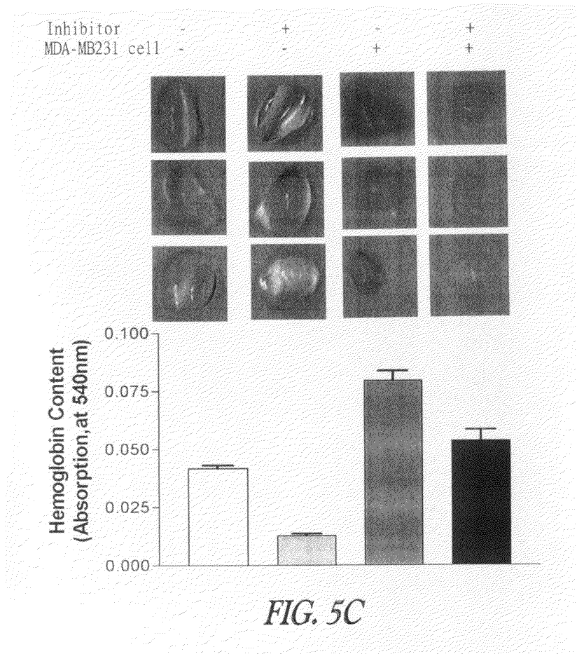

The effects of AEPI-1 on angiogenesis were further evaluated in mouse aortic sprouting assays. First, AEPI-1 was added to the beginning of the experiment to determine its effect on initiation of vessel sprouts (FIG. 5A). AEPI-1 inhibited FGF-2 induced vessel sprouts in a dose dependent manner (FIG. 5A). In the second assay, AEPI-1 was added at the third day after aortic sprouts were already formed (FIG. 5B), to ascertain whether asparaginyl endopeptidase activity was required for continued extension of vessel growth. Addition of AEPI-1 after sprouts were already formed significantly reduced extension of established vascular outgrowth (FIG. 5B). These data suggest that legumain has a continuing role in vascular formation throughout vessel development. These data also suggest that legumain's activation of proteases and its association with integrins may play an important role in angiogenesis.

The effect of AEPI-1 on angiogenesis was further investigated in vivo using a Matrigel plug assay. Matrigel containing FGF-2 was injected subcutaneously into Balb C nu/nu mice. When AEPI-1 was present, FGF-2 induced vessel formation was significantly inhibited in the plug (FIG. 5C) as indicated by the gross appearance, hemoglobin content, and histology (data not shown) of the plug.

The effect of AEPI-1 on angiogenesis induced by cancer cells was then investigated by including human breast cancer cells (MDA-MB-231 cells) in the Matrigel plugs. The cancer cells produced a spectrum of angiogenic factors and induced robust angiogenic vessel growth (FIG. 5C). However, this cancer cell-induced angiogenesis was essentially completely inhibited by AEPI-1 (FIG. 5C). These data indicate that inhibition of asparaginyl endopeptidase activity affects angiogenesis even when such angiogenesis is induced by a wide range of angiogenic factors.

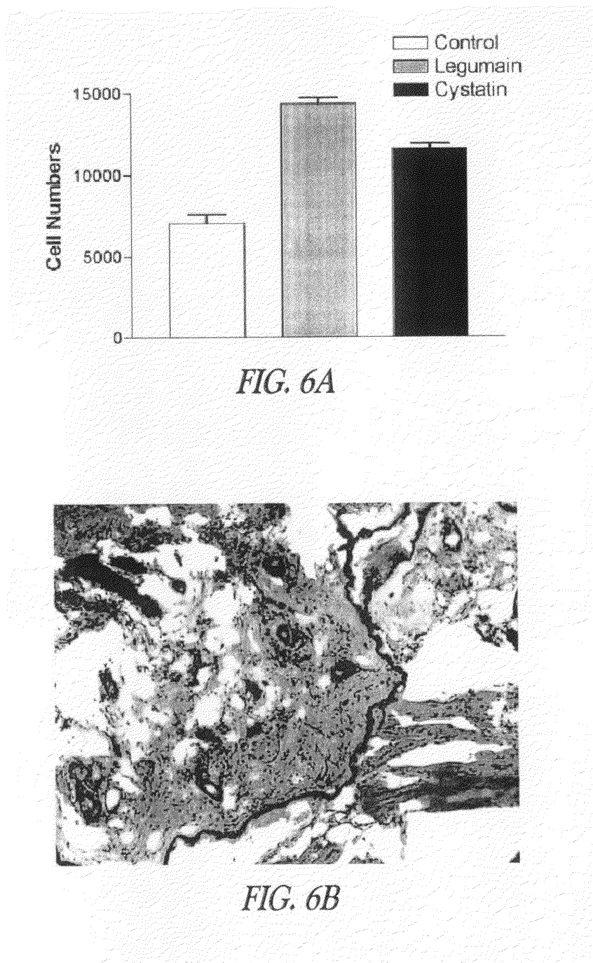

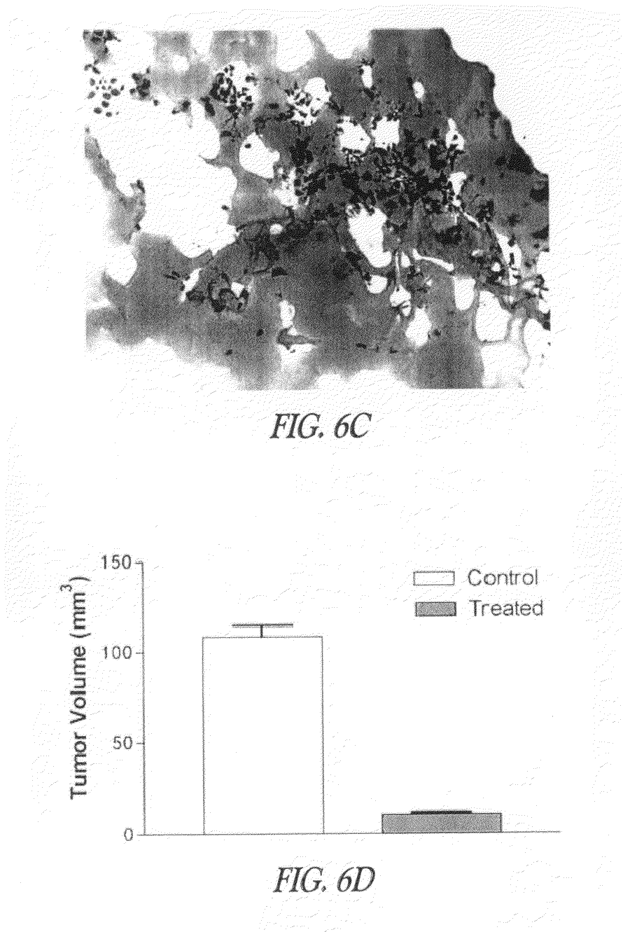

AEPI-1 inhibits tumor invasive growth. The function of legumain in tumor invasion was then examined using an invasion assay that included recombinant legumain protein, which promoted cancer cell invasion. The legumain-promoted cell invasion was partially inhibited by cystatin (FIG. 6A). These data are consistent with a role for legumain in activating gelatinase A and cathepsins that are implicated in extracellular matrix degradation.

The effect of AEPI-1 on tumor growth and invasion was then evaluated in a human mammary carcinoma model established in Balb C nu/nu mice. The MDA-MB-231 cells grew aggressively in vivo and formed a tumor that was similar to a human infiltrating ductal carcinoma with apparent glandular differentiation. The tumor locally invaded surrounding tissues and skeletal muscles.

As shown in FIG. 6C, AEPI-1 not only completely suppressed tumor growth, it also blocked the tumor differentiation into a more invasive form. Two weeks after tumor cell implantation, the treated tumors failed to grow, while the untreated control tumor developed into sizable invasive tumors.

Collectively, these data indicate that the legumain:αvβ3 complex has a significant role in tumor development and further show that asparaginyl endopeptidase inhibitors are effective in suppressing angiogenesis and tumor invasive growth. Therefore, asparaginyl endopeptidase inhibitors are promising new candidates for cancer therapy.

### Example 6

### Cell-Impermeable Prodrug Eradicates Multiple Drug Resistant Neoplasms

This Example shows that LEG-3 is a cell-impermeable pro-drug with reduced toxicity and markedly enhanced efficacy relative to the cell impermeable doxorubicin (Dox). The structure of LEG-3 is shown below as formula IB.

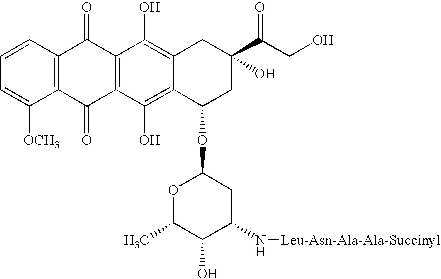

### Material and Methods

Reagents and cell lines. Rabbit polyclonal antisera against human legumain as well as 293 cells stably expressing human legumain were kindly provided by Dr. D. Roodman (Department of Medicine and Hematology, University of Texas Health Science Center, San Antonio, Tex.). A legumain substrate peptide was purchased from Bachem, Inc (King of Prussia, Pa.). Doxorubicin was purchased from Sigma (St. Luis, Mo.) and DMEM media was from Invitrogen (Carlsbad, Calif.). The CT26 murine colon carcinoma, C1300 mouse neuroblastoma cell lines, the human HT1080 fibrosarcoma cells, and 4T1 breast cancer cells (CRL-2539) were purchased from ATCC.

Antibody preparation. Anti-legumain antibodies were prepared by immunizing rabbits with KLH conjugated peptide CGMKRASSPVPLPP (SEQ ID NO: 16). A cysteine is added to the legumain sequence. The anti-legumain antibodies were affinity purified from resultant antisera using peptide antigen coupled to Ultralink Iodoacetyl Gel from Pierce (Rockford, Ill.). The bound antibodies were eluted by glycine buffer (100 nM, pH 2.7) and neutralized immediately by adding one-tenth volume of 1 M Tris pH 7.5.

Western blot. Proteins were dissolved in 2×SDS sample buffer for SDS-PAGE analysis using gradient Tris-glycine gels (8-16%). After electrophoresis, the proteins were transferred to nitrocellulose membranes and blocked with nonfat milk. The anti-legumain antiserum was used as the first antibody and was incubated with the membranes for 1 hr (1:1,000 dilution). The blot was washed three times with PBS, incubated with strepavidin-peroxidase for 15 min, and developed by the enhanced chemiluminescence method (Sigma, St. Louis, Mo.).

Flow cytometry analysis. Single cell suspensions were prepared from organs and tumor tissues as reported by Liu et al. Cancer Res. 63: 2957-64 (2003). Rabbit anti-Legumain antisera diluted 1:5,000 or antigen purified anti-legumain antibody at 0.5 μg/ml in PBS are used to detect legumain. This is followed by FITC-conjugated goat anti-rabbit IgG diluted 1:5000 in PBS (BD Pharmingen, La Jolla, Calif.). For CD14 staining, the PE conjugated anti-mouse CD14 antibody diluted 1:3000 in PBS was used (BD Pharmingen, La Jolla, Calif.).

Immunohistochemical analysis. Immunohistochemical staining was performed on 5-μm thick frozen sections on poly-L-lysine slides. For endothelial identification, biotinylated rat anti-mouse CD31 monoclonal antibody (MEC 13.3) was used with Texas red conjugated strepavidin as the secondary reporting reagent. For staining of legumain, rabbit polyclonal anti-legumain antisera was used at 1:500 dilution or antigen purified anti-legumain polyclonal antibody at 0.5 μg/ml, and visualized with FITC conjugated goat anti-rabbit antibody. For the identification of tumor associated microphage, rat anti-mouse CD68 antibody was used and followed by an anti-rat antibody conjugated with Texas red. For identification of collagen I, a biotinylated rabbit anti-mouse collagen I antibody was used at 1:250 dilution and visualized Texas red conjugated strepavidin. The slides were analyzed by laser scanning confocal microscope (Bio-Rad, Hercules, Calif.).

Prodrug synthesis. The synthesis of the succinyl version of the prodrug utilized the azide method to protect the peptide from racemization. In principle, the N-protected amino acids or peptide-esters are converted by hydrazine derivatization to an acid hydrazide. Subsequent reaction with HNO₂ or derivatives leads to anacylazide. Thus, the Succinyl-Ala-Ala-Asn-Leu-N₂H₃ (SEQ ID NO:21) peptide was prepared by using liquid phase synthesis. It was directly used to synthesize the target compound. An example of the synthesis is as follows: Solution A: 1040 mg Succinyl-Ala-Ala-Asn-Leu-N₂H₄⁺F⁻ (SEQ ID NO:22) was dissolved in a small amount of DMF cooled to −10° C., and 1.5 ml of 4 N HCl, dioxane was added followed by 2.1 mM isoamylnitrite. The mixture was stirred for 30-40 min at −10° C. and then the pH carefully adjusted to 7.5 with diisopropyl-ethylamine. Solution B: 1210 mg doxorubicin acetate was dissolved in a small amount of DMF at room temperature, the pH adjusted to 7.5 with DIPEA and then chilled to −10° C. Solution A and solution B were combined, the pH readjusted to 7.5 and monitored throughout the reaction. The reaction mixture was allowed to warm to 4° C., and allowed to stand overnight. HPLC analyses indicate approximately 80% completion of the reaction within 24-48 hrs. The reaction mixture was then diluted 10-fold with 0.1% TFA (in H₂O) and applied directly onto preparative HPLC. A linear acetonitrile gradient was used to elute the target compound. Fractions were analyzed for purity, combined, and lyophilized. HPLC, AAA, MS analyses were performed on the lyophilized powder.

Cytotoxicity assays. The WST-1 cell proliferation reagent (Roche Molecular Chemicals, Germany) was used to determine cell proliferation by quantization of cellular metabolic activity. Control 293 cells and legumain⁺293 cells were cultivated in microtiter plates (5×10³ cells per well in 100 μl) and were incubated with serial concentrations of LEG prodrug or Dox for 48 h. Subsequently, 10 μl of WST-1 solution (1 mg/ml WST-1, 25 μM -methyldibenzopyrazine methyl sulfate) was added per well, and mixtures were incubated for an additional 4 h. The tetrazolium salt WST-1 (4-[3-(4-Iodophenyl)-2-(4-nitrophenyl)-2H-5-tetrazolio-1,3-benzene disfonate) was cleaved by the mitochondrial succinate-tetrazolium-reductase system to formazan in cells in direct correlation with the number of metabolically viable cells in the culture. The amount of formazan salt was quantified in three replicates by absorbance at 450 nm using a micro-plate reader (Molecular Devices, Palo Alto, Calif.). All results were derived from replicate experiments with similar results.

Dox and LEG-3 uptake assay. Legumain⁺ 293 cells or control 293 cells (2.5×10⁵ cells/well) were seeded in six-well plates. The culture plates were then incubated for 24 h at 37° C. and 5% CO₂, and the medium in each well was replaced with 2 ml of serum-free, antibiotic-free medium containing various concentrations of Dox or LEG-type prodrug compounds. The cells were incubated 1.5 h then washed three times with 2 ml of cold PBS. At this point, cell nuclear positivity for Dox could be analyzed by fluorescent microscopy. For quantitative assays the cells were then lysed by adding 0.5 ml of water and gently rotated on an orbital shaker for 5 min at room temperature. The lysed cells were added to 1.5 ml acidified ethanol, and incubated at 4° C. in the dark for 3 h. Total Dox and LEG content was measured fluorometrically using a Perkin-Elmer LS-50-B spectrofluorometer (excitation: 470 nm; emission: 590 nm). Fluorescence intensity was translated to drug concentration by use of a standard curve prepared from Dox and LEG solutions in cell lysates that were not previously exposed to the drug. Results are expressed as the mean±SD of at least three replicates for each experiment.

Determination of marrow toxicity. Groups of healthy Balb C mice (n=4) were injected i.p. with a single dose of LEG-3 (49.4 μmol/kg or 4.94 μmol/kg) or Dox (3.4 μmol/kg). On day 7, retro-orbital sinus blood samples were collected into 10 mM EDTA, and counted by hemocytometer after lysis of red blood cells with an acidified methyl violet solution.

Determination of Maximum Tolerated Dosage (MTD). Four six-week-old Balb/c mice were used for each experimental group. The mice were weighed individually and the average weight of the group is used to determine the exact doses. Mice were given i.p. injection once a day for five days. The MTD is defined as the maximum drug dose administered to non-tumor-bearing mice once daily for five consecutive days without mortality.

Tissue distribution. LEG compounds or Dox was injected i.p. into mice, 12 h later the animals were perfused and the Dox autofluorescence was measured following homogenization in 50% ethanol and then diluted with an equal volume of 50% ethanol containing 0.6 M HCl. Fluorescence measurements were obtained with excitation at 470 nm and emission at 590 nm, concentrations were derived by conversion from a Dox standard curve. Tissues from saline injected mice provided controls. Blood samples were to 0.75 ml with PBS, centrifuged, the pellets washed with PBS and Dox extracted with ethanol, 0.3 M HCl.

Animal models. The CT26 syngeneic murine colon carcinoma model was generated and maintained in The Scripps Research Institute animal facility. This model was produced in Balb/c mice aged 4 to 6 weeks. Mice were injected with 5×10⁵ CT26 tumor cells per subcutaneous site on the back. The C1300 mouse neuroblastoma model was generated in A/J mice by subcutaneous injection of 5×10⁵ C1300 cells per site on the back. Treatment was initiated when the tumors reached 4 mm in diameter through bolus intraperitoneal (syngeneic tumors) or intravenous (human tumors) injections of the indicated reagents. Treatment was three times per week for two weeks. The human HT1080 fibrosarcoma was xenografted in Balb/c nu/nu mice obtained from The Scripps Research Institute breeding colony; and HT1080 cells (1×10⁶ cells/site) were inoculated subcutaneously into the backs of mice. The MDA-PCa-2b human prostate carcinoma model was generated in WEHI nude mice and these cells (10⁶ cells) were also injected subcutaneously.

A lung metastasis model was induced experimentally by injecting a cell suspension of 4T1 breast cells (1×10⁵) suspended in 0.1 ml serum free medium into the tail vein of six-week-old female BALB/c mice. The mice were treated with 100-μl intraperitoneal injections of either PBS alone (control group) or LEG-3 (100 μg/100 μl) daily for two weeks.

Primary 4T1 tumors are generated by subcutaneous injection of 5×10⁵ 4T1 cells in the right flank of six-week-old BALB/c mice. Two different groups of four animals were treated between days 9 and 27 after tumor induction. The treatments were 100-μl intravenous injections of PBS alone (control group) or LEG-3 100 μg/100 μl in PBS by intraperitoneal injection every two days for two weeks. Tumor volumes of treated animals were measured every two-day starting on day 9 by microcaliper measurements (volume=width×length×width/2). As soon as the tumor volume reached 1400 mm³ in the control groups (on day 30th), euthanasia was performed and lungs were removed and fixed in the Bouin's solution. Lung metastases were counted by anatomy microscope. Statistical significance between treatment groups was determined by two-tailed Student's t tests using Microsoft EXCEL software.

Tumor growth and signs of physical discomfort were monitored daily. Such signs included any gross evidence of tumor necrosis, local tumor ulceration as well as evidence of toxicity including changes in the animal's mobility, response to stimulus, piloerection, eating, and weight. These procedures have been reviewed and approved by the Institutional Animal Care and Use Committee at The Scripps Research Institute. All the experiments were conducted in The Scripps Research Institute facilities which are accredited by the Association for the Assessment and Accreditation of Laboratory Animal Care. The Scripps Research Institute maintains an assurance with the Public Health Service, and is registered with the United States Department of Agriculture and is in compliance with all regulations relating to animal care and welfare.

Statistical analysis. Statistical significance of data was determined by the two-tailed Student's t test, except for statistical significance of survival curves, which used the Logrank Test using GraphPad Prism version 3.00 (GraphPad Software, San Diego, Calif.).

### Results

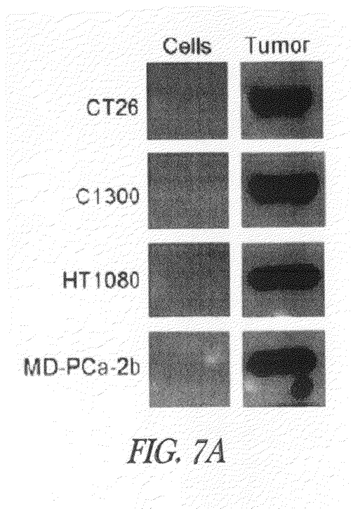

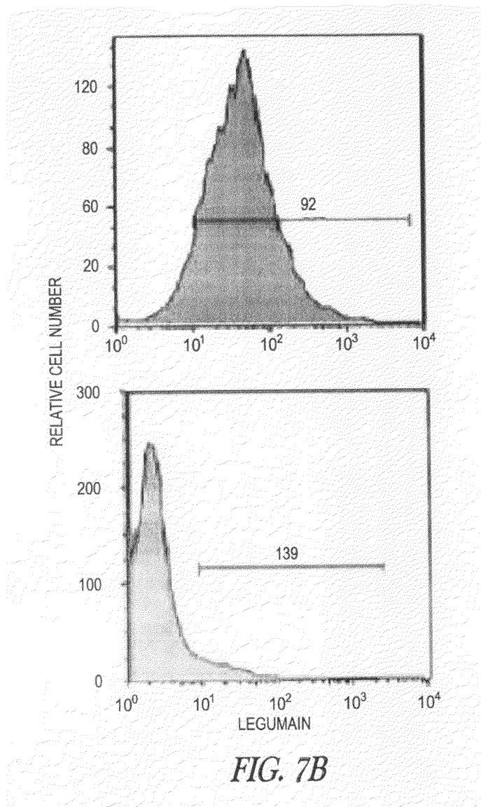

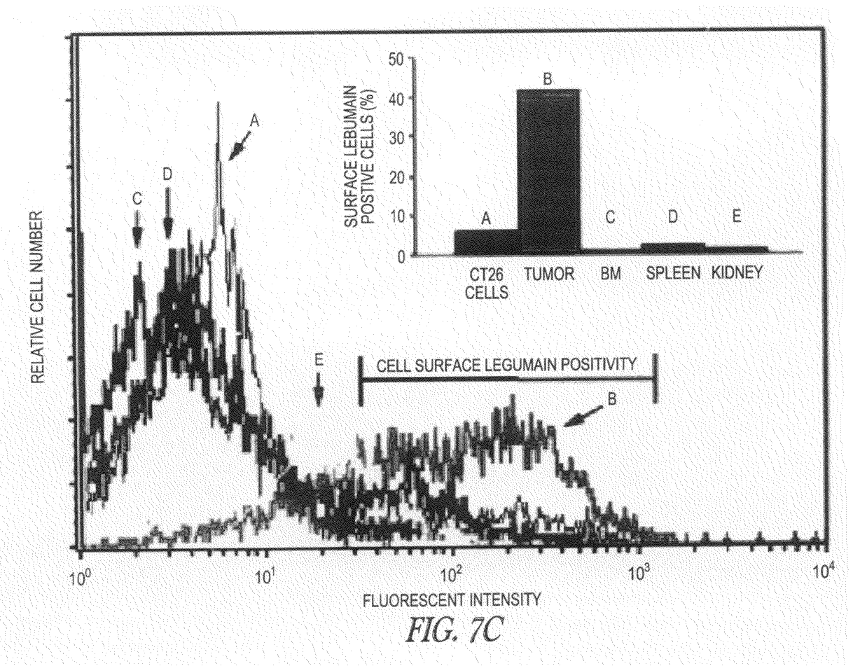

Legumain is highly expressed by cells in the tumor microenvironment. As shown above, legumain is expressed in vivo by tumor cells and proliferating endothelial cells intracellularly as well as on their cell surfaces. However, legumain is not detectable in or on CT26, C1300, HT1080, and MDA-PCa-2b as well as eight other tumor cell lines in culture, but is expressed by these same cells after generating tumors in vivo (FIG. 7A). Further, the legumain endopeptidase is not detectable in large scale survey panels of tumor cell lines in culture, except for THP-1 cells, based on a search of the NCl tumor cell expression database.

Using immunohistochemical analysis, legumain is usually present in tissue sections of human colon carcinomas, neuroblastomas, fibrosarcomas, and prostate carcinomas, representative of the types of neoplasms investigated in the present study. Legumain expression was not detectable in the normal tissues of origin for these neoplasms. The remarkable local expression of legumain not only by neoplastic cells but also by stromal cells in association with tumor development in vivo suggests that such expression is in response to novel local aspects of the tumor microenvironment.

Legumain is found on the CT26 tumor cell surfaces where it is effectively removed by 30 min collagenase digest at 57° C. (FIG. 7B). These data indicate that legumain is found on the cell surface. This is surprising not only because legumain was previously identified as being an intracellular protein but also because legumain has no transmembrane domain and no prior evidence exists that suggests secretion or plasmalemma localization of this lysosomal protease.

Flow cytometry was used to further analyze the extracelluar localization of legumain in tumors and normal organs. Flow cytometry permitted analysis of cell surface legumain in single cell suspensions prepared from tumors, bone marrow, spleen, and kidney, as well as cultured tumor cells. Despite demonstrable intracellular legumain by renal tubular epithelial cells, less than 2% of isolated viable cells were very weakly positive for cell surface legumain. Spleen cells have considerably less legumain than renal cells; however, approximately 5% of spleen cells are weakly positive for cell surface legumain. Furthermore, cell surface legumain is not detectable on cells derived from bone marrow nor is it found on cultured CT26 cells. In contrast, 40% of intact viable cells derived from in vivo CT26 tumors were strongly positive for cell surface legumain (FIG. 7C). A similar pattern was observed for all tumors examined (data not shown) indicating that cell surface and extracelluar legumain is uniquely abundant only in tumors.

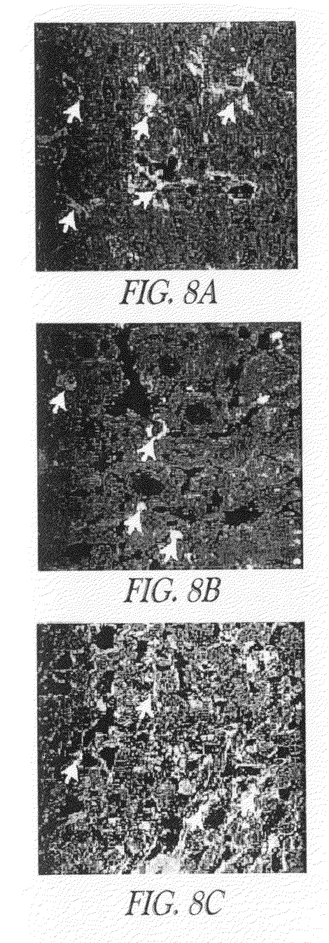

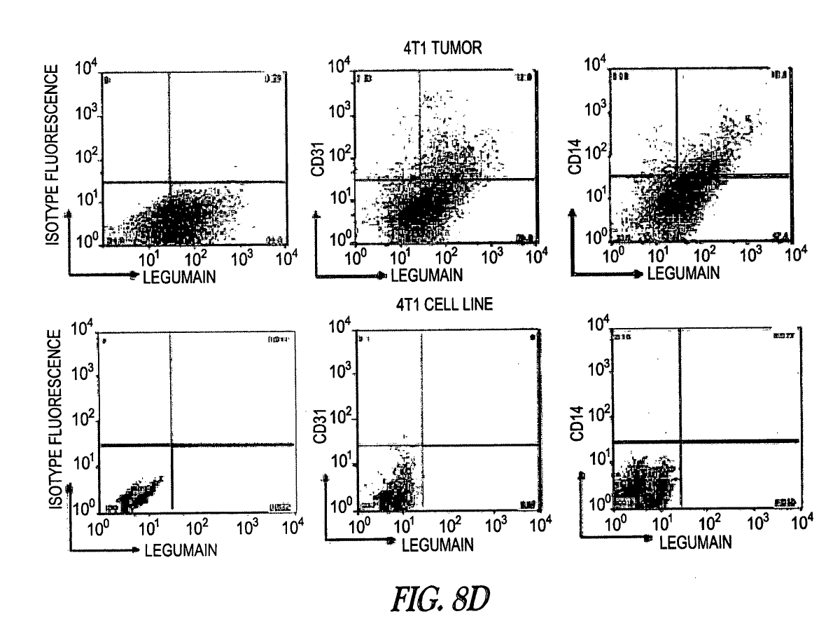

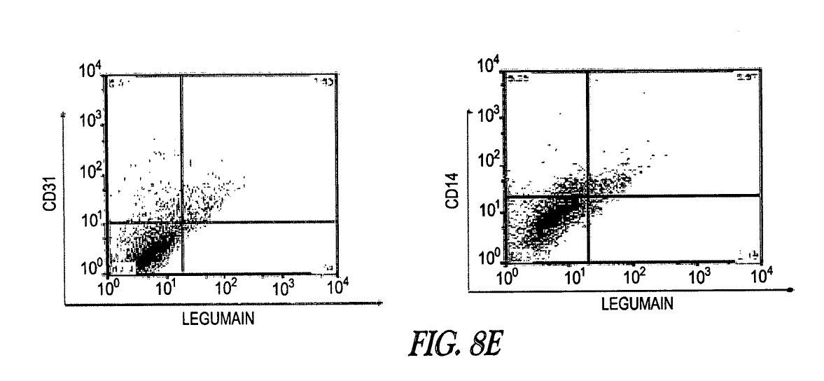

Legumain is also expressed by tumor vascular endothelial cells (FIG. 8A, white arrows). In addition, legumain is also expressed by on tumor-associated macrophages (TAMs) as observed by dual staining of tissue sections from CT26 tumors with anti-legumain and anti-CD68 antibodies (FIG. 8B, white arrows). Secreted legumain is present in the tumor stroma associating with extracellular matrix proteins, such as collagen I (FIG. 8C, white arrows). Legumain expression was not detected in normal peripheral blood monocytes. Using flow cytometry legumain was also found on the surface of viable endothelial cells and tumor-associated macrophages using both anti-legumain antibody and anti-CD31 antibody or anti-CD14 antibody respectively (FIG. 8D). Interestingly, legumain on endothelial cell and tumor associated macrophage surfaces is resistant to removal by collagenase (FIG. 8E), suggesting a mode of cell surface association that is distinct from that of tumor cells where legumain is removed by collagen.

LEG-3 is activated by tumoral legumain. The ability of the cell impermeable targeting tumor microenvironment prodrug LEG-3 (FIG. 9A) to kill tumor cells was evaluated first in cell culture (FIG. 9B). The median effective concentration, which is the amount of LEG-3 or Dox required for 50% cell death (EC₅₀), was determined for both legumain-transfected and control non-producing cells (Table 4).

TABLE 4
EC50 (μM) of Dox and LEG-3.

| | 293 | Legumain + | HTP |
|---|---|---|---|
| | cells | 293 cells | cells |
| Dox | 1.2 | 1.4 | 1.5 |
| LEG-3 | >100 | 3.2 | 9.6 |
| LEG-3 + cystatin | >100 | 64.4 | 59.2 |
| LEG-4 | >100 | >100 | >100 |

LEG-3 was virtually non-cytotoxic to cells not expressing legumain. However, for cells that were transfected with legumain cDNA and express cell surface legumain, cytotoxicity was significant and EC₅₀ levels were close to those observed for Dox, indicating an efficient conversion of the LEG-3 prodrug.

The requirements for activation were demonstrated using an alternate peptide sequence not hydrolyzed by legumain (FIG. 9A-B) and also by inhibition of LEG-3 activation following inhibition of legumain function (FIG. 9C).

LEG-3 is activated extracellularly. Tumor cell uptake of LEG-3 was compared to Dox. When added to cell cultures, Dox rapidly entered cells. In contrast, the LEG-3 remained extracellular, consistent with its observed lack of cytotoxicity. In contrast, when LEG-3 was added to legumain expressing cells in culture, the end product Dox was found in cells (FIG. 9D-E).

Legumain is selectively functional in the tumor microenvironment. The maximum tolerable dose (MTD) of Dox and a molar equivalent amount of LEG-3 were given intravenously as a single intravenous bolus into mice bearing tumors after receiving CT26 tumor cells.

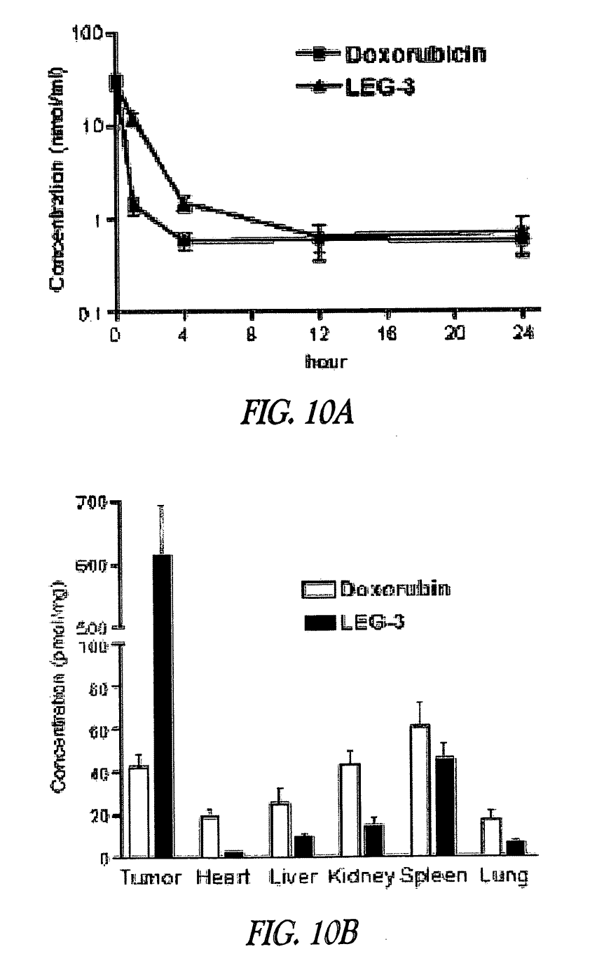

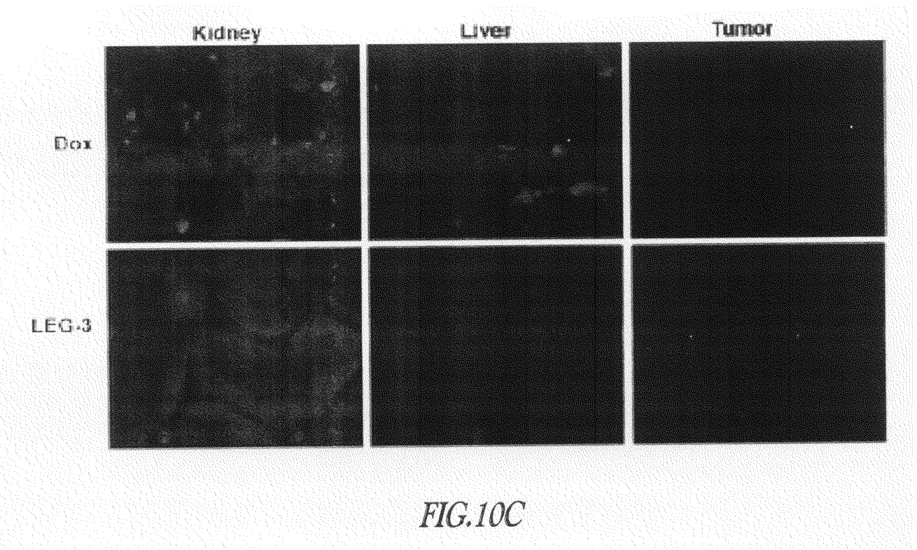

When Dox was administered, the plasma concentration very rapidly declined and then a low Dox concentration was detected that slowly cleared (FIG. 10A). There was significant Dox content in many tissues including tumor, heart, kidney, liver, and brain. These observations are consistent with observations by other investigators (Mosure et al., Cancer Chemother. Pharmacol. 40: 251-58 (1997); Dubois et al., Cancer Res. 62: 2327-31 (2002)).

In contrast, the initial decline of infused LEG-3 was much slower (FIG. 10A) than observed for Dox. Given the hydrophilic nature of LEG-3, such slow decline in LEG-3 plasma concentration may be attributed to reduced tissue uptake. LEG-3 concentrations in cardiac, liver, kidney, spleen and lung tissues were reduced when compared to Dox levels in those tissues (FIG. 10B). However, twelve hours post-infection, the content of LEG-3 was more than 10-fold greater in tumor tissues than that observed for Dox (FIG. 10B). Because of its reduced normal tissue uptake and its reduced toxicity, larger quantities of LEG-3 could be administered, which resulted in higher drug content in cells within tumors compared to that achieved for Dox administration.

To confirm the tissue distribution, Drug accumulation in tissues and tumor was visualized by Dox autofluorescence (FIG. 10C). These data further indicate that legumain is selectively found in the tumor microenvironment and that LEG-3 is processed to its cell permeable Leu-Dox product because Dox is detected within the cytoplasm of tumor cells after injection of LEG-3. Moreover, Dox appears to be further processed and translocated to the nucleus because intranuclear Dox fluorescence was detected in tumor cells of LEG-3 injected mice.

In vivo toxicity of LEG-3 and Dox. LEG-3 is significantly less toxic than Dox when evaluated in vivo. When given in five repeated intravenous administrations, LEG-3 had a much higher cumulative MTD and a reduced LD₅₀ compared to Dox (Tables 5-6). Thus, significantly more LEG-3 can be administered without toxic effects.

TABLE 5
In vivo toxicity of LEG-3 compared to DOX. Estimated MTD and
LD₅₀ of Dox and LEG-3 (μmol/kg) in Balb/c male mice.

| | |
|---|---|
| Dox | LEG-3 |

| | MTD | LD₅₀ | MTD | LD₅₀ |
|---|---|---|---|---|
| i.p. | 9.8 | 17.2 | >197.4 | >197.4 |
| i.p. 5× | 2.8 | 3.4 | 98.7 | >197.4 |
| i.v. | 18.2 | 25.8 | >197.4 | >197.4 |
| i.v. 5× | 3.2 | 5.1 | 74.0 | 197.4 |

TABLE 6
In vivo toxicity of LEG-3 compared to DOX. Comparison
of gross toxicity of mice treated with Dox and LEG-3.

| | | | | | | |
|---|---|---|---|---|---|---|
| Controls | Average weight loss (%) | 4 | | | | |
| Dox | Dose (μmol/kg) | 1.72 | 5.4 | | | |
| | Average weight loss (%) | 24 | 28 | | | |
| | Death (%) | 35 | 75 | | | |
| LEG-3 | Dose (μmol/kg) | 1.97 | 4.9 | 49.4 | 98.7 | 197.4 |
| | Average weight loss (%) | 3 | 5 | 7 | 8 | 16 |
| | Death (%) | 0 | 0 | 0 | 10 | 50 |

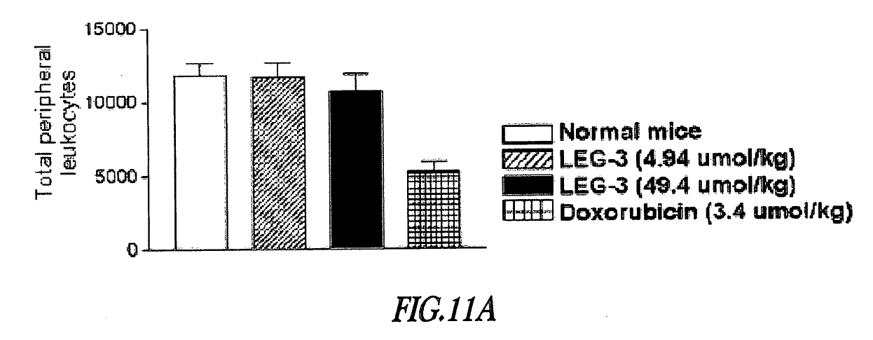

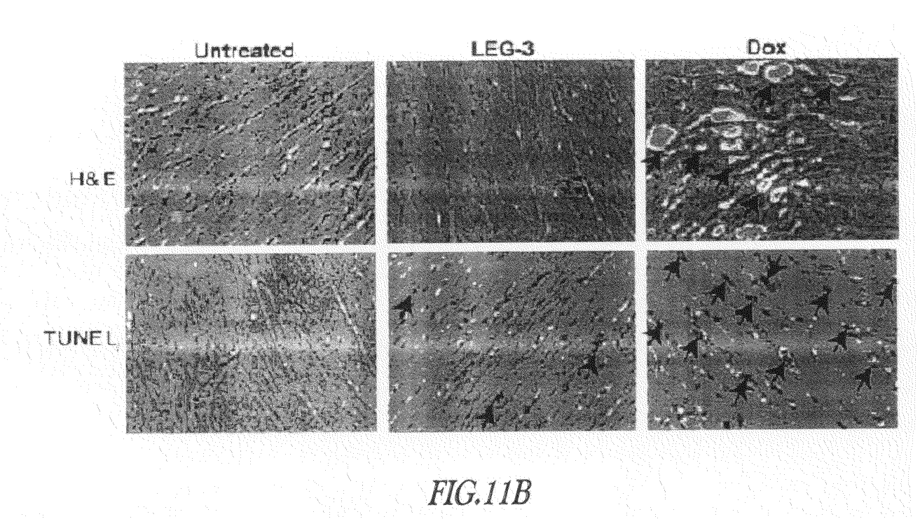

The cytotoxic effect of LEG-3 on peripheral blood leukocyte counts (WBC) was assessed and compared to the cytotoxic effects of Dox. There were no reductions of total WBC counts in mice treated six times in twelve days with 4.94 μmol/kg LEG-3 and only a slight reduction observed in mice treated with a 10-fold higher dose. In contrast, there was greater than 50% reduction in WBC counts in the group of mice receiving as little as 3.4 μmol/kg Dox on the same injection schedule (FIG. 11A). LEG-3 produced little evidence of myelosuppression compared to its parent compound Dox.

Bone marrow and cardiac toxicity of LEG-3 was also examined and compared to Dox. In cardiac tissue, mice treated with 49.4 μmol/kg of LEG-3 exhibited no histological evidence of cardiac toxicity. However, profound cardiac myocyte vacuolization and cell death was observed in mice treated with Dox at concentrations of 3.4 μmol/kg (FIG. 11B). Moreover, cardiac myocytes from mice treated with Dox demonstrated marked apoptosis, whereas this was not observed for LEG-3 treated mice (FIG. 11B).

In vivo efficacy is dependent on legumain specific activity. To further characterize the requirements for legumain activation of LEG-3 in vivo, two LEG-type compounds differing in the composition of the peptidyl element of the compound were evaluated. These LEG-type compounds were LEG-2 and LEG-4, shown below.

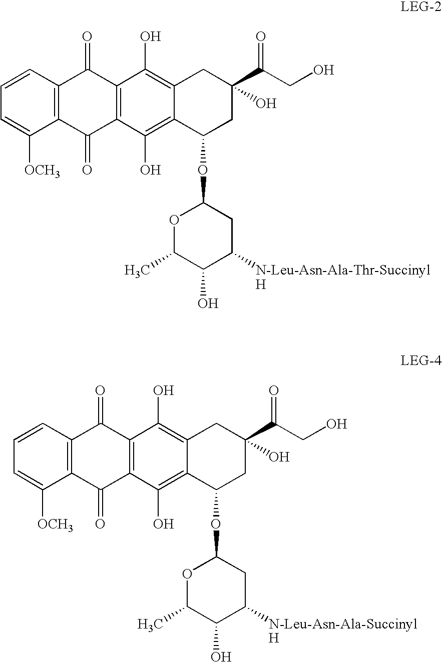

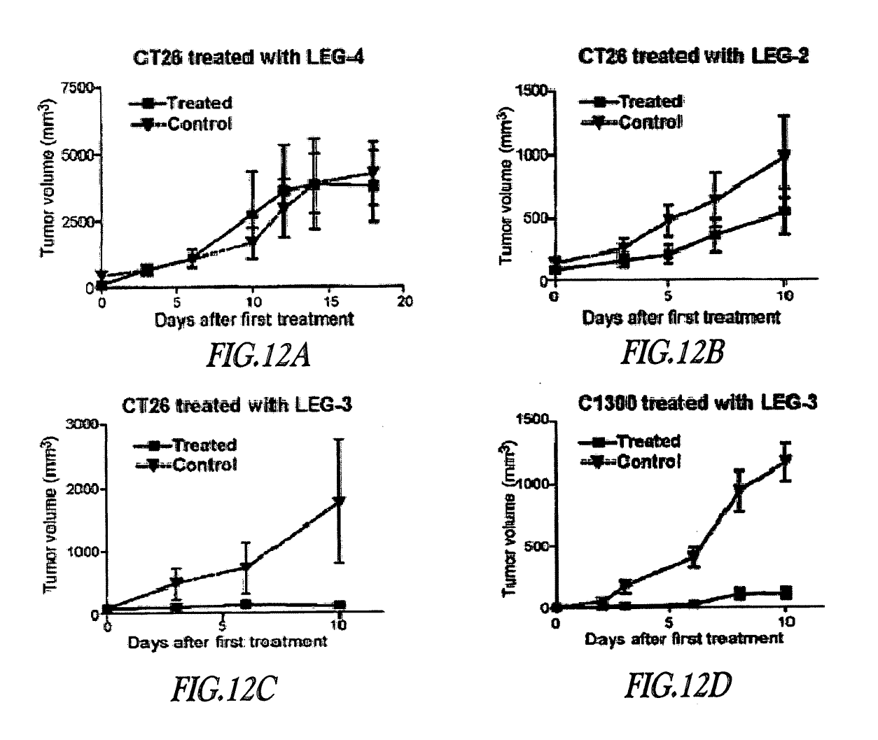

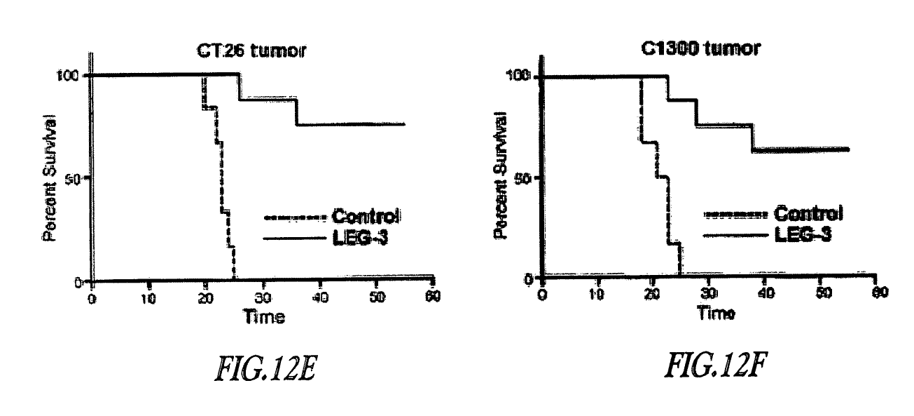

LEG-2 and LEG-4 were analyzed for in vivo efficacy in tumor bearing mice. The LEG-4 compound is cleaved by legumain (data not shown) and was devoid of tumoricidal activity (FIG. 12A). However, LEG-2 and LEG-3 are cleaved by legumain and possessed demonstrable in vivo tumoricidal efficacy (FIG. 12B-D).

LEG-3 has effective tumoricidal activity against diverse tumors in vivo. The therapeutic efficacy of LEG-3 was evaluated in both syngeneic rodent tumor models and human tumor xenograft models. When administered six times in 12 days at a well tolerated dose (49.4 μM/kg), LEG-3 effectively arrested growth of murine CT26 colon carcinoma in Balb/c mice and produced complete tumor eradication in some mice (FIG. 12C). A similar level of efficacy was also observed for the murine C1300 neuroblastoma in A/J mice (FIG. 12D), where LEG-3 treatment induced massive tumor cell death (FIG. 12C-D), tumor eradication and marked enhancement of survival (FIG. 12E-F).

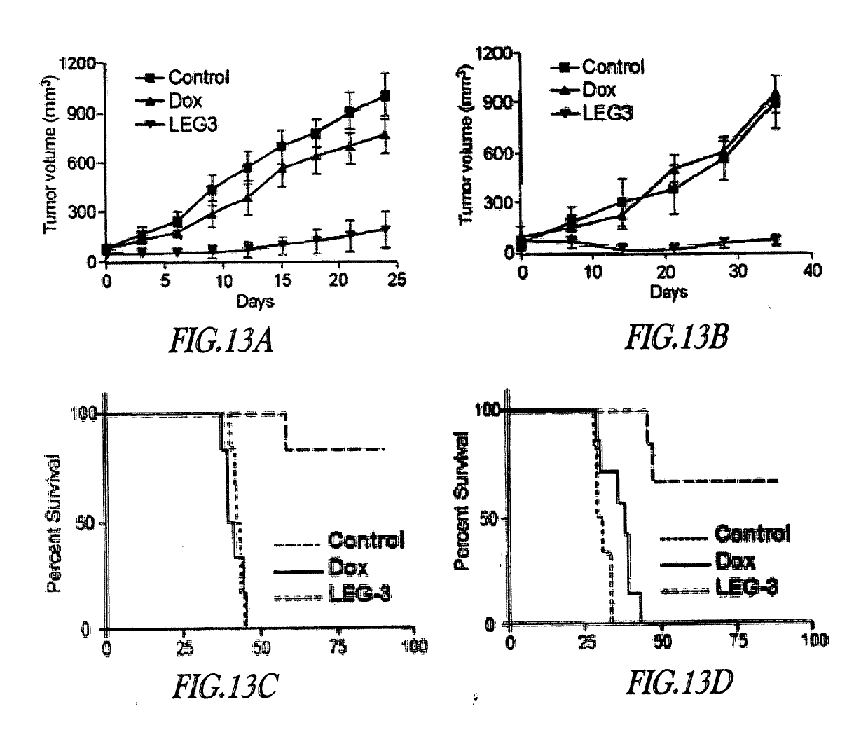

The in vivo efficacy of LEG-3 on human tumor xenografts in athymic nu/nu mice was assessed and compared to Dox. As described above, legumain protein was not detectable in either HT1080 or MDA-PCa-2b cells when those cells were maintained in culture. However, robust legumain expression was observed by immunohistochemistry of in vivo tumors propagated from these cells. Indeed, LEG-3 produced potent tumoricidal activity against the HT1080 fibrosarcoma, a fast-growing tumor and a model tumor that is known to be sensitive to Dox therapy (FIG. 13A).

On the other hand, human prostate carcinomas are frequently resistant to Dox therapy. For example, an MDA-PCa-2b prostate carcinoma that is a known Dox resistant tumor (Letsch et al., Clin. Cancer Res. 9: 4505-13 (2003)), failed to respond to Dox in vivo. However, administration of LEG-3 led to complete growth arrest (FIG. 13B) of these Dox-insensitive prostrate tumor cells. LEG-3 frequently resulted in complete tumor eradication with marked enhancement of survival of the HT1080-as well as the MDA-PCa-2b-tumor bearing mice (FIG. 13C-D). Toxicity of LEG-3 based on weight loss and mortality was negligible (FIG. 13E).

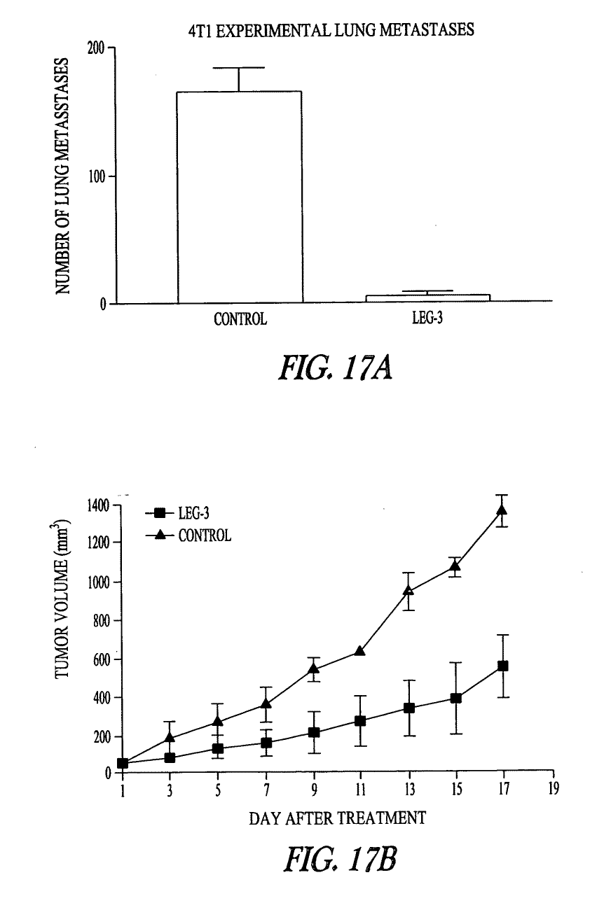

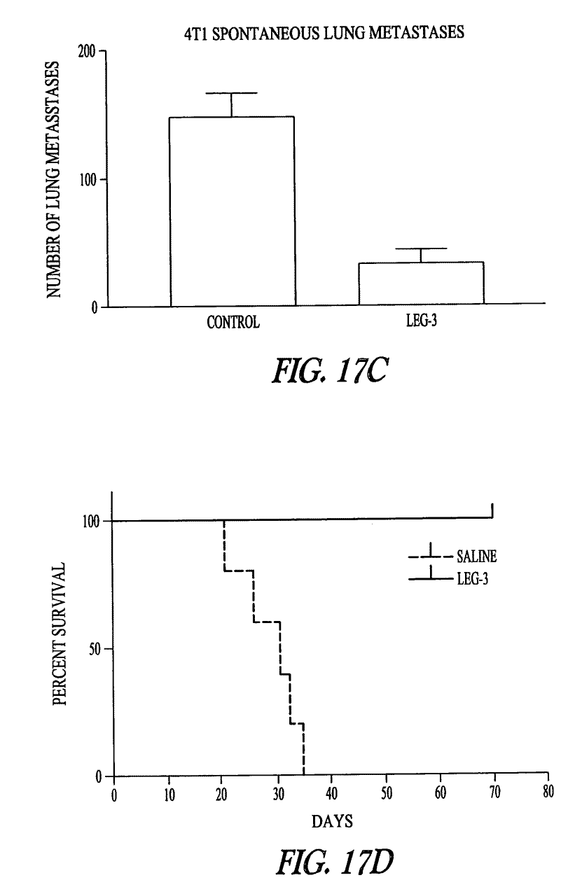

As shown in FIG. 17, LEG-3 suppresses experimental and spontaneous metastasis. When experimental lung metastasis was induced in mice using 4T1 metastatic breast cancer cells the treatment group, which received LEG-3 exhibited substantially no metastasis (FIG. 17A). In contrast, the control group exhibited high levels of metastasis (p<0.005, n=8; FIG. 17A). In addition, mouse mammary carcinoma growth was suppressed by LEG-3 (n=8; FIG. 17B) and LEG-3 treatment significantly reduced spontaneous lung metastasis in 4T1 tumors. (p<0.005; FIG. 17C). Animal survival was also substantially improved by LEG-3 treatment (FIG. 17D).

Therefore, the cell impermeable targeting tumor microenvironment activated prodrug (TMEAP) strategy described herein was highly effective against a variety of tumor types in vivo.

The present prodrugs include a peptidyl targeting agent for legumain which exhibits specificity in its catalytic function at a pH<6.8 and is selectively expressed extracellularly in the tumor microenvironment. Due to its extracellular activity, legumain can convert an effectively designed cell impermeable prodrug (e.g. LEG-3) to a cell permeable prodrug (Leu-Dox) which in turn is converted to the active tumoricidal end product Dox and translocated to cell nuclei for induction of cell death.

There are distinct advantages to this strategy in that tissue uptake can occur only in the tumor or comparable pathologic microenvironment. To achieve continuous growth and remodeling, the tumor microenvironment is enriched with a variety of proteases. There are nearly five hundred proteases identified so far in the human genome, and many have been associated with the local tumor microenvironment and appear important for tumor invasion and metastasis. Drug access to solid tumors is relatively efficient, limited only by diffusion barriers. Compounds like LEG-3 can therefore be converted and activated in the tumor microenvironment so that they produce substantial bystander effects not only on the tumor cells but also the associated endothelial and stromal cells. Whereas, most cytotoxic drugs are designed to be cell permeable, the present prodrugs have diminished cell permeability, increased hydrophilic properties, and increased drug solubility to minimize tissue uptake of LEG-3. This correlates with the slower clearance from the blood as well as greatly diminished tissue accumulation. In contrast, tumor cells exhibit increased uptake of the present prodrugs.

Thus, the invention provides anti-tumor prodrugs with improved properties.

### Example 7

### Selective Depletion of Macrophages Delays Tumor Growth

An established procedure was tested to observe the effects of selectively killing macrophages upon macrophage on tumor development in 4T1 breast cancer models. Cl₂MDP liposomes were used as agents that specifically target and kill macrophages.

Mice (Balb c, n=4) were treated with Cl₂MDP liposomes while control mice (n=4) were untreated. All mice were then challenged with syngeneic 4T1 breast cancer cells.

The Cl₂MDP liposome treatment resulted macrophage depletion and a significant delay of tumor growth. In particular, untreated cancer cells formed 4T1 breast cell tumors in about 7 days. In contrast, Cl₂MDP liposome treatment delayed tumor development substantially to about 45 days after tumor cell injection.

Therefore, macrophages have an important role of in the development of tumors.

### Example 8

### Legumain Activated Prodrug Selectively Target Tumor-Associated Macrophages

This Example illustrates that liposome formulations of the inventive prodrugs can selectively deplete tumor associated macrophages (TAM) from tumors.

### Methods

LEG-3 was synthesized as described in previous Examples.

To prepare liposome encapsulated LEG-3, stock solutions of phosphatidylcholine and cholesterol were prepared by dissolving phosphatidylcholine (PC) and cholesterol (0.8 mg) in 1 mL of chloroform each. These stock solutions can be stored in -20° C. under N₂ with the cap tightened and sealed with parafilm. The phospholipid layer was prepared in a molar ratio of 98 phosphatidylcholine: 2 cholesterol by adding 155 μL of phosphatidylcholoine to 193 μL of cholesterol in a 15×85 mm glass tube. The lipid mixture was placed under a gentle stream of N₂ gas until all chloroform was evaporated. To this mixture was added between 0.5-1 ml of Leg-3 compound (1 mg/100 μl, 0.9% saline). The mixture was incubated at room temperature for at least 30 minutes, then vortexed gently and intermittently until the phospholipid layer went into solution and was no longer seen on the bottom of tube. The mixture was then sonicated for 10-20 minutes, checking periodically to see if the solution was less opaque. The preparation was then transferred to a 1.5 mL tube and centrifuged at 10,000 g for 10 minutes. A pink lipid layer formed at the top of the tube. This layer contained the liposomes. This liposome-containing layer was transferred to another 1.5 mL tube and washed 2-3 times with sterile 0.9% saline using centrifugation at 25,000 g for 10 minutes at 4° C.

The liposomes were resuspended in sterile 0.9% saline prior to injection into mice.

### Results

Unlike the Cl₂MDP liposome, administration of LEG-3 liposomes selectively killed tumor-associated macrophages (TAMs) without harm M1 type of macrophages. In particular, CD14 positive macrophages were not depleted from the spleen in mice that were treated with LEG-3 liposomes. Therefore, the normal innate immune functions of macrophages were unaffected.

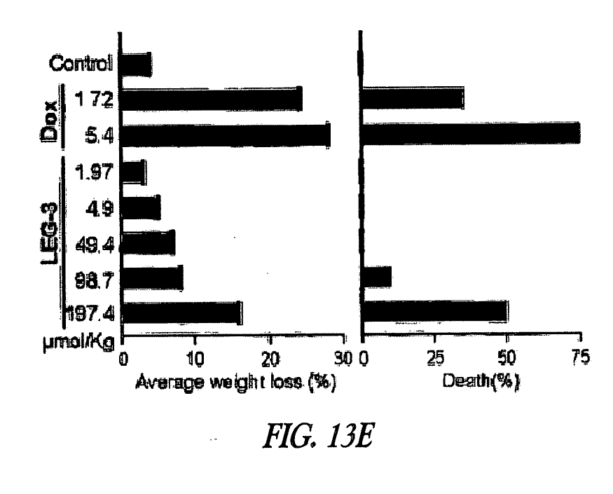

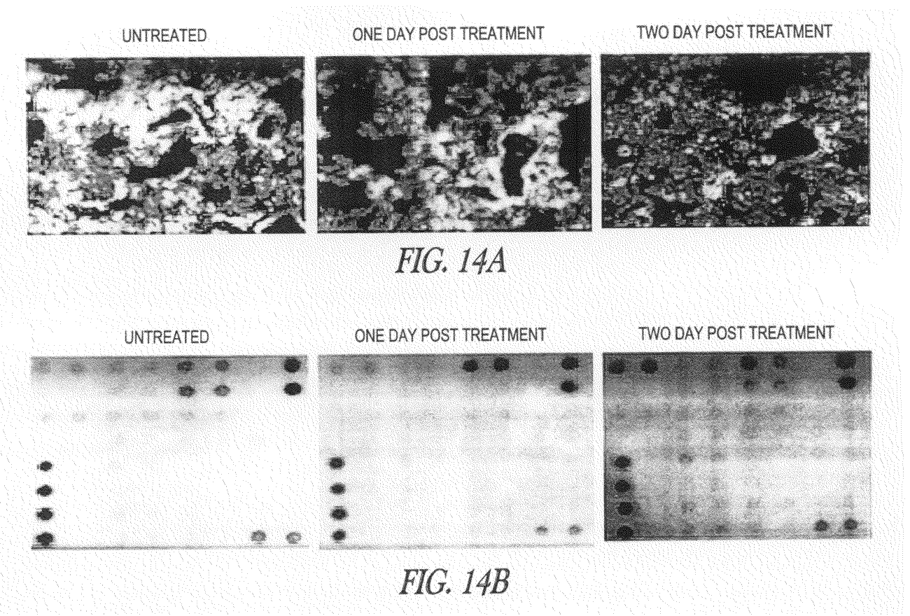

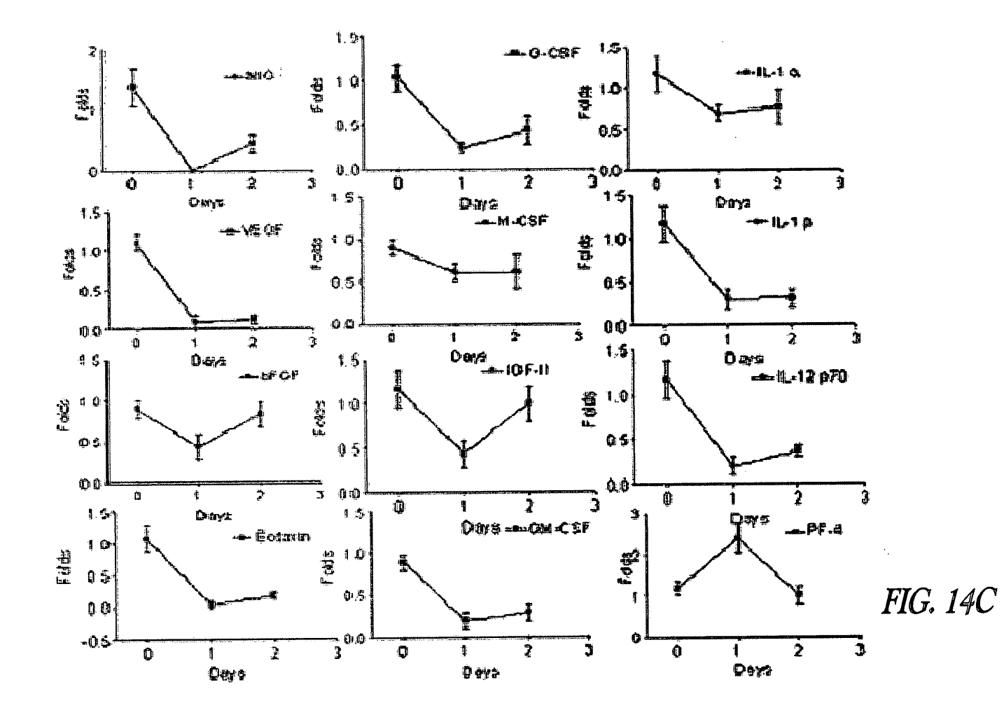

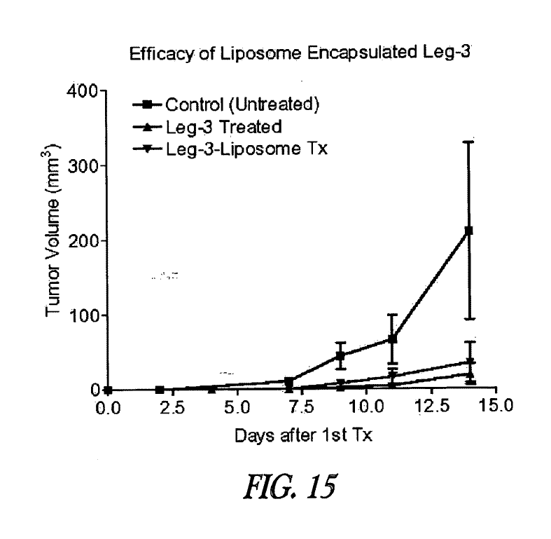

In contrast, the LEG-3 liposome preparation selectively depleted TAMs in tumors (FIG. 14A). This TAM depletion resulted in significant reduction of angiogenic growth factors (FIGS. 14B and C). Treatment with liposome encapsulated LEG-3 at low dosage (5 umol/kg) also resulted in significant growth suppression of 4T1 tumor in mice (FIG. 15). This effect was substantially due to the depletion of macrophages and the resulting anti-angiogenic effect. Therefore, the LEG-3 prodrug kills TAM effectively and reduces the levels of angiogenic factors in tumors.

However, some evidence suggested that TAM infiltrates recover quickly and so does the production of angiogenic factors. These data suggest that to effectively attack tumors targeting TAM, a metronomic dosing of the TME activated prodrug be optimal.

### Example 9

### AEP1 Inhibits Invasive Tumor Cell Growth and Metastasis

This Example illustrates that AEPI-1 inhibits angiogenesis as well as tumor invasive growth and metastasis. AEPI-1 is cell-impermeable. These studies indicate that AEPI-1 inhibits angiogenesis, tumor invasive growth and tumor cell metastasis. Since AEPI-1 is cell-impermeable its efficacy is likely due to inhibition of extracellular legumain in the tumor microenvironment.

### Materials and Methods

The structure of AEPI-1 (abbreviated Cbz-Ala-Ala-Aza-Asn-(S,S)-EPCOOEt) is as follows.

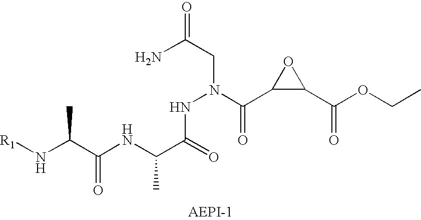

wherein R₁ is Cbz.

Tumor induction was performed by subcutaneous injection of 5×10⁵ 4T1 cells in the right flank mammary fat pat of six-week-old BALB/c mice. Two different groups of eight animals were treated on day 7 after tumor induction. Treatment involved 100 ul intraperitoneal injections of either PBS alone (control group) or AEPI-1 (treatment group). The treatment group was given 50 μg/AEPI-1 in saline by intraperitoneal injection every two days for two weeks. Tumor volumes of treated animals were measured everyday starting on day 6 by micro-caliper measurements (volume=width×length×width/2). As soon as the tumor volume reached 1150 mm³ in the control groups (on day 25), euthanasia was performed, lungs were removed and fixed in the Bouin's solution. Lung metastases were counted by anatomy microscope. The statistical significance between treatment and control groups was determined by two-tailed Student's t tests using Microsoft EXCEL software.

4T1 Lung Experimental Metastasis was generated by suspending 4T1 cells (1×10⁵) in 0.1 ml serum-free medium and injecting these cells into the tail vein of mice under the anesthesia. Treatment involved 100 μl intraperitoneal injection of either saline alone (control group) or AEPI-1 (treatment group). The AEPI-1 treatment group was given 100 μg AEPI-1 in saline (100 ul) by intraperitoneal injection daily for two weeks. All mice were euthanized by CO₂ and lungs were removed, weighted and fixed in the Bouin's solution. Lung metastases were counted by anatomy microscope.

### Results

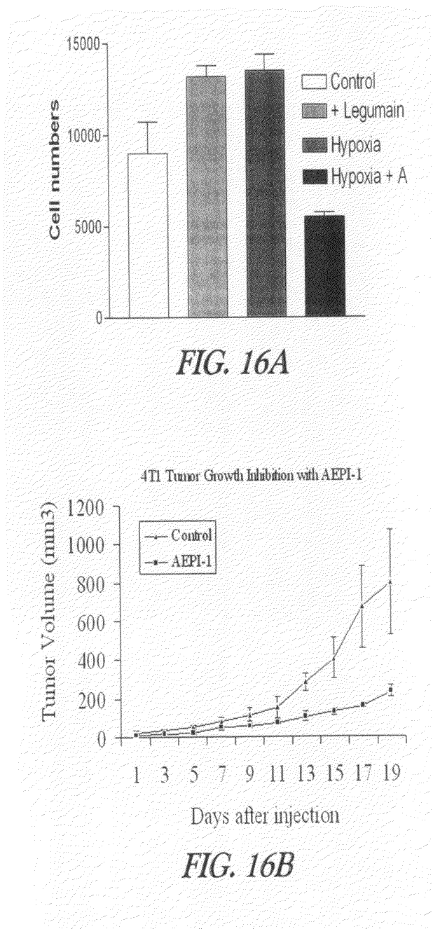

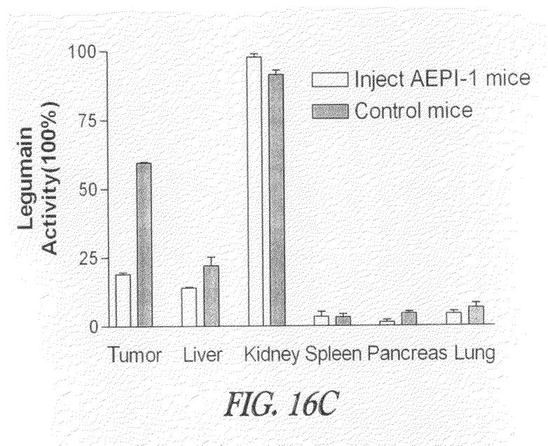

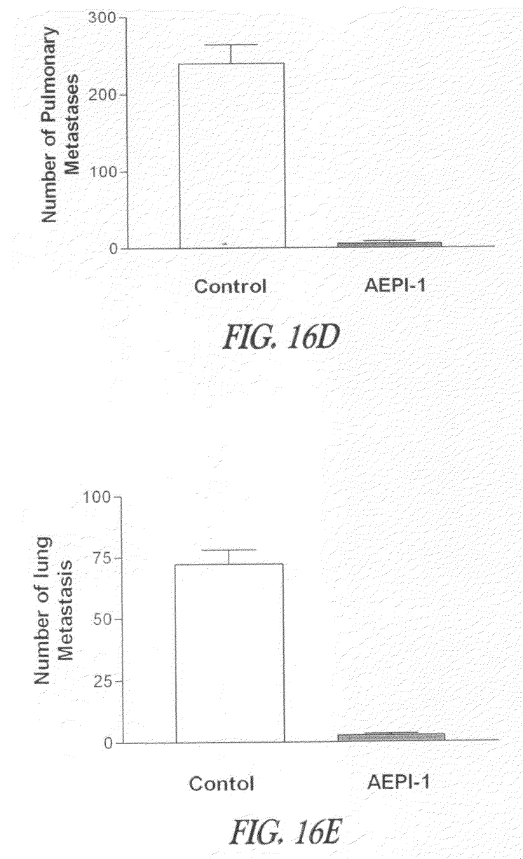

As shown in FIG. 16A-E, AEPI-1 suppresses tumor metastasis and invasive growth. FIG. 16A shows that while legumain promoted tumor cell invasion in vitro (second bar from left), AEPI-1 inhibited hypoxia induced breast cancer cell migration (fourth bar from left). FIG. 16B shows that AEPI-1 inhibits the growth of 4T1 urine mammary carcinoma. FIG. 16C shows that systemic administration of AEPI-1 inhibited legumain activity in 4T1 tumors, but does not affect legumain activity in kidney, indicating that AEPI-1 is substantially impermeable to cells and therefore not affecting intracellular legumain. FIG. 16D shows that AEPI-1 inhibited experimental lung metastasis of 4T1 mammary carcinoma. FIG. 16E shows that AEPI-1 inhibited spontaneous lung metastasis of 4T1 mammary carcinoma.

### Example 10

### Paclitaxel Prodrugs

This Example illustrates synthesis of paclitaxel prodrugs that can be used to specifically target tumors.

Paclitaxel is a natural product isolated from Taxus brevifolia, and has been widely used to treat solid tumors such as ovarian, breast and non small cell lung cancers. Its mechanism of action involves mitotic arrest in cancer cells through stabilization of microtubules via inhibition of depolymerization. Despite its contribution to chemotherapy, paclitaxel suffer a number of undesirable difficulties, such as poor selectivity for killing normal versus cancer cells, the development of multidrug resistance, and a severe lack of solubility in aqueous systems. This Example illustrates synthesis of paclitaxel prodrugs that overcome some of these disadvantages.

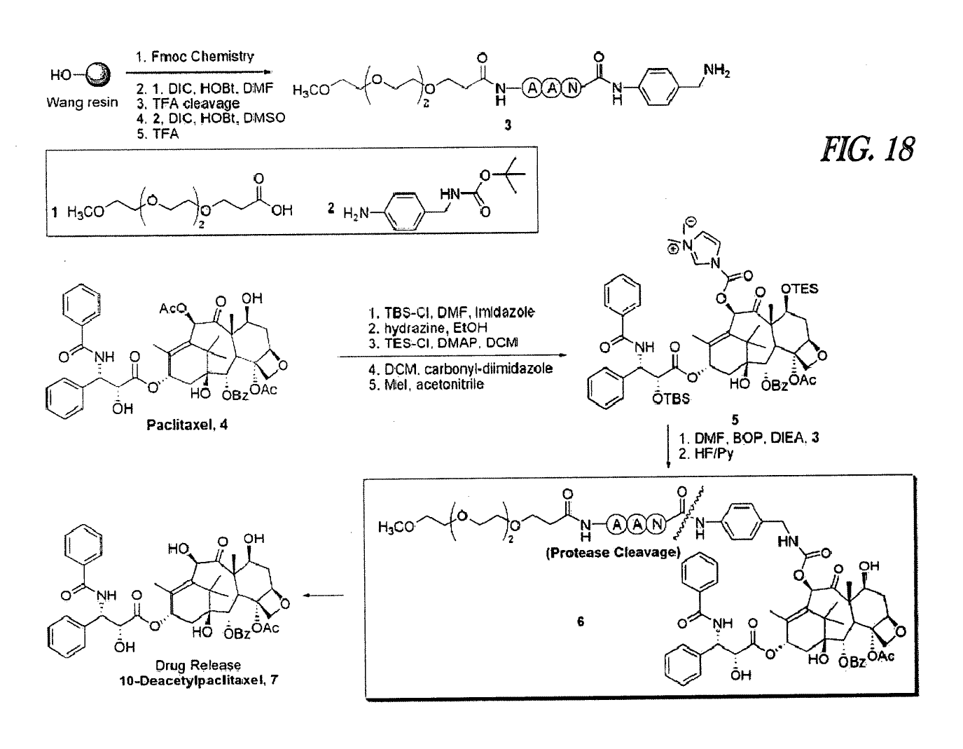

A prodrug containing paclitaxel, compound 6, is synthesized as shown in FIG. 18. The attractive features of this molecule are as follows: (1) The molecule is tripartite in that it contains a 4-aminobenzylcarbamate linker for traceless drug release, paclitaxel for tumor cytotoxicity and a PEG-tripeptide for both selective delivery of paclitaxel and additional water solubility effects. (2) Activation of the paclitaxel portion of the molecule can only occur upon cleavage of Asparagine-linker. (3) The molecule itself is modular such that the synthetic route can easily accommodate alternative drugs, peptides or water enhancing linkers.

Synthesis of the tripeptide portion of prodrug (3) will begin with the preparation of the Ala-Ala-Asn peptide which will be accomplished using standard stepwise solid phase peptide synthesis protocols for Fmoc chemistry using DIC/HOBt on Wang resin (see FIG. 18). The m-dPEG™ acid (1) will be coupled on the N-terminus to increase solubility of the peptide-paclitaxel conjugate and the PEG-peptide bound resin will be then washed and global side-chain deprotection/cleavage will performed in 5% anisole, 5% triisopropylsilane, 90% TFA. Noteworthy in our synthesis is the 4-(Boc-aminomethyl)-aniline (2) that will be incorporated for a traceless drug release via a 1,6-elimination (Jeffrey et al., Bioconjug. Chem. 17: 831-40 (2006)). The crude peptide will be triturated, lyophilized and purified by preparative RP-HPLC to provide compound 3 (FIG. 18).

For the preparation of paclitaxel prodrug conjugate 6, peptide 3 is joined to paclitaxel without compromising its cytotoxicity. To do this, the C-10 hydroxyl moiety embedded in the taxol framework is used. This site is selected as a key point for attachment because this region is more tolerant to functionalization. Amino acids, sugars and small polymers have been joined to C-10 site of paclitaxel. To complete the synthesis, the methylimidazolinium taxane salt 5 is made as illustrated in FIG. 18. Thus, 2′-O-TBS-7-O-TES-10-O-deacetyl-paclitaxel will be synthesized in three steps from paclitaxel (4), and upon reaction with carbonyl-diimidazole and methyl iodide, compound 5 will be prepared. Attachment of peptide 3, to form 6 will be done by addition of peptide 3 to compound 5 in the presence of BOP reagent, followed by HF/pyridine treatment to remove the TES and TBS protecting groups. The crude peptide-paclitaxel conjugate will be purified by preparative RP-HPLC and lyophilized to provide prodrug 6.

As shown in FIG. 18, asparagine cleavage by the legumain protease will induce a traceless release of 10-deacetylpaclitaxel 7 via a 1,6-elimination. Prodrug 6 possesses the same cytotoxicity as paclitaxel and is an excellent candidate for selective prodrug delivery to carcinoma cells.

Compounds that are made by these procedures are shown below.

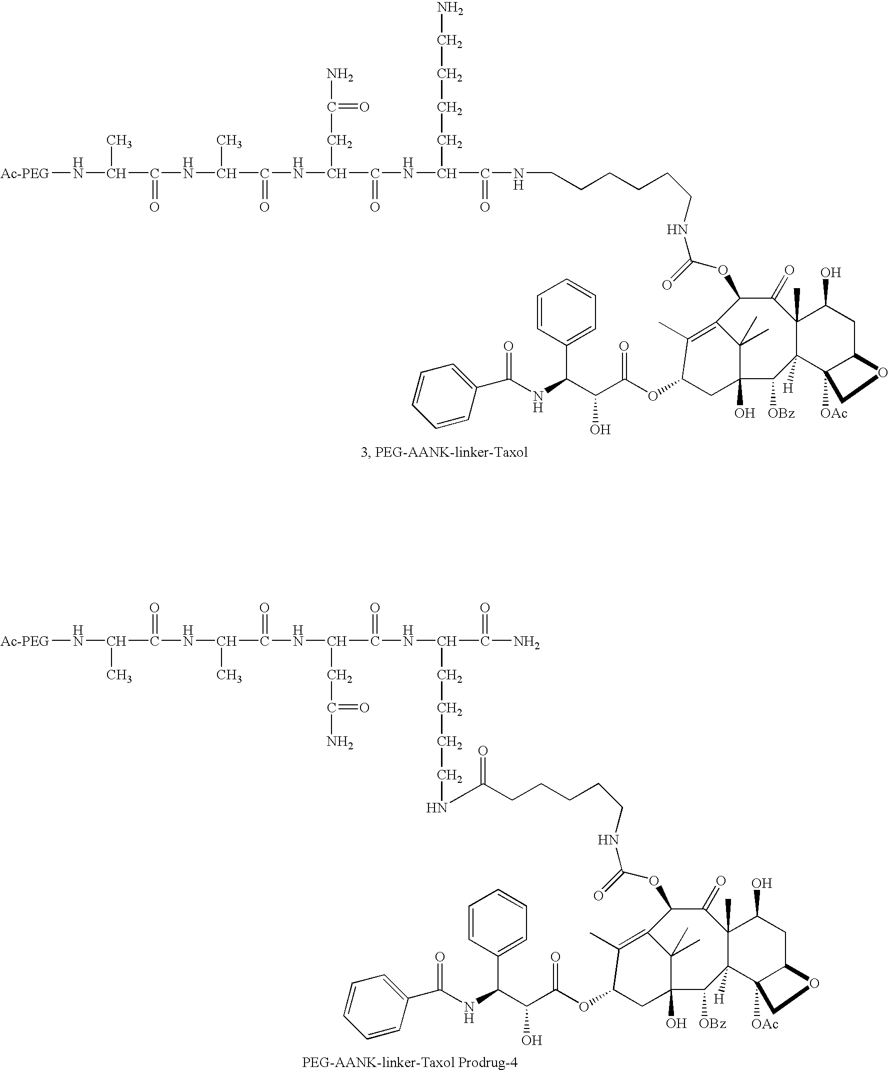

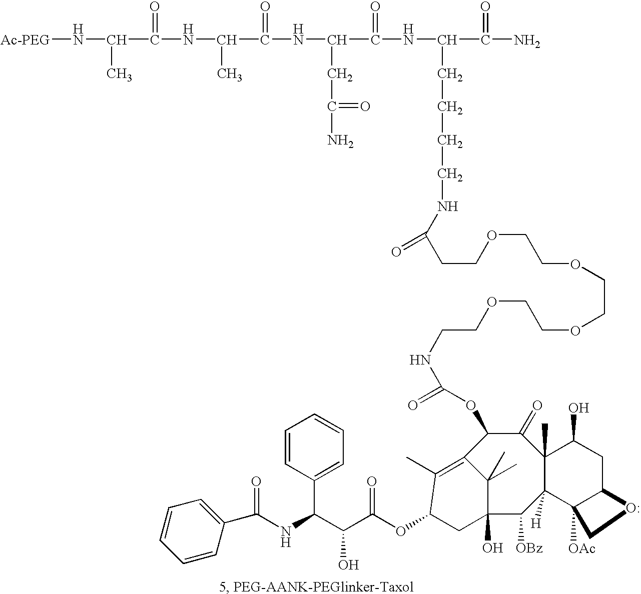

### Example 11

### Legumain Activity Inhibits Apoptosis

This Example describes experiments showing that legumain inhibits apoptosis, particularly in cancer cells. Both tumor and tumor stromal cells have to survive under substantially lower oxygen tension within the tumor micro-environment in contrast to adjacent normal tissues. The inventors have observed that expression of the legumain asparaginyl endopeptidase is induced by hypoxia.

Over-expression of legumain protects cells against programmed cell death. Legumain expression protects cells from multiple programmed cell death pathways. This mechanism is operative in multiple cell types in the tumor microenvironment as well as against a spectrum of biologic and therapeutic cytotoxic stimuli. Cells that over-express legumain have normal appearance. However they exhibited a significant survival advantage when treated with inducer of apoptosis (e.g., TNF-α).

Preliminary experiments indicated that 293 cells that express legumain are resistant to TNF-α-induced apoptosis compared to wild type 293 cells and that legumain protects against apoptosis in a dose dependent manner as measured in a tetracycline regulated expression system. Other data indicate that legumain expression protects cells against apoptosis by preserving mitochondria integrity. This is confirmed with a tetracycline inducible system in which legumain expression is induced by tetracycline. Over-expression of legumain also protects human cancer cells against apoptosis.

In 293 cells, TNF-α treatment resulted in increased lysosomal permeabilization and cathepsin B diffusion into cytoplasm. The integrity of mitochondria is compromised and cytochrome c is diffused into cytoplasm. As expected TNF-α treatment resulted in apoptosis of these cells. In contrast, although cells that over-express legumain exhibit cathepsin B leakage, the integrity of mitochondria is still preserved and the cells are resistant to apoptosis.

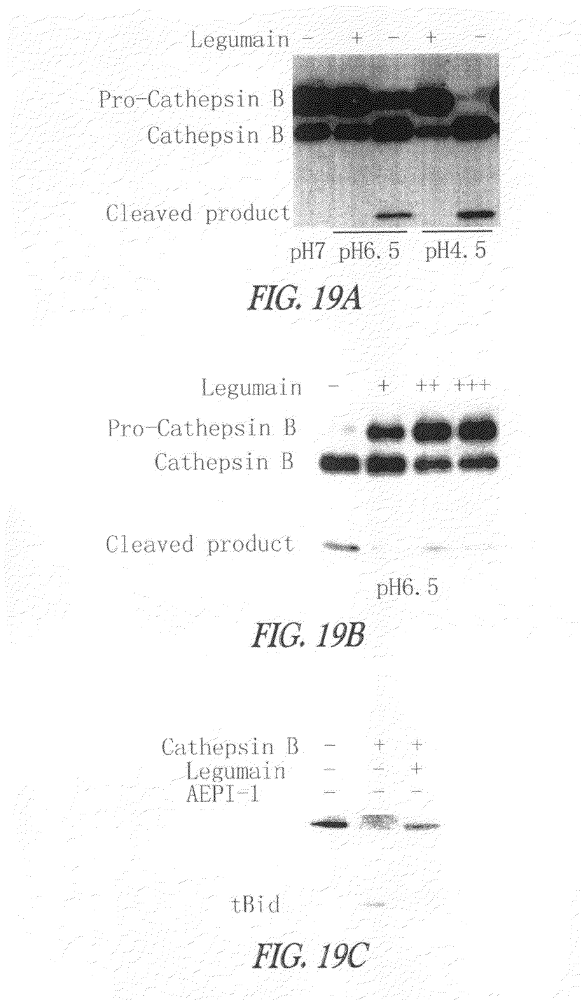

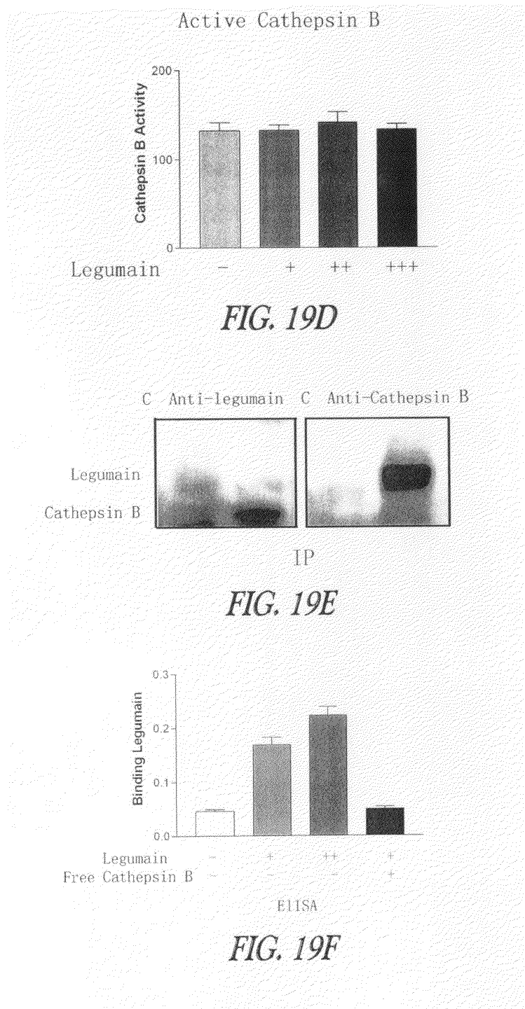

Legumain binds cathepsin B and inhibits cathepsin B auto-activation and Bid activation. In contrast to activation of cathepsin L by legumain, FIG. 19A demonstrates that legumain suppresses cathepsin B auto-activation. This inhibition is dose dependent (FIG. 19B). These data are consistent with the legumain knockout study where cathepsin B activity is enhanced while cathepsin L activity is decreased. Important for regulation of apoptosis, legumain also affects cathepsin B activity toward its physiologic substrates influencing, for example, Bid activation (FIG. 19C). Interestingly cathepsin B activity toward small peptidyl substrates is not affected (FIG. 19D). Direct binding of legumain with cathepsin B is demonstrated with co-immunoprecipitation studies (FIG. 19E) and competitive binding analysis (FIG. 19F). These findings indicate that legumain can uniquely modulate the activity of cathepsin B.

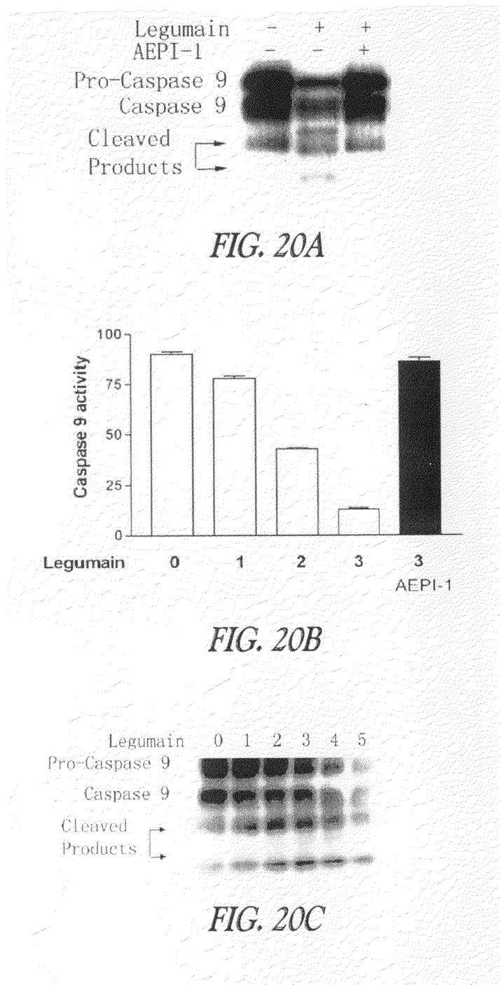

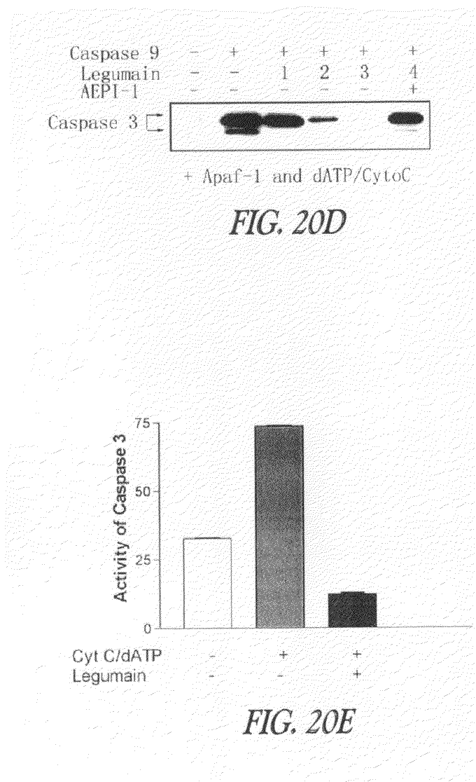

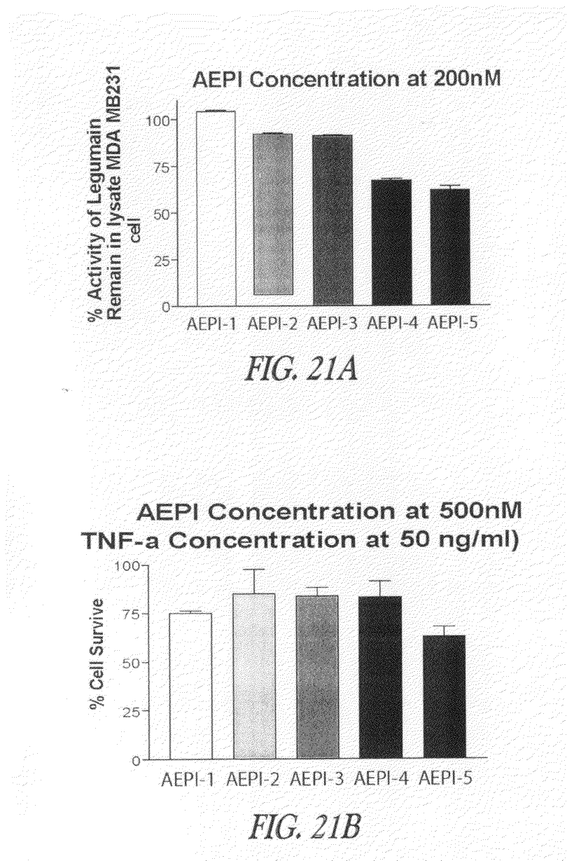

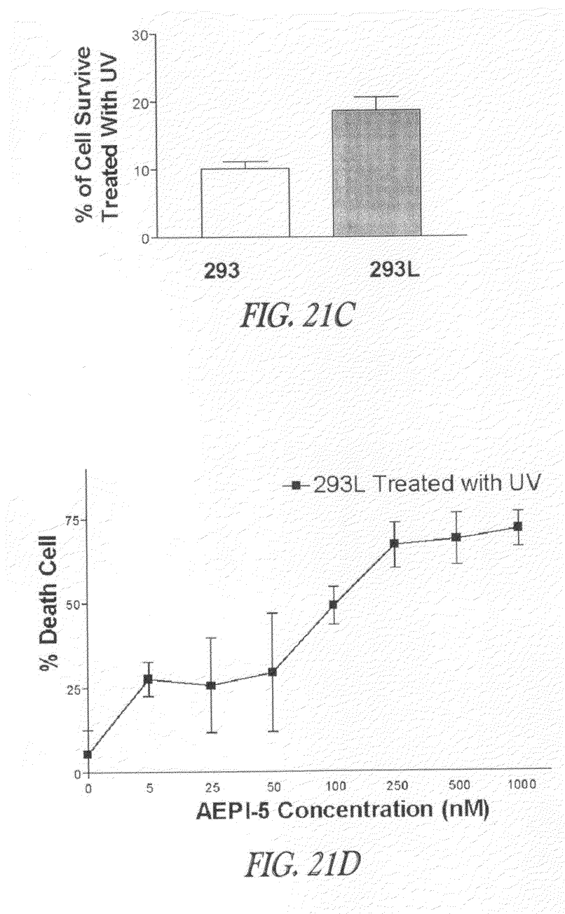

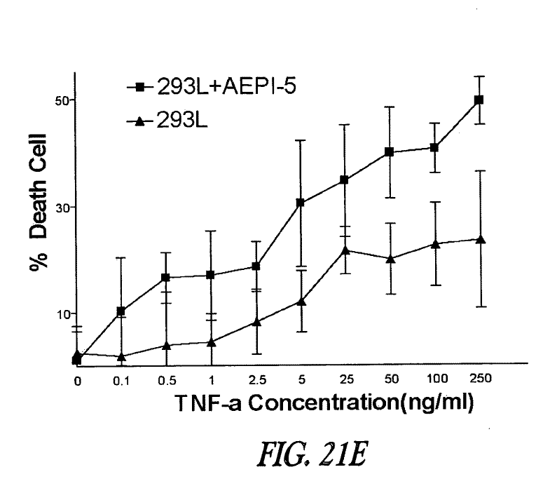

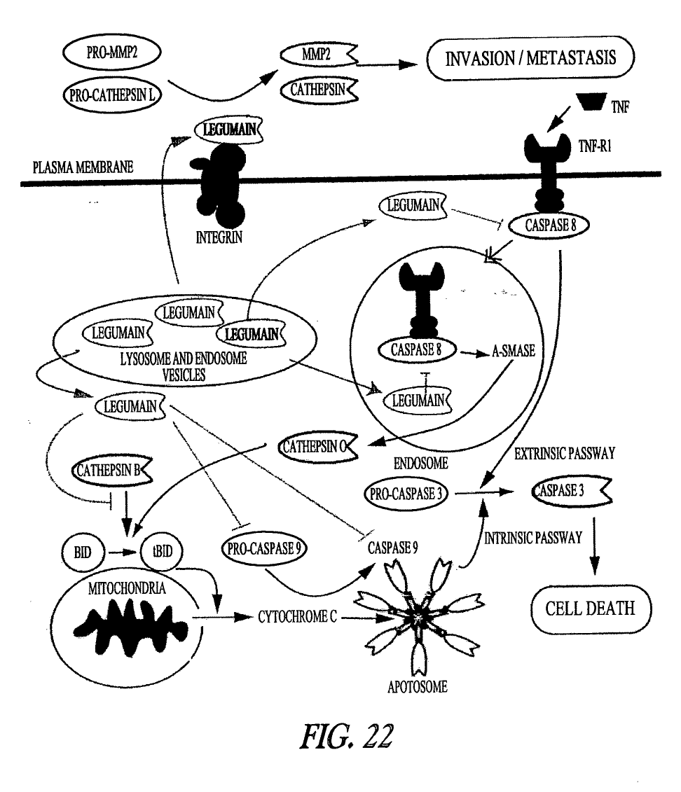

Legumain catalytically inactivates caspase 9. In addition to suppression of cathepsin B, legumain efficiently inactivates caspase 9 and depletes the cell of pro-caspase 9 (FIG. 20A). Legumain cleavage of caspase 9 resulted in the lost of caspase 9 activity; moreover this loss of caspase 9 activity is inhibited by AEPI-1 (FIG. 21B). Increasing amount of legumain in cells resulted in reduction of pro-caspase 9 and active caspase 9 (FIG. 20C). Loss of pro-caspase 9 led to reduced caspase 3 activation in a reconstituted apoptosome system (FIG. 20D), which was further confirmed by detection of reduced caspase 3 activity (FIG. 20E). These observations indicate that asparaginyl endopeptidases play a critical role in cell survival and tumorigenesis. Inhibition of legumain activity and expression sensitizes tumor cells to chemotherapeutic agents suggesting new therapeutic possibilities for cancer intervention and treatment.

### Example 12

### Cell-Permeable AEPIs Increase Tumor Cell Apoptosis

This Example demonstrates that while legumain protects tumor cells against apoptosis, legumain inhibitors (e.g., the AEPIs disclosed herein) increase apoptosis in tumor cells.

### Materials and Methods

### Synthesis of asparaginyl endopeptidase inhibitor (AEPI)₁. Aza-peptide

1 was synthesized as illustrates in Scheme 1. Briefly, dipeptide 4 was synthesized by coupling of Cbz-L-alanine and L-analine methyl ester, and then converted to the hydrazide 4 by treating with anhydrous hydrazine in methanol. The hydrazide 5 was alkylated with ethyl bromoacetate and the resultant product 6 underwent ammonolysis to afford amide 7. The latter product was coupled with epoxy-succinic acid mono ethyl ester giving the desired compound 1.

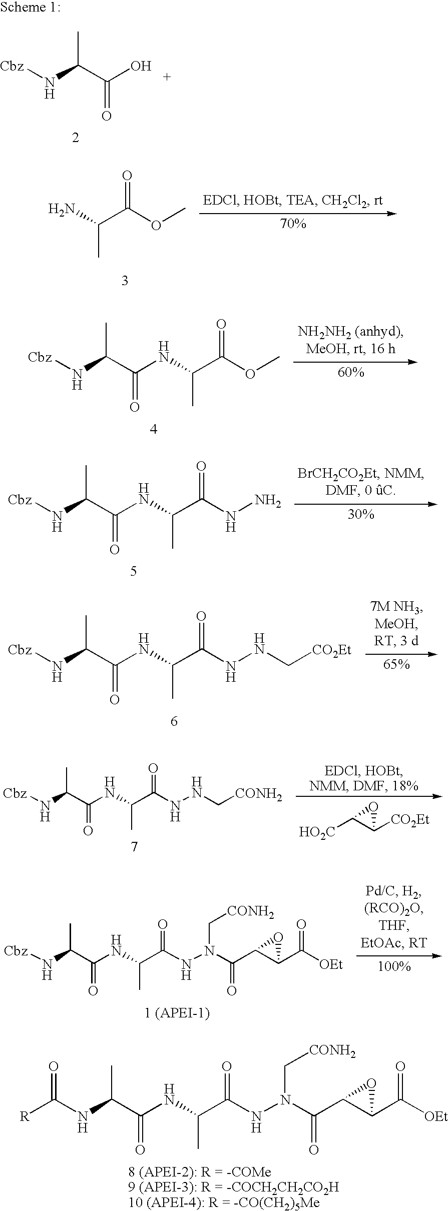

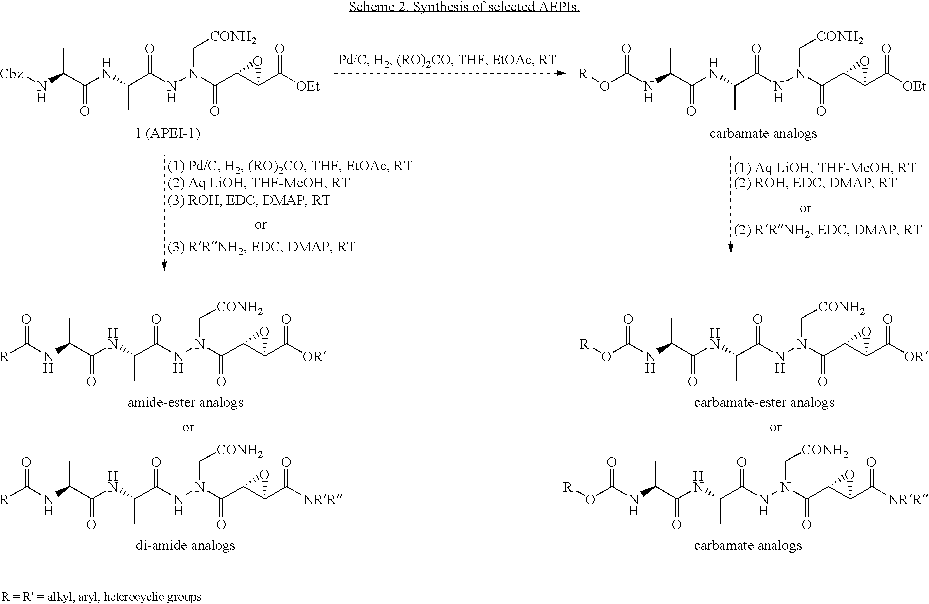

### Synthesis of New AEPI Analogs

While AEPI-1 is useful as a soluble inhibitor of asparaginyl endopeptidases, it lacks the cell permeability for inhibition of intracellular legumain that is involved in apoptosis. New AEPI analogs are developed that differ from the parent AEP1 molecule at one or both of the protecting group sites. For example, the benzyloxycarbonyl (abbreviated herein as cbz) and/or the ethyl ester protecting group sites can be changed to alter the solubility and cell permeability properties of the AEP1 molecules.

In particular, these protecting groups can be replaced with various alkyl, aryl or heterocyclic groups. Synthesis of new analogs will be achieved starting with AEPI-1. Initial experiments are focused on the cbz group, which is replaced with both ionizable and non-ionizable protecting groups. For example, compounds AEPI-5, 6 and 7 (8-10, in Scheme 1 above) were made by removing the cbz group and reacting the resultant amine functional group in situ with acetic anhydride, succinic anhydride or heptanoyl anhydride (see Scheme 2). Similarly, various aromatic, aliphatic and heterocyclic anhydrides can be used to provide AEPI-1 analogs. If anhydrides are not available, NH-succinyl ester of an acid can be used to modify the primary amine after hydrogenolysis, in situ. In addition, aromatic, aliphatic and heterocyclic carbonates can be used to prepare the analogous carbamates. The ethyl ester function can be replaced by hydrolysis of AEPI-1, or its analogs, under basic conditions using LiOH to acid, followed by esterifying the latter with various aromatic, aliphatic and heterocyclic alcohols. Additionally, the acid can be amidated using amines.

The resultant AEPIs will be assessed in following assays.

Assays to Determine the types of cancers that Benefit from Intracellular Inhibition of Legumain. The effects of legumain expression on the cellular response to apoptosis inducing agents can be assessed in a panel of human tumor cells. Human breast cancer cells (MDA-MB-231 and MCF-7), pancreatic cancer cell (Panc-1), prostate cancer cells (LnCap), and fibrosarcoma (HT1080) have been or will be used for this study. Additional human tumor cells are available from ATCC and can be used for same purpose. Legumain expression is achieved with transient expression in cells with high transfection efficiency and retrovirus vector as described herein. Stable cell lines that express legumain may be constructed using selected cell lines. The following apoptosis inducing agent will be used, TNF (CHX), TRAIL, Staurosporin, Actinomycin, etoposide, doxorubicin, paclitaxel, methotrexate, UV (or radiation). Other agents are available and may be used as well.

The following protocols will be used to assess cytotoxic and cytostatic effect. These assays can be modified to compare wild type cells and cells expressing legumain and/or with AEPI.

AEPI-5 is sufficiently cell permeable to perform many of the in vitro assays and preliminary results have been obtained with this AEPI-1 compound (see, FIG. 21).

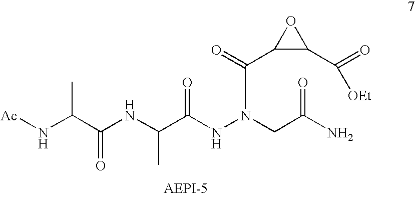

Chemo-sensitizing Cytotoxic Assay. Cancer cells were prepared for the assay as follows. The cells were centrifuged at 800 rpm for 10 minutes, resuspended in DMEM to a density of 1.0×10⁵ cells/ml. Approximately 5 ml of cells were used per assay plate. Cells (100 ul) were added to a 96-well flat bottom plate (10,000 cells/well, all the inner wells) and 100 ul of media was added to the outer wells. The plates were incubated at 37° C. overnight. AEPI dilutions were prepared using a 96 well round bottom plate and medium as the diluent. After overnight incubation, the media was removed from the cancer cell assay plate, and the selected AEPI was added at the selected dilution or concentration. An additional 200 ul of media was added to the outer wells. Plates were incubated overnight at 37° C. XTT reagent (or other cell viability reagent) was prepared in phenol red free media. The media was removed from cells and 200 ul of fresh media containing the XTT reagent was added to all wells. The plates were incubated for 30 min to 4 hrs and cell viability was determined by reading the plates according to manufacturer specifications.

Chemo-sensitizing Cytostatic Assay. Cancer cells were centrifuged at 800 rpm for 10 minutes and the cells were resuspended in DMEM to a density of 4.0×10⁴ cells/ml. Approximately, 5 ml of cells are needed per assay plate. Cells (100 ul) were added to a 96-well flat bottom plate (4,000 cells/well, in the inner wells) and 100 ul of media was added to the outer wells. The plates were incubated at 37° C. overnight. The prodrug compounds were diluted (in a 96 well round bottom plate) to generate serial dilutions if the prodrug. The old media was removed from the cancer cell assay plate, the AEPI in 100 ul media was added to the effective concentration determined in AEPI inhibition assay and 200 ul of media was added to the outer wells. Cell viability was assessed after incubation for about 12-46 hrs.

AEPI Inhibitory Assay. Cancer cells were centrifuged at 800 rpm for 10 minutes and resuspended in DMEM to a density of 1.0×10⁵ cells/ml. Approximately 5 ml of cells is used per assay plate. Cells (100 ul) were added to a 96-well plat bottom plate (10,000 cells/well, all the inner wells) and 100 ul of media was added to the outer wells. The cells were incubated at 37° C. overnight. The inhibitor compounds were diluted in a 96 well round bottom plate and the old media was removed from the cancer cell assay plate. The inhibitor (100 ul) was added to the cell assay plate and 200 ul of media was added to the outer wells. One to three days after the cancer cells were exposed to the inhibitor, the AEP activity was assessed as follows. Cells were detached with Trypsin-free cell-cell detachment media and transfer to Eppendorf tubes. Cells were then disrupted and the AEPI substrate was added to the cell lysate. This assay mixture was then incubated for an appropriate period of time and the amount of activated substrate was measured with fluorometer.

AEPI Cytotoxic Assay. Cancer cells were centrifuged at 800 rpm for 10 minutes and resuspended in DMEM to a density of 1.0×10⁵ cells/ml. Approximately 5 ml of cells is used per assay plate. Cells (100 ul) were added to a 96-well plat bottom plate (10,000 cells/well, all the inner wells) and 100 ul of media was added to the outer wells. These plates were incubated at 37° C. overnight. The inhibitor compounds were diluted in a 96 well round bottom plate and the old media was removed from the cancer cell assay plate. The inhibitor (100 ul) was added to the cell assay plate and 200 ul of media was added to the outer wells. One to three days after the cancer cells were exposed to the inhibitor, the viability of the cells was assessed using XTT reagent.

Evaluation of Effects of AEPI in Combination with Chemotherapeutic Agents Using Human Neoplastic Models in vivo. The safety and efficacy of cell permeable AEPI will be evaluated with the following in vivo testing. The analyses of tumor arrest and eradication will be performed in rodent models with immunodeficient rodent models with established human breast tumors (MCF7) and prostate cancer (LnCap). Full scale analysis will be performed of AEPI with and without chemotherapeutic agents using a variety of protocols to evaluate the efficacy, appropriate dosages, administration regimens (intervals between administrations and numbers of administrations per therapeutic treatment), maximum tolerable dose per administration and per series of administrations. Statistical analyses of data will guide and validate progress. Drug safety will be monitored by monitoring the behavior, locomotion, response to stimuli, weight, physical characteristics (bleeding, disturbed movement, weakness, piloerection, hair loss, etc.) and other physiological signals. Pathological analyses will also be performs.

After establishing the in vivo maximum tolerable and optimal single dosages of AEPI, the optimized in vivo dosage, interval schedule, and duration of therapy for eradication of prototypic human tumor models in rodents by AEPI will be developed. Protocols for various procedures are described in more detail below.

### Tumor Generation Models.

LnCap human prostate carcinoma tumors. LnCap cells can generate tumors in WEHI nude mice. The LnCap cells can be maintained in tissue culture. The tumors are generated by injecting 10⁶ cells mixed with 100 ul of Matrigel subcutaneously into WEHI nude mice. Tumors are slow growing and are visible 20 days post injection.

MCF7 human breast tumors. MCF7 cells can generate tumors in WEHI nude mice. The cells can be maintained in tissue culture. The tumors are generated by injecting 10⁶ cells subcutaneously in WEHI nude mice. Tumors are slow growing and are visible 15 days post injection.

Hs 766T human pancreatic carcinomas. The Hs 766T is derived from a pancreatic carcinoma that is metastasized to lymph node. The cells can be maintained in culture. The tumors are generated by injecting 5×10⁶ cells subcutaneously in Balb/C nu/nu mice. Tumors are visible 4 weeks post injection.

HT1080 human fibrosarcoma. HT1080 human fibrosarcoma cells can be cultured. The tumors can be generated in Balb/C nu/nu by injection 10⁶ cells subcutaneously and the tumors are visible 14 post injection.

Additional models may be included if needed.

### Initial Testing for Efficacy and Toxicity.

Initial testing. This protocol will be used for assessing efficacy of newly synthesized batch of AEPI compounds, new formulations, and new tumor models. Tumors will be observed for arrest of tumor growth and maintenance of growth arrest. Human tumors in immunodeficient mice will be used for this purpose. This protocol will be used for analysis of the in vivo efficacy of AEPIs with and without chemotherapeutic agents. Currently, a dosage of 50 mg/kg is believed to be effective and non-toxic. A fixed schedule can be used for the testing of the compound(s). The experimental duration will be dependent on tumor growth rate. In general, the testing will be terminated 21 days from date of drug administration. However for some rapidly growing tumors, termination will be as short as 12 days, and for slow growing tumors the testing will be terminated at up to 6 weeks from the date of drug administration. If evidence of toxicity is observed (see below), the affected group will be euthanized. Different routes of drug administration will be evaluated, i.e., intravenous versus intraperitoneal. Preliminary studies indicate that the AEPI compounds have no toxicity at 50 mg/kg in mice. These tests can be performed again on new compounds as well as on the compounds described herein to confirm the results and to permit statistical analysis.

Treatment is initiated through bolus intravenous injection 7-10 days after tumor cell implantation or when the tumors reach about 4 mm in diameter. There will be 3 injections on alternate days representing a single “set” of therapeutic administrations. If successful, approximately two to four more sets of drug administrations will be administered. Tumor size, tumor growth rate and physical signs of toxicity will be monitored daily. Physical signs of toxicity may include local ulcerations over the tumor, changes in mobility, differences in stimuli responses, changes in eating patterns, piloerection, withdrawal from cage mates, hair loss, and weight changes in the various animals. Tumor sizes will be analyzed statistically in the treated and untreated tumor bearing animals to determine the efficacy of each compound and an appropriate duration of treatment. The experiment will be terminated at intervals relating to the tumor growth rate for single series of administrations, at about 28 days for animals received two series of administrations, and at about 42 days for animals receiving four series of administrations. All healthy tumor-free animals will be monitored for up to an additional 4 weeks. Any animals meeting the criteria for euthanasia will be euthanized.

Toxicity Free Maximal Tolerated Dose (MTD). Normal animals, Balb/C mice and Fisher rats will be used. Escalating single dosages will be administered and toxicity analysis will be performed at 42 days. Normal animals (8 per group) will be used to determine the MTD is concordant with guidelines of the National Cancer Institute. An escalating single dose of the selected drug will be administered to a series of animal groups and the animals will be observed for 35 days for any evidence of toxicity. Toxicity will be defined as no lethality within 15 days of treatment, no evidence of other toxic manifestations (see description of toxic effects elsewhere herein). There should be no weight loss and the rate of body weight growth should recover within 12 days. A non-toxic dosage is defined as an absence of indicia of toxicity as defined elsewhere herein for criteria for euthanasia.

Maximally tolerated total dose (MTTD). Normal animals, Balb/C mice and Fisher rats will be used. Animals will receive one-half single MTD three times a day for about 12 days or until evidence of toxicity is observed. Animals will be monitored for all indicia of toxicity. Animals will be euthanized at 42 days after the first administration and the effects of this dosage regimen will be evaluated pathologically and histologically.

Dose range analysis for arrest of tumor growth. MCF7, LnCap, Hs 766T, and HT1080 models will be used. This protocol will be used to search for short term effective maximal therapeutic efficacy dose. Serial increased dosages defined by the studies described above up to the MTTD will be explored for efficacy, as indicated by eradication of tumors and by non-recurrence within four weeks.

Treatment will be initiated when the tumors have reached 4 mm diameter (or at about 7-10 days after tumor cell implantation) by bolus intravenous injection of the selected reagents. The treatment will include three injections daily or one injection per day for three days. The treatment may be repeated after three days rest for one or more times. Tumor growth and physical signs will be monitored daily including any gross evidence of tumor necrosis, local tumor ulceration as well as evidence of toxicity including changes in mobility, differences in stimuli responses, changes in eating habits, and weight loss or gain for each animal. Tumor sizes in the treated and untreated tumor bearing animals will be compared to evaluate the efficacy of the compounds administered. The experiment will be terminated at about 21 days.

Number and duration of each series of administrations to arrest tumor growth rate, reduce tumor size and cause tumor regression. MCF7 and LnCap models will be used. This protocol will be based on dosage that determined in initial dosage range analysis and toxicity studies. Treatment will be initiated when the tumors are about 4 mm in diameter through bolus intravenous injection of the drug(s). Drugs will be administered three times per day on alternate days each week, or daily for one week. After a rest for up to one week, this administration regimen will be repeated two to four times to encourage tumor growth arrest and to analyze tumor regression. Tumor growth and physical signs of toxicity or tumor regression will be monitored daily. Tumor size in treated and untreated tumor bearing mice will be compared to determine efficacy of the compound. The experiment will be continued for 35 days following the last treatment. Euthanasia will be performed if needed and at the termination of the study.

Statistical Analyses. Statistical significance will be determined by the two-tailed Student's t test. Survival curves will be analyzed with the Logrank test.

### REFERENCES

Albini et al., Cancer Res, 47: 3239-3245, 1987.
Alvarez-Fernandez et al., J Biol Chem, 274: 19195-19203, 1999.
Asgian et al., J Med Chem, 45: 4958-4960, 2002.
Barrett et al., Biol Chem, 382: 727-733, 2001.
Beck et al., Eur J Immunol, 31: 3726-3736, 2001.
Bretscher et al., Curr Opin Cell Biol, 10: 537-541, 1998.
Cancer Facts & Figures, 2003: American Cancer Society.
Castino et al., Int J Cancer, 97: 775-779, 2002.
Chakravarty et al., J Med Chem, 26: 638-644, 1983.
Chambers et al., J Natl Cancer Inst, 89: 1260-1270, 1997.
Chang et al., Trends Cell Biol, 11: S37-43, 2001.
Chen et al., J Biol Chem, 272: 8090-8098, 1997.
Chen et al., Biochem J, 335: 111-117, 1998.
Chen et al., Biol Chem, 382: 777-783, 2001.
Choi et al., J Biol Chem, 274: 27747-27753, 1999.
Choi et al., J Bone Miner Res, 16: 1804-1811, 2001.
Coulibaly et al., Int J Cancer, 83: 526-531, 1999.
de Groot et al., J Med Chem, 42: 5277-5283, 1999.
de Jong et al., Cancer Chemother Pharmacol, 31: 156-160, 1992.
Denmeade et al., Cancer Res, 58: 2537-2540, 1998.
Dubowchik et al., Bioorg Med Chem Lett, 8: 3347-3352, 1998a.
Dubowchik et al., Bioorg Med Chem Lett, 8: 3341-3346, 1998b.
Foghsgaard et al., J Cell Biol, 153: 999-1010, 2001.
Gravesen et al., Microbiology, 146 (Pt 6): 1381-1389, 2000.
Hanahan et al., Cell, 100: 57-70, 2000.
Itoh et al., J Biol Chem, 273: 24360-24367, 1998.
Mai et al., J Biol Chem, 275: 12806-12812, 2000.
Manoury et al., Nature, 396: 695-699, 1998.
Nabi et al., J Cell Sci, 112: 1803-1811, 1999.
Nagase et al., Biol Chem, 378: 151-160, 1997.
Payne et al., Cancer Cell, 3: 207-212, 2003.
Satchi et al., Br J Cancer, 85: 1070-1076, 2001.
Sexton et al., Melanoma Res, 7: 97-101, 1997.
Spom et al., Lancet, 347: 1377-1381, 1996.
Stetler-Stevenson et al., J Clin Invest, 103: 1237-1241, 1999.
Theissen et al., Methods Mol Biol, 85: 123-133, 1997.
Werb et al., Cell, 91: 439-442, 1997.
Wrang et al., J Neurosci Res, 65: 54-58, 2001.
Yano et al., Surg Today, 31: 385-389, 2001.
Yonezawa et al., J Biol Chem, 263: 16504-16511, 1988.
Zhang et al., Mol Biotechnol, 10: 155-165, 1998.
Zhu et al., Leuk Lymphoma, 39: 343-354, 2000.
U.S. Pat. No. 6,004,933
U.S. Patent Publication Number US 2003/0054387
PCT International Publication Number WO 00/64945
PCT International Publication Number WO 03/016335

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A protease inhibitor consisting of any one of formulae III, IV, V or VI:

| | | |
|---|---|---|
| R₁-(Xaa4)ₙ-Asn-Y | (SEQ ID NO:26) | III |
| R₁-(Xaa4)ₙ-Xaa5-Y | (SEQ ID NO:27) | IV |
| R₁-Xaa4-azaAsn-Y | | V |
| R₁-Xaa4-azaXaa5-Y | | VI |

or a combination thereof, wherein:
wherein R₁ is hydrogen, a hydrophilic group, a hydrophobic group, a photosensitizing agent, a label or an amino protecting group;
n is an integer of 2 to about 5;
each Xaa4 and Xaa5 is an amino acid or an amino acid mimetic;
Y is alkyl, alkenyl epoxide, fluoromethylketone or a Michael acceptor, optionally substituted with 1-3 halo or hydroxy, alkylamino, dialkylamino, alkyldialkylamino, or cycloalkyl, alkylcycloalkyl, alkenylcycloalkyl, aryl; (C₅-C₁₂)arylalkyl or (C₅-C₁₂)arylalkenyl,
wherein the aryl groups of the arylalky or arylalkenyl can be 0-4 heteroatoms selected from N, O and S, and are optionally substituted with halo, cyano, nitro, haloalkyl, amino, aminoalkyl, dialkylamino, alkyl, alkenyl, alkynyl, alkoxy, haloalkoxy, carboxyl, carboalkoxy, alkylcarboxamide, (C₅-C₆)aryl, —O—(C₅-C₆)aryl, arylcarboxamide, alkylthio or haloalkylthio; and
wherein each of the inhibitors of formulae III, IV, V and VI bind to a protease expressed in a tumor microenvironment.

2. The protease inhibitor of claim 1, wherein the Michael acceptor is N-Acetyl-Phe-NHCH₂CH═CH-E or Xaa4-NHCH₂CH═CHCOOCH₃ where E is an electron withdrawing group selected from the group consisting of CO₂CH₃, SO₂CH₃, CO₂H, CN, CONH₂ and C₆H₄-p-NO₂; and Xaa4 is an amino acid.

3. The inhibitor of claim 1 wherein the inhibitor is an asparaginyl endopeptidase inhibitor selected from the group consisting of:
R₁-Ala-Ala-AzaAsn-EP-COOEt
R₁-Ala-Ala-Asn-EP-COOR₃
R₁-Ala-AzaAsn-EP-COOEt.
R₁-Xaa4-Xaa4-AzaAsn-EP-COOEt
R₁-Xaa4-Xaa4-AzaAsn-EP-COOR₃
R₁-Xaa4-AzaAsn-EP-COOEt
R₁-Xaa4-AzaAsn-EP-COOR₃
R₁-AzaAsn-EP-COOEt
R₁-AzaAsn-EP-COOR₃
wherein: AzaAsn is azido-asparagine; EP is epoxy; and R₃ is an aryl alcohol, an aliphatic alcohol or a heterocyclic alcohol.

4. The inhibitor of claim 1, having the structure:

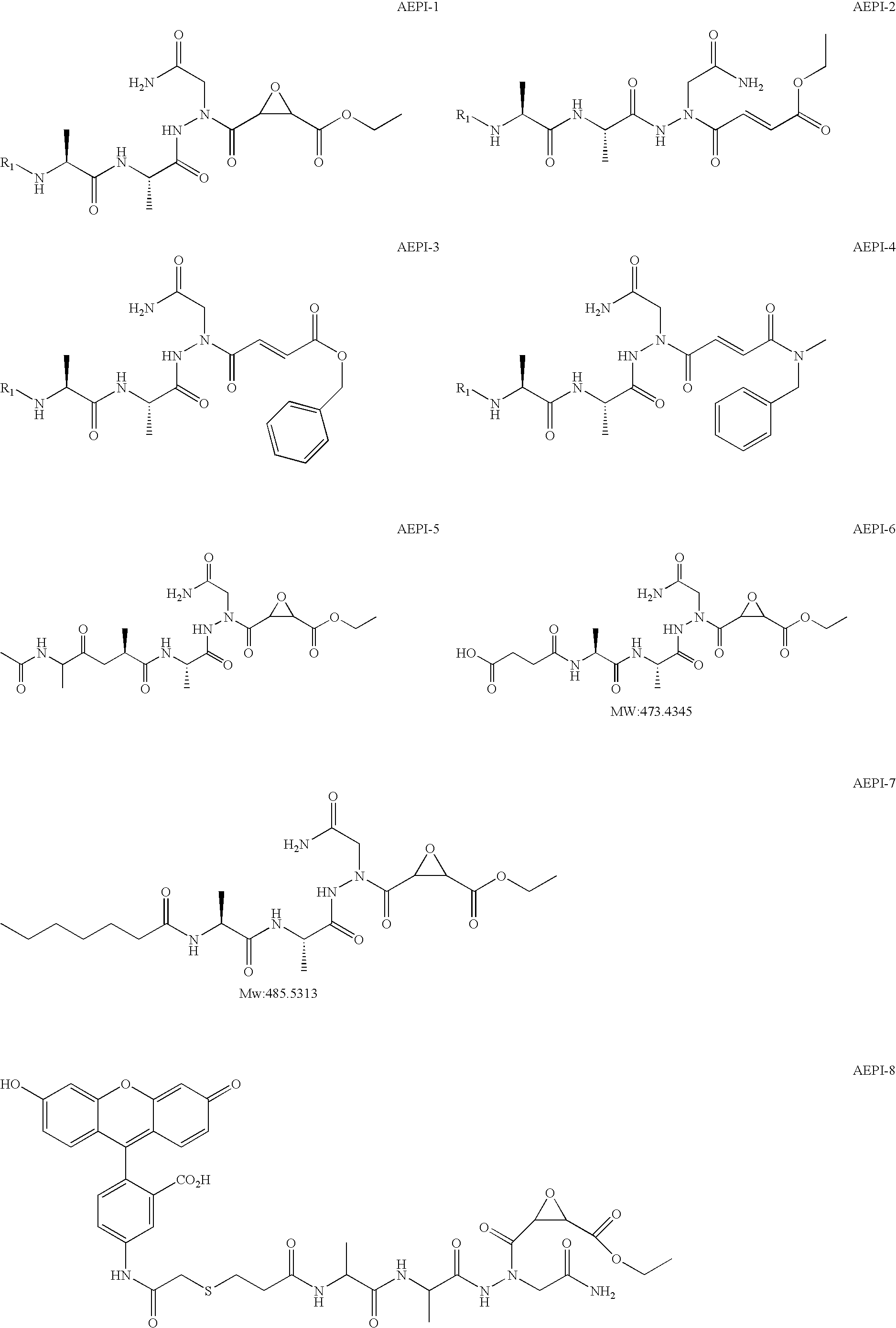

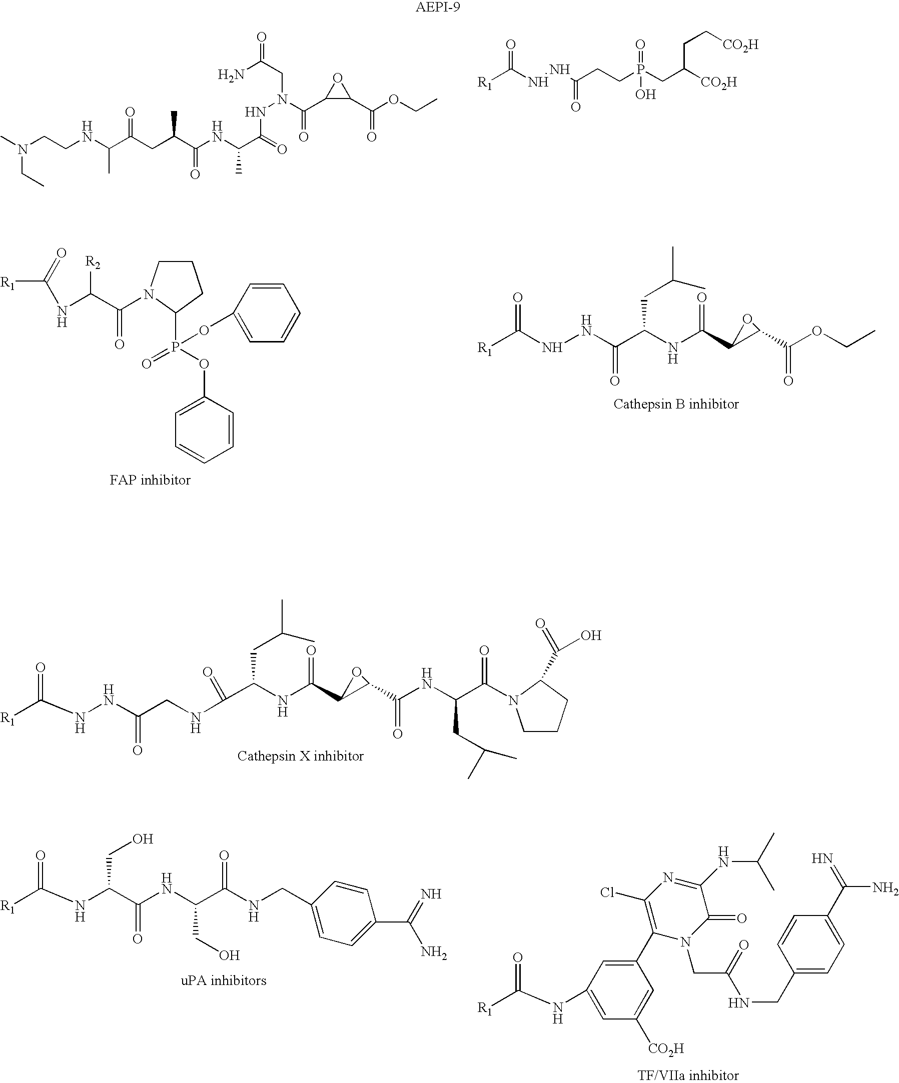

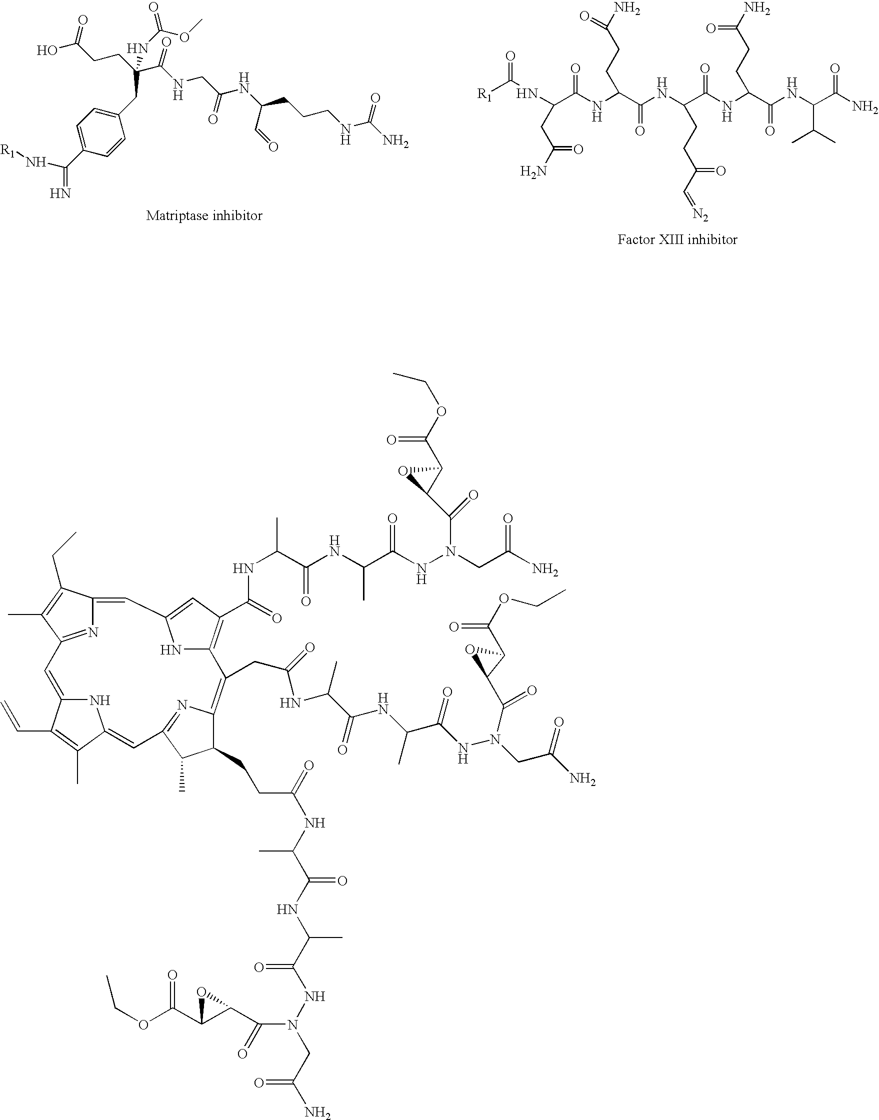

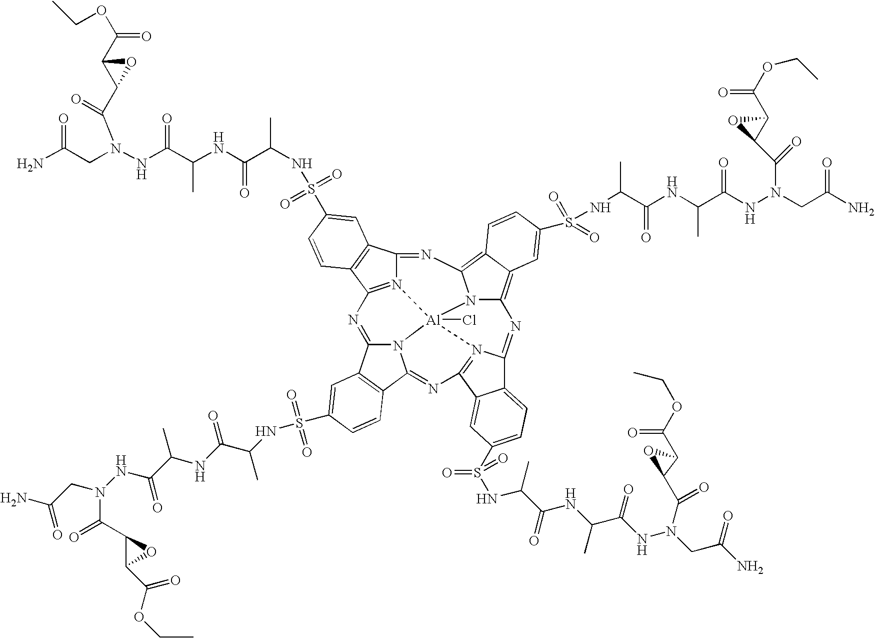

5. The inhibitor of claim 1, wherein the R₁ is a sugar, dicarboxylic acid, oligosaccharide, glycan, polyalkylene oxide, lower alkyl carboxylate, carboxyalkyl, carboxyalkylene carboxylate, charged amino acid, alkyl, aryl, alkylene aryl, arylalkyl, beta-alanyl or hydrophobic amino protecting group.

6. The inhibitor of claim 1, wherein R₁ a photosensitizing agent selected from the group consisting of aminolevulinic acid, aluminum phthalocyanine tetrasulfonate and chlorin e6.

7. The inhibitor of claim 1, wherein the inhibitor is a compound of the following structure:

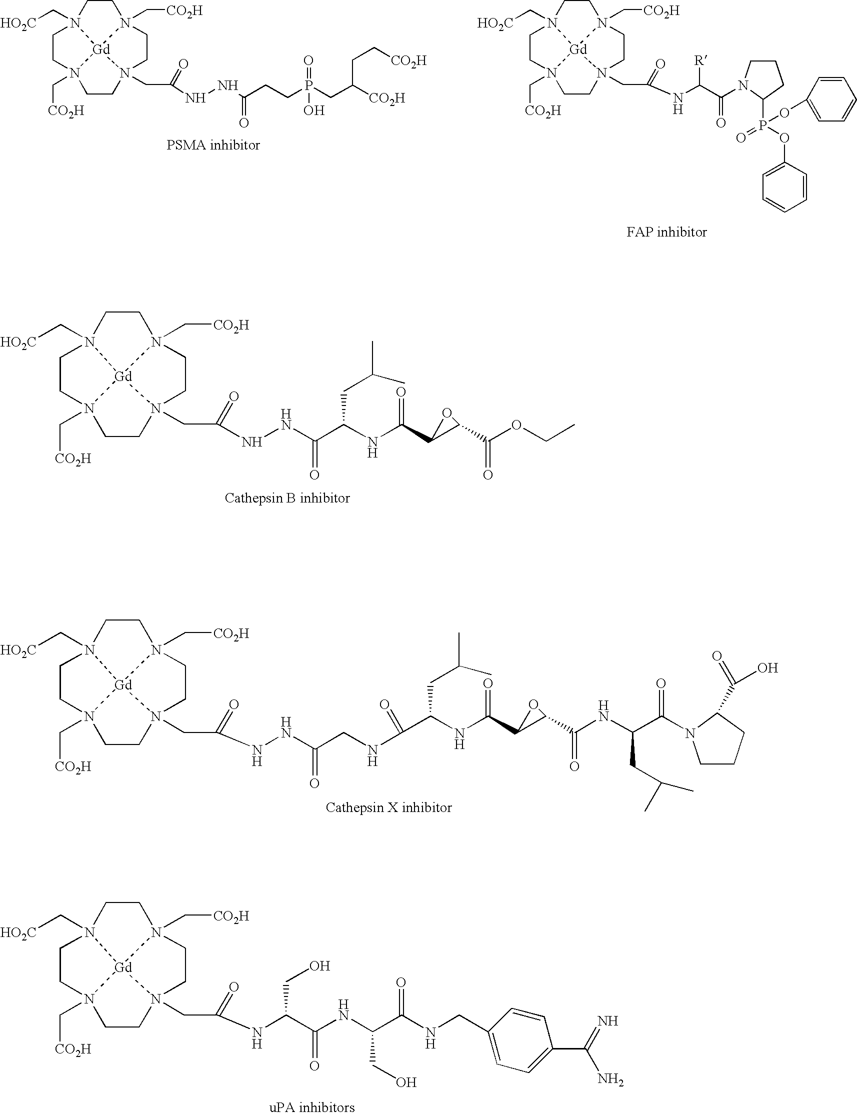

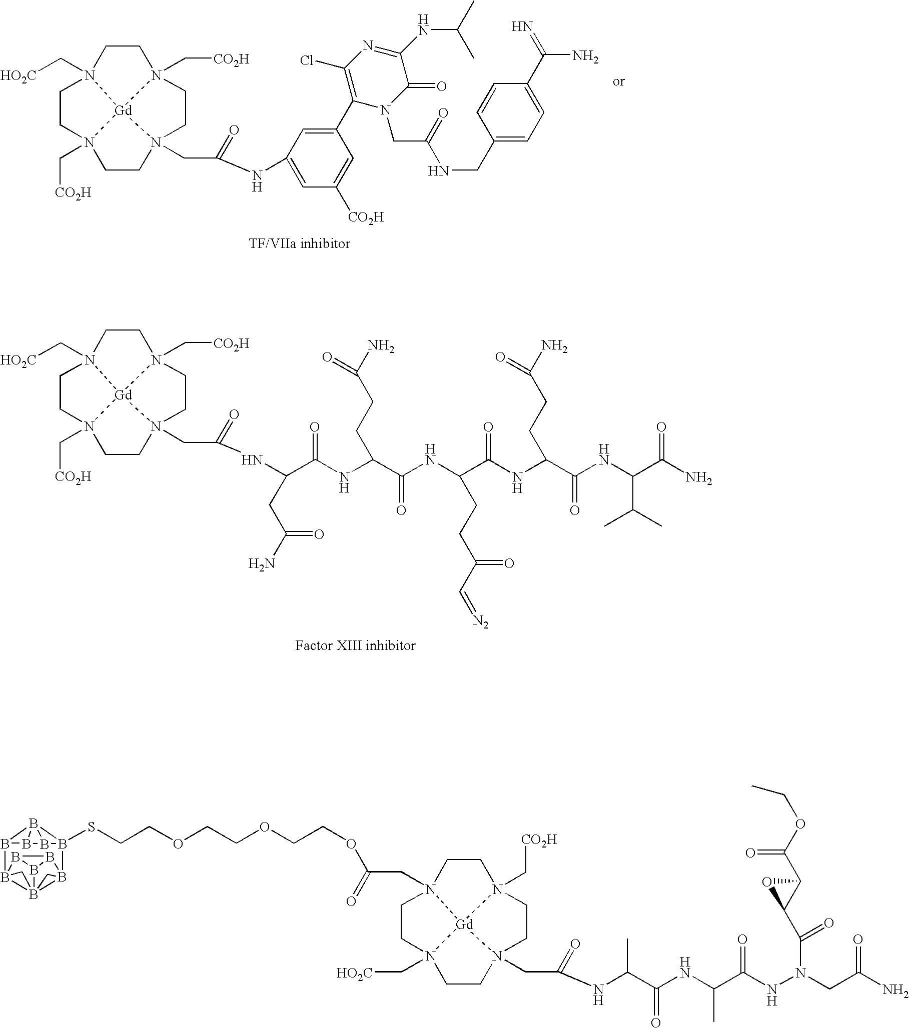

8. A prodrug with the structure:
R₁-peptide-drug
wherein:
R₁ is hydrogen, a hydrophilic group, a hydrophobic group, a label or an amino protecting group;
peptide is a peptidyl amino acid sequence that can be cleaved by a protease expressed by cells in a tumor; and
drug is a therapeutic agent or toxin;
wherein the drug is inactive until the peptide is cleaved by the protease.

9. The prodrug of claim 8, consisting of SEQ ID NO:3 or SEQ ID NO:23:

| | |
|---|---|
| R₁-(Xaa1)ₙ-Xaa2-Asn-(Xaa3)-drug | (SEQ ID NO:3) |
| R₁-(Xaa1)ₙ-Xaa2-Xaa3-drug | (SEQ ID NO:23) |

wherein:
n is an integer of about 0 to about 50;
Xaa1 and Xaa2 are separately any amino acid;
Xaa3 is either nothing or an amino acid that has no substantial effect on the activity of the drug; and
drug is a therapeutic agent whose activity is diminished or blocked by attachment of a peptide to the drug.

10. The prodrug of claim 8, wherein the drug is a cytotoxin, photosensitizing agent, aldesleukin, 5-aminolevulinic acid, aluminum phthalocyanine tetrasulfonate, asparaginase, bleomycin sulfate, camptothecin, carboplatin, carmustine, chlorine e6, cisplatin, cladribine, lyophilized cyclophosphamide, non-lyophilized cyclophosphamide, cytarabine, dacarbazine, dactinomycin, daunorubicin, diethylstilbestrol, doxorubicin, epoetin alfa, epirubicin, esperamycin, etidronate, etoposide, filgrastim, floxuridine, fludarabine phosphate, fluorouracil, goserelin, granisetron hydrochloride, idarubicin, ifosfamide, immunoglobulin, interferon alpha-2a, interferon alpha-2b, leucovorin calcium, leuprolide, levamisole, mechiorethamine, medroxyprogesterone, melphalan, methotrexate, mitomycin, mitoxantrone, octreotide, ondansetron hydrochloride, paclitaxel, pamidronate, pegaspargase, plicamycin, sargramostim, streptozocin, taxol, thiotepa, teniposide, vinblastine, or vincristine.

11. The prodrug of claim 8, wherein R₁ is succinyl, glucuronide, polyalkylene glycol, acetyl or t-butoxycarbonyl.

12. The prodrug of claim 8, wherein the peptide amino acid sequence comprises Asn-Leu, Ala-Asn-Leu, Thr-Asn-Leu, Boc-Ala-Ala-Asn-Leu (SEQ ID NO:4), Ala-Ala-Asn-Leu (SEQ ID NO:5), Ala-Thr-Asn-Leu (SEQ ID NO:6), or succinyl-Ala-Ala-Asn-Leu-doxorubicin (SEQ ID NO:8).

13. The prodrug of claim 8, wherein the prodrug comprises Boc-Ala-Ala-Asn-Leu-doxorubicin (SEQ ID NO:7), Succinyl-Ala-Ala-Asn-Leu-doxorubicin (SEQ ID NO:8), N-(-t-Butoxycarbonyl-Ala-Thr-Asn-Leu)doxorubicin (SEQ ID NO:9), Succinyl-Ala-Thr-Asn-Leu-doxorubicin (SEQ ID NO: 10), N-(-t-Butoxycarbonyl-Ala-Asn-Leu)doxorubicin (SEQ ID NO:11), Succinyl-Ala-Asn-Leu-doxorubicin (SEQ ID NO:12), N-(-t-Butoxycarbonyl-Thr-Leu)doxorubicin (SEQ ID NO: 13), Succinyl-Thr-Leu-doxorubicin (SEQ ID NO: 14), Succinyl-Ala-Ala-Asn-Leu-N₂H₃ (SEQ ID NO:21), N-(-t-Butoxycarbonyl-L-Ala-L-Ala-L-Asn)taxel, N-(succinyl-L-Ala-L-Ala-L-Asn)taxel or has any one of the following structures: 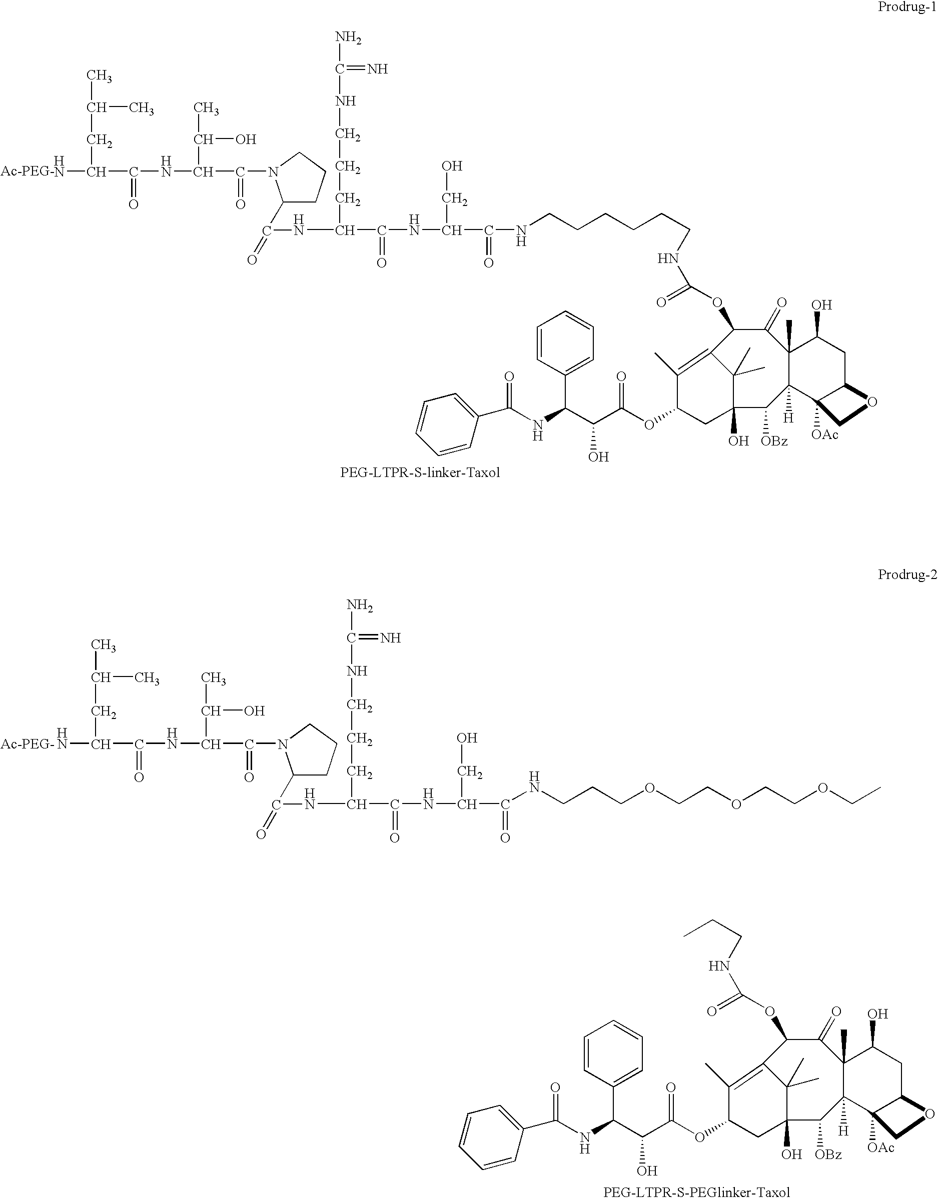 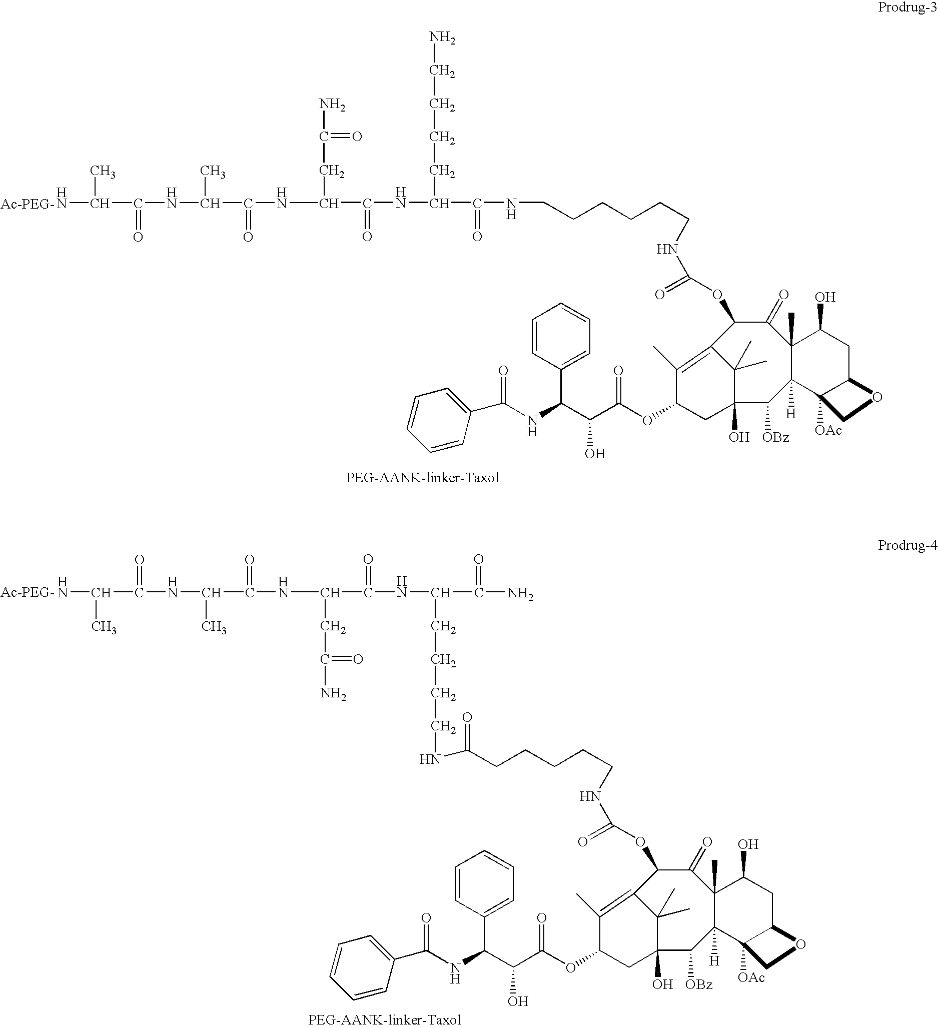 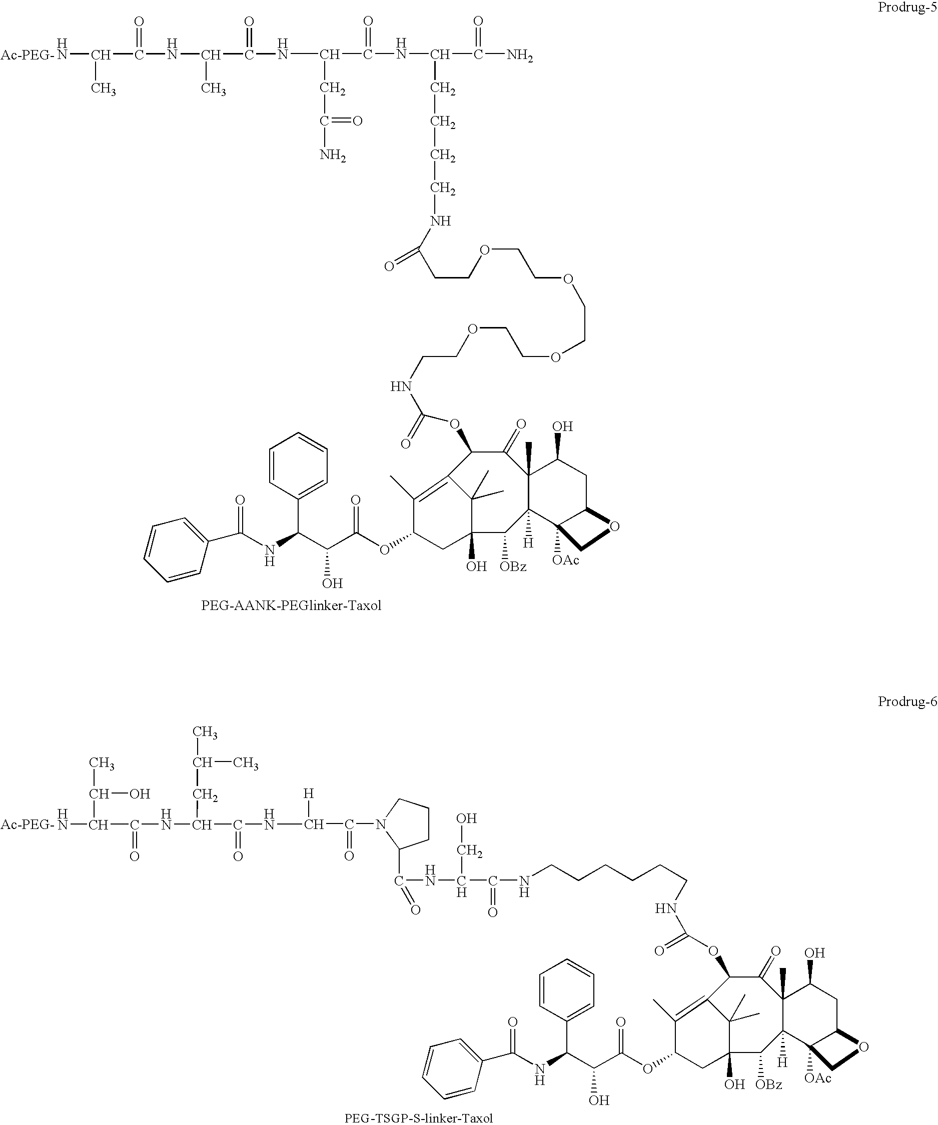 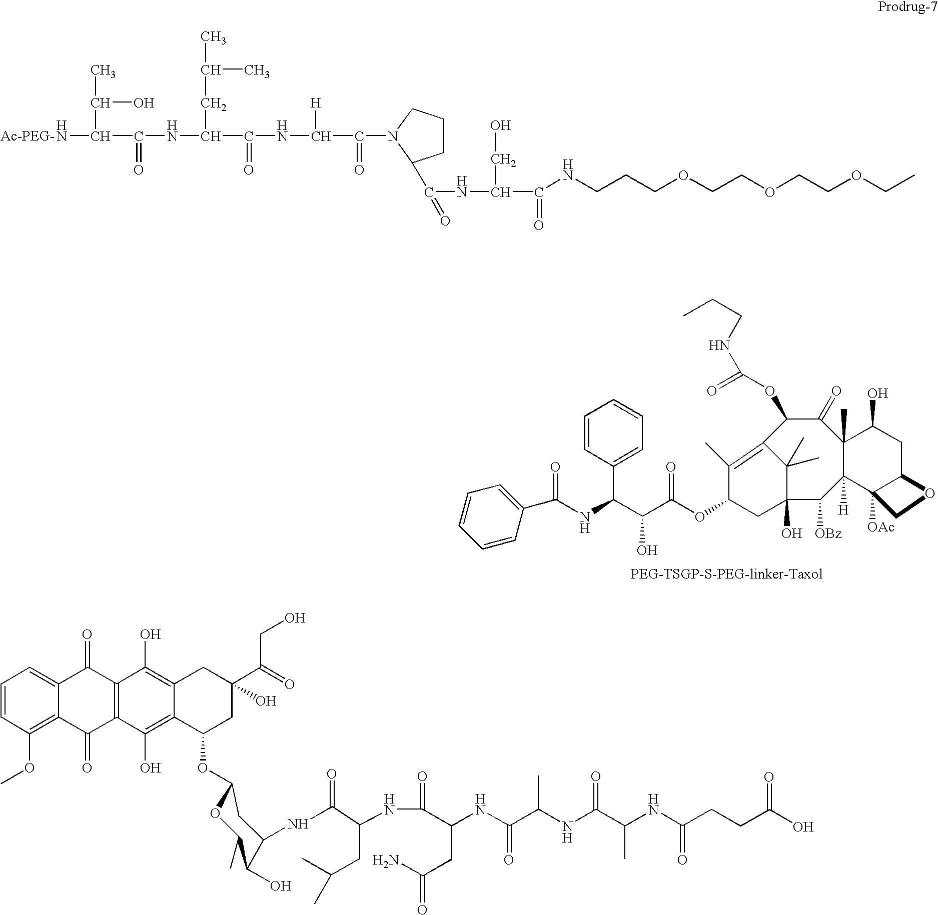 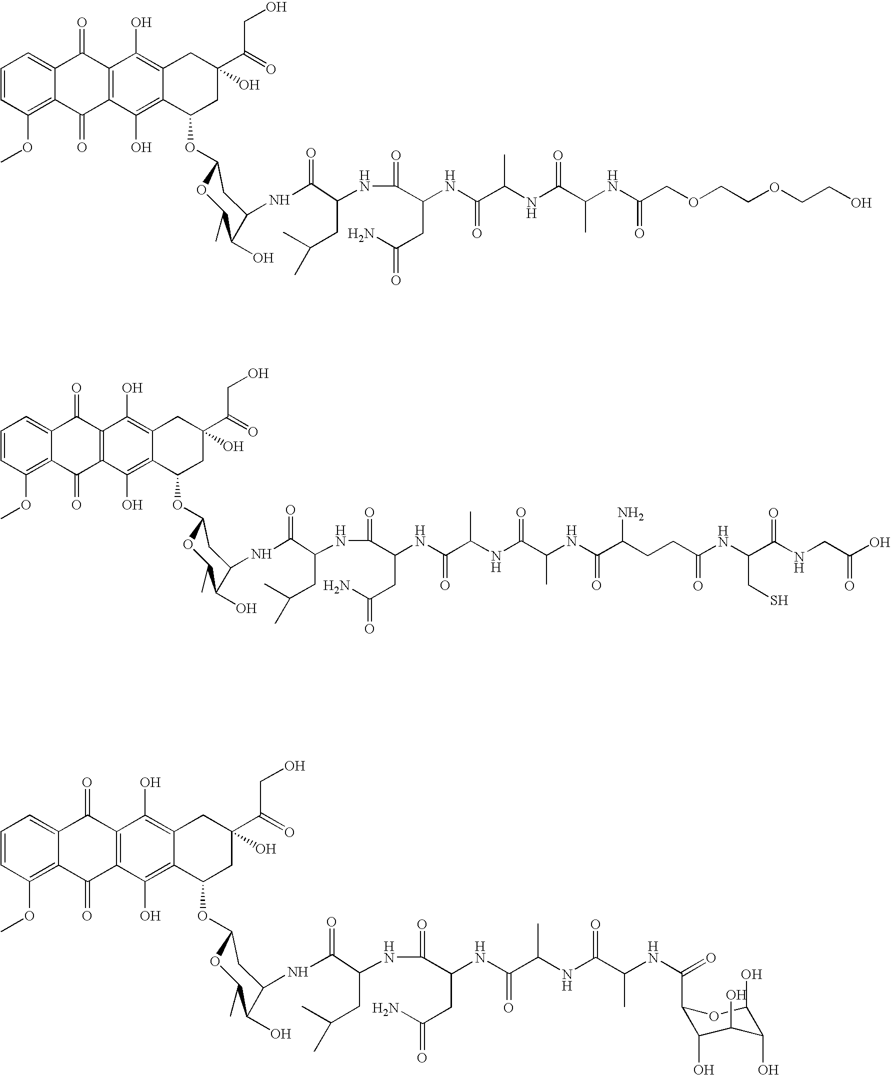 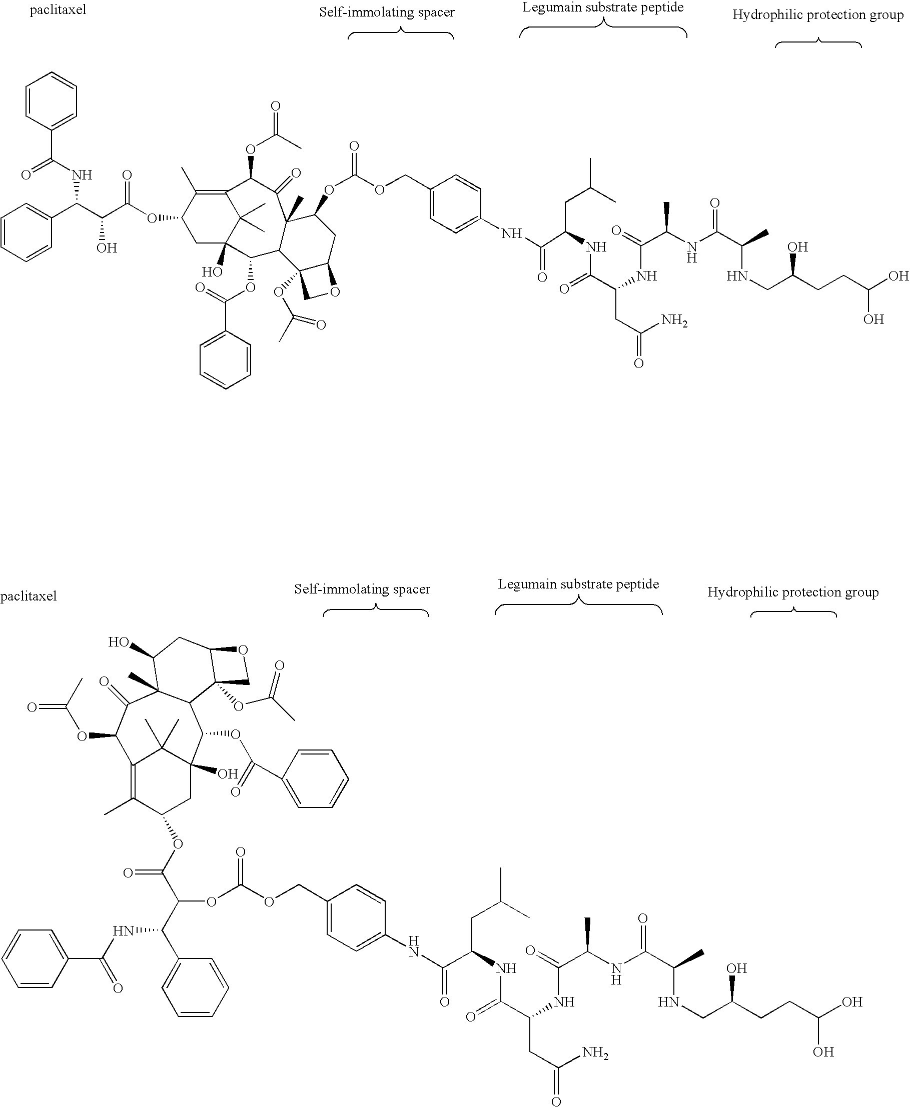 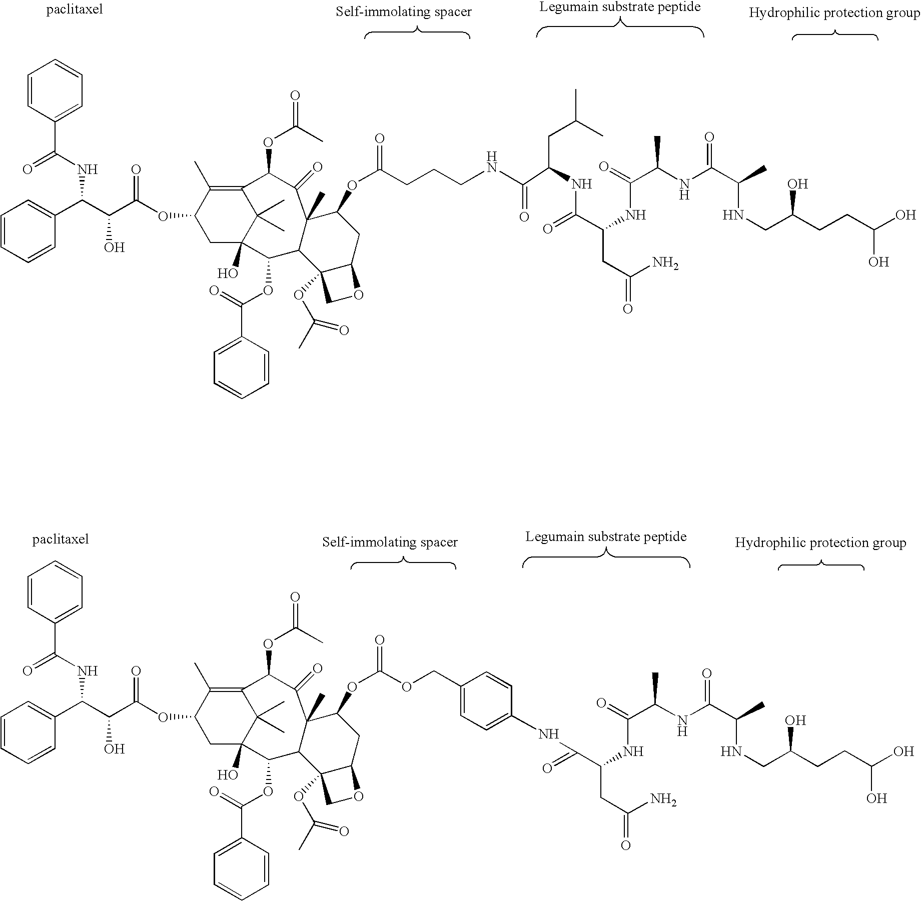 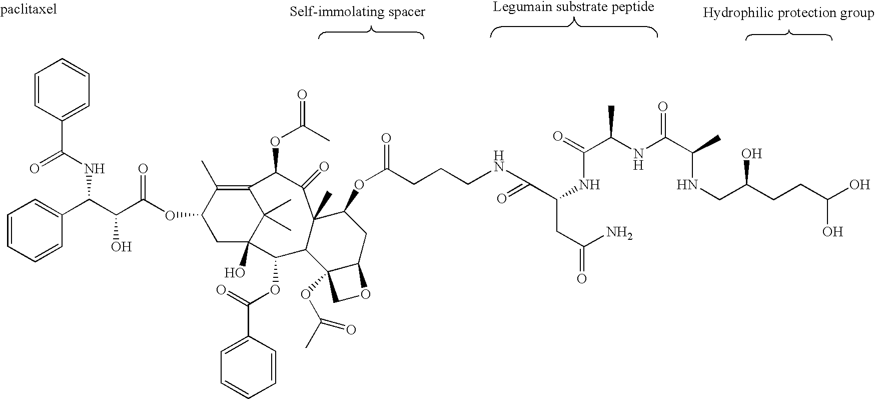
or a combination thereof.

14. An anti-legumain antibody that binds to an epitope consisting essentially of CGMKRASSPVPLPP (SEQ ID NO: 16).

15. A composition comprising a pharmaceutically acceptable carrier and an effective amount of the inhibitor of claim 1 and, optionally, another chemotherapeutic agent.

16. A composition comprising a pharmaceutically acceptable carrier and an effective amount of the prodrug of claim 8 and, optionally, another chemotherapeutic agent.

17. A composition comprising a pharmaceutically acceptable carrier and an effective amount of the anti-legumain antibody of claim 14 and, optionally, another chemotherapeutic agent.

18. A method for diagnosing or monitoring cancer in an animal, comprising administering to the animal an inhibitor of claim 1, wherein R₁ is a label, and detecting whether the inhibitor accumulates in a tissue.

19. A method of detecting and treating cancer in a mammal, comprising
a. administering to the mammal the inhibitor of claim 1, wherein R₁ is a label, to detect whether the mammal has cancer and to detect which type of tumor specific protease is associated with the cancer, and
b. administering a prodrug with the structure:
R₁-peptide-drug
wherein: R₁ is hydrogen, a hydrophilic group, a hydrophobic group, a label or an amino protecting group; peptide is a peptidyl amino acid sequence that can be cleaved by a protease expressed by cells in a tumor; and drug is a therapeutic agent or toxin; wherein the drug is inactive until the peptide is cleaved by the protease, and wherein the prodrug has a cleavage site for the tumor specific protease associated with the cancer.

20. The method of claim 19, wherein the method inhibits processing of cancer cell extracellular matrices and enhances penetration and retention of the second chemotherapeutic agent at a low dosage.

21. The method of claim 19, further comprising administering to the mammal the inhibitor of claim 1 in a therapeutically effective amount.

22. The method of claim 19, further comprising administering to the mammal a therapeutically effective amount of the anti-legumain antibody of claim 14 or another chemotherapeutic agent.

23. The method of claim 19, wherein the therapeutically effective amount is sufficient to inhibit tumor stromal cells from producing angiogenic factors and growth factors, or the therapeutically effective amount is sufficient to inhibit tumor metastasis, the therapeutically effective amount is sufficient to promote tumor cell apoptosis.

24. The method of claim 22, wherein the stromal cells are tumor associated macrophages or angiogenic endothelial cells.

25. The method of claim 19, wherein the cancer is a solid cancer, a metastatic cancer, an invasive cancer.

26. The method of claim 19, wherein the prodrug is cleaved by a cell surface legumain:integrin complex, and wherein the integrin substantially increases legumain activity.

27. The method of claim 25, wherein the integrin increases legumain activity by about 10-fold to about 200-fold.

28. A method for inhibiting activation of metalloprotease-2 and/or cathepsin L in a mammal comprising administering to the mammal the inhibitor of claim 1 to promote apoptosis in tumor cells, inhibit angiogenesis in a tumor, inhibit tumor invasion in the mammal and/or inhibit metastasis in the mammal.

29. A method for inhibiting tumor associated macrophages, osteoclasts or foam cells in a mammal comprising administering to the mammal the inhibitor of claim 1, and/or an anti-legumain antibody that binds to an epitope consisting essentially of CGMKRASSPVPLPP (SEQ ID NO:16), and/or a prodrug with the structure:
R₁-peptide-drug
wherein:
R₁ is hydrogen, a hydrophilic group, a hydrophobic group, a label or an amino protecting group;
peptide is a peptidyl amino acid sequence that can be cleaved by legumain; and
drug is a therapeutic agent or toxin;
wherein the drug is inactive until the peptide is cleaved by the protease.

30. The method of claim 29, wherein the foam cells are inhibited to treat atherosclerosis or the macrophages are inhibited to treat cancer or inflammation.

31. A method for detecting metastatic cancer in a test sample, comprising contacting a test tissue suspected of comprising cancer cells with of the anti-legumain antibody of claim 14, and detecting whether the antibody binds to the test tissue.

32. The method of claim 31, which further comprises quantifying and comparing amounts of the antibody bound to the test tissue with amounts of the antibody bound to a control tissue that is not cancerous.

33. A method for diagnosing or monitoring cancer in an animal, comprising administering to the animal the anti-legumain antibody of claim 14, and detecting whether the antibody accumulates in a tissue.

34. A composition comprising a liposomal carrier and a therapeutically effective amount of a prodrug of claim 8.

35. A method of inhibiting the inactivation of caspases in a mammal comprising administering to the mammal the inhibitor of claim 1 to promote apoptosis in tumor cells in the mammal.


SEQUENCE LISTING (30 sequences)

SEQ ID NO: 1 - protein, 433 aa, Homo sapiens
MVWKVAVFLSVALGIGAIPIDDPEDGGKHWVVIVAGSNGWYNYRHQADACHAYQIIHRNGIPDEQIVVMMYDDIAYSEDNPTPGIVINRPNGTDVYQGVPKDYTGEDVTPQNFLAVLRGDAEAVKGIGSGKVLKSGPQDHVFIYFTDHGSTGILVFPNEDLHVKDLNETIHYMYKHKMYRKMVFYIEACESGSMMNHLPDNINVYATTAANPRESSYACYYDEKRSTYLGDWYSVNWMEDSDVEDLTKETLHKQYHLVKSHTNTSHVMQYGNKTISTMKVMQFQGMKRKASSPVPLPPVTHLDLTPSPDVPLTIMKRKLMNTNDLEESRQLTEEIQRHLDARHLIEKSVRKIVSLLAASEAEVEQLLSERAPLTGHSCYPEALLHFRTHCFNWHSPTYEYALRHLYVLVNLCEKPYPLHRIKLSMDHVCLGHY

SEQ ID NO: 2 - DNA, 1981 bp, Homo sapiens
ggcacgagggaggctgcgagccgccgcgagttctcacggtcccgccggcgccaccaccgcggtcactcaccgccgccgccgccaccactgccaccacggtcgcctgccacaggtgtctgcaattgaactccaaggtgcagaatggtttggaaagtagctgtattcctcagtgtggccctgggcattggtgccattcctatagatgatcctgaagatggaggcaagcactgggtggtgatcgtggcaggttcaaatggctggtataattataggcaccaggcagacgcgtgccatgcctaccagatcattcaccgcaatgggattcctgacgaacagatcgttgtgatgatgtacgatgacattgcttactctgaagacaatcccactccaggaattgtgatcaacaggcccaatggcacagatgtctatcagggagtcccgaaggactacactggagaggatgttaccccacaaaatttccttgctgtgttgagaggcgatgcagaagcagtgaagggcataggatccggcaaagtcctgaagagtggcccccaggatcacgtgttcatttacttcactgaccatggatctactggaatactggtttttcccaatgaagatcttcatgtaaaggacctgaatgagaccatccattacatgtacaaacacaaaatgtaccgaaagatggtgttctacattgaagcctgtgagtctgggtccatgatgaaccacctgccggataacatcaatgtttatgcaactactgctgccaaccccagagagtcgtcctacgcctgttactatgatgagaagaggtccacgtacctgggggactggtacagcgtcaactggatggaagactcggacgtggaagatctgactaaagagaccctgcacaagcagtaccacctggtaaaatcgcacaccaacaccagccacgtcatgcagtatggaaacaaaacaatctccaccatgaaagtgatgcagtttcagggtatgaaacgcaaagccagttctcccgtccccctacctccagtcacacaccttgacctcacccccagccctgatgtgcctctcaccatcatgaaaaggaaactgatgaacaccaatgatctggaggagtccaggcagctcacggaggagatccagcggcatctggatgccaggcacctcattgagaagtcagtgcgtaagatcgtctccttgctggcagcgtccgaggctgaggtggagcagctcctgtccgagagagccccgctcacggggcacagctgctacccagaggccctgctgcacttccggacccactgcttcaactggcactcccccacgtacgagtatgcgttgagacatttgtacgtgctggtcaacctttgtgagaagccgtatccacttcacaggataaaattgtccatggaccacgtgtgccttggtcactactgaagagctgcctcctggaagcttttccaagtgtgagcgccccaccgactgtgtgctgatcagagactggagaggtggagtgagaagtctccgctgctcgggccctcctggggagcccccgctccagggctcgctccaggaccttcttcacaagatgacttgctcgctgttacctgcttccccagtcttttctgaaaaactacaaattagggtgggaaaagctctgtattgagaagggtcatatttgctttctaggaggtttgttgttttgcctgttagttttgaggagcaggaagctcatgggggcttctgtagcccctctcaaaaggagtctttattctgagaatttgaagctgaaacctctttaaatcttcagaatgattttattgaagagggccgcaagccccaaatggaaaactgtttttagaaaatatgatgatttttgattgcttttgtatttaattctgcaggtgttcaagtcttaaaaaataaagatttataacagaacccaaaaaaaaaaaaaaaaaaaaaaaaaaaaaaa

SEQ ID NO: 3 - protein, 53 aa, Artificial Sequence
  A synthetic peptide
XXXXXXXXXXXXXXXXXXXXXXXXXXXXXXXXXXXXXXXXXXXXXXXXXXXNX

SEQ ID NO: 4 - protein, 4 aa, Artificial Sequence
  A synthetic peptide
AANL

SEQ ID NO: 5 - protein, 4 aa, Artificial Sequence
  A synthetic peptide
AANL

SEQ ID NO: 6 - protein, 4 aa, Artificial Sequence
  A synthetic peptide
ATNL

SEQ ID NO: 7 - protein, 4 aa, Artificial Sequence
  A synthetic peptide
AANL

SEQ ID NO: 8 - protein, 4 aa, Artificial Sequence
  A synthetic peptide
AANL

SEQ ID NO: 9 - protein, 4 aa, Artificial Sequence
  A synthetic peptide
ATNL

SEQ ID NO: 10 - protein, 4 aa, Artificial Sequence
  A synthetic peptide
ATNL

SEQ ID NO: 11 - protein, 3 aa, Artificial Sequence
  A synthetic peptide
ANL

SEQ ID NO: 12 - protein, 3 aa, Artificial Sequence
  A synthetic peptide
ANL

SEQ ID NO: 13 - protein, 2 aa, Artificial Sequence
  A synthetic peptide
TL

SEQ ID NO: 14 - protein, 2 aa, Artificial Sequence
  A synthetic peptide
TL

SEQ ID NO: 15 - protein, 5 aa, Artificial Sequence
  A synthetic peptide
ALNAA

SEQ ID NO: 16 - protein, 14 aa, Artificial Sequence
  A synthetic peptide
CGMKRASSPVPLPP

SEQ ID NO: 17 - protein, 146 aa, Homo sapiens
MAGPLRAPLLLLAILAVALAVSPAAGSSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

SEQ ID NO: 18 - protein, 26 aa, Homo sapiens
MAGPLRAPLLLLAILAVALAVSPAAG

SEQ ID NO: 19 - protein, 120 aa, Homo sapiens
SSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

SEQ ID NO: 20 - protein, 98 aa, Homo sapiens
MMCGAPSATQPATAETQHIADQVRSQLEEKENKKFPVFKAVSFKSQVVAGTNYFIKVHVGDEDFVHLRVFQSLPHENKPLTLSNYQTNKAKHDELTYF

SEQ ID NO: 21 - protein, 4 aa, Artificial Sequence
  A synthetic peptide
AANL

SEQ ID NO: 22 - protein, 4 aa, Artificial Sequence
  A synthetic peptide
AANL

SEQ ID NO: 23 - protein, 52 aa, Artificial Sequence
  A synthetic peptide
XXXXXXXXXXXXXXXXXXXXXXXXXXXXXXXXXXXXXXXXXXXXXXXXXXXX

SEQ ID NO: 24 - protein, 4 aa, Artificial Sequence
  A synthetic peptide
ATNL

SEQ ID NO: 25 - protein, 4 aa, Artificial Sequence
  A synthetic peptide
AANL

SEQ ID NO: 26 - protein, 6 aa, Artificial Sequence
  A synthetic peptide
XXXXXN

SEQ ID NO: 27 - protein, 6 aa, Artificial Sequence
  A synthetic peptide
XXXXXX

SEQ ID NO: 28 - protein, 5 aa, Artificial Sequence
  A synthetic peptide
QXVXG

SEQ ID NO: 29 - protein, 4 aa, Artificial Sequence
  A synthetic peptide
AANL

SEQ ID NO: 30 - protein, 4 aa, Artificial Sequence
  A synthetic peptide
AANL